(19) United States
(12) Patent Application Publication
(10) Pub. No.: US 2019/0100534 A1
(43) Pub. Date: Apr. 4, 2019

(54) Cephem Compounds with Latent Reactive Groups

(71) Applicant: Gladius Pharmaceuticals, Inc., Montreal (CA)

(72) Inventors: Larry D. SUTTON, Atchison, KS (US); Robert ZAMBONI, Beaconsfield (CA); Patrick BUREAU, Kirkland (CA); Avid HASSANPOUR, Montreal (CA); Laura MAMANI LAPARRA, Montreal (CA); Marc VIDAL, Montreal (CA); Simon WOO, Pierrefonds (CA); Nancy ZHOU, Montreal (CA); Richard M. KEENAN, Devon, PA (US)

(21) Appl. No.: 16/151,479

(22) Filed: Oct. 4, 2018

Related U.S. Application Data
(60) Provisional application No. 62/568,104, filed on Oct. 4, 2017.

(51) Int. Cl. C07D 501/57; C07D 501/46; A61P 31/04

(52) U.S. Cl. CPC C07D 501/57; A61P 31/04; C07D 501/46

(57) ABSTRACT

Cephem and penem compounds having a styrylmethylene moiety at the 3-position in the cephem or penem ring to which a positively charged leaving group is bonded and wherein the leaving group contains a vicinal diol or is bonded to a unsubstituted or substituted catechol. The leaving group can be a positively charge nitrogen leaving group. Cephems include cephalosporins, cephamycins, carbacephems, and oxacephems. Penems include penems, carbapenems and oxapenems. Preferred cephems are cephalosporins. Preferred penems are carbapenems. Compounds exhibit antibiotic activity against Gram-negative bacteria and/or Gram-positive bacteria. Compounds exhibit antibiotic activity against bacteria which exhibit multi-drug resistance. Compounds of the invention exhibit antibiotic activity against bacterial strains which produce extended spectrum beta-lactamases (ESBL), which produce AmpC beta-lactamases or which produce a carbapenemase. Pharmaceutical compositions comprising one or more cephems or penems or methods of treatment of bacterial infections with such compounds and compositions.

### BACKGROUND OF THE INVENTION

The invention and utilization of antibiotics is the cornerstone of many modern medical technologies. [1-3] However, the selective pressure from decades of antibiotic utilization has resulted in increasing antimicrobial-resistant bacteria which was first declared a medical emergency in 1992. [4] This happened at a time when there was a withdrawal from the discovery and development of antibiotics. [5, 6] Today antimicrobial resistance is so severe that wild type pathogens are no longer relevant and new categories describe infecting pathogens in terms of their resistance to antibiotics: usual drug resistance, multi-drug resistance, extreme drug resistance and pan-drug resistance. [7] The need for new antibiotics is so great that fears are increasing that we are entering a post-antibiotic era in human history. [2, 8-13]

The beta-lactam class of antibiotics has been one of the most important and successful classes of antimicrobials for the treatment of human infections caused by bacterial pathogens since they were first developed and used in 1941. [4] The most important and prevalent method employed by bacteria to resist the effects of beta-lactam antibiotics is the production of beta-lactamases, enzymes that inactivate beta-lactam antibiotics before they can bind their targets. [14-16]

Cephalosporins were first developed about 50 years ago in response to the need for beta-lactam antibiotics that were impervious to early beta-lactamases (penicillinases). Soon thereafter, bacteria evolved beta-lactamases capable of hydrolyzing these first-generation cephalosporins. [17, 18]

In response, a wide variety of chemical modifications have been assessed to restore effectiveness of cephalosporins, including modifications at the 7-side chain, [7, 19, 20] and modifications at the 3-methyl position of the cephem ring.

U.S. Pat. Nos. 888,377 and 9,975,905 and PCT published application WO 2014/024503 relate to cephems and related compounds modified with certain latent reactive groups. The compounds of this patent exhibit time-dependent inhibition of beta-lactamases. Certain of the compounds of this patent contain a styryl group or a substituted styryl group to conjugate the latent reactive group with the cephem ring.

Each of these patents is incorporated by reference herein in its entirety for cephems, cephalosporins and compounds of related structures therein and methods of synthesis of cephems and cephalosporins.

U.S. Pat. Nos. 7,384,928; 7,696,354; 8,883,773; 9,085,589; 9,145,425; 9,290,515; 9,238,65; and 9,334,289 relate to cephem and or cephalosporin compounds having a catechol or pseudocatechol group. None of the compounds in these patents includes a styryl group substituted at the cephem ring. Each of these patents is incorporated by reference herein in its entirety for cephem and cephalosporin structures therein and methods of synthesis of cephems and cephalosporins.

U.S. Pat. No. 9,340,566 relates to certain cephalosporin antibiotics having a moiety containing a five-member ring with a positively charged N substituted on the cephem ring. This patent is incorporated by reference herein in its entirety for cephalosporin structures therein and methods of synthesis of cephalosporins.

U.S. Pat. Nos. 4,465,632; 4,689,292; 4,729,993; 4,978659; 5,334,590; 5,342,933; 5,350,746; 5,382,575; 6,677,331; 6,825,187; 7,468,364; 7,632,828; 8318,716; 9,145,425; and 9,937,151 relate to carbapenem compounds. None of the compounds in these patents includes a styryl group substituted at the carbapenem ring. Each of these patents is incorporated by reference herein in its entirety for carbapenem structures therein and methods of synthesis of carbapenems.

U.S. Pat. Nos. 4,218,459; 4,298,741; 5,055,463; 5,138,050; and 5,395,931 relate to carbapenem compounds having a 6-amido group and provide some detail of synthesis of such compounds. None of the compounds in these patents includes a styryl group substituted at the carbapenem ring. Each of these patents is incorporated by reference herein in its entirety for carbapenem structures therein and methods of synthesis of carbapenems and in particular 6-amido substituted carbapenems. U.S. Pat. No. 4,347,355 relates to inhibitors of transpeptidase which are beta-lactams of general structure:

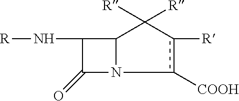

where R, among others, is an acyl group, and R′ is hydrogen, lower alkoxy, lower alkoxyalkyl, lower alkyl, phenylthio or lower alkylmercapto. Compounds in this patent do not include a styryl group substituted at the carbapenem ring. This patent is incorporated by reference herein its entirety for structures of substituted carbepenem compounds therein and methods of synthesis of such compounds.

U.S. Pat. Nos. 5,036,063; 5,116,832; 5,703,068; and 6,271,222 relate to penem compounds. Compounds in these patents do not include a styryl group substituted at the penem ring. Each of these patents is incorporated by reference herein in its entirety for penem structures therein and methods of synthesis of penems.

Despite the plethora of structural modifications of cephalosporin and related cephem and penem antimicrobials, there remains an urgent unmet medical need for more effective antimicrobials to treat infections caused by drug-resistant Gram-negative bacterial pathogens, especially those which are carbapenemase and extended-spectrum beta-lactamase producers. There remains a particularly urgent need for more antimicrobials to treat infections caused by bacteria that are resistant to carbapenems, including Enterobartenaceae (e.g., Escherichia coli), Acinetobacter baumannii, and most particularly Pseudomonas aeruginosa. [11, 21(a)-(g)]

### SUMMARY OF THE INVENTION

The invention relates to certain cephem and penem compounds having a styrylmethylene moiety at the 3-position in the lactam ring to which a leaving group having a positively charged nitrogen is bonded and wherein the leaving group contains a vicinal diol or is bonded to a unsubstituted or substituted catechol. The compounds contain a latent reactive group which is released on hydrolysis/cleavage of the lactam ring and is believed to function to inhibit beta-lactamases. More specifically, the cephems are cephalosporins, cephamycins, carbacephems, and oxacephems. More specifically, the penems are penems, carbapenems or oxapenems. Preferred cephems are cephalosporins. Preferred penems are carbapenems.

In embodiments, compounds of the invention exhibit antibiotic activity against Gram-negative bacteria. In embodiments, compounds of the invention exhibit antibiotic activity against Gram-positive bacteria. In specific embodiments, compounds of the invention exhibit antibiotic activity against bacteria which exhibit multi-drug resistance. In embodiments, compounds of the invention exhibit antibiotic activity against bacteria which produce various beta-lactamases.

In more specific embodiments, compounds of the invention exhibit antibiotic activity against a variety of Enterobacteriaceae. In more specific embodiments, compounds of the invention exhibit antibiotic activity against strains of Escherichia coli; Klebsiella; Proteus, Citrobacter; Serratia; and/or Enterobacter. In more specific embodiments, compounds of the invention exhibit antibiotic activity against strains of Klebsiella pneumoniae; Klebsiella oxytoca; Proteus mirabilis; Citrobacter freundii; Serratia marcescens; Enterobacter aerogenes; and/or Enterobacter cloacae.

In additional embodiments, compounds of the invention exhibit antibiotic activity against bacterial strains which produce extended spectrum beta-lactamases (ESBL). In additional embodiments, compounds of the invention exhibit antibiotic activity against bacterial strains which produce AmpC beta-lactamases. In additional embodiments, compounds of the invention exhibit antibiotic activity against bacterial strains which produce a carbapenemases.

Also provided are pharmaceutical compositions containing one or more cephem or penem compound disclosed herein. Pharmaceutical compositions herein comprise a therapeutically effective amount of a cephem or penem compound of this invention. Pharmaceutical compositions herein comprise a therapeutically effective amount of a cephem or penem compound of any chemical formula herein. In embodiments, pharmaceutical compositions herein comprise a therapeutically effective amount of a cephalosporin of any chemical formula herein. In embodiments, pharmaceutical compositions herein comprise a therapeutically effective amount of a cephalomycin of any chemical formula herein. In embodiments, pharmaceutical compositions herein comprise a therapeutically effective amount of a carbapenem of any chemical formula herein. In embodiments, pharmaceutical compositions herein comprise a combined therapeutically effective amount of two or more cephem or penem compounds of this invention32. In an embodiment, the pharmaceutical composition comprises a beta-lactamase inhibitor or a beta-lactamase antibiotic other than a compound of any one of the formulas herein. In embodiments, pharmaceutical compositions herein optionally contain other appropriate active ingredients including, for example, one or more beta-lactamase inhibitor other than a compound of Formula I, one or more beta-lactamase antibiotic other than a compound of Formula I, one or more monobactam, one or more carbapenem other than a compound of Formula I or one or more aminoglycoside antibiotic. Pharmaceutical compositions herein optionally further comprise one or more pharmaceutically acceptable carriers or excipients as are known in the art.

Also provided are methods of treating a bacterial infection in a subject, which may be a human or non-human animal, and particularly a non-human mammal, by administering one or more compound of any one of the formulas herein or pharmaceutical compositions containing one or more compound of any of the formulas herein.

In embodiments, the invention provides cephems and penems of Formula I:

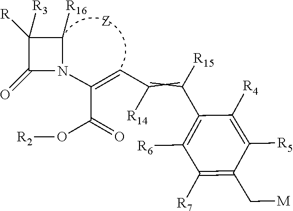

or salts, or solvates thereof,
where:

R is an acyl amino group (R₁CO—NH—) or an alkyl group optionally substituted with a group selected from a halogen, a hydroxy group or a protected hydroxyl group;

R₂ is hydrogen, unsubstituted or substituted alkyl, unsubstituted or substituted aryl, alkoxy-substituted alkyl, acylalkoxy-substituted alkyl, acyloxy-substituted alkyl, a pharmaceutically acceptable cation, when the CO₂ group to which R₂ is attached is negatively charged, or a carboxyl protecting group;

R₃ is hydrogen, a C1-C3-alkoxy group;

—Z— is a linker between the two indicated atoms, which forms a 5- or 6-member carbocyclic or heterocyclic ring with the atoms to which it is linked; R₁₆ is hydrogen, a C1-C3 alkyl group or a C1-C3 alkoxy group;

R₄, R₅, R₆, and R₇ are independently selected from hydrogen, halogen, cyano, nitro, C1-C3 alkyl, C1-C3 haloalkyl, amino, C1-C3 alkylamino, and C1-C3 dialkylamino;

M is:

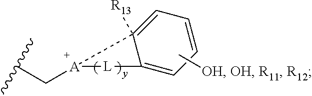

where:

R₁₁ and R₁₂ are independently selected from hydrogen, hydroxyl, halogen, carboxyl or ester thereof, acyl, optionally substituted aryl, optionally substituted aryl alkyl, C1-C3 alkyl, C1-C3 alkoxy, cyano, and nitro groups and if R₁₁ and R₁₂ are substituted on adjacent ring carbons R₁₁ and R₁₂ together with the carbons to which they are attached form an optionally substituted 5- or 6-member carbocyclic, or heterocyclic ring which may be saturated, partially unsaturated or aromatic;

R₁₃, if present, is selected from hydrogen, hydroxyl, halogen, C1-C3 alkyl, cyano, and nitro groups and if R₁₁ or R₁₂ is substituted on a ring carbon adjacent to R₁₃, R₁₃ and R₁₁ or R₁₂ together with the carbons to which they are attached form an optionally substituted 5- or 6-member carbocyclic, or heterocyclic ring which may be saturated, partially unsaturated or aromatic;

A⁺ is a leaving group containing a positively charged nitrogen; and L is an optional divalent linker moiety containing 1-6 carbon atoms and optionally one, two or three heteroatoms (specifically N, S or O), where y is 1 or 0 to indicate presence or absence of the linker;

wherein the phenyl ring of the styryl group is cis or trans or a mixture of cis or trans with respect to the cepham ring;

wherein the A⁺ group is bonded to the indicated phenyl ring of M through linker L or is a partially or fully unsaturated heterocyclic ring fused to the indicated phenyl ring, and

wherein the indicated phenyl ring in M is a catechol ring having two hydroxyl groups substituted on any two adjacent ring carbons.

Beta-lactam ring systems of the compounds of Formula I include those of cephems, cephamycins, carbacephems, oxacephems, penems, carbapenems and oxapenems.

In specific embodiments, the compound of the invention is a pharmaceutically acceptable salt of the compound of Formula I. The salt of the compound may be formed at the carboxylate of the group at the 2-position of the cephem/penem ring; and/or at a carboxylate and/or or at an amino group in the group at the 7-position (cephem) or 6-position (penem) of the ring; and/or at the positively charged nitrogen of the M group. The compound can also be zwitterionic. In specific embodiments, the compound of the invention is a pharmaceutically acceptable solvate of the compound or salt of Formula I. In specific embodiments, the compound of the invention is a pharmaceutically acceptable hydrate of the compound or salt of Formula I.

In specific embodiments, —Z— is a saturated one or two atom linker between the indicated atoms, which forms a 5- or 6-member ring. In embodiments, —Z— is two carbon atoms, a carbon and a sulfur atom, a carbon and a nitrogen, or a carbon and an oxygen, where any remaining valences are satisfied by substitution of atoms with hydrogen or organic substituents, e.g., C1-C3 alkyl groups, and where a sulfur atom, if present, is optionally oxidized (SO or SO₂). More specifically, —Z— is: —S—, —SO—, —SO₂—, —CH₂— —CHR₁₇—, —O—, —S—CH₂—, —SO₂—CH₂—, —CH₂—CH₂—, or —O—CH₂—, where R₁₇ is hydrogen or a C1-C3 alkyl group. More specifically R₁₇ is hydrogen. More specifically, R₁₇ is methyl.

In specific embodiments, R is an optionally substituted alkyl group. In specific embodiments, R is a hydroxy substituted alkyl group, a hydroxy substituted alkyl group where the hydroxyl group is protected or a halogen substituted alkyl group. More specifically, R is a hydroxy substituted C1-C6 alkyl group, where the hydroxyl group is optionally protected. More specifically, R is a halogen substituted C1-C6 alkyl group. More specifically, R is a fluorine substituted C1-C6 alkyl group. Yet more specifically, R is a hydroxyl substituted C1-C3 alkyl group, where the hydroxyl group is optionally protected. In specific embodiments, the C1-C6 alkyl or the C1-C3 alkyl is substituted with an hydroxy or a halogen on the 1-position of the alkyl group. In a specific embodiment, R is 1-hydroxyethyl, where the hydroxyl group is optionally protected. In a specific embodiment, R is a 1-fluoroethyl. In a specific embodiment, R is 1-hydroxyethyl.

In specific embodiments, R is an acylamino group, particularly an acylamino group of a cephalosporin, cephamycin, carbacephem or oxacephem antibiotic. In an embodiment R is R₁—CO—NH—, where R₁ is any of various organic groups, including without limit, optionally substituted alkyl, optionally substituted alkenyl, optionally substituted carbocyclic, optionally substituted aryl, optionally substituted heterocyclic, or optionally substituted heteroaryl. In specific embodiments, R₁CONH— is an acylamino group of a known beta-lactam antibiotic. A wide variety of beta-lactam antibiotics is known in the art. Acylamino groups of representative known beta-lactam antibiotics are described hereinafter. In an embodiment, R₁ is an optionally substituted benzyl group.

In embodiments, the compounds of the invention are compounds of Formula IA:

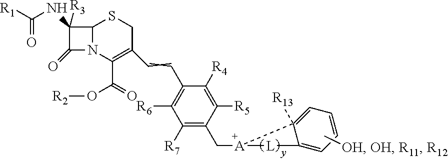

or salts, or solvates thereof,
where variables are as defined in Formula I and embodiments thereof.

In specific embodiments of Formula IA:

### R₁ is:

(1) methylene, substituted with two groups selected from hydrogen, halogen, cyano, amino, alkyl amino, dialkylamino, unsubstituted or substituted aryl group, unsubstituted or substituted heterocyclic group, unsubstituted or substituted thioalkyl group, unsubstituted or substituted thioaryl group, or unsubstituted or substituted thioheterocyclic group, wherein at least one of the groups on the methylene is a group other than hydrogen;

(2) —C(R₂₀)═C—(O)_{z}—R₂₁, where z is 1 or 0 to indicate the presence or absence of the oxygen; or

(3) —C(R₂₀)=N˜O—R₂₂, wherein the N˜O bond is in the syn or anti conformation;

where:
R₂₀ is an unsubstituted or substituted heterocyclic group;
R₂₁ is hydrogen, unsubstituted or substituted C1-C4 straight chain or branched alkyl, unsubstituted or substituted C3-C6 cycloalkyl, unsubstituted or substituted aryl, unsubstituted or substituted heterocycyl, unsubstituted or substituted aryl-substituted C1-C4 alkyl or unsubstituted or substituted heterocycyl-substituted C1-C4 alkyl;
R₂₂ is hydrogen, hydroxyl, unsubstituted or substituted C1-C4 straight chain or branched alkyl, unsubstituted or substituted C3-C6 cycloalkyl, unsubstituted or substituted C1-C4 straight chain or branched alkoxy, unsubstituted or substituted aryl, unsubstituted or substituted heterocycyl, unsubstituted or substituted aryl-substituted C1-C4 alkyl, unsubstituted or substituted heterocycyl-substituted C1-C4 alky,
or R₂₂ is:
Q-substituted C1-C6 straight-chain or branched alkyl or C(Ra)(Rb)Q, where Q is COOH, or a salt thereof, or CH₂—N(OH)CORc, where:
Ra, Rb and Rc are independently hydrogen, C1-C6 alkyl, C2-C7 alkenyl, C2-C7 alkynyl, C3-C6 cycloalkyl, unsubstituted or substituted phenyl, unsubstituted or substituted benzyl, or
Ra and Rb together form a 3-6 member carbocyclic or heterocyclic ring, wherein the heterocyclic can contain one or two heteroatoms, N, NR_{N}, O, or S; and
Rc can further be OH, amino, C-C6 alkylamino, or C1-C6 alkoxy.

In specific embodiments, the compounds of Formula IA are cephalosporins. In specific embodiments, R₃ is hydrogen. In specific embodiments, R₃ is methoxy. In specific embodiments, the compounds of Formula IA are cephamycins. In specific embodiments, compounds of Formula IA have a cyclic quaternary ammonium group. In specific embodiments, compounds of Formula IA have a 5- or 6-member cyclic quaternary ammonium group. In specific embodiments, compounds of Formula IA have an isoquinoline diol or a quinolone diol group.

In specific embodiments of Formula IA, the positively charged nitrogen of the A⁺ group is bonded to the methylene moiety of the styrylmethylene group as illustrated. In specific embodiments of Formula I or IA, the R₁—COOH precursor is selected from R1-4-R1-16 of Scheme 5A, or R1-17 or R1-18 or R1-19-R1-22 of Scheme 5B. In specific embodiments of Formula I or IA, the R₁—COOH precursor is selected from R1-23-R1-50 of Schemes 5B-5D.

In specific embodiments, M is M_{X}:

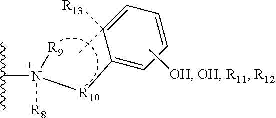

where:
R₈ is hydrogen or unsubstituted or substituted C1-C6 alkyl;
R₉ is selected from hydrogen, unsubstituted or substituted C1-C6 alkyl;
R₁₀ is a divalent —(CH₂)ₙ— moiety, where n is 1-6 wherein one or two CH₂ groups are replaced with —O—, —S—, —CO—, —N(RN)CO—, or —CON(RN)—, where RN is hydrogen or a C1-C3 alkyl;
or R₉ and R₁₀ together with the nitrogen to which they are attached form a 5- or 6-member heterocyclic ring as shown by the dotted line, which ring is partially or fully unsaturated, unsubstituted or substituted, and optionally contains one or two additional heteroatoms: O, S, N, NR_{N} or a combination thereof in the ring, where RN is hydrogen or a C1-C3 alkyl;
where R₁₁ and R₁₂ are independently selected from hydrogen, hydroxyl, halogen, carboxyl or ester thereof, acyl, optionally substituted aryl, optionally substituted aryl alkyl, C1-C3 alkyl, C1-C3 alkoxy, cyano, and nitro groups or, if R₁₁ and R₁₂ are substituted on adjacent ring carbons, R₁₁ and R₁₂ together with the carbons to which they are attached form an optionally substituted 5- or 6-member carbocyclic, or heterocyclic ring, which may be saturated, partially unsaturated or aromatic;
R₁₃, if present, is selected from hydrogen, hydroxyl, halogen, C1-C3 alkyl, cyano, and nitro groups, or, if R₁₁ or R₁₂ is substituted on a ring carbon adjacent to R₁₃, R₁₃ and R₁₁ or R₁₂ together with the carbons to which they are attached form an optionally substituted 5- or 6-member carbocyclic, or heterocyclic ring which may be saturated, partially unsaturated or aromatic;
where dotted lines indicate optional bonds;
where the phenyl ring at the right of the formula is substituted with at least two hydroxyl groups on adjacent ring carbons and is bonded to the positively charged N through the R₁₀ moiety or is fused to the 5- or 6-member ring formed by R₉ and R₁₀.

In specific embodiments of M groups, optional substitution is substitution with one or more C1-C3 alkyl groups, C1-C3 alkoxy groups, hydroxyl, halogen, carboxylate or esters thereof, nitro, or cyano. Specific halogens include chlorine or fluorine.

Exemplary M groups containing a positively charged N are illustrated in Scheme 7 and in the compounds of Scheme 8. The M group in any one of the formulas herein can be selected from any one of the M groups illustrated in Scheme 7 or Scheme 8.

Exemplary R₁—COOH compounds (R₁ precursors) which are useful for introduction of R₁ groups into the compounds of the invention are illustrated in Schemes 5A-5D. In specific embodiments of Formula I and IA, the R₁—COOH precursor is selected from R1-4-R1-16 of Scheme 5A, or R1-17 or R1-18 or R1-19-R1-22 of Scheme 5B. In specific embodiments of Formula I or IA, R₁—COOH precursor is selected from R1-23-R1-50 of Schemes 5B-5D.

In specific embodiments of Formula I or IA R¹ is of formula:

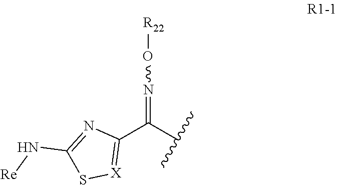

where R₂₂ is as defined for R₁ in Formula IA, X is N or CR_{X}, where R_{X} is hydrogen, C1-C3 alkyl or halogen, and particularly chlorine, and Re is hydrogen or (Rp)₂PO—, where each Rp is independently hydrogen, or C1-C3 alkyl or more specifically both Rp are methyl. The R¹ group is in the E or Z conformation with respect to the oxime group. More specifically the R1-1 group is in the Z conformation. More specifically the R1-1 group is in the E conformation.

In specific embodiments of Formulas I, and IA, R¹ is of formula:

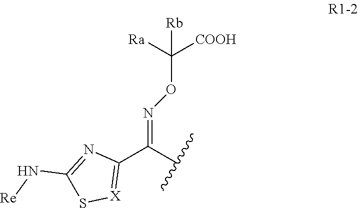

where Ra and Rb are as defined for R₁ in Formula IA; X is N or CR_{X}, where R_{X} is hydrogen, C1-C3 alkyl or halogen, and particularly chlorine; and Re is hydrogen or (Rp)₂PO—, where each Rp is independently hydrogen, or C1-C3 alkyl or more specifically both are methyl. More specifically, Ra and Rb are hydrogen or methyl and Re is hydrogen. Yet more specifically, Ra and Rb are both hydrogen or both methyl groups and Re is hydrogen. In more specific embodiments X is CH or N.

In specific embodiments of Formula I or IA, R¹ is of formula:

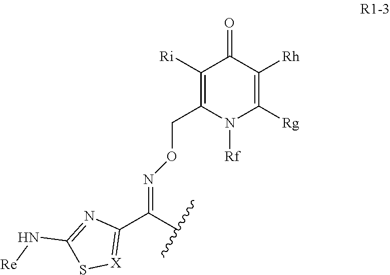

where X is N or CR_{X}, where R_{X} is hydrogen, C1-C3 alkyl or halogen, and particularly chlorine; Re is hydrogen or (Rp)₂PO—, where each Rp is independently hydrogen, or C1-C3 alkyl or more specifically both are methyl; Rf is hydrogen, hydroxyl, C1-C3 alkyl, C1-C3 alkoxy, unsubstituted or substituted phenyl or unsubstituted or substituted benzyl; and Rg, Rh and Ri are independently selected from hydrogen, hydroxyl, amino, and alkyl amino. More specifically, one or two of Rg, Rh, Ri are hydroxyl and the others are hydrogen. More specifically, Re is hydrogen. More specifically, Rf is hydroxyl or alkoxy. More specifically, Rf is hydroxyl.

In specific embodiments of Formulas I and IA, R₄-R₇ are hydrogen. In specific embodiments of Formulas I and IA, R₅-R₇ are hydrogen. In specific embodiments of Formulas I and IA, R₄ is a halogen, a cyano group or a nitro group. In specific embodiments of Formulas I and IA, R₄ is a nitro group.

In specific embodiments of the invention, antibacterial compounds include those illustrates in the chemical structures of Scheme 8 as well as various salts and solvates thereof.

In embodiments, compounds of the invention are those of Formula VA:

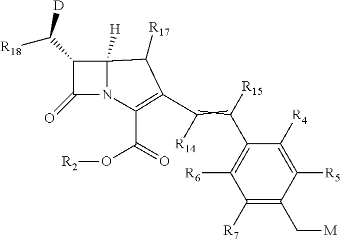

or salts or solvates thereof,
where variables are as defined in Formula I or IA or any embodiments thereof; R₁₈ is an alkyl group and D is hydroxyl, halogen or a protected OH group. In specific embodiments of Formula VA, D is OH. In specific embodiments of Formula VA, R₁₇ is hydrogen. In specific embodiments of Formula VA, R₁₇ is methyl. In specific embodiments of Formula VA, R₁₄ and R₁₅ are both hydrogen. In specific embodiments of Formula VA, all of R₄-R₇ are hydrogens. In specific embodiments of Formula VA, R₂ is hydrogen or when CO₂ is negatively charged, R₂ is a pharmaceutically acceptable cation.

In specific embodiments of Formula VA, M is a leaving group comprising a vicinal diol or an unsubstituted catechol or substituted catechol group. More specifically, M is as defined in Formula I or IA. In specific embodiments of compounds of Formula VA, M has a cyclic quaternary ammonium group. In specific embodiments of compounds of Formula VA, M has a isoquinoline diol or quinolone diol groups.

More specifically M in Formula VA is M_{X}:

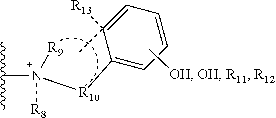

where variables are as defined for Formula IA and any embodiment thereof. More specifically, M in Formula VA is one of the M groups illustrated in Scheme 7 or in Scheme 8.

Other embodiments of the invention will be apparent to one of ordinary skill in the art on review of the Description, Schemes and Examples that follow.

### DETAILED DESCRIPTION OF THE INVENTION

The invention relates to certain cephem and penem compounds having a styrylmethylene moiety at the 3-position in the cephem or penem ring to which a positively charged leaving group is bonded and wherein the leaving group contains a vicinal diol or is bonded to a unsubstituted or substituted catechol. In specific embodiments, the leaving group has a positively charged nitrogen and wherein the leaving group contains a vicinal diol or is bonded to a unsubstituted or substituted catechol. The compounds herein contain a latent reactive group which is released on hydrolysis/cleavage of the lactam ring and is believed to function to inhibit beta-lactamases. More specifically, the cephems are cephalosporins, cephamycins, carbacephems, and oxacephems. More specifically, the penems are penems, carbapenems or oxapenems. Preferred cephems are cephalosporins. Preferred penems are carbapenems.

In embodiments, compounds of the invention exhibit antibiotic activity against Gram-negative bacteria. In embodiments, compounds of the invention exhibit antibiotic activity against Gram-positive bacteria. In specific embodiments, compounds of the invention exhibit antibiotic activity against bacteria which exhibit multi-drug resistance. In embodiments, compounds of the invention exhibit antibiotic activity against bacteria which produce various beta-lactamases. In embodiments, compounds of the invention, in particular, exhibit antibiotic activity against beta-lactamase producing Gram-negative bacteria. In embodiments, compounds of the invention exhibit antibiotic activity against a wide range of Pseudomonas aeruginosa strains, including clinical isolates. In embodiments, compounds of the invention exhibit antibiotic activity against Gram-negative bacteria. In embodiments, compounds of the invention exhibit antibiotic activity against Gram-positive bacteria. In specific embodiments, compounds of the invention exhibit antibiotic activity against bacteria which exhibit multi-drug resistance. In embodiments, compounds of the invention exhibit antibiotic activity against bacteria which produce various beta-lactamases. In embodiments, compounds of the invention in particular exhibit antibiotic activity against beta-lactamase producing Gram-negative bacteria. In embodiments, compounds of the invention exhibit antibiotic activity against a wide range of Pseudomonas aeruginosa strains, including clinical isolates.

In more specific embodiments, compounds of the invention exhibit antibiotic activity against a variety of Enterobacteriaceae. In more specific embodiments, compounds of the invention exhibit antibiotic activity against strains of Escherichia coli; Klebsiella; Proteus, Citrobacter, Serratia; and/or Enterobacter. In more specific embodiments, compounds of the invention exhibit antibiotic activity against strains of Klebsiella pneumoniae; Klebsiella oxytoca; Proteus mirabilis; Citrobacter freundii; Serratia marcescens; Enterobacter aerogenes; and/or Enterobacter cloacae.

In additional embodiments, compounds of the invention exhibit antibiotic activity against bacterial strains which produce extended spectrum beta-lactamases (ESBL). In additional embodiments, compounds of the invention exhibit antibiotic activity against bacterial strains which produce ESBL selected from TEM, SHV, and CTX-M, In additional embodiments, compounds of the invention exhibit antibiotic activity against bacterial strains which produce ESBL selected from TEM-26, SHV-1, CTX-M-14, and CTX-M-15. In additional embodiments, compounds of the invention exhibit antibiotic activity against bacterial strains which produce AmpC beta-lactamases [22]. In additional embodiments, compounds of the invention exhibit antibiotic activity against bacterial strains which produce AmpC beta-lactamases selected from DHA enzymes, particularly DHA-1. In additional embodiments, compounds of the invention exhibit antibiotic activity against bacterial strains which produce a carbapenemases. In additional embodiments, compounds of the invention exhibit antibiotic activity against bacterial strains which produce a carbapenemases selected from KPC, VIM, IMP, NDM, and OXA. In additional embodiments, compounds of the invention exhibit antibiotic activity against bacterial strains which produce a carbapenemase selected from KPC-2, KPC-3, VIM-1, IMP-1, NDM-1, OXA-48 and OXA-58.

Also provided are pharmaceutical compositions containing one or more cephem or penem compound disclosed herein. Pharmaceutical compositions herein comprise a therapeutically effective amount of a cephem compound of this invention.

Pharmaceutical compositions herein comprise a therapeutically effective amount of a cephem or penem compound of any chemical formula herein. In embodiments, pharmaceutical compositions herein comprise a therapeutically effective amount of a cephalosprin of any chemical formula herein. In embodiments, pharmaceutical compositions herein comprise a therapeutically effective amount of a cephalomycin of any chemical formula herein. In embodiments, pharmaceutical compositions herein comprise a therapeutically effective amount of a carbapenem of any chemical formula herein. In embodiments, pharmaceutical compositions herein comprise a combined therapeutically effective amount of two or more cephem or penem compounds of this invention. In embodiments, pharmaceutical compositions herein comprise a combined therapeutically effective amount of two or more cephem or penem compounds of any formula herein and in particular any compound of Formula I, IA or VA.

In embodiments, pharmaceutical compositions herein optionally contain other appropriate active ingredients including, for example, one or more beta-lactamase inhibitors, other than a compound of Formula I. In embodiments, pharmaceutical compositions herein optionally contain other appropriate active ingredients including, for example, one or more beta-lactamase inhibitors, other than a cephem. In embodiments, pharmaceutical compositions herein contain other appropriate active ingredients including, for example, one or more beta-lactamase antibiotics other than a compound of Formula I. In embodiments, pharmaceutical compositions herein contain other appropriate active ingredients including, for example, one or more monobactam. In embodiments, pharmaceutical compositions herein contain other appropriate active ingredients including, for example, one or more carbapenem other than a compound of Formula I. In embodiments, pharmaceutical compositions herein contain other appropriate active ingredients including, for example, aminoglycoside antibiotics. Pharmaceutical compositions herein optionally further comprise one or more pharmaceutically acceptable carriers or excipients as are known in the art.

Also provided are methods of treating a bacterial infection in a subject, which may be a human or non-human animal, and particularly a non-human mammal, by administering one or more compound of formulas herein or pharmaceutical compositions containing one or more compound of formulas herein.

The invention relates, in particular, to cephem and penem compounds which exhibit strong antibacterial activity against certain multi-drug resistant bacteria. In specific embodiments, compounds herein exhibit low microgram/mL Minimum Inhibitory Concentrations (MIC) against bacteria which produce extended spectrum beta-lactamases.

In specific embodiments, compounds herein exhibit MICs of less than 100 microg/mL, of less than 50 microg/mL, less than 30 microg/mL, less than 25 micorg/mL, less than 10 microg/mL, less than 5 microg/mL, less than 1 microg/mL or less than 0.5 microg/mL against bacteria which are resistant to at least one cephalosporin antibiotic.

In specific embodiments, compounds herein exhibit MICs of less than 100 microg/mL, of less than 50 microg/mL, less than 30 microg/mL, less than 25 micorg/mL, less than 10 microg/mL, less than 5 microg/mL, less than 1 microg/mL or less than 0.5 microg/mL against bacteria which are resistant to at least one cephem or penem antibiotic.

In specific embodiments, compounds herein exhibit MICs of less than 100 microg/mL, of less than 50 microg/mL, less than 30 microg/mL, less than 25 microg/mL, less than 10 microg/mL, less than 5 microg/mL, less than 1 microg/mL or less than 0.5 microg/mL against bacteria which are resistant to at least one carbapenem.

In specific embodiments, compounds herein exhibit MICs of less than 100 microg/mL, of less than 50 microg/mL, less than 30 microg/mL, less than 25 micorg/mL, less than 10 microg/mL, less than 5 microg/mL, less than 1 microg/mL or less than 0.5 microg/mL against bacteria which produce at least one ESBL.

More specifically the invention relates to cephem and penem compounds substituted with a styrylmethylene group which is bonded to a positively charged leaving group and which is in turn bonded to a vicinal diol group, or more specifically a catechol moiety. In specific embodiments, the positively charged leaving group has a positively charged nitrogen.

In embodiments, the invention provides cephems and penems of Formula I:

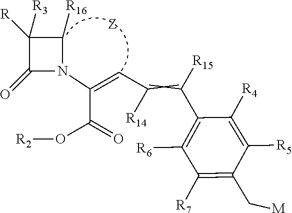

or salts, or solvates thereof,
where:
R is an acyl amino group (R₁CO—NH—) or an alkyl group optionally substituted with a group selected from a halogen, a hydroxy group or a protected hydroxyl group;
R₂ is hydrogen, unsubstituted or substituted alkyl, unsubstituted or substituted aryl, a pharmaceutically acceptable cation, when the CO₂ group to which R₂ is attached is negatively charged, or a carboxyl protecting group;
R₃ is hydrogen, a C1-C3-alkoxy group;
—Z— is a linker between the two indicated atoms, which forms a 5- or 6-member carbocyclic or heterocyclic ring with the atoms to which it is linked;
R₁₆ is hydrogen, a C1-C3 alkyl group or a C1-C3 alkoxy group;
R₄, R₅, R₆, and R₇ are independently selected from hydrogen, halogen, cyano, nitro, C1-C3 alkyl, C1-C3 haloalkyl, amino, C1-C3 alkylamino, and C1-C3 dialkylamino;

### M is:

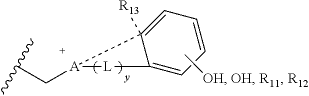

where R₁₁ and R₁₂ are independently selected from hydrogen, hydroxyl, halogen, carboxyl or ester thereof, acyl, optionally substituted aryl, optionally substituted aryl alkyl, C1-C3 alkyl, C1-C3 alkoxy, cyano, and nitro groups or, if R₁₁ and R₁₂ are substituted on adjacent ring carbons, R₁₁ and R₁₂ together with the carbons to which they are attached form an optionally substituted 5- or 6-member carbocyclic, or heterocyclic ring, which may be saturated, partially unsaturated or aromatic;
R₁₃, if present, is selected from hydrogen, hydroxyl, halogen, C1-C3 alkyl, cyano, and nitro groups, or, if R₁₁ or R₁₂ is substituted on a ring carbon adjacent to R₁₃, R₁₃ and R₁₁ or R₁₂ together with the carbons to which they are attached form an optionally substituted 5- or 6-member carbocyclic, or heterocyclic ring which may be saturated, partially unsaturated or aromatic;
A⁺ is a leaving group containing a positively charged nitrogen;
L is an optional divalent linker moiety containing 1-6 carbon atoms and optionally one, two or three heteroatoms (N, S or O), where y is 1 or 0 to indicate presence or absence of the linker;
wherein the phenyl ring of the styryl group is cis or trans or a mixture of cis or trans with respect to the cepham ring;
wherein the A⁺ group is bonded to the indicated phenyl ring of M through linker L or is a partially or fully unsaturated heterocyclic ring fused to the indicated phenyl ring, and
wherein the indicated phenyl ring in M is a catechol having two hydroxyl group substituted on adjacent ring carbons.

Beta-lactam ring systems of the compounds of Formula I include those of cephems, cephamycins, carbacephems, oxacephems, penems, carbapenems or oxapenems.

In specific embodiments, the compound of the invention is a pharmaceutically acceptable salt of the compound of Formula I. In specific embodiments, the compound of the invention is a pharmaceutically acceptable solvate of the compound or salt of Formula I. In specific embodiments, the compound of the invention is a pharmaceutically acceptable hydrate of the compound or salt of Formula I.

In specific embodiments, —Z— is a saturated one or two atom linker between the indicated atoms, which forms a 5- or 6-member ring. Z can be two carbon atoms, a carbon and a sulfur atom, a carbon and a nitrogen, or a carbon and an oxygen, where any remaining valences are satisfied by substitution of atoms with hydrogen or organic substituents, e.g., C1-C3 alkyl groups, and where a sulfur atom, if present, can be oxidized (SO or SO₂).

More specifically, —Z— is:

—S—, —SO—, —SO₂—, —CH₂— —CHR₁₇—, —O—, —S—CH₂—, —SO₂—CH₂—, —CH₂—CH₂—, or —O—CH₂—, where R₁₇ is hydrogen or a C1-C3 alkyl group. More specifically R₁₇ is hydrogen. More specifically, R₁₇ is methyl.

In specific embodiments, R is an optionally substituted alkyl group. In specific embodiments, R is a hydroxy substituted alkyl group, a hydroxy substituted alkyl group where the hydroxyl group is protected or a halogen substituted alkyl group. More specifically, R is a hydroxy substituted C1-C6 alkyl group, where the hydroxyl group is optionally protected. More specifically, R is a halogen substituted C1-C6 alkyl group. More specifically, R is a fluorine substituted C1-C6 alkyl group. Yet more specifically, R is a hydroxyl substituted C1-C3 alkyl group, where the hydroxyl group is optionally protected. In specific embodiments, the C1-C6 alkyl or the C1-C3 alkyl is substituted with a hydroxy or a halogen on the 1-position of the alkyl group. In a specific embodiment, R is 1-hydroxyethyl, where the hydroxyl group is optionally protected. In a specific embodiment, R is a 1-fluoroethyl. In a specific embodiment, R is 1-hydroxyethyl.

In a specific embodiment R is:

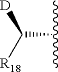

where D and R₁₈ are as defined above. More specifically D is OH or protected OH and R₁₈ is C1-C3 alkyl.

In specific embodiments, R is an acylamino group, particularly an acylamino group of a cephalosporin, cephamycin, carbacephem or oxacephem antibiotic. In an embodiment R is R₁—CO—NH—, where R₁ is any of various organic groups, including without limit, optionally substituted alkyl, optionally substituted alkenyl, optionally substituted carbocyclic, optionally substituted aryl, optionally substituted heterocyclic, or optionally substituted heteroaryl. In specific embodiments, R₁CONH— is an acylamino group of a known beta-lactam antibiotic. A wide variety of beta-lactam antibiotics is known in the art. Acylamino groups of representative known beta-lactam antibiotics are described hereinafter. In an embodiment, R₁ is an optionally substituted benzyl group.

In specific embodiments, R₁ is:

(1) methylene, substituted with two groups selected from hydrogen, halogen, cyano, amino, alkyl amino, dialkylamino, unsubstituted or substituted aryl group, unsubstituted or substituted heterocyclic group, unsubstituted or substituted thioalkyl group, unsubstituted or substituted thioaryl group, or unsubstituted or substituted thioheterocyclic group;

(2) —C(R₂₀)═C—(O)_{z}—R₂₁, where z is 1 or 0 to indicate the presence or absence of the oxygen; or

(3) —C(R₂₀)=N˜O—R₂₂, wherein the N˜O bond is in the syn or anti conformation;

where:
R₂₀ is an unsubstituted or substituted heterocyclic group;
R₂₁ is hydrogen, unsubstituted or substituted C1-C4 straight chain or branched alkyl, unsubstituted or substituted C3-C6 cycloalkyl, unsubstituted or substituted aryl, unsubstituted or substituted heterocycyl, unsubstituted or substituted aryl-substituted C1-C4 alkyl or unsubstituted or substituted heterocycyl-substituted C1-C4 alkyl;
R₂₂ is hydrogen, hydroxyl, unsubstituted or substituted C1-C4 straight chain or branched alkyl, unsubstituted or substituted C3-C6 cycloalkyl, unsubstituted or substituted C1-C4 straight chain or branched alkoxy, unsubstituted or substituted aryl, unsubstituted or substituted heterocycyl, unsubstituted or substituted aryl-substituted C1-C4 alkyl, unsubstituted or substituted heterocycyl-substituted C1-C4 alky,
or R₂₂ is:
Z-substituted C1-C6 straight-chain or branched alkyl or C(Ra)(Rb)Z, where Z is COOH, or a salt thereof, or CH₂—N(OH)CORc, where:
Ra, Rb and Rc are independently hydrogen, C1-C6 alkyl, C2-C7 alkenyl, C2-C7 alkynyl, C3-C6 cycloalkyl, unsubstituted or substituted phenyl, unsubstituted or substituted benzyl, or
Ra and Rb together form a 3-6 member carbocyclic or heterocyclic ring, wherein the heterocyclic can contain one or two heteroatoms, N, NR_{N}, O, or S; and
Rc can further be OH, amino, C-C6 alkylamino, or C1-C6 alkoxy.

In specific embodiments of Formula I, R¹ is of formula:

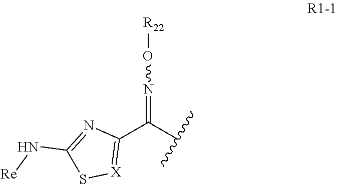

where R₂₂ is as defined for R₁ in Formula I, X is N or CR_{X}, where R_{X} is hydrogen, C1-C3 alkyl or halogen, and particularly chlorine, and Re is hydrogen or (Rp)₂PO—, where each Rp is independently hydrogen, or C1-C3 alkyl or more specifically both Rp are methyl. The R¹ group is in the E or Z conformation with respect to the oxime group. More specifically the R1-1 group is in the Z conformation.

In specific embodiments of Formula I, R¹ is of formula:

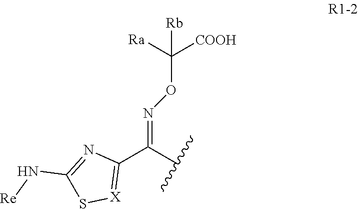

where Ra and Rb are as defined for R₁ in Formula I, X is N or CR_{X}, where R_{X} is hydrogen, C1-C3 alkyl or halogen, and particularly chlorine, and Re is hydrogen or (Rp)₂PO—, where each Rp is independently hydrogen, or C1-C3 alkyl or more specifically both are methyl. More specifically, Ra and Rb are hydrogen or methyl and Re is hydrogen. Yet more specifically, Ra and Rb are both hydrogen or both methyl groups and Re is hydrogen. In more specific embodiments X is CH or N.
In specific embodiments of Formula I, R¹ is of formula:

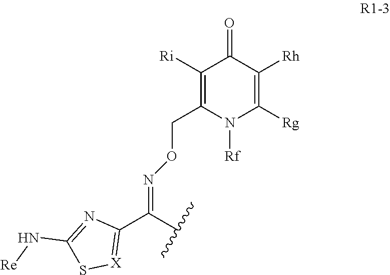

where X is N or CR_{X}, where R_{X} is hydrogen, C1-C3 alkyl or halogen, and particularly chlorine, and Re is hydrogen or (Rp)₂PO—, where each Rp is independently hydrogen, or C1-C3 alkyl or more specifically both are methyl. Rf is hydrogen, hydroxyl, C1-C3 alkyl, C1-C3 alkoxy, unsubstituted or substituted phenyl or unsubstituted or substituted benzyl. Rg, Rh and Ri are independently selected from hydrogen, hydroxyl, amino, alkyl amino. More specifically, one or two of Rg, Rh, Ri are hydroxyl and the others are hydrogen. More specifically, Re is hydrogen. More specifically, Rf is hydroxyl or alkoxy. More specifically, Rf is hydroxyl.

In specific embodiments of Formula I, R₄-R₇ are hydrogen. In specific embodiments of Formula I, R₅-R₇ are hydrogen. In specific embodiments of Formula I, R₄ is a halogen, a cyano group or a nitro group. In specific embodiments of Formula I, R₄ is a nitro group.

In specific embodiments of the invention, antibacterial compounds include those illustrates in the chemical structures of Scheme 8 as well as various salts and solvates thereof.

In specific embodiments of Formula I:

### Z is —S—CH₂—; or

Z is —S—CH₂—, R₃ is hydrogen and R₁₆ is hydrogen; or
Z is —S—CH₂—, R₃ is hydrogen, R₁₆ is hydrogen and R is acylamino; or
Z is —S—CH₂—, R₃ is alkoxy, R₁₆ is hydrogen and R is acylamino; or
Z is —S—CH₂—, R₃ is methoxy, R₁₆ is hydrogen and R is acylamino; or
Z is —CH₂—CH₂—; or
Z is —CH₂—CH₂—, R₃ is hydrogen and R₁₆ is hydrogen; or
Z is —CH₂—CH₂—, R₃ is hydrogen, R₁₆ is hydrogen and R is acylamino; or
Z is —CH₂—CH₂—, R₃ is alkoxy, R₁₆ is hydrogen and R is acylamino; or
Z is —CH₂—CH₂—, R₃ is methoxy, R₁₆ is hydrogen and R is acylamino; or

### Z is —O—CH₂—; or

Z is —O—CH₂—, R₃ is hydrogen and R₁₆ is hydrogen; or
Z is —O—CH₂—, R₃ is hydrogen, R₁₆ is hydrogen and R is acylamino; or
Z is —O—CH₂—, R₃ is alkoxy, R₁₆ is hydrogen and R is acylamino; or
Z is —O—CH₂—, R₃ is methoxy, R₁₆ is hydrogen and R is acylamino; or

### Z is —S—; or

Z is —S—, R₃ is hydrogen and R₁₆ is hydrogen; or
Z is —S—, R₃ is hydrogen, R₁₆ is hydrogen and R is acylamino; or
Z is —S—, R₃ is alkoxy, R₁₆ is hydrogen and R is acylamino; or
Z is —S—, R₃ is methoxy, R₁₆ is hydrogen and R is acylamino; or
Z is —S—, R₃ is hydrogen, R₁₆ is hydrogen and R is OH-substituted alkyl; or
Z is —S—, R₃ is hydrogen, R₁₆ is hydrogen and R is OH-substituted C1-C3 alkyl; or
Z is —S—, R₃ is hydrogen, R₁₆ is hydrogen and R is 1-hydroxyethyl; or

### Z is —CHR₁₇; or

### Z is —CH₂—; or

### Z is —CHCH₃—; or

Z is —CHR₁₇⁻, R₁₇ is hydrogen, R₃ is hydrogen and R₁₆ is hydrogen; or
Z is —CHR₁₇—, R₁₇ is hydrogen, R₃ is hydrogen, R₁₆ is hydrogen and R is acylamino; or
Z is —CHR₁₇—, R₁₇ is hydrogen, R₃ is hydrogen, R₁₆ is hydrogen and R is OH-substituted alkyl; or
Z is —CHR₁₇—, R₁₇ is hydrogen, R₃ is hydrogen, R₁₆ is hydrogen and R is OH-substituted C1-C3 alkyl; or
Z is —CHR₁₇—, R₁₇ is hydrogen, R₃ is hydrogen, R₁₆ is hydrogen and R is 1-hydroxyethyl; or
Z is —CHR₁₇—, R₁₇ is hydrogen, R₃ is alkoxy, and R₁₆ is hydrogen; or
Z is —CHR₁₇—, R₁₇ is hydrogen, R₃ is alkoxy, R₁₆ is hydrogen and R is acylamino; or
Z is —CHR₁₇—, R₁₇ is hydrogen, R₃ is methoxy, R₁₆ is hydrogen and R is acylamino; or
Z is —CHR₁₇⁻, R₁₇ is methyl, R₃ is hydrogen and R₁₆ is hydrogen; or
Z is —CHR₁₇—, R₁₇ is methyl, R₃ is hydrogen, R₁₆ is hydrogen and R is acylamino; or
Z is —CHR₁₇—, R₁₇ is methyl, R₃ is hydrogen, R₁₆ is hydrogen and R is OH-substituted alkyl; or
Z is —CHR₁₇—, R₁₇ is methyl, R₃ is hydrogen, R₁₆ is hydrogen and R is OH-substituted C1-C3 alkyl; or
Z is —CHR₁₇—, R₁₇ is methyl, R₃ is hydrogen, R₁₆ is hydrogen and R is 1-hydroxyethyl; or
Z is —CHR₁₇—, R₁₇ is methyl, R₃ is alkoxy, and R₁₆ is hydrogen; or
Z is —CHR₁₇—, R₁₇ is methyl, R₃ is alkoxy, R₁₆ is hydrogen and R is acylamino; or
Z is —CHR₁₇—, R₁₇ is hydrogen, R₃ is methoxy, R₁₆ is hydrogen and R is acylamino; or

### Z is —O—; or

Z is —O—, R₃ is hydrogen and R₁₆ is hydrogen; or
Z is —O—, R₃ is hydrogen, R₁₆ is hydrogen and R is acylamino; or
Z is —O—, R₃ is alkoxy, R₁₆ is hydrogen and R is acylamino; or
Z is —O—, R₃ is methoxy, R₁₆ is hydrogen and R is acylamino; or
Z is —O—, R₃ is hydrogen, R₁₆ is hydrogen and R is OH-substituted alkyl; or
Z is —O—, R₃ is hydrogen, R₁₆ is hydrogen and R is OH-substituted C1-C3 alkyl; or
Z is —O—, R₃ is hydrogen, R₁₆ is hydrogen and R is 1-hydroxyethyl.

In further specific embodiments of Formula I:

R₃ is hydrogen;
R₃ is methoxy;
R₁₄ and R₁₅ are both hydrogen;
R₁₆ is hydrogen;
R₂ is R₂ is hydrogen, or when —CO₂ is negatively charged R₂ is a pharmaceutically acceptable cation;
one of R₄, R₅, R₆ and R₇ is a non-hydrogen substituent and the remaining variables are each hydrogen;
one of R₄, R₅, R₆ and R₇ is a nitro group and the remaining variables are each hydrogen;
one of R₄, R₅, R₆ and R₇ is a hydroxyl and the remaining variables are each hydrogen;
one of R₄, R₅, R₆ and R₇ is a methyl group and the remaining variables are each hydrogen; or
R₄, R₅, R₆, R₇, R₁₄ and R₁₅ are independently selected from hydrogen, hydroxy, halogen, cyano, nitro, C1-C3 alkyl and C1-C3 haloalkyl.

More specifically M in Formula I is M_{X}:

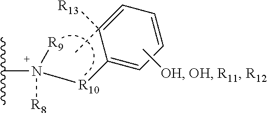

where:
R₈ is hydrogen or unsubstituted or substituted C1-C6 alkyl;
R₉ is selected from hydrogen, unsubstituted or substituted C1-C6 alkyl;
R₁₀ is a divalent —(CH₂)ₙ— moiety, where n is 1-6 wherein one or two CH₂ groups are replaced with —O—, —S—, —CO—, —N(RN)CO—, or —CON(RN)—, where RN is hydrogen or a C1-C3 alkyl;
or R₉ and R₁₀ together with the nitrogen to which they are attached form a 5- or 6-member heterocyclic ring as shown by the dotted line, which ring is partially or fully unsaturated, unsubstituted or substituted, and optionally contains one or two additional heteroatoms: O, S, N, NR_{N} or a combination thereof in the ring, where RN is hydrogen or a C1-C3 alkyl;
R₁₁ and R₁₂ are independently selected from hydrogen, hydroxyl, halogen, carboxyl or ester thereof, acyl, optionally substituted aryl, optionally substituted aryl alkyl, C1-C3 alkyl, C1-C3 alkoxy, cyano, and nitro groups or, if R₁₁ and R₁₂ are substituted on adjacent ring carbons, R₁₁ and R₁₂ together with the carbons to which they are attached form an optionally substituted 5- or 6-member carbocyclic, or heterocyclic ring, which may be saturated, partially unsaturated or aromatic;
R₁₃, if present, is selected from hydrogen, hydroxyl, halogen, C1-C3 alkyl, cyano, and nitro groups, or, if R₁₁ or R₁₂ is substituted on a ring carbon adjacent to R₁₃, R₁₃ and R₁₁ or R₁₂ together with the carbons to which they are attached form an optionally substituted 5- or 6-member carbocyclic, or heterocyclic ring which may be saturated, partially unsaturated or aromatic;
where dotted lines indicate optional bonds;
where the phenyl ring at the right of the formula is substituted with two hydroxyl groups on adjacent ring carbons and is bonded to the positively charged N through the R₁₀ moiety or is fused to the 5- or 6-member ring formed by R₉ and R₁₀.

In specific embodiments of M_{X}:

R₈ is hydrogen; or
R₈ is methyl; or
R₈ is unsubstituted C1-C6 alkyl; or
R₉ and R₁₀ together with the nitrogen to which they are attached form a 5- or 6-member heterocyclic ring which is partially or fully unsaturated, unsubstituted or substituted; or
R₉ and R₁₀ together with the nitrogen to which they are attached form a 5- or 6-member heterocyclic ring which is partially or fully unsaturated, unsubstituted or substituted and which contains a second nitrogen atom; or
R₁₁ and R₁₂ are independently selected from hydrogen, hydroxyl, halogen, and C1-C3 alkyl; or
R₁₁ and R₁₂ together with the carbons to which they are attached form an optionally substituted 5- or 6-member carbocyclic, or heterocyclic ring, which may be saturated, partially unsaturated or aromatic.

In specific embodiments of M_{X}:

R₉ and R₁₀ together with the nitrogen to which they are attached form a 5- or 6-member heterocyclic ring as shown by the dotted line, which ring is partially or fully unsaturated, unsubstituted or substituted, and optionally contains one or two additional heteroatoms: O, S, N, NR_{N} or a combination thereof in the ring, where RN is hydrogen or a C1-C3 alkyl; or
R₉ and R₁₀ together with the nitrogen to which they are attached form a 5- or 6-member heterocyclic ring as shown by the dotted line, which ring is fully unsaturated, unsubstituted or substituted, and optionally contains one or two additional heteroatoms: O, S, N, NR_{N} or a combination thereof in the ring, where RN is hydrogen or a C1-C3 alkyl; or
R₉ and R₁₀ together with the nitrogen to which they are attached form a 5- or 6-member heterocyclic ring as shown by the dotted line, which ring is fully unsaturated, and unsubstituted, and does not contain additional heteroatoms; or
R₉ and R₁₀ together with the nitrogen to which they are attached form a 5- or 6-member heterocyclic ring as shown by the dotted line, which ring is saturated, unsubstituted, and optionally contains one or two additional heteroatoms: O, S, N, NR_{N} or a combination thereof in the ring, where RN is hydrogen or a C1-C3 alkyl; or
R₉ and R₁₀ together with the nitrogen to which they are attached form a 5- or 6-member heterocyclic ring as shown by the dotted line, which ring is saturated, unsubstituted, and does not contain any additional heteroatom in the ring.

In embodiments, the compounds of the invention are those of Formula IA:

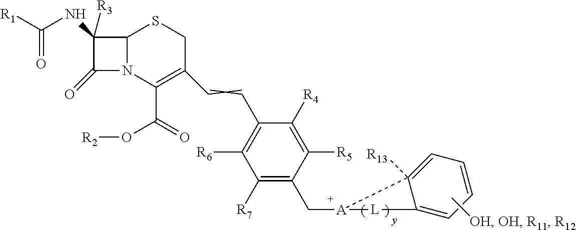

or salts, or solvates thereof,
wherein variables are as defined in Formula I and any embodiments thereof.

In specific embodiments of Formula IA:

### R₁ is:

(1) methylene, substituted with two groups selected from hydrogen, halogen, cyano, amino, alkyl amino, dialkylamino, unsubstituted or substituted aryl group, unsubstituted or substituted heterocyclic group, unsubstituted or substituted thioalkyl group, unsubstituted or substituted thioaryl group, or unsubstituted or substituted thioheterocyclic group, wherein at least one of the groups on the methylene is a group other than hydrogen;

(2) —C(R₂₀)═C—(O)_{z}—R₂₁, where z is 1 or 0 to indicate the presence or absence of the oxygen; or

(3) —C(R₂₀)═N˜O—R₂₂, wherein the N˜O bond is in the syn or anti conformation;

where:
R₂₀ is an unsubstituted or substituted heterocyclic group;
R₂₁ is hydrogen, unsubstituted or substituted C1-C4 straight chain or branched alkyl, unsubstituted or substituted C3-C6 cycloalkyl, unsubstituted or substituted aryl, unsubstituted or substituted heterocycyl, unsubstituted or substituted aryl-substituted C1-C4 alkyl or unsubstituted or substituted heterocycyl-substituted C1-C4 alkyl;
R₂₂ is hydrogen, hydroxyl, unsubstituted or substituted C1-C4 straight chain or branched alkyl, unsubstituted or substituted C3-C6 cycloalkyl, unsubstituted or substituted C1-C4 straight chain or branched alkoxy, unsubstituted or substituted aryl, unsubstituted or substituted heterocycyl, unsubstituted or substituted aryl-substituted C1-C4 alkyl, unsubstituted or substituted heterocycyl-substituted C1-C4 alky,
or R₂₂ is:
Q-substituted C1-C6 straight-chain or branched alkyl or C(Ra)(Rb)Q, where Q is COOH, or a salt thereof, or CH₂—N(OH)CORc, where:
Ra, Rb and Rc are independently hydrogen, C1-C6 alkyl, C2-C7 alkenyl, C2-C7 alkynyl, C3-C6 cycloalkyl, unsubstituted or substituted phenyl, unsubstituted or substituted benzyl, or
Ra and Rb together form a 3-6 member carbocyclic or heterocyclic ring, wherein the heterocyclic can contain one or two heteroatoms, N, NR_{N}, O, or S; and
Rc can further be OH, amino, C-C6 alkylamino, or C1-C6 alkoxy.

In specific embodiments, the compounds of Formula IA are cephalosporins. In specific embodiments, R₃ is hydrogen. In specific embodiments, R₃ is methoxy. In specific embodiments, the compounds of Formula IA are cephamycins. In specific embodiments, compounds of Formula IA have a cyclic quaternary ammonium group. In specific embodiments, compounds of Formula IA have a 5- or 6-member cyclic quaternary ammonium group. In specific embodiments, compounds of Formula IA have an isoquinoline diol or a quinolone diol group.

More specifically M in Formulas I or IA is M_{X}:

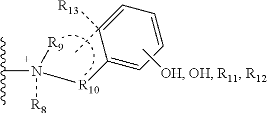

where:
R₈ is hydrogen or unsubstituted or substituted C1-C6 alkyl;
R₉ is selected from hydrogen, unsubstituted or substituted C1-C6 alkyl;
R₁₀ is a divalent —(CH₂)ₙ— moiety, where n is 1-6 wherein one or two CH₂ groups are replaced with —O—, —S—, —CO—, —N(RN)CO—, or —CON(RN)—, where RN is hydrogen or a C1-C3 alkyl;
or R₉ and R₁₀ together with the nitrogen to which they are attached form a 5- or 6-member heterocyclic ring as shown by the dotted line, which ring is partially or fully unsaturated, unsubstituted or substituted, and optionally contains one or two additional heteroatoms: O, S, N, NR_{N} or a combination thereof in the ring, where RN is hydrogen or a C1-C3 alkyl;
where R₁₁ and R₁₂ are independently selected from hydrogen, hydroxyl, halogen, carboxyl or ester thereof, acyl, optionally substituted aryl, optionally substituted aryl alkyl, C1-C3 alkyl, C1-C3 alkoxy, cyano, and nitro groups or, if R₁₁ and R₁₂ are substituted on adjacent ring carbons, R₁₁ and R₁₂ together with the carbons to which they are attached form an optionally substituted 5- or 6-member carbocyclic, or heterocyclic ring, which may be saturated, partially unsaturated or aromatic;
R₁₃, if present, is selected from hydrogen, hydroxyl, halogen, C1-C3 alkyl, cyano, and nitro groups, or, if R₁₁ or R₁₂ is substituted on a ring carbon adjacent to R₁₃, R₁₃ and R₁₁ or R₁₂ together with the carbons to which they are attached form an optionally substituted 5- or 6-member carbocyclic, or heterocyclic ring which may be saturated, partially unsaturated or aromatic;
where dotted lines indicate optional bonds;
where the phenyl ring at the right of the formula is substituted with two hydroxyl groups on adjacent ring carbons and is bonded to the positively charged N through the R₁₀ moiety or is fused to the 5- or 6-member ring formed by R₉ and R₁₀.

In specific embodiments of Formula I or IA, the positively charged nitrogen of the A⁺ group is bonded to the methylene moiety of the styrylmethylene group as illustrated. In specific embodiments of Formula I or IA, the R₁—COOH precursor is selected from R1-4-R1-16 of Scheme 5A, or R1-17 or R1-18 or R1-19-R1-22 of Scheme 5B. In specific embodiments of Formula I or IA, the R₁—COOH precursor is selected from R1-23-R1-50 of Schemes 5B-5D.

In specific embodiments of Formulas I or IA, M is M_{X} and R₁ is selected from R1-1, R1-2 or R1-3. In specific embodiments of Formulas I or IA, M is one of the M groups illustrated in Scheme 7 or 8 and R₁ is selected from R1-1, R1-2 or R1-3.

In embodiments, the compounds of the invention are those of Formula IIA:

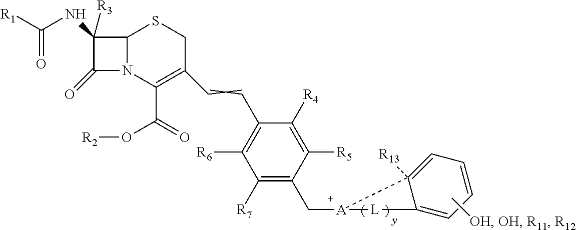

or salts, or solvates thereof,
wherein variables are as defined in Formula I and embodiments thereof.

In specific embodiments of Formula IIA:

### R₁ is:

(1) methylene, substituted with two groups selected from hydrogen, halogen, cyano, amino, alkyl amino, dialkylamino, unsubstituted or substituted aryl group, unsubstituted or substituted heterocyclic group, unsubstituted or substituted thioalkyl group, unsubstituted or substituted thioaryl group, or unsubstituted or substituted thioheterocyclic group, wherein at least one of the groups on the methylene is a group other than hydrogen;

(2) —C(R₂₀)═C—(O)_{z}—R₂₁, where z is 1 or 0 to indicate the presence or absence of the oxygen; or

(3) —C(R₂₀)=N˜O—R₂₂, wherein the N˜O bond is in the syn or anti conformation;

where:
R₂₀ is an unsubstituted or substituted heterocyclic group;
R₂₁ is hydrogen, unsubstituted or substituted C1-C4 straight chain or branched alkyl, unsubstituted or substituted C3-C6 cycloalkyl, unsubstituted or substituted aryl, unsubstituted or substituted heterocycyl, unsubstituted or substituted aryl-substituted C1-C4 alkyl or unsubstituted or substituted heterocycyl-substituted C1-C4 alkyl;
R₂₂ is hydrogen, hydroxyl, unsubstituted or substituted C1-C4 straight chain or branched alkyl, unsubstituted or substituted C3-C6 cycloalkyl, unsubstituted or substituted C1-C4 straight chain or branched alkoxy, unsubstituted or substituted aryl, unsubstituted or substituted heterocycyl, unsubstituted or substituted aryl-substituted C1-C4 alkyl, unsubstituted or substituted heterocycyl-substituted C1-C4 alky,
or R₂₂ is:
Q-substituted C1-C6 straight-chain or branched alkyl or C(Ra)(Rb)Q, where Q is COOH, or a salt thereof, or CH₂—N(OH)CORc, where:
Ra, Rb and Rc are independently hydrogen, C1-C6 alkyl, C2-C7 alkenyl, C2-C7 alkynyl, C3-C6 cycloalkyl, unsubstituted or substituted phenyl, unsubstituted or substituted benzyl, or
Ra and Rb together form a 3-6 member carbocyclic or heterocyclic ring, wherein the heterocyclic can contain one or two heteroatoms, N, NR_{N}, O, or S; and
Rc can further be OH, amino, C-C6 alkylamino, or C1-C6 alkoxy.

In specific embodiments, the compounds of Formula IIA are cephalosporins. In specific embodiments, R₃ is hydrogen. In specific embodiments, R₃ is methoxy. In specific embodiments, the compounds of Formula IIA are cephamycins. In specific embodiments, compounds of Formula IIA have a cyclic quaternary ammonium group. In specific embodiments, compounds of Formula IIA have a 5- or 6-member cyclic quaternary ammonium group. In specific embodiments, compounds of Formula IIA have an isoquinoline diol or a quinolone diol group.

In specific embodiments of Formula IIA, R₁ is selected from R1-1, R1-2 or R1- as illustrated above. In specific embodiments of Formula IIA, M is M_{X}. In specific embodiments of Formula IIA, M is one of the M groups illustrated in Scheme 7 or Scheme 8. In specific embodiments of Formula IIA, M is M_{X} and R₁ is selected from R1-1, R1-2 or R1-3. In specific embodiments of Formula IIA, M is one of the M groups illustrated in Scheme 7 or Scheme 8 and R₁ is selected from R1-1, R1-2 or R1-3.

In specific embodiments of Formula IIA, the positively charged nitrogen of the A⁺ group is bonded to the methylene moiety of the styrylmethylene group as illustrated.

In specific embodiments of Formula IIA, the R₁—COOH precursor is selected from R1-4-R1-16 of Scheme 5A, or R1-17 or R1-18 or R1-19-R1-22 of Scheme 5B. In specific embodiments of Formula I or IIA, the R₁—COOH precursor is selected from R1-23-R1-50 of Schemes 5B-5D.

In more specific embodiments, the invention provides compounds of Formula IIIA:

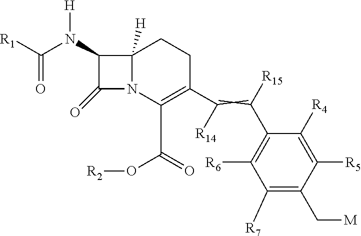

or salts or solvates thereof,
wherein:
R₁-R₇ is as defined in Formula I, IA or IIA, or any embodiment thereof, and M is a leaving group comprising a vicinal diol or an unsubstituted catechol or substituted catechol group. More specifically, M is as defined in Formula I, IA or IIA or any embodiments thereof.

In more specific embodiments, the invention provides compounds of Formula IIIB:

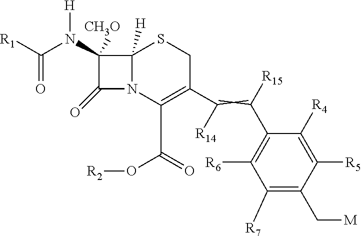

or salts or solvates thereof,
wherein:
R₁-R₇ is as defined in Formula I or IIA, or any embodiment, thereof and M is a leaving group comprising a vicinal diol or an unsubstituted catechol or substituted catechol group. More specifically, M is as defined in Formula I and any embodiments thereof.

In more specific embodiments, the invention provides compounds of Formula IIIC:

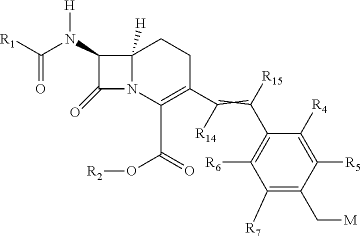

or salts or solvates thereof,
wherein:
R₁-R₇ is as defined in Formula I or IIA, or any embodiment, thereof and M is a leaving group comprising a vicinal diol or an unsubstituted catechol or substituted catechol group. More specifically, M is as defined in Formula I and any embodiments thereof.

In more specific embodiments, the invention provides compounds of Formula IIID:

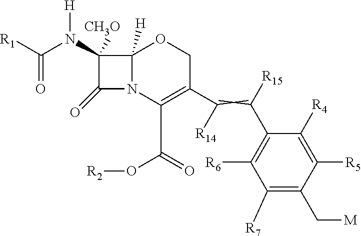

In specific embodiments of Formulas IIIA, IIIB, IIIC or IIID:

R₁₄ and R₁₅ are both hydrogen; and/or
R₂ is hydrogen or R₂ is a pharmaceutically acceptable cation; and/or
R₄-R₇ are all hydrogens or one of R₄-R₇ is selected from halogen, hydroxy, cyano, nitro, or C1-C3 alky and the rest of R₄-R₇ are hydrogens.

In specific embodiments of Formulas I, IA, IIA or IIIA-IIID, M groups are positively charged and may be in the form of a salt or zwitterionic species. In specific embodiments of Formulas I, IA, IIA or IIIA-IIID, M groups contain a positively charge N atom, e.g., the M group is a positively charged amine which may be a straight-chain, branched or cyclic amine. In specific embodiments of Formulas I, IA, IIA or IIIA-IIID, M groups are positive charged heterocyclic groups having at least one heterocyclic ring with at least one N in the ring. In specific embodiments of Formulas I, IA, IIA or IIIA-IIID, M groups are positive charged heterocyclic groups having at least one heterocyclic ring with at least one N and at least one additional heteroatom (N, O or S) in the ring. In specific embodiments of Formulas I, IA, IIA or IIIA-IIID, the positively charged nitrogen of the M group is bonded to the methylene of the styrylmethylene group as illustrated in the formulas.

In specific embodiments, compounds of Formulas I, IA, IIA or IIIA-IIID have a cyclic quaternary ammonium group in M. In specific embodiments, compounds of Formulas I, IA, IIA or IIIA-IIID have isoquinoline diol or quinolone diol groups in M. Exemplary M groups having a positively charged N are illustrated in Scheme 7 and the compounds of Scheme 8. M in each of Formulas I, IA, IIA, or IIIA-IIID can be any one of these specific M groups.

Exemplary R₁—COOH compounds (R₁ precursors) which are useful for introduction of R₁ groups into the compounds of the invention are illustrated in Schemes 5A-5D. In specific embodiments of Formulas I, IA, IIA, or IIIA-IIID, R₁—COOH precursor is selected from R1-4-R1-16 of Scheme 5A, or R1-17 or R1-18 or R1-19-R1-22 of Scheme 5B. In specific embodiments of Formula II, R₁—COOH precursor is selected from R1-23-R1-50 of Schemes 5B-5D.

In specific embodiments of Formulas IIIA-IIID, R₁ is selected from R1-1, R1-2 or R1- as illustrated above. In specific embodiments of Formulas IIIA-IIID, M is M_{X}. In specific embodiments of Formulas IIIA-IIID, M is one of the M groups illustrate in Scheme 8. In specific embodiments of Formulas IIIA-IIID, M is M_{X} and R₁ is selected from R1-1, R1-2 or R1-3. In specific embodiments of Formulas IIIA-IIID, M is one of the M groups illustrated in Scheme 7 or the compounds of Scheme 8 and R₁ is selected from R1-1, R1-2 or R1-3.

In more specific embodiments, the invention provides compounds of Formula IVA:

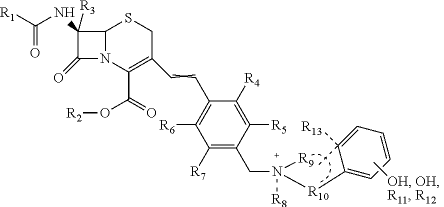

or salts or solvates thereof,
wherein R₁-R₇ and R₁₁-R₁₃ are as defined for Formulas I, IA, IIA, IIIA-IIID or any embodiment thereof and
R₈ is hydrogen or unsubstituted or substituted C1-C6 alkyl;
R₉ is selected from hydrogen, unsubstituted or substituted C1-C6 alkyl;
R₁₀ is a divalent —(CH₂)ₙ— moiety, where n is 1-6 wherein one or two CH₂ groups are replaced with —O—, —S—, —CO—, —N(RN)CO—, or —CON(RN)—, where RN is hydrogen or a C1-C3 alkyl;
or R₉ and R₁₀ together with the nitrogen to which they are attached form a 5- or 6-member heterocyclic ring as shown by the dotted line, which ring is partially or fully unsaturated, unsubstituted or substituted, and optionally contains one or two additional heteroatoms: O, S, N, NR_{N} or a combination thereof in the ring, where RN is hydrogen or a C1-C3 alkyl;
where dotted lines indicate optional bonds;
where the phenyl ring at the right of the formula is substituted with two hydroxyl groups on adjacent ring carbons and is bonded to the positively charged N through the R₁₀ moiety or is fused to the 5- or 6-member ring formed by R₉ and R₁₀.

In specific embodiments, the compounds of Formula IVA are cephalosporins. In specific embodiment, the compounds of Formula IVA are cephamycins. In specific embodiments, compounds of Formula IVA have a cyclic quaternary ammonium group. In specific embodiments, compounds of Formula IVA have isoquinoline diol or quinolone diol groups.

Exemplary M groups containing a positively charged N are illustrated in Scheme 7 and in the compounds of Scheme 8.

Exemplary R₁—COOH compounds (R₁ precursors) which are useful for introduction of R₁ groups into the compounds of the invention are illustrated in Schemes 5A-5D. In specific embodiments of Formula III, R₁—COOH precursor is selected from R1-4-R1-16 of Scheme 5A, or R1-17 or R1-18 or R1-19-R1-22 of Scheme 5B. In specific embodiments of Formula III, R₁—COOH precursor is selected from R1-23-R1-50 of Schemes 5B-5D.

In specific embodiments of Formula IVA, R₁ is selected from R1-1, R1-2 or R1- as illustrated above. In specific embodiments of Formula IVA, M is M_{X}. In specific embodiments of Formula IVA, M is one of the M groups illustrated in Scheme 7 or in Scheme 8. In specific embodiments of Formula IVA, M is M_{X} and R₁ is selected from R1-1, R1-2 or R1-3. In specific embodiments of Formula IVA, M is one of the M groups illustrated in Scheme 7 or Scheme 8 and R₁ is selected from R1-1, R1-2 or R1-3.

In specific embodiments of Formulas I, IA, IIA, IIIA-IIID or IVA, R₄-R₇ are hydrogen. In specific embodiments of Formulas I, IA, IIA, IIIA-IIID or IVA, R₅-R₇ are hydrogen. In specific embodiments of Formulas I, IA, IIA, IIIA-IIID or IVA, R₄ is a halogen, a cyano group or a nitro group. In specific embodiments of Formulas I, IA, IIA, IIIA-IIID or IVA, R₄ is a nitro group.

In embodiments, compounds of the invention are those of Formula VA:

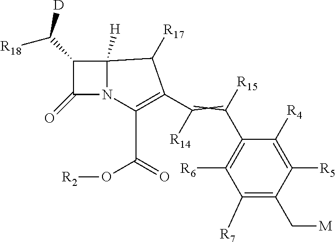

or salts or solvates thereof,
where variables are as defined in Formula I and R₁₈ is an alkyl group and D is hydroxyl, halogen or a protected OH group. In specific embodiments, R₁₄ and R₁₅ are both hydrogen. More specifically, R₁₇ is a C1-C3 alkyl. More specifically, R₁₈ is a C1-C2 alkyl. Preferred D is OH, preferred R₁₇ is methyl and preferred R₁₈ is methyl.

In embodiments, compounds of the invention are those of Formula VB:

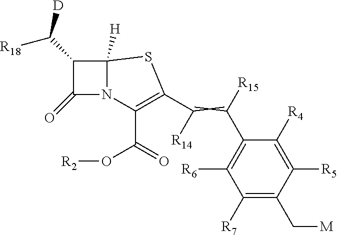

or salts or solvates thereof,
where variables are as defined in Formula I and R₁₈ is an alkyl group and D is hydroxyl, halogen or a protected OH group. More specifically, R₁₈ is a C1-C2 alkyl. In specific embodiments, R₁₄ and R₁₅ are both hydrogen. Preferred D is OH and preferred R₁₈ is methyl.

In embodiments, compounds of the invention are those of Formula VC:

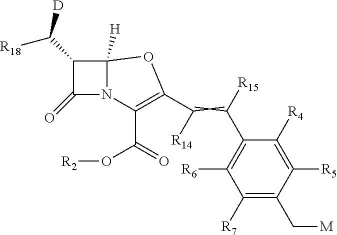

or salts or solvates thereof,
where variables are as defined in Formula I and R₁₈ is an alkyl group and D is hydroxyl, halogen or a protected OH group. More specifically, R₁₈ is a C1-C2 alkyl. In specific embodiments, R₁₄ and R₁₅ are both hydrogen. Preferred D is OH and preferred R₁₈ is methyl.

In specific embodiments of Formulas VA-VC:

all of R₄-R₇ are hydrogens or one of R₄-R₇ is hydroxy, halogen, nitro, cyano or methyl and the remaining R₄-R₇ are hydrogen; and/or
R₂ is hydrogen or when —CO₂ is negatively charged, R₂ is a pharmaceutically acceptable cation; and/or
R₁₈ is C1-C6 alkyl or C1-C3 alkyl or C1-C2 alkyl; and/or
D is OH or halogen or more specifically fluorine.

In specific embodiments of Formulas VA-VC, M is a leaving group comprising a vicinal diol or an unsubstituted catechol or substituted catechol group. More specifically, M is as defined in Formula IIIA. In specific embodiments of compounds of Formula VA, M has a cyclic quaternary ammonium group. In specific embodiments of compounds of Formula VA, M has a isoquinoline diol or quinolone diol groups.

In specific embodiments of Formulas VA-VC, M is M_{X}. In specific embodiments of Formulas VA-VC, M is one of the M groups illustrated in Scheme 7 or in compounds of Scheme 8. In specific embodiments of Formula VA-VC, M is M_{X} and D is OH or halogen or more specifically fluorine. In specific embodiments of Formulas VA-VC, M is one of the M groups illustrated in Scheme 7 or in the compounds of Scheme 8 and D is OH or halogen or more specifically fluorine. In specific embodiments of Formula VA-VC, M is M_{X}, D is OH or halogen or more specifically fluorine and R₁₈ is C1-C2 alkyl or more specifically is methyl. In specific embodiments of Formulas VA-VC, M is one of the M groups illustrated in Scheme 7 or in the compounds of Scheme 8, D is OH or halogen or more specifically fluorine and R₁₈ is C1-C2 alkyl or more specifically is methyl.

In specific embodiments of Formulas VA-VC, R₄-R₇ are hydrogen. In specific embodiments of Formulas VA-VC, R₅-R₇ are hydrogen. In specific embodiments of Formulas VA-VC, R₄ is a halogen, a cyano group or a nitro group. In specific embodiments of Formulas VA-VC, R₄ is a nitro group.

In embodiments, M in Formulas VA-VC is M_{X}:

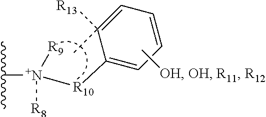

where:
R₈ is hydrogen or unsubstituted or substituted C1-C6 alkyl;
R₉ is selected from hydrogen, unsubstituted or substituted C1-C6 alkyl;
R₁₀ is a divalent —(CH₂)ₙ— moiety, where n is 1-6 wherein one or two CH₂ groups are replaced with —O—, —S—, —CO—, —N(RN)CO—, or —CON(RN)—, where RN is hydrogen or a C1-C3 alkyl;
or R₉ and R₁₀ together with the nitrogen to which they are attached form a 5- or 6-member heterocyclic ring as shown by the dotted line, which ring is partially or fully unsaturated, unsubstituted or substituted, and optionally contains one or two additional heteroatoms: O, S, N, NR_{N} or a combination thereof in the ring, where RN is hydrogen or a C1-C3 alkyl;
where R₁₁ and R₁₂ are independently selected from hydrogen, hydroxyl, halogen, carboxyl or ester thereof, acyl, optionally substituted aryl, optionally substituted aryl alkyl, C1-C3 alkyl, C1-C3 alkoxy, cyano, and nitro groups or, if R₁₁ and R₁₂ are substituted on adjacent ring carbons, R₁₁ and R₁₂ together with the carbons to which they are attached form an optionally substituted 5- or 6-member carbocyclic, or heterocyclic ring, which may be saturated, partially unsaturated or aromatic;
R₁₃, if present, is selected from hydrogen, hydroxyl, halogen, C1-C3 alkyl, cyano, and nitro groups, or, if R₁₁ or R₁₂ is substituted on a ring carbon adjacent to R₁₃, R₁₃ and R₁₁ or R₁₂ together with the carbons to which they are attached form an optionally substituted 5- or 6-member carbocyclic, or heterocyclic ring which may be saturated, partially unsaturated or aromatic;
where dotted lines indicate optional bonds;
where the phenyl ring at the right of the formula is substituted with two hydroxyl groups on adjacent ring carbons and is bonded to the positively charged N through the R₁₀ moiety or is fused to the 5- or 6-member ring formed by R₉ and R₁₀.

In specific embodiments of the compounds of Formulas I, IA, IIA, IIIA-IIID, IVA, and VA-VC, M is selected from:

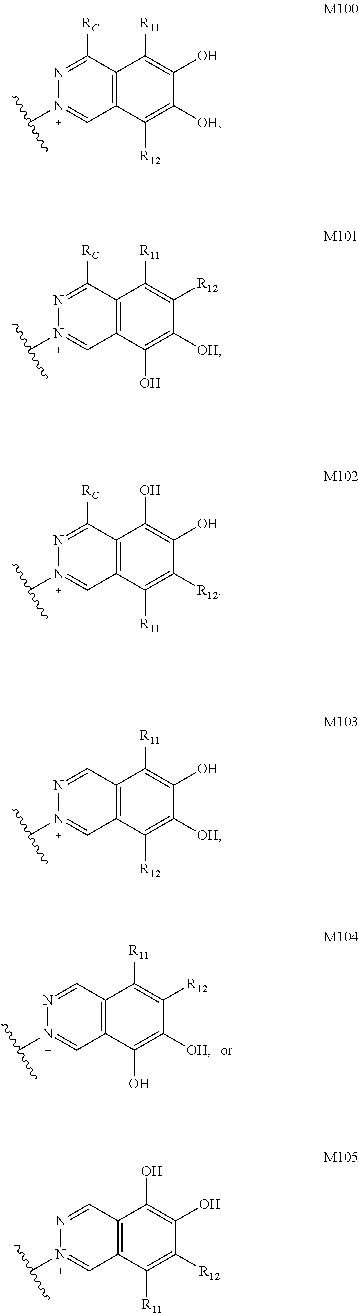

where:

R_{C} is selected from hydrogen, OH, halogen, C1-C3 alkyl, C1-C3 alkoxy, and carboxylate or an ester thereof;

R₁₁ and R₁₂ are selected from hydrogen, OH, halogen, C1-C3 alkyl, C1-C3 alkoxy, carboxylate or an ester thereof or R₁₁ and R₁₂ together with the carbons to which they are attached form a fused phenyl ring which is optionally substituted with one or more, OH, halogen, C1-C3 alkyl, C1-C3 alkoxy, carboxylate or an ester thereof.

In specific embodiments of M100-M105, R_{C} is OH, Cl or methyl. In specific embodiments of M100-M105, R₁₁ and R₁₂ are OH, Cl, —COOH, —COOCH₃ or methyl. In specific embodiments of M100-M105, R₁₁ and R₁₂ are both halogens and specifically both Cl.

In specific embodiments of the compounds of Formulas I, IA, IIA, IIIA-IIID, IVA, and VA-VC, M is selected from:

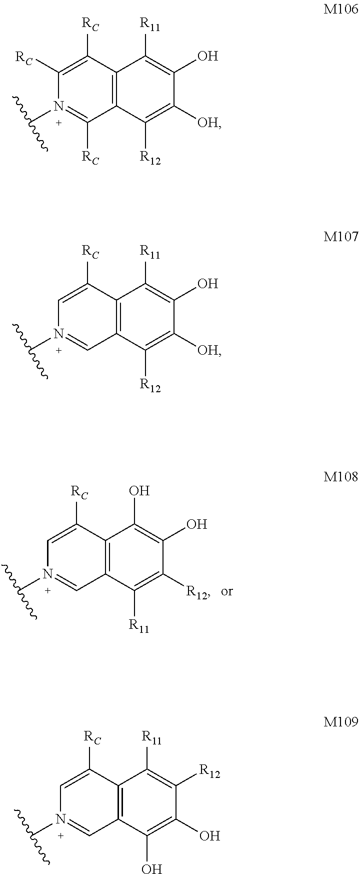

where:

each R_{C} is independently selected from hydrogen, OH, halogen, C1-C3 alkyl, C1-C3 alkoxy, and carboxylate or an ester thereof;

R₁₁ and R₁₂ are independently selected from hydrogen, OH, halogen, C1-C3 alkyl, C1-C3 alkoxy, carboxylate or an ester thereof or R₁₁ and R₁₂, when substituted on adjacent ring carbons, together with the carbons to which they are attached form a fused phenyl ring which is optionally substituted with one or more, OH, halogen, C1-C3 alkyl, C1-C3 alkoxy, carboxylate or an ester thereof.

In specific embodiments of M106-M109, R_{C} is OH, Cl or methyl. In specific embodiments of M106-M109, R_{C} is hydrogen. In specific embodiments of M106-M109, R₁₁ and R₁₂ are hydrogen, OH, Cl, —COOH, —COOCH₃ or methyl. In specific embodiments of M106-M109, R₁₁ and R₁₂ are both halogens and specifically are both C1. In specific embodiments of M106-M109, R_{C} is hydrogen and R₁₁ and R₁₂ are independently, hydrogen, OH, Cl, —COOH, —COOCH₃ or methyl. In specific embodiments of M106-M109, R₁₁ and R₁₂ are both hydrogens. In specific embodiments of M106-M109, R₁₁ and R₁₂ are both hydrogens or are both chlorines and R_{C} is hydroxy.

In specific embodiments of the compounds of Formulas I, IA, IIA, IIIA-IIID, IVA, and VA-VC, M is selected from:

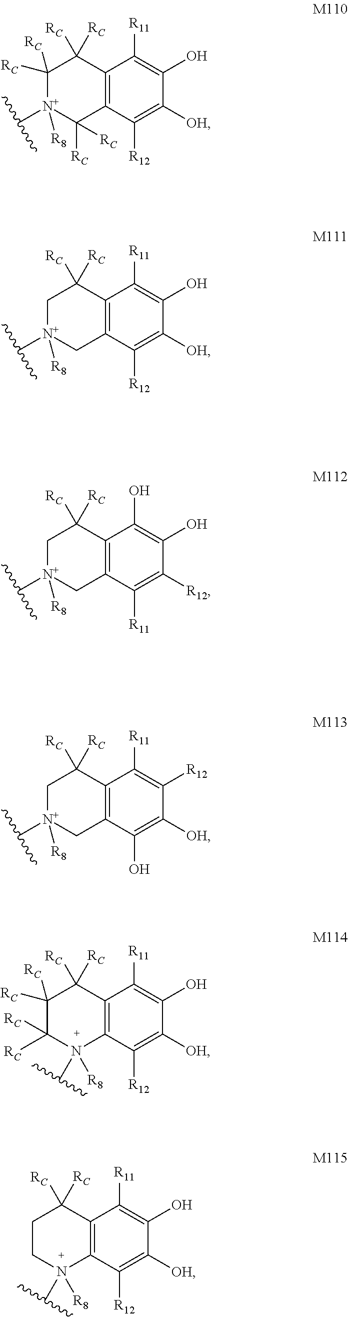

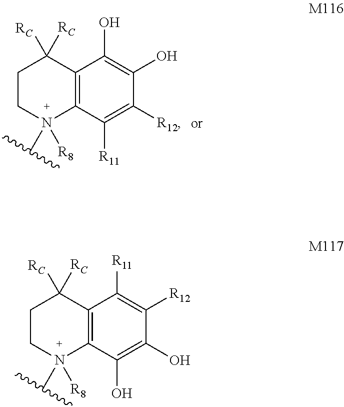

where:

R₈ is a C1-C6 alkyl or a C3-C6 cycloalkyl;

each R_{C} is independently selected from hydrogen, OH, halogen, C1-C3 alkyl, C1-C3 alkoxy, and carboxylate or an ester thereof;

R₁₁ and R₁₂ are independently selected from hydrogen, OH, halogen, C1-C3 alkyl, C1-C3 alkoxy, carboxylate or an ester thereof or R₁₁ and R₁₂, when R₁₁ and R₁₂ are substituted on adjacent ring carbons, together with the carbons to which they are attached form a fused phenyl ring which is optionally substituted with one or more, OH, halogen, C1-C3 alkyl, C1-C3 alkoxy, carboxylate or an ester thereof. More specifically, the fused ring, if present, is substituted with one or two hydroxy, and when two hydroxys, preferably the hydroxys are vicinal hydroxys.

In specific embodiments of M110-M117, R₈ is methyl. In specific embodiments of M110-M117, each R_{C} is independently hydrogen, OH, Cl or methyl. In specific embodiments of M110-M117, each R_{C} is hydrogen. In specific embodiments of M111-M117, one R_{C} is hydrogen and the other is OH, Cl or methyl. In specific embodiments of M110-M117, R₁₁ and R₁₂ are independently hydrogen, OH, Cl, —COOH, —COOCH₃ or methyl. In specific embodiments of M110-M117, R₁₁ and R₁₂ are both halogens and specifically are both Cl. In specific embodiments of M110-M113, R₁₁ and R₁₂ are both hydrogen. In specific embodiments of M110-M117, R_{C} is hydrogen and R₁₁ and R₁₂ are independently, hydrogen, OH, Cl, —COOH, —COOCH₃ or methyl. In specific embodiments of M110-M117, R₈ is methyl, R_{C} is hydrogen and R₁₁ and R₁₂ are independently, hydrogen OH, Cl, —COOH, —COOCH₃ or methyl.

In specific embodiments of the compounds of Formulas I, IA, IIA, IIIA-IIID, IVA, and VA-VC, M is selected from:

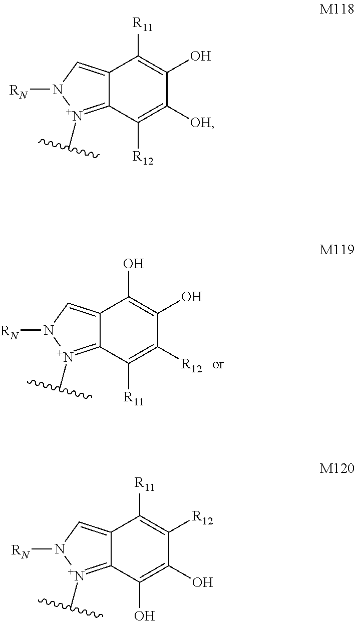

RN is selected from hydrogen, or C1-C3 alkyl;

R₁₁ and R₁₂ are independently selected from hydrogen, OH, halogen, C1-C3 alkyl, C1-C3 alkoxy, carboxylate or an ester thereof or R₁₁ and R₁₂, when on adjacent ring carbons, together with the carbons to which they are attached form a fused phenyl ring which is optionally substituted with one or more, OH, halogen, C1-C3 alkyl, C1-C3 alkoxy, carboxylate or an ester thereof.

In specific embodiments of M118-M120, RN is hydrogen. In specific embodiments of M114-M116, RN is methyl. In specific embodiments of M118-M120, R₁₁ and R₁₂ are independently hydrogen, OH, Cl, —COOH, —COOCH₃ or methyl. In specific embodiments of M118-M120, R₁₁ and R₁₂ are both halogens and specifically are both Cl. In specific embodiments of M118-M120, both R₁₁ and R₁₂ are hydrogen. In specific embodiments of M118-M120, RN is methyl, R₁₁ and R₁₂ are independently, hydrogen, OH, Cl, —COOH, —COOCH₃ or methyl. In specific embodiments of M118-M120, RN is hydrogen, R₁₁ and R₁₂ are independently, hydrogen, OH, Cl, —COOH, —COOCH₃ or methyl.

In specific embodiments of the compounds of Formulas I, IA, IIA, IIIA-IIID, IVA, and VA-VC, M is selected from:

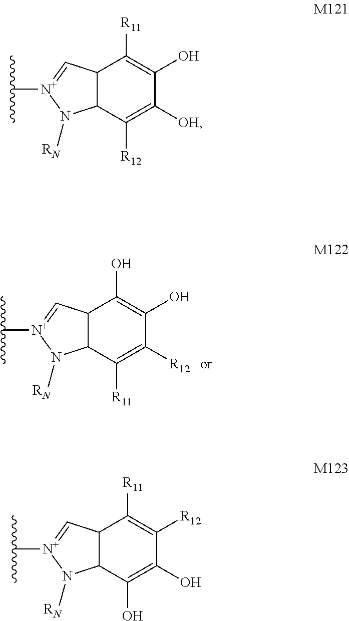

where

R_{N} is selected from hydrogen, or C1-C3 alkyl;

R₁₁ and R₁₂ are independently selected from hydrogen, OH, halogen, C1-C3 alkyl, C1-C3 alkoxy, carboxylate or an ester thereof or R₁₁ and R₁₂, when on adjacent ring carbons, together with the carbons to which they are attached form a fused phenyl ring which is optionally substituted with one or more, OH, halogen, C1-C3 alkyl, C1-C3 alkoxy, carboxylate or an ester thereof.

In specific embodiments of M117-M119, R_{N} is hydrogen. In specific embodiments of M117-M119, R_{N} is methyl. In specific embodiments of M121-M123, R₁₁ and R₁₂ are independently hydrogen, OH, Cl, —COOH, —COOCH₃ or methyl. In specific embodiments of M121-M123, R₁₁ and R₁₂ are both halogens and specifically are both Cl. In specific embodiments of M121-M123, both R₁₁ and R₁₂ are hydrogen. In specific embodiments of M121-M123, R_{N} is methyl, R₁₁ and R₁₂ are independently, hydrogen, OH, Cl, —COOH, —COOCH₃ or methyl. In specific embodiments of M121-M123, R_{N} is hydrogen, R₁₁ and R₁₂ are independently, hydrogen, OH, Cl, —COOH, —COOCH₃ or methyl.

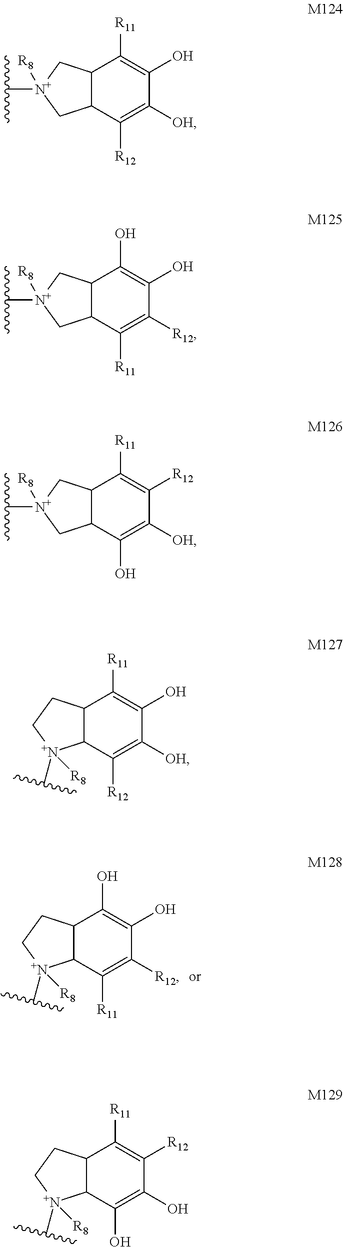

where:

R₈ is a C1-C6 alkyl or a C3-C6 cycloalkyl;

R₁₁ and R₁₂ are independently selected from hydrogen, OH, halogen, C1-C3 alkyl, C1-C3 alkoxy, carboxylate or an ester thereof or R₁₁ and R₁₂, when substituted on adjacent ring carbons, together with the carbons to which they are attached form a fused phenyl ring which is optionally substituted with one or more, OH, halogen, C1-C3 alkyl, C1-C3 alkoxy, carboxylate or an ester thereof. More specifically, the fused ring if present is substituted with one or two hydroxyl groups, when two hydroxy groups are present they are preferably vicinal hydroxyl groups.

In specific embodiments of M124-M129, R₈ is methyl. In specific embodiments of M124-M129, R₁₁ and R₁₂ are independently hydrogen, OH, Cl, —COOH, —COOCH₃ or methyl. In specific embodiments of M124-M129, R₁₁ and R₁₂ are both halogens and specifically are both Cl. In specific embodiments of M124-M129, R₁₁ and R₁₂ are both hydrogen. In specific embodiments of M124-M129, R₈ is methyl and R₁₁ and R₁₂ are independently, OH, Cl, —COOH, —COOCH₃ or methyl. In specific embodiments of M124-M129, R₈ is methyl, and R₁₁ and R₁₂ are independently, hydrogen, OH, Cl, —COOH, —COOCH₃ or methyl.

In specific embodiments of the compounds of Formulas I, IA, IIA, IIIA-IIID, IVA, and VA-VC, M is selected from:

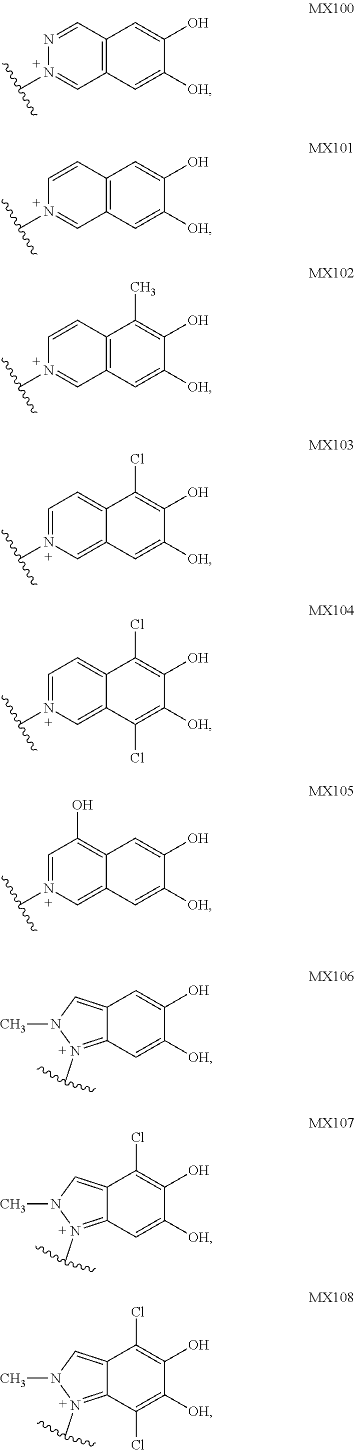

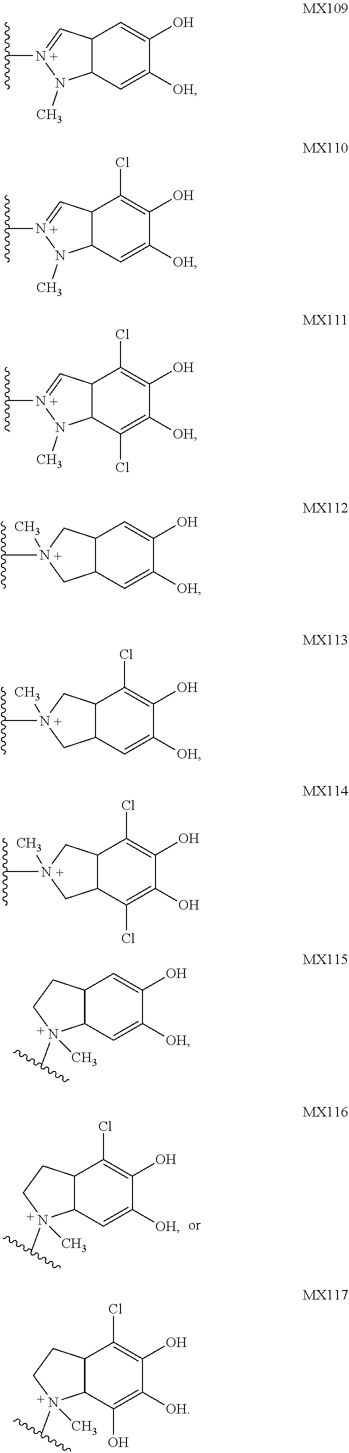

In embodiments, of Formulas I, IA, IIA, IIIA-IIID, IVA, or VA-VC, M is MX100, MX101, MX106, MX109, MX112, or MX115. In embodiments, of Formulas I, IA, IIA, IIIA-IIID, IVA, or VA-VC, M is MX103, MX107, MX110, MX113, or MX116. In embodiments, of Formulas I, IA, IIA, IIIA-IIID, IVA, or VA-VC, M is MX104, MX108, MX111, MX114, or MX117. In embodiments, of Formulas I, IA, IIA, IIIA-IIID, IVA, or VA-VC, M is MX100, MX102, or MX10. In embodiments, of Formulas I, IA, IIA, IIIA-IIID, IVA, or VA-VC, M is MX101-MX105. In embodiments, of Formulas I, IA, IIA, IIIA-IIID, IVA, or VA-VC, M is MX106-MX108. In embodiments, of Formulas I, IA, IIA, IIIA-IIID, IVA, or VA-VC, M is MX109-MX111. In embodiments, of Formulas I, IA, IIA, IIIA-IIID, IVA, or VA-VC, M is MX112-MX114. In embodiments, of Formulas I, IA, IIA, IIIA-IIID, IVA, or VA-VC, M is MX115-MX117.

In specific embodiments of Formulas I, IA, IIA, IIIA-IIID, IVA, or VA-VC, the compound is the E isomer (at the styryl group).

In specific embodiments of Formulas I, IA, IIA, IIIA-IIID, IVA, VA-VC, the compound is the Z isomer (at the styryl group).

In more specific embodiments, compounds of the invention include those of Formulas:

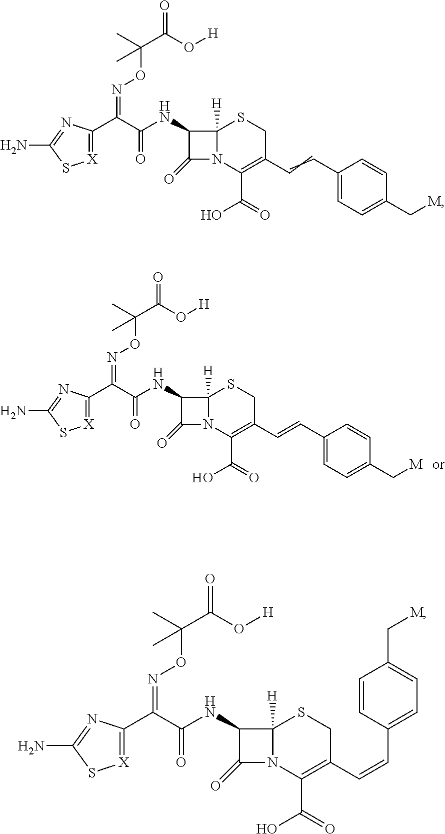

where X is N or CH and M is selected any one of the M groups illustrated in Scheme 7 or in the compounds of Scheme 8. In specific embodiments, M for the above formulas M is selected from:

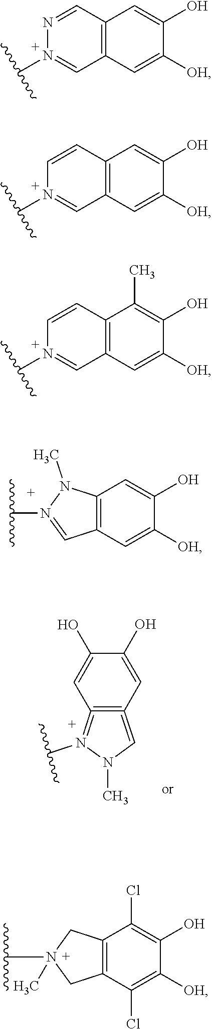

where each cation is paired with an appropriate cation, which is optionally a pharmaceutically acceptable cation. In more specific embodiments, the cation is a halide, an acetate, a trifluoroacetate, a sulfate or a bisulfate.

In specific embodiments, the cephem compounds are of Formula VI:

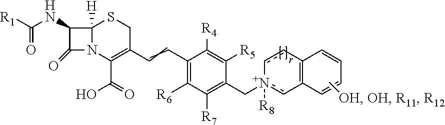

or a salt or solvate thereof
wherein:
R₁, R₄-R₇, R₈ (if present), R₁₁ and R₁₂ are as defined for Formula I, IA or IIA and any embodiment thereof and r is 0 or 1 (representing a 5 or 6-member ring and dashed lines indicate bonds that may be present or absent). In specific embodiments, R₈ is present and is a C1-C6 alkyl group or a C1-C3 alkyl group, or a C3-C6 cycloalkyl group. In specific embodiments r is 0. In specific embodiments, r is 1. In specific embodiments, r is 1 and the six-member ring is aromatic. In specific embodiments, r is 1 and the six-member ring contains no double bonds (is saturated) or one double bond (is partially unsaturated). In specific embodiments, r is 1 and the six-member ring contains no double bonds.

In specific embodiments, the cephem compounds are of Formula VIA:

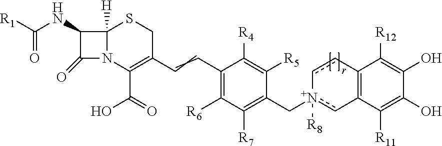

or a salt or solvate thereof
wherein:
R₁, R₄-R₇, R₈ (if present), R₁₁ and R₁₂ are as defined for Formula I or any embodiment thereof and r is 0 or 1 (representing a 5- or 6-member ring and dashed lines indicate bonds that may be present or absent. In specific embodiments, R₈ is present and is a C1-C6 alkyl group or a C1-C3 alkyl group, or a C3-C6 cycloalkyl group. In specific embodiments r is 0. In specific embodiments, r is 1. In specific embodiments, r is 1 and the six-member ring is aromatic. In specific embodiments, r is 1 and the six-member ring contains no double bonds or one double bond. In specific embodiments, r is 1 and the six-member ring contains no double bonds.

In specific embodiments, the cephem compounds are of Formula VIB:

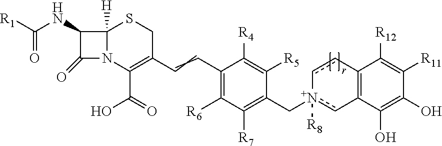

or a salt or solvate thereof
wherein:
R₁, R₄-R₇, R₈ (if present), R₁₁ and R₁₂ are as defined for Formula I or any embodiment thereof and r is 0 or 1 (representing a 5- or 6-member ring and dashed lines indicate bonds that may be present or absent. In specific embodiments, R₈ is present and is a C1-C6 alkyl group or a C1-C3 alkyl group, or a C3-C6 cycloalkyl group. In specific embodiments r is 0. In specific embodiments, r is 1. In specific embodiments, r is 1 and the six-member ring is aromatic. In specific embodiments, r is 1 and the six-member ring contains no double bonds or one double bond. In specific embodiments, r is 1 and the six-member ring contains no double bonds.

For each of Formulas VI and VIA-VIB, the nitrogen cation can be paired with an appropriate anion, which is optionally a pharmaceutically acceptable anion. In more specific embodiments, the cation is a halide, an acetate, a trifluoroacetate, a sulfate or a bisulfate.

In specific embodiments the cephem compounds are of Formula VII:

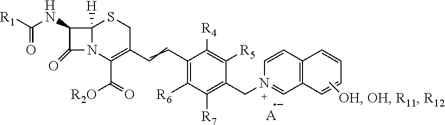

or a salt or solvate thereof, where variables are as defined for Formula I or any embodiment thereof.

In specific embodiments, the cephem compounds are of Formula VIIA:

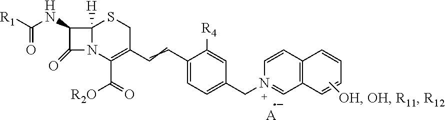

or a salt or solvate thereof, where variables are as defined for Formula I or IA or any embodiment thereof. More specifically R₄ is hydroxy, C1-C3 alkyl, halogen, hydrogen, CN or NO₂. More specifically R₄ is halogen, hydroxyl, methyl or nitro. More specifically, R₄ is hydrogen.

In specific embodiments, the cephem compounds are of Formula VIIB:

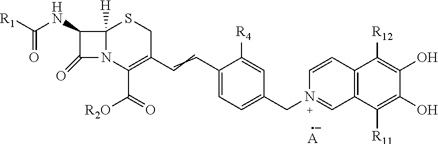

or a salt or solvate thereof, where variables are as defined for Formula I or IA or any embodiment thereof. More specifically R₄ is hydroxy, C1-C3 alkyl, halogen, hydrogen, CN or NO₂. More specifically R₄ is halogen, hydroxyl, methyl or nitro. More specifically, R₄ is hydrogen. More specifically, R₁₁ and R₁₂ are both hydrogen or one of R₁₁ or R₁₂ is hydrogen and the other is halogen or one or R₁₁ or R₁₂ is chlorine and the other of R₁₁ or R₁₂ is hydrogen or both R₁₁ and R₁₂ are halogens, or both of R₁₁ and R₁₂ are chlorines.

In specific embodiments, the cephem compounds are of Formula VIIC:

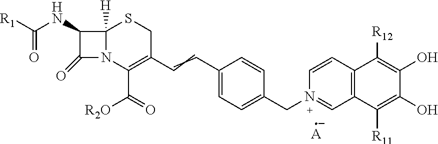

or a salt or solvate thereof, where variables are as defined for Formula I. More specifically R₁₁ and R₁₂ are hydrogen, or halogen. More specifically, R₁₁ and R₁₂ are both hydrogen or one of R₁₁ or R₁₂ is hydrogen and the other is halogen or one or R₁₁ or R₁₂ is chlorine and the other of R₁₁ or R₁₂ is hydrogen or both R₁₁ and R₁₂ are halogens, or both of R₁₁ and R₁₂ are chlorines.

In specific embodiments, the cephem compounds are of Formula VIID:

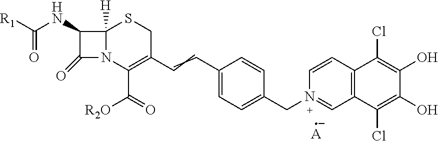

or a salt or solvate thereof, where variables are as defined for Formula I.

In specific embodiments, the cephem compounds are of Formula VIIE:

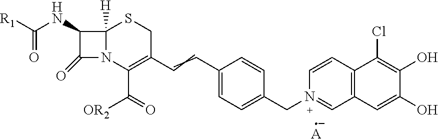

or a salt or solvate thereof, where variables are as defined for Formula I or any embodiments thereof.

In specific embodiments, the cephem compounds are of Formula VIIF:

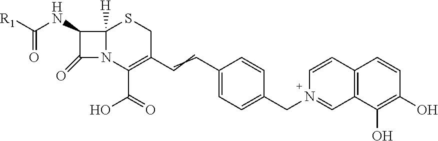

or a salt or solvate thereof, where variables are as defined for Formula I or any embodiments thereof.

In specific embodiments, the cephem compounds are of Formula VIIG:

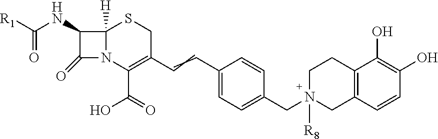

or a salt or solvate thereof, where variables are as defined for Formula I or any embodiment thereof. In specific embodiments, R₈ is a C1-C6 alkyl group. In specific embodiments, R₈ is a C3-C6 cycloalkyl group. In specific embodiments, R₈ is a C1-C3 alkyl group.

In Formulas VI, VIA, VIB, and VIIA-VIIG, the double bond indicated by the crossed double bond is in the E configuration. In Formulas VI, VIA, VIB, and VIIA-VIIG, the double bond indicated by the crossed double bond is in the Z configuration.

In specific embodiments, of Formulas VI, VIA, VIB, and VIIA-VIIG, R₁ is R1-1, R1-2 or R1-3.

In specific embodiments of Formulas VI, VIA, VIB, and VIIA-VIIG, R₁ has Formula R1-2 and more specifically in R1-2, Ra and Rb are both alkyl groups. More specifically Ra and Rb are both methyl groups. More specifically Re is hydrogen. More specifically X is CH.

In specific embodiments of Formulas VI, VIA, VIB, and VIIA-VIIG, R₁ is unsubstituted or substituted benzyl.

In specific embodiments of Formulas herein, the cationic leaving group can be paired with an appropriate anion, A⁻. In specific embodiments, the anion A⁻ is a pharmaceutically acceptable anion. In specific embodiments, the anion A⁻ is a halide, more specifically a chloride, a sulfate or bisulfate, an acetate, or trifluoroacetate.

In specific embodiments, cephem compounds are of Formulas:

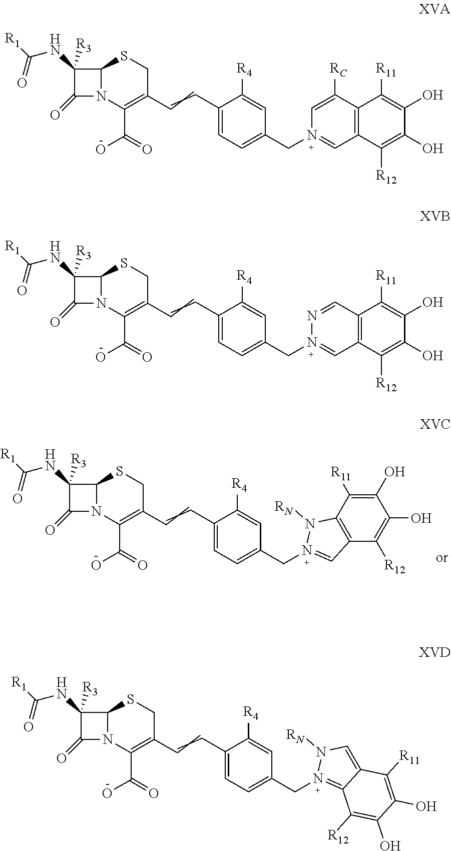

where:
R₁—CO—NH— is an acyl amino group;
R₃ is hydrogen or methoxy;
R₄ is hydrogen, hydroxyl, halogen, nitro, or cyano;
R_{N} is hydrogen or a C1-C3 alkyl;
R_{C} is hydrogen, hydroxyl, halogen or C1-C3 alkyl; and
R₁₁ and R₁₂, independently, are hydrogen, halogen, hydroxyl, C1-C3 alkyl, C1-C3 alkoxy, carboxylate or ester thereof.

In specific embodiments, penems of the invention have Formulas:

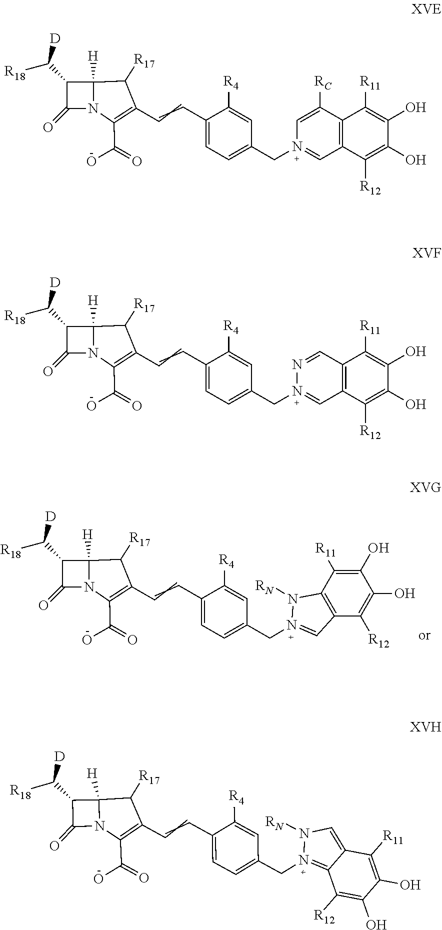

where:
D is hydroxyl, protected hydroxyl or halogen;
R₁₈ is C1-C3 alkyl;
R₁₇ is hydrogen or C1-C3 alkyl;
R₄ is hydrogen, hydroxyl, halogen, nitro, or cyano;
R_{N} is hydrogen or a C1-C3 alkyl;
R_{C} is hydrogen, hydroxyl, halogen or C1-C3 alkyl; and
R₁₁ and R₁₂, independently, are hydrogen, halogen, hydroxyl, C1-C3 alkyl, C1-C3 alkoxy, carboxylate or ester thereof.

Formulas XVA-XVD are shown in a zwitterionic form. It will be appreciated that other salt forms of the compounds of these formulas can be readily made. The carboxylate anion of these formulas can alternatively be paired with an appropriate pharmaceutically acceptable cation or can be in the form of —COOR₂, where R₂ is hydrogen or an organic group, such as an alkyl, arylalkyl or aryl group. The positively charged nitrogen of the formula may be paired with an appropriate pharmaceutically acceptable anion. Compounds of Formulas XVA-XVD may be in the E conformation at the styryl double bond or in the Z conformation at this double bond or the compounds may have a mixture of conformations at this double bond.

In specific embodiments of Formulas XVA-XVD:

R₄ is hydrogen,
R₄ is nitro;
R₃ is hydrogen;
R₃ is methoxy;
R₄ is hydrogen and R₃ is hydrogen;
R_{N} is methyl;
R₄ is hydrogen, R₃ is hydrogen and R_{N} is methyl;
R₁₁ and R₁₂ are both hydrogen,
One of R₁₁ or R₁₂ is halogen and the other is hydrogen;
One of R₁₁ or R₁₂ is chlorine and the other is hydrogen;
Both of R₁₁ and R₁₂ are halogen;
Both of R₁₁ and R₁₂ are chlorine;
One of R₁₁ or R₁₂ is methyl and the other is hydrogen;
R_{C} is hydrogen;
R_{C} is hydroxyl;
R_{C} is hydroxyl and one or both of R₁₁ and R₁₂ are hydrogen;
R_{C} is hydroxyl and one or both of R₁₁ and R₁₂ are halogen;

### R₁ is R1-1, R1-2 or R1-3;

R₃, and R₄ are hydrogen and R₁ is R1-1, R1-2 or R1-3;
R₃, R₄, R_{C}, R₁₁ and R₁₂ are all hydrogen and R_{N} is methyl;
R₃, R₄, R₁₁ and R₁₂ are all hydrogen, R_{C} is hydroxyl and R_{N} is methyl;
R₄, R₁₁ and R₁₂ are all hydrogen, R_{N} is methyl and R₁ is R1-1, R1-2 or R1-3; or
R₃, R₄, R₁₁ and R₁₂ are all hydrogen, R_{C} is hydroxyl, R_{N} is methyl and R₁ is R1-1, R1-2 or R1-3.

In specific embodiments of Formulas XVE-XVH:

R₄ is hydrogen,
R₄ is nitro;
R₁₇ is hydrogen;
R₁₇ is methyl;
R₄ is hydrogen and R₁₇ is hydrogen;
R₄ is hydrogen and R₁₇ is methyl;
R_{N} is methyl;
R₄ is hydrogen and R_{N} is methyl;
R₁₁ and R₁₂ are both hydrogen,
One of R₁₁ or R₁₂ is halogen and the other is hydrogen;
One of R₁₁ or R₁₂ is chlorine and the other is hydrogen;
Both of R₁₁ and R₁₂ are halogen;
Both of R₁₁ and R₁₂ are chlorine;
One of R₁₁ or R₁₂ is methyl and the other is hydrogen;
R_{C} is hydrogen;
R_{C} is hydroxyl;
R_{C} is hydroxyl and one or both of R₁₁ and R₁₂ are hydrogen;
R_{C} is hydroxyl and one or both of R₁₁ and R₁₂ are halogen;
D is hydroxy;
D is protected hydroxyl;
D is fluorine;
R₁₈ is methyl;
D is hydroxyl and R₁₈ is methyl;
D is hydroxyl, R₁₈ is methyl, R₁₇ is hydrogen;
D is hydroxyl, R₁₈ is methyl and R₁₇ is methyl;
D is hydroxyl, R₁₈ is methyl; R₁₇ is methyl and R₄ is hydrogen;
R₁₁ and R₁₂ are hydrogen and R_{N} is methyl;
D is hydroxyl, R₁₈ is methyl, R₁₇ is methyl, R₄ is hydrogen and R_{N} is methyl;
D is hydroxyl, R₁₈ is methyl, R₁₇ is methyl, R₄ is hydrogen, and R_{C} is hydroxyl:
D is hydroxyl, R₁₈ is methyl, R₁₇ is methyl, R₄ is hydrogen, R_{C} is hydroxyl and R_{N} is methyl;
R_{N} is methyl and R₁₁ and R₁₂ are both halogen; or
R_{N} is methyl and R₁₁ and R₁₂ are both hydrogen;

In specific embodiments, the cephem compounds are of Formulas:

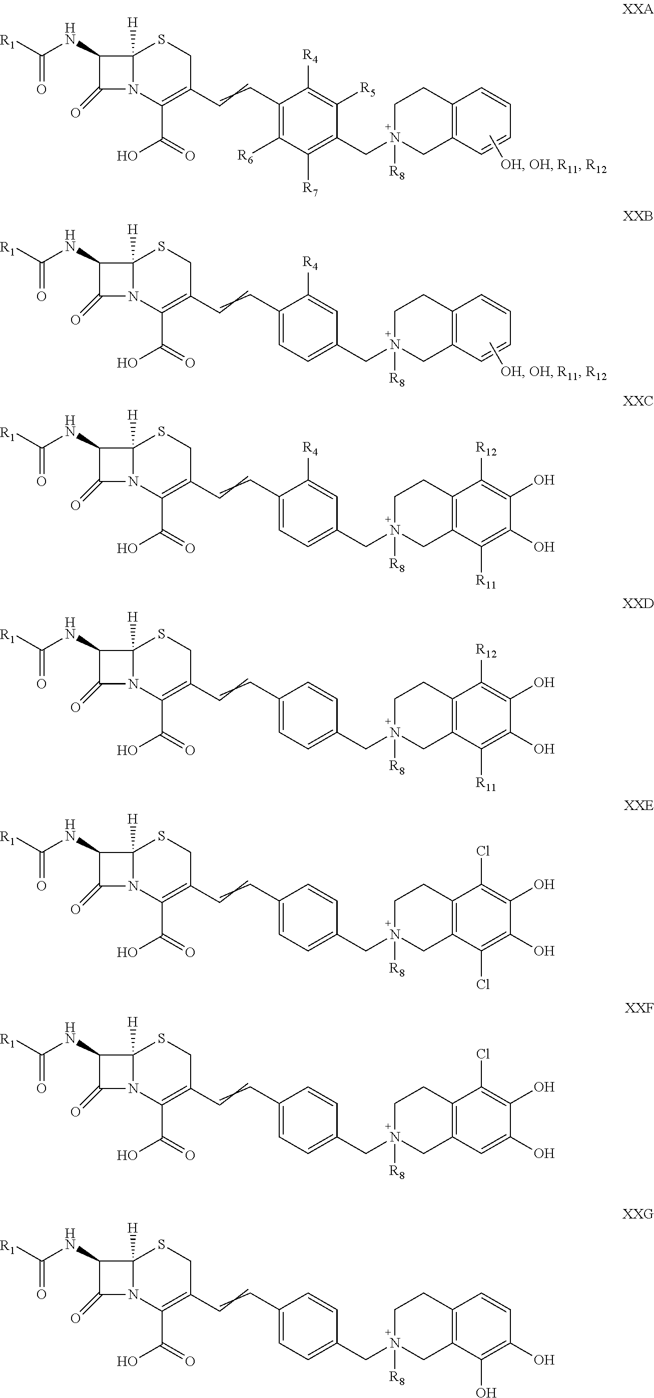

or a salt or solvate thereof, where R₁ is a defined in Formula I and R₈ is a C1-C6 alkyl group or a C1-C3 alkyl group or a C3-C6 cycloalkyl group. In Formulas XXA to XXG, the double bond indicated by the crossed double bond can be in the E configuration. In Formulas XXA to XXG, the double bond indicated by the crossed double bond can be in the Z configuration.
In specific embodiments of Formulas XXA-XXG, R₄ if present is halogen, cyano or nitro.
In specific embodiments of Formulas XXA-XXG, R₈ is a C1-C3 alkyl group or a C3-C6 cycloalkyl group.
In specific embodiments, of Formulas XXA to XXG, R₁ is R1-1, R1-2 or R1-3.
In specific embodiments of Formulas XXA to XXG, R₁ has Formula R1-2 and more specifically in R1-2, Ra and Rb are both alkyl groups. More specifically Ra and Rb are both methyl groups. More specifically Re is hydrogen. More specifically X is CH.
In specific embodiments of Formulas XXA to XXG, R₁ is unsubstituted or substituted benzyl.

In specific embodiments of Formulas XXA to XXG, the indicated cation is paired with an appropriate anion A⁻. In embodiments, A⁻ is a pharmaceutically acceptable anion. In specific embodiments of Formulas XXA to XXG, the anion A⁻ is a halide, more specifically chlorine, sulfate, bisulfate, acetate, or trifluoroacetate.

In specific embodiments, the cephem compounds are of Formulas:

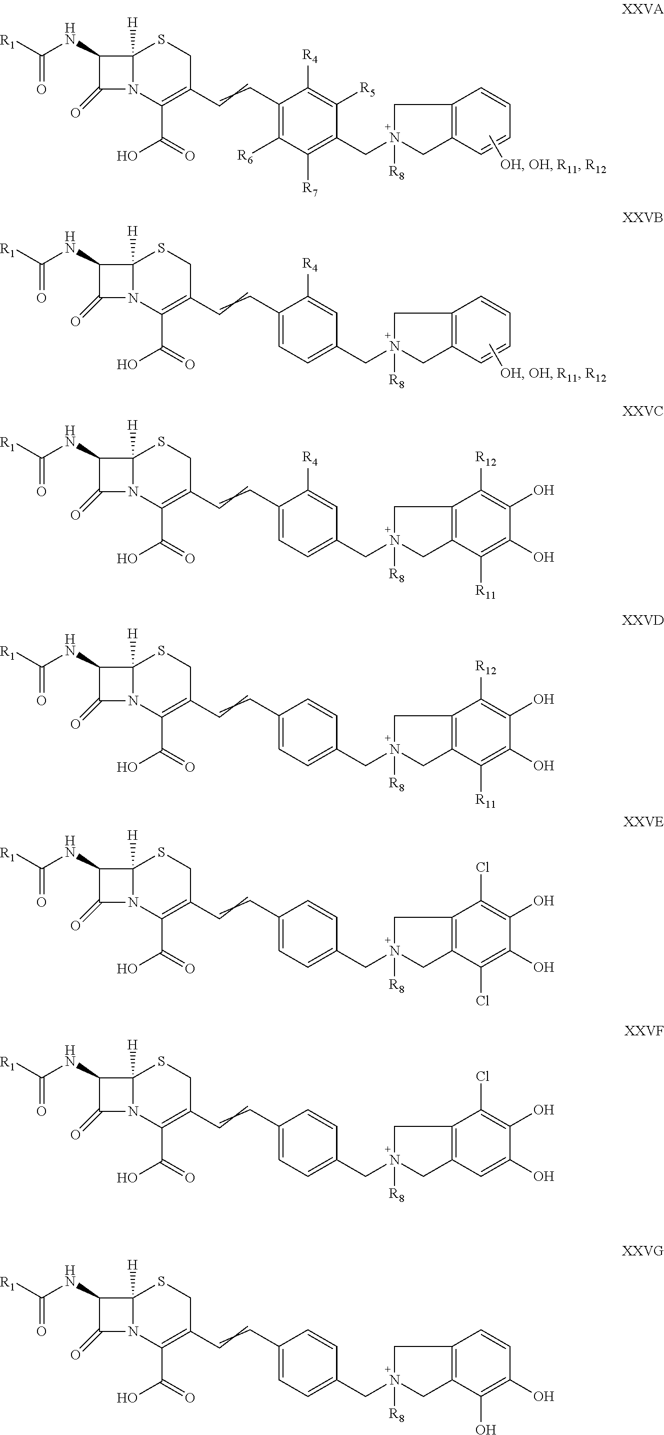

or a salt or solvate thereof, where R₁, R₁₁ and R₁₂ are as defined for Formula I, IA or any embodiment thereof and R₈ is a C1-C6 alkyl or a C3-C6 cycloalkyl.

In Formulas XXVA to XXVG, the double bond indicated by the crossed double bond is in the E configuration. In Formulas XXVA to XXVG, the double bond indicated by the crossed double bond is in the Z configuration. In Formulas XXVA to XXVG, the double bond indicated by the crossed double bond is a mixture of the E and Z configuration.

In specific embodiments of Formulas XXVA to XXVG, R₄, if present, is hydrogen, halogen, cyano or nitro. In specific embodiments, R₈ is a C1-C6 alkyl group, a C1-C3 alkyl group or a C3-C6 cycloalkyl group. In specific embodiments, R₈ is methyl. In specific embodiments R₁₁ and R₁₂ are both hydrogen. In specific embodiments R₁₁ and R₁₂ are selected from hydrogen, methyl, carboxy or an ester thereof, or a halogen. In specific embodiments, one or R₁₁ and R₁₂ is a hydroxy, a carboxy or an ester thereof, a methyl or a halogen and the other is a hydrogen.

In specific embodiments, of Formulas XXVA to XXVG, R₁ is R1-1, R1-2 or R1-3. In specific embodiments of Formulas XXVA to XXVG, R₁ has Formula R1-2 and more specifically in R1-2, Ra and Rb are both alkyl groups. More specifically Ra and Rb are both methyl groups. More specifically Re is hydrogen. More specifically X is CH. In specific embodiments of Formulas XXVA to XXVG, R₁ is unsubstituted or substituted benzyl.

In specific embodiments of Formulas XXVA to XXVG, the indicated cation is paired with an appropriate anion A⁻. In specific embodiments, A⁻ is a pharmaceutically acceptable anion. In specific embodiments of Formulas XXVA to XXVG, the anion A⁻ is a halide, more specifically chlorine, acetate, sulfate, bisulfate or trifluoroacetate.

In specific embodiments of any formula herein, R₄-R₇ are hydrogen. In specific embodiments of any formula herein, R₅-R₇ are hydrogen. In specific embodiments of any formulas herein, R₄ is a halogen, a cyano group or a nitro group. In specific embodiments any formulas herein, R₄ is a nitro group.

In specific embodiments, the compound of the invention is a pharmaceutically acceptable salt of a compound of any formula herein. In specific embodiments, the compound of the invention is a pharmaceutically acceptable solvate of a compound of any formula herein. In specific embodiments, the compound of the invention is a pharmaceutically acceptable hydrate of a compound of any formula herein.

In specific embodiments of any formula herein, M is any one of the M1-M36 groups illustrated in Scheme 7. In specific embodiments of any formula herein, M is any one of the M1-M36 groups illustrated in Scheme 7 and R₁ is an acylamino group. In specific embodiments of any formula herein, M is any one of the M1-M36 groups illustrated in Scheme 7 and R₁ is R1-1, R1-2 or R1-3. In specific embodiments of any formula herein M is any one of the M1-M36 groups illustrated in Scheme 7 and R is R₁₈—C(D)-, where D is OH, halogen or protected OH. In specific embodiments of any formula herein, M is any one of the M1-M36 groups illustrated in Scheme 7 and R is R₁₈—C(D)-, where D is OH, halogen or protected OH and R₁₈ is preferably methyl.

In specific embodiments of any formula herein, M is any one of the M100-M129 groups. In specific embodiments of any formula herein, M is any one of the M100-M129 groups and R₁ is an acylamino group. In specific embodiments of any formula herein, M is any one of the M100-M129 groups and R₁ is R1-1, R1-2 or R1-3. In specific embodiments of any formula herein, M is any one of the M100-M129 groups and R is R₁₈—C(D)-, where D is OH, halogen or protected OH. In specific embodiments of any formula herein, M is any one of the M100-M129 groups and R is R₁₈—C(D)-, where D is OH, halogen or protected OH and R₁₈ is preferably methyl.

In specific embodiments of any formula herein, M is any one of the MX100-MX117 groups. In specific embodiments of any formula herein, M is any one of the MX100-MX117 groups and R₁ is an acylamino group. In specific embodiments of any formula herein, M is any one of the MX100-MX117 groups and R₁ is R1-1, R1-2 or R1-3. In specific embodiments of any formula herein, M is any one of the MX100-MX117 groups and R is R₁₈—C(D)-, where D is OH, halogen or protected OH. In specific embodiments of any formula herein, M is any one of the MX100-MX117 groups and R is R₁₈—C(D)-, where D is OH, halogen or protected OH and R₁₈ is preferably methyl.

### Synthesis of Compounds of Formula I

Cephem compounds of the invention and particularly cephalosporins of the invention are generally synthesized as illustrated for Formula II in Scheme 1 by reaction of the derivatized cephalosporin of formula X with a quaternary amine salt of formula XI therein. The compound of formula X is the E or the Z isomer (at the styrene moiety) or a mixture of E and Z isomers. The compound of Formula II retains the E/Z configuration of the starting compound of Formula X. Thus, isomers of Formula I having the E configuration free of the corresponding Z isomer are prepared employing the corresponding E isomer of the intermediate cephem of Formula X. Isomers of Formula I having the Z configuration free of the corresponding E isomer are prepared employing the corresponding Z isomer of the intermediate cephem of Formula X. In specific embodiments, HBr salts of the amines of Formula XI are employed. It will be appreciated that various amine salts can be employed. The variables of the compounds of Scheme 1 are defined in the descriptions of Formulas I, IA, IIA or any embodiment thereof. The compound of Formula II in the Schemes is shown as an amine salt (with optionally protection at the R₁ and R₂ groups). Formula IIA in Scheme 1, illustrates unprotected compound as an amine salt. When R₂ of Formula I is or contains a carboxylate (—COO⁻), the compound can be a zwitterion (as exemplified in Formula IIB in Scheme 1).

Dependent upon the nature of the R₁ and R₂ groups and the amine with which the cephem intermediates is reacted, potentially reactive moieties of these groups in the compound of Formula X may be protected from undesired reaction with the compound of Formula XI. Protecting groups that may be present in starting materials can be removed as shown in the examples herein or as understood in the art by known methods.

Scheme 2 illustrates the synthesis of the intermediate compound of Formula X. The carboxylic acid precursor of the 7-amido group (which is optionally protected) of Formula XII is converted to an acyl chloride XIII. The acyl chloride is reacted with the 7-amino compound XIV to form intermediate X. Variables in Scheme 2 are as defined in Formula I, IA and any embodiment thereof. PR₁ and PR₂ indicate optionally protected R₁ and R₂ groups, where R₁ and R₂ are as defined in Formula I, IA or any embodiment thereof.

Scheme 3 illustrates an exemplary synthesis of a more specific intermediate of Formula X where the R₁ group is a substituted oxime. The precursors to X and compound X itself are optionally protected with carboxyl, hydroxyl or amine protecting groups. One or ordinary skill in the art can readily select appropriate protecting groups useful for synthesis of the compounds of Formula I herein. Variables in Scheme 3 are as defined in Formula I. PR₁ and PR₂ indicate optionally protected R₁ and R₂ groups.

Exemplary R₁ groups include those of formulas R1-1, R1-2 and R1-3.

Penems of the invention can be prepared in view of what is known in the art with respect to synthesis of the various penem rings and in view of the synthetic methods provided herein. For example, U.S. Pat. Nos. 4,465,632; 4,689,292; 4,729,993; 4,978659; 5,334,590; 5,342,933; 5,350,746; 5,382,575; 6,677,331; 6,825,187; 7,468,364; 7,632,828; 8318,716; 9,145,425; and 9,937,151 relate to synthesis of carbapenem compounds. Methods therein can be routinely adapted in view of descriptions herein and what is known in the art for synthesis of carbapenems herein. U.S. Pat. Nos. 4,218,459; 4,298,741; 4,347,355; 5,055,463; 5,138,050 and 5,395,931 relate to synthesis of carbapenem compounds having a 6-amido group. Methods therein can be routinely adapted in view of descriptions herein and what is known in the art for synthesis of carbapenems herein. U.S. Pat. Nos. 5,036,063; 5,116,832; 5,703,068; and 6,271,222 relate to penem compounds, where Z in Formula I is —S—. Methods therein can be routinely adapted in view of descriptions herein and what is known in the art for synthesis of carbapenems herein.

Compounds of the invention of Formulas I and various cephem and penem intermediates useful for their synthesis can be routinely prepared by one of ordinary skill in the art in view of the descriptions herein and specific examples provided or by routine modification of such methods by choice of starting amine (or amine salt) and use of cephem intermediates as described.

U.S. Pat. Nos. 4,840,945; 5,036,034; 7,129,232; and US published application 20050043531 provide additional methods of synthesis of cephalosporin compounds and in particular provide examples of useful acylamino groups. Each of these references is incorporated by reference herein in its entirety for such descriptions.

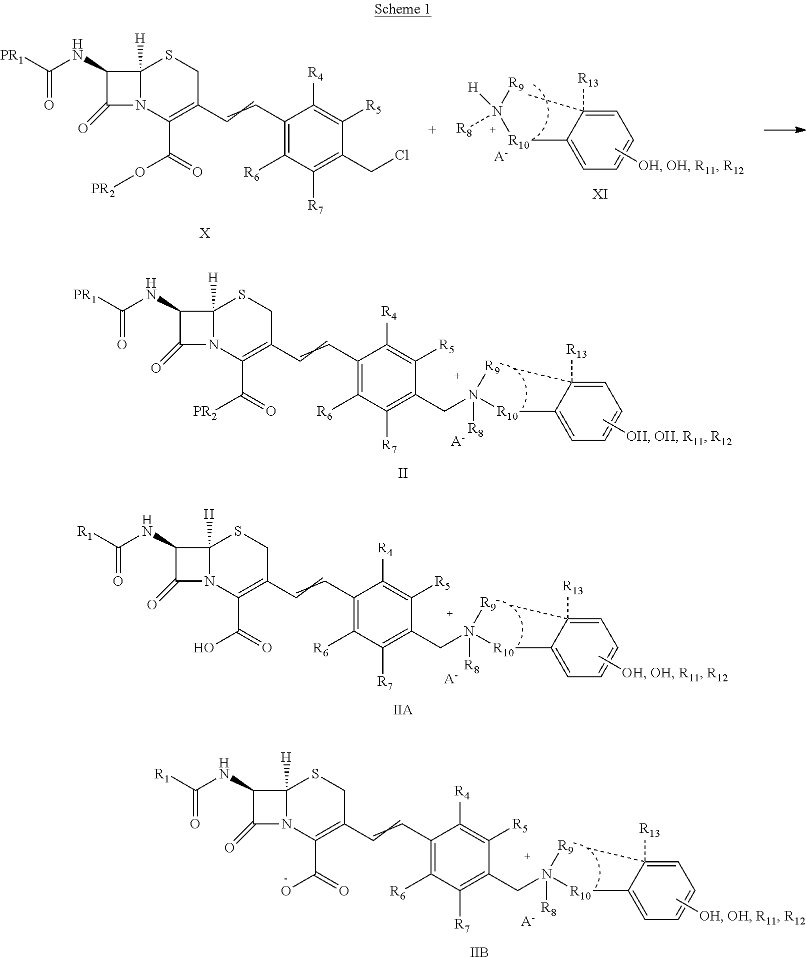

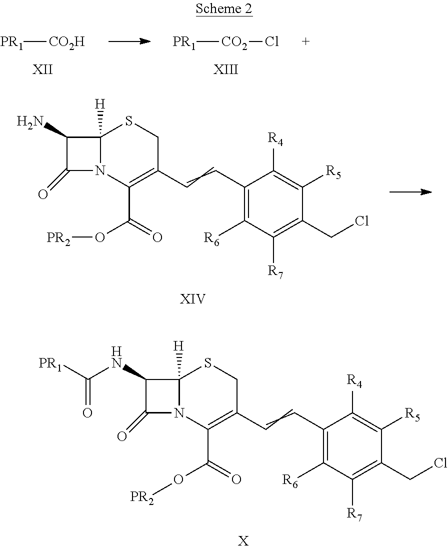

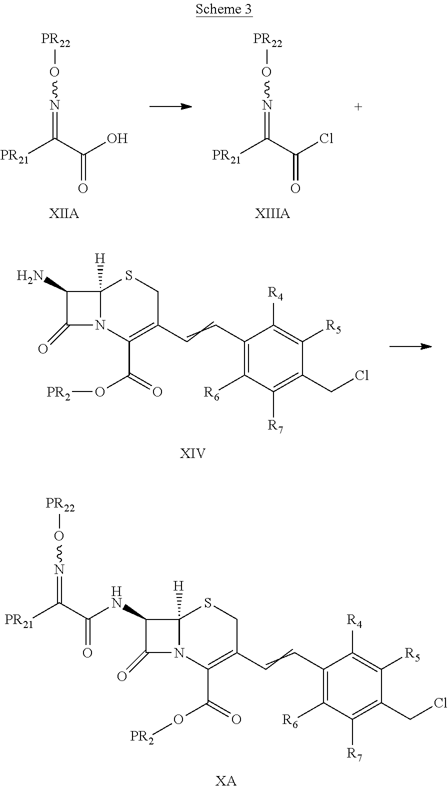

Amine salt XI is prepared from the corresponding amine by methods that are well known in the art and which are exemplified in examples herein. The amines have a catechol moiety, i.e. a phenyl ring with a pair of vicinal diols. The catechol moiety is bonded to the amine. When the amine is a heterocycyl amine, the heterocycle can be fused to the phenyl ring of the catechol. Exemplary amines and salts thereof useful in syntheses of compounds of the invention are shown in Schemes 4A-4C and elsewhere in the Schemes and Examples herein, where variables are as defined in Formula I and IA and any embodiments thereof. In some cases, the amines and salts thereof are commercially available, known in the art, or can be prepared by methods well known in the art. Exemplary methods for the preparation of amines and salts thereof are provided in the Examples.

It will be appreciated that compounds of the invention can be prepared using methods exemplified herein or by routine adaptation of such methods with appropriate choice of starting materials. Methods exemplified herein can be used to prepare various cephem compounds, including cephalosporins, cephamycins and the like. Compounds with various R₁ groups can be prepared from known precursors, such as the carboxylic acid precursors illustrated in Schemes 5A-5D, by know methods. Compounds with various R₂ groups can be prepared from known precursors by known methods. Methods provided herein can be routinely adapted to the synthesis of penems, carbapenems and oxapenems in view of synthetic methods for these compounds that are well-known in the art.

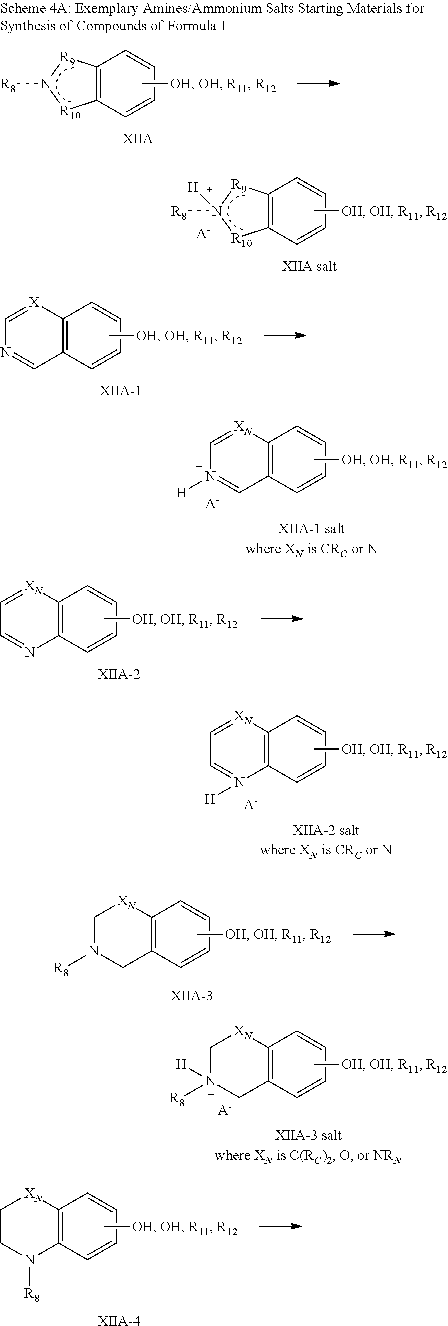

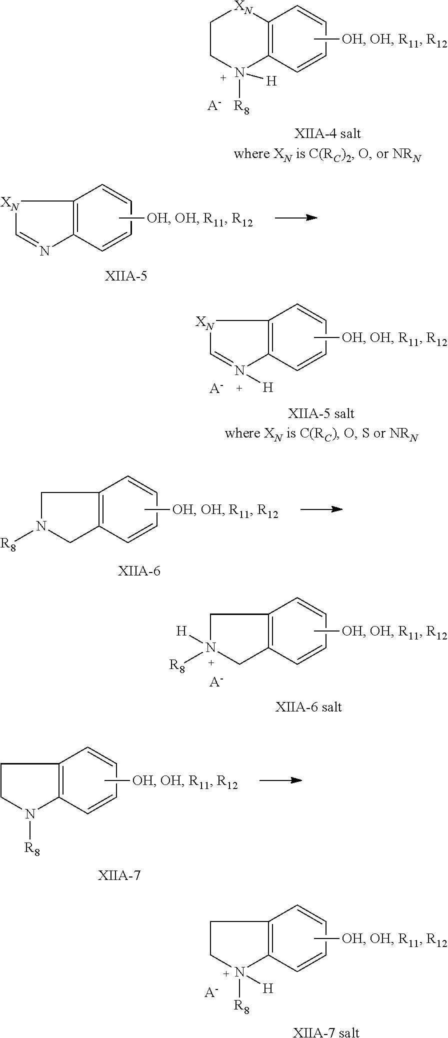

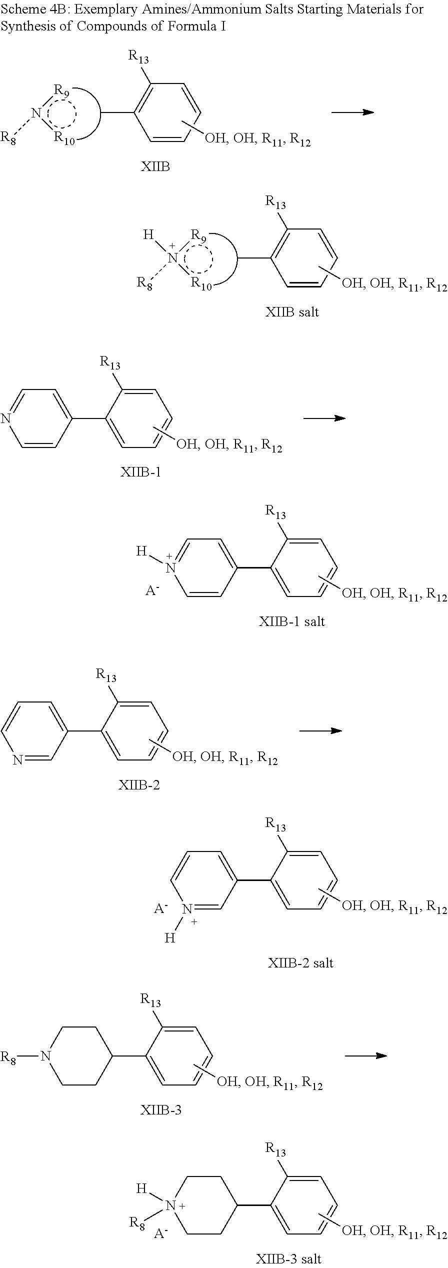

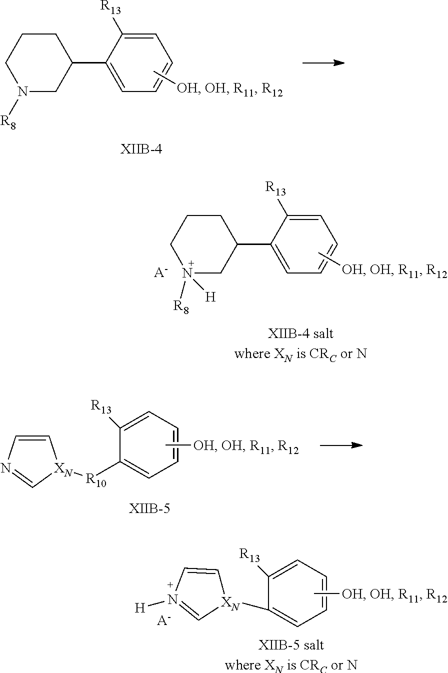

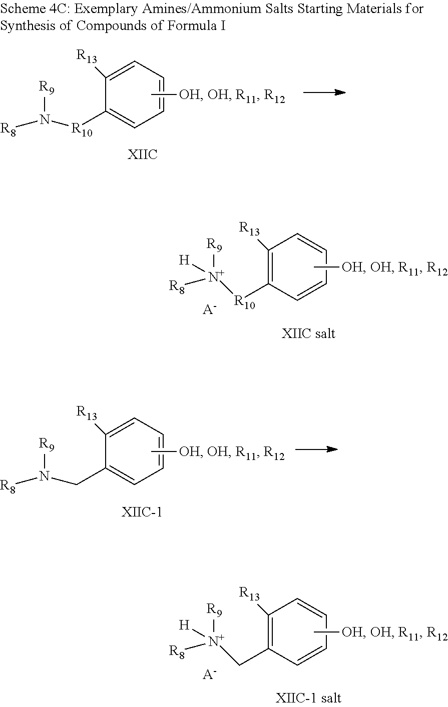

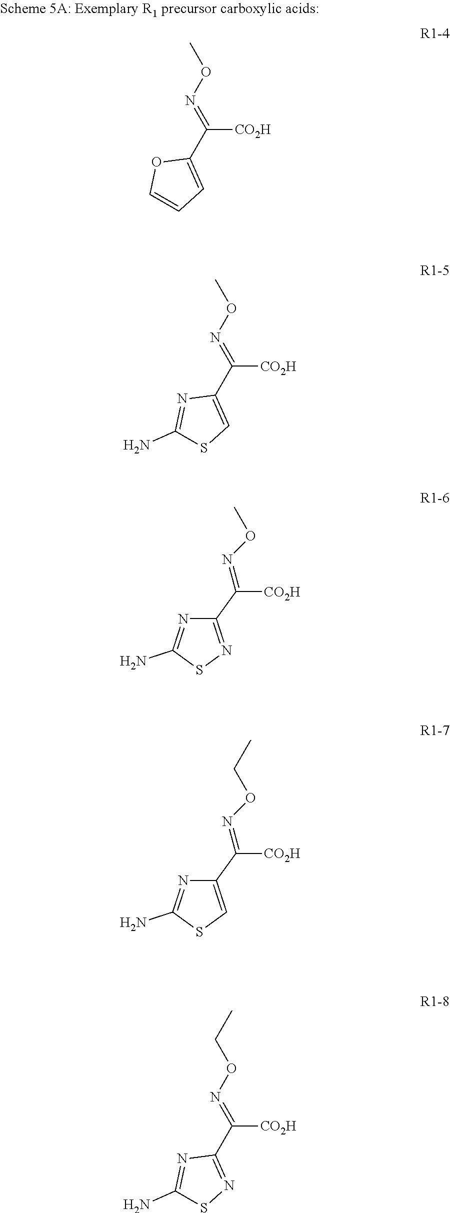

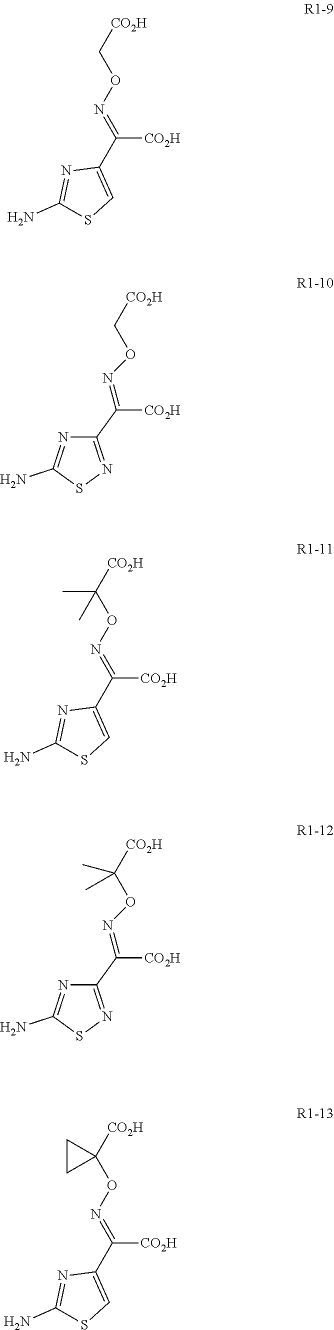 [SMILES: Nc1nc(/C(=N/OCC(=O)O)C(=O)O)cs1 ; Nc1nc(/C(=N/OCC(=O)O)C(=O)O)ns1 ; CC(C)(O/N=C(\C(=O)O)c1csc(N)n1)C(=O)O ; CC(C)(O/N=C(\C(=O)O)c1nsc(N)n1)C(=O)O ; Nc1nc(/C(=N/OC2(C(=O)O)CC2)C(=O)O)cs1]

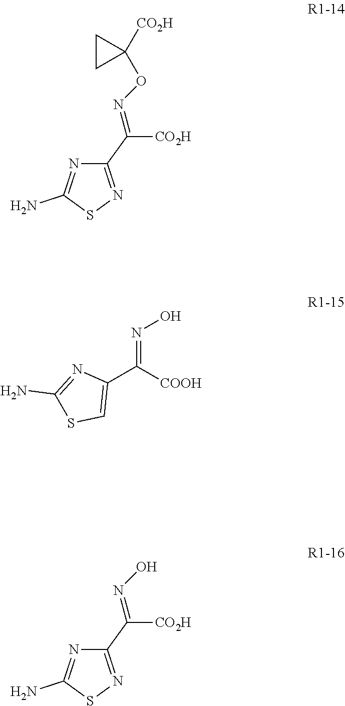 [SMILES: Nc1nc(/C(=N/OC2(C(=O)O)CC2)C(=O)O)ns1 ; Nc1nc(/C(=N/O)C(=O)O)cs1 ; Nc1nc(/C(=N/O)C(=O)O)ns1]

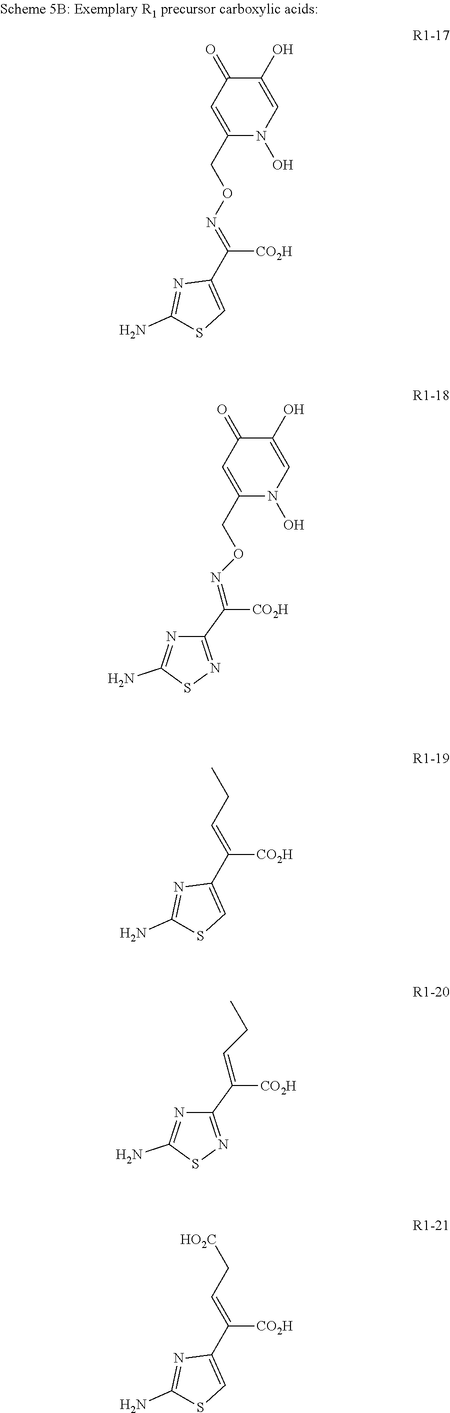 [SMILES: Nc1nc(/C(=N/OCc2cc(=O)c(O)cn2O)C(=O)O)cs1 ; Nc1nc(/C(=N/OCc2cc(=O)c(O)cn2O)C(=O)O)ns1 ; CC/C=C(\C(=O)O)c1csc(N)n1 ; CC/C=C(\C(=O)O)c1nsc(N)n1 ; Nc1nc(/C(=C/CC(=O)O)C(=O)O)cs1]

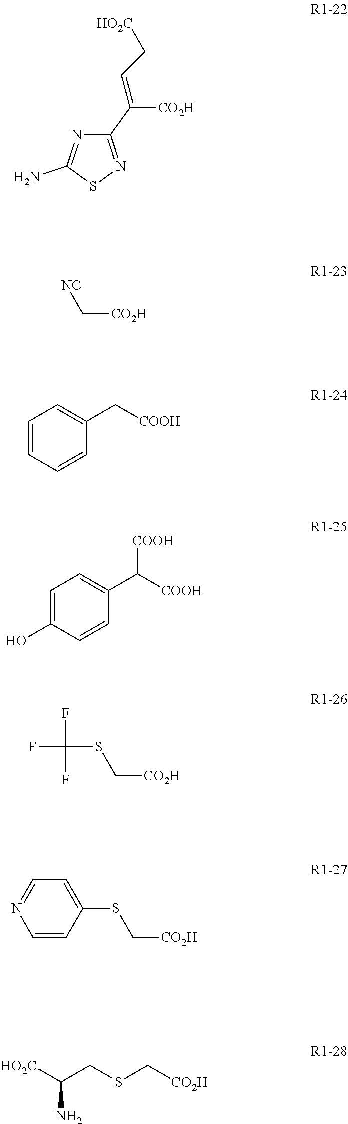

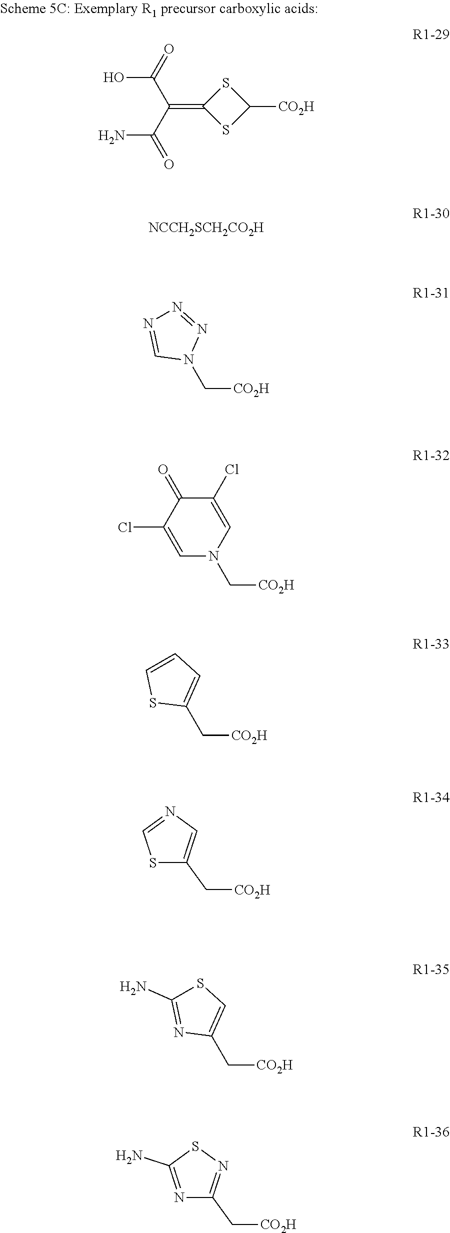

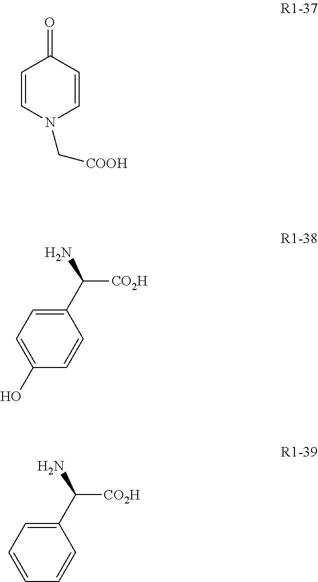 [SMILES: O=C(O)Cn1ccc(=O)cc1 ; N[C@@H](C(=O)O)c1ccc(O)cc1 ; N[C@@H](C(=O)O)c1ccccc1]

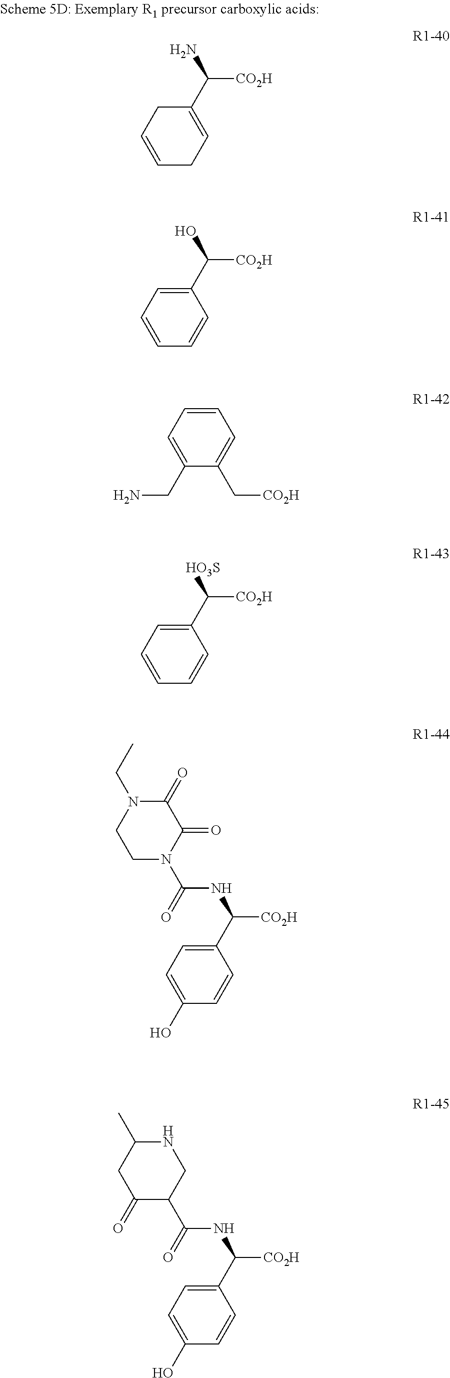 [SMILES: N[C@@H](C(=O)O)C1=CCC=CC1 ; O=C(O)[C@H](O)c1ccccc1 ; NCc1ccccc1CC(=O)O ; O=C(O)[C@@H](c1ccccc1)S(=O)(=O)O ; CCN1CCN(C(=O)N[C@@H](C(=O)O)c2ccc(O)cc2)C(=O)C1=O ; CC1CC(=O)C(C(=O)N[C@@H](C(=O)O)c2ccc(O)cc2)CN1]

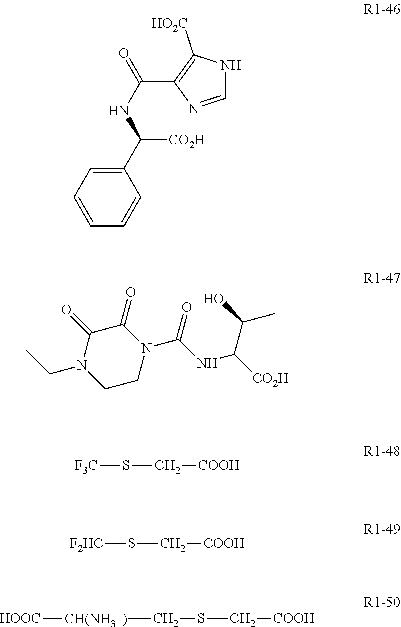

Scheme 6 illustrates more specifically the synthesis of certain compounds herein. In Scheme 6, R₄-R₆, R₁₁ and R₁₂ are as defined in Formulas I, IA and any embodiments thereof. In compounds X10 and X11, R₃₀-R₃₂ are independently selected from hydrogen, halogen, carboxyl and esters thereof, C1-C3 alkyl, C2-C7 alkenyl, C2-C7 alkynyl, optionally substituted phenyl, optionally substituted benzyl, optionally substituted phenyl, alkyl, hydroxyl, nitro, cyano, amino, monoalkylamino, dialkylamino, —COR_{D}, where R_{D} is hydrogen or C1-C3 alkyl. In compounds X12-X15, R₃₀-R₃₅, if present, are independently selected from hydrogen, halogen, hydroxy, carboxyl and esters thereof, C1-C3 alkyl, C2-C7 alkenyl, C2-C7 alkynyl, hydroxyl, nitro, cyano, amino, monoalkylamino, dialkylamino, —COR_{D}, where R_{D} is hydrogen or C1-C3 alkyl. In embodiments, optional substitution for phenyl, benzyl, and phenylalkyl groups includes substitution with one or more hydroxyl, C1-C3 alkyl, C1-C3 alkoxy, and carboxyl or esters thereof. In alternative embodiments, any two adjacent of R₃₀-R₃₂ together form a 5- or 6-member carbocyclic or heterocyclic ring that is saturated, partially unsaturated or aromatic. Reactions A-D are illustrated with 6,7-diols, the corresponding 5,6-diols and 7,8-diols can be prepared by analogous methods with appropriate choice of starting amine salt. Reactions E and F are illustrated with 5, 6-diols, the corresponding 4,5-diols and 6, 7-diols can be prepared by analogous methods with appropriate choice of starting amine salt. Amine salts of particular use are those in which R₁₁ land R₁₂ are independently hydrogen or chloride. The amine salts in Scheme 6 are illustrated as HBr salts. It will be appreciated by one of ordinary skill in the art that amine salts with other anions, e.g., HCl salts, acetate salts, trifluoroacetate salts can be employed in synthetic methods herein.

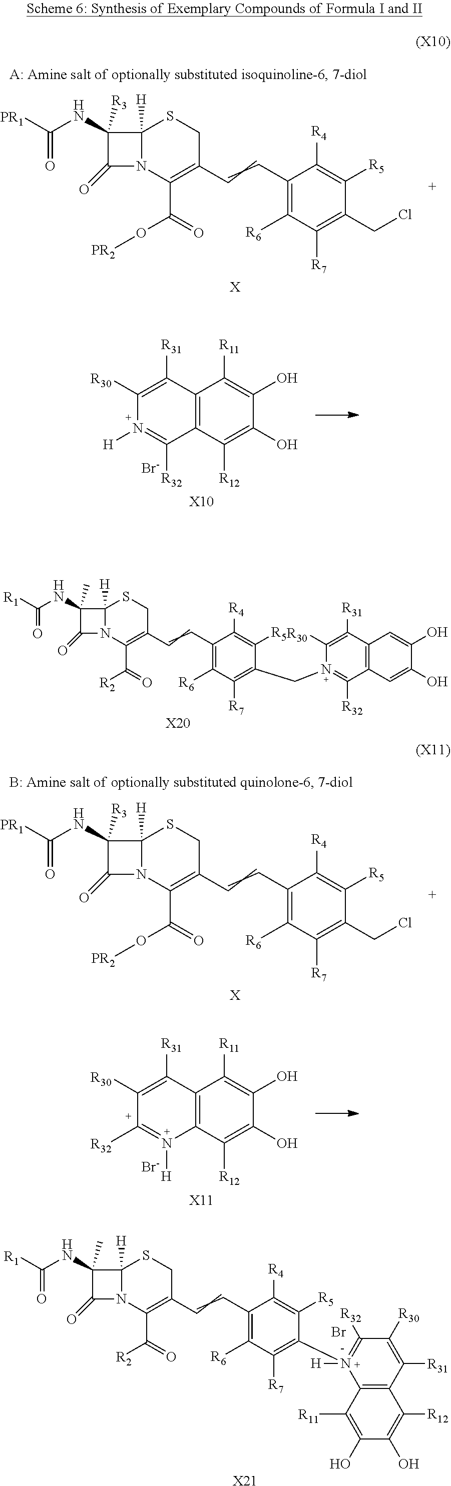

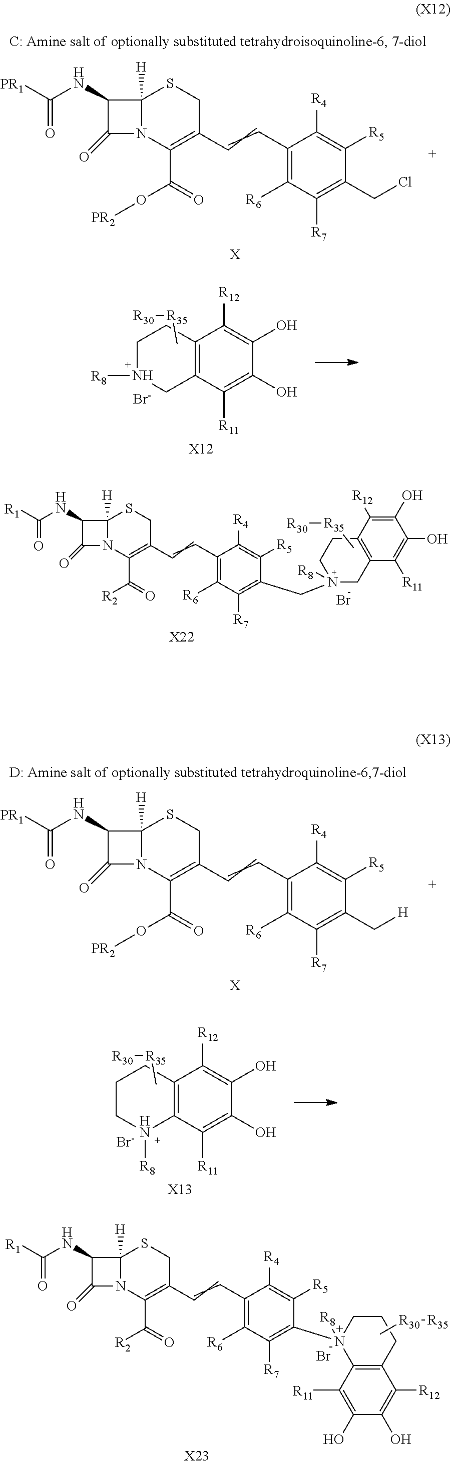

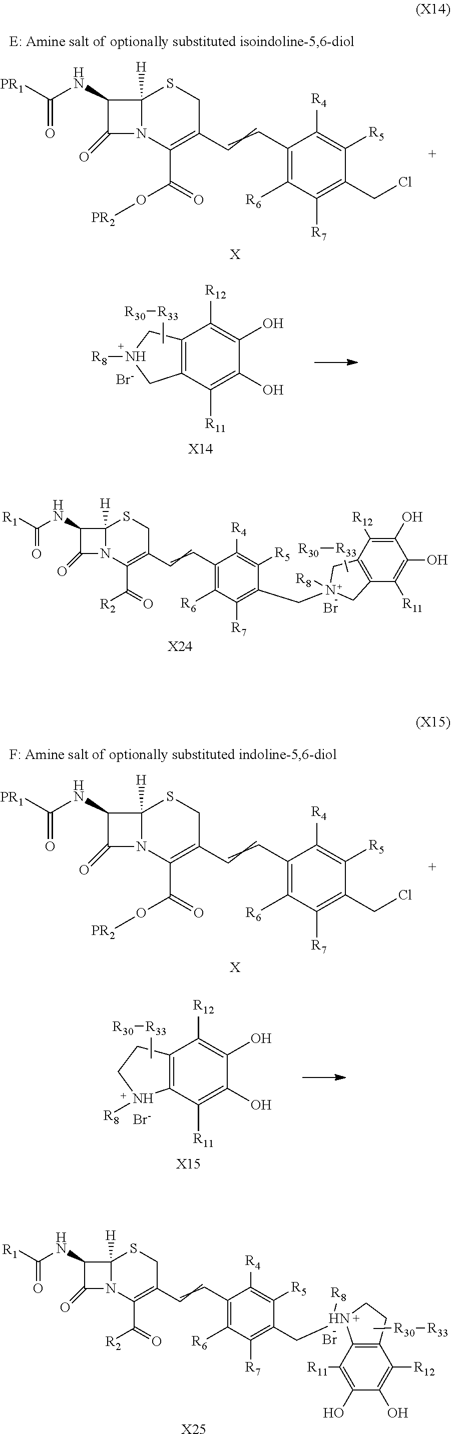

In embodiments, the invention provides intermediates useful at least for synthesis of compounds of cephems of Formula I herein of Formula XXX:

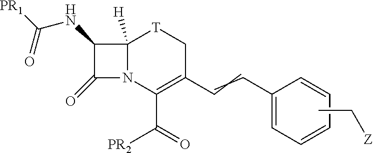

or salts or solvates thereof
where:
T is —S—, —SO—, —SO₂—, —CH₂—, or —O—,
Z is Cl, Br or I, where the —CH₂—Z group is substituted at any carbon in the phenyl ring;
R₁ and R₂ are as defined for Formula I or IA or any embodiment thereof, where the PR₁ and PR₂ indicates that potentially reactive functional groups in the R₁ and R₂ groups are optionally protected as needed to carry out the various syntheses. More specifically in R₁ and R₂ groups one or more NH₂ groups, OH groups, SH groups or COOH groups may be protected. In specific embodiments Z is Cl. In specific embodiments, R₁ is selected from those of the R₁COOH groups illustrated in Schemes 5A-5D. In specific embodiments, PR₂ is a protecting group for a carboxylate. Of particular interest are those compounds of Formula XXX which are E isomers free of Z isomer or those which are Z isomer free of E isomer. In specific embodiments of Formula XXX, T is —S—.

In more specific embodiments, the invention provides intermediates useful at least for synthesis of compounds of cephalosporins of Formula I herein of Formula XXXI:

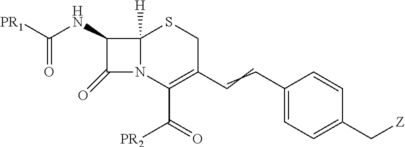

or salts or solvates thereof
where:

### Z is Cl, Br or I,

R₁ and R₂ are as defined for Formula I or IA or any embodiment thereof, where the PR₁ and PR₂ indicates that potentially reactive functional groups in the R₁ and R₂ groups are optionally protected as needed to carry out the various syntheses. More specifically in R₁ and R₂ groups one or more NH₂ groups, OH groups, SH groups or COOH groups may be protected. In specific embodiments Z is Cl. In specific embodiments, R₁ is selected from those of the R₁COOH groups illustrated in Schemes 5A-5D. In specific embodiments, PR₂ is a protecting group for a carboxylate. Of particular interest are those compounds of Formula XXXI which are E isomers free of Z isomer or those which are Z isomer free of E isomer.

In embodiments, the invention provides intermediates useful at least for synthesis of compounds of penems of Formula I herein of Formula XXXII:

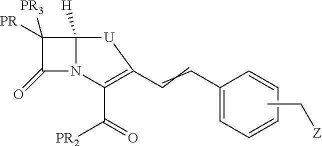

or salts or solvates thereof
where:

### U is —S—, —CHR₁₇—, or —O—,

Z is Cl, Br or I, where the —CH₂—Z group is substituted at any carbon in the phenyl ring;
R, R₃, and R₂ are as defined for Formula I, IA, VA or any embodiment thereof, where the PR, PR₂ and PR₃ indicate that potentially reactive functional groups in the R, R₂ and R₃ groups are optionally protected as needed to carry out the various syntheses. More specifically in R, R₂ and R₃ groups one or more NH₂ groups, OH groups, SH groups or COOH groups may be protected. In specific embodiments Z is Cl. In specific embodiments, R is selected from those of the R₁COOH groups illustrated in Schemes 5A-5D. In specific embodiments, PR₂ is a protecting group for a carboxylate. Of particular interest are those compounds of Formula XXXII which are E isomers free of Z isomer or those which are Z isomer free of E isomer. In specific embodiments of Formula XXXII, T is —CHR₁₇—, where R₁₇ is hydrogen or methyl. In a specific embodiment, the —CH₂—Z group is substituted at the position para in the phenyl ring with respect to the bond to the double bond.

Scheme 7 illustrates exemplary M groups for each of the formulas herein containing the M variable.

In each M moiety of Scheme 7:

R_{C} represents hydrogen or a substituent at an indicated ring position or hydrogens or one or more substituents at any possible ring position wherein the non-hydrogen substituents are independently selected from C1-C6 alkyl, C1-C6 alkylamino, C1-C6 alkoxy, nitro, CN, OH, halogen, amino, and a phenyl ring substituted with vicinal diol groups (on adjacent ring positions) and optionally substituted with one or two additional R₁₁ or R₁₂ which are independently selected from hydrogen, hydroxyl, halogen, C1-C3 alkyl, cyano, and nitro groups;
R_{N} is hydrogen or a C1-C3 alkyl;
R₈ is a C1-C6 alkyl group or a C3-C6 cycloalkyl group;
a is 0 or 1 to indicate the presence or absence of the L₁ or L₂ groups, when the L₁ or L₂ group is absent a single bond links the indicated rings;
L₁ is selected from —(CH₂)p-, where p is 1-3, —CO—NR_{N}—, —NR_{N}—CO—, —CH₂—CO—CH₂—, —CH₂—CO—NR_{N}—, and —NR_{N}—CO—CH₂—, where R_{N} is hydrogen or a C1-C3 alkyl; and
L₂ is selected from —(CH₂)p-, where p is 1-3,
—CO—NR_{N}—, —NR_{N}—CO—, —CH₂—CO—CH₂—, —CH₂—CO—NR_{N}—, or —NR_{N}—CO—CH₂—, —CO—NR_{N}—CH₂—CO—NR_{N}—, and —CO—NR_{N}—CH₂—CH₂—NR_{N}—, where R_{N} is hydrogen or a C1-C3 alkyl.

In embodiments of M moieties of Scheme 7, each R_{N} in a moiety is hydrogen. In embodiment of M moieties of Scheme 7, each R_{N} that is not in L₁ or L₂ is independently hydrogen or methyl. In embodiment of M moieties of Scheme 7, each R_{N} that is on a ring nitrogen is hydrogen. In embodiments of M moieties of Scheme 7, a is 0 and L₁ is absent. In embodiments of M moieties of Scheme 7, a is 0 and L₂ is absent. In embodiments of M moieties of Scheme 7, a is 1 and L₁ is present. In embodiments of M moieties of Scheme 7, a is 1 and L₂ is present. In embodiments of M moieties of Scheme 7, each R_{C} is hydrogen. In embodiments of M moieties of Scheme 7, each R_{C} is independently hydrogen or methyl. In embodiments of M moieties of Scheme 7, R_{C} represents hydrogens at all ring positions. In embodiments of M moieties of Scheme 7, R_{C} represents one or two non-hydrogen substituents and hydrogens at all remaining ring positions. In embodiments of M moieties of Scheme 7, R_{C} represents substitution with a single non-hydrogen substituent on the indicted ring. In specific embodiments, R_{C} represents substitution with a single non-hydrogen substituent on the indicted ring which substituent is OH. In specific embodiments, R_{C} represents substitution with a single non-hydrogen substituent on the indicted ring which substituent is a methyl group. In specific embodiments, R_{C} represents substitution with a single non-hydrogen substituent on the indicted ring which substituent is a halogen. In specific embodiments, R_{C} represents substitution with a single non-hydrogen substituent on the indicted ring which substituent is a chlorine. In specific embodiments, R_{C} represents substitution with a single non-hydrogen substituent on the indicted ring which substituent is a nitro group. In specific embodiments, R_{C} represents substitution with a single non-hydrogen substituent on the indicted ring which substituent is a phenyl ring carrying a vicinal diol. In specific embodiments, R_{C} represents substitution with a single non-hydrogen substituent on the indicted ring which substituent is a phenyl ring carrying a vicinal diol and one or two additional substituents selected from methyl or chlorine.

In specific embodiments of M moieties of Scheme 7, R₈ is a straight chain or branched alky having 1-4 carbon atoms. In specific embodiments of M moieties of Scheme 7, R₈ is a straight chain or branched alky having 1-3 carbon atoms. In specific embodiments of M moieties of Scheme 7, R₈ is a cycloalkyl group. In specific embodiments of M moieties of Scheme 7, both R₁₁ and R₁₂ are hydrogens. In specific embodiments of M moieties of Scheme 7, R₁₁ and R₁₂ are halogens or hydrogen. In specific embodiments of M moieties of Scheme 7, R₁₁ and R₁₂ are both halogens. In specific embodiments of M moieties of Scheme 7, R₁₁ are both chlorine. In specific embodiments of M moieties of Scheme 7, R₁₁ and R₁₂ are chlorine or hydrogen. In specific embodiments of each M moiety of Scheme 7, R₁₁ and R₁₂ are hydrogen or methyl groups.

In specific embodiments of M30 and M31, L₁ is present and selected from: —CH₂—, —CO—NH—, or —NH—CO—. In specific embodiments of M30 and M31, L₁ is absent. In specific embodiments of M32 and M33, L₂ is present and is —(CH₂)p- (where p is 1 or 2), —CO—NH—, —NH—CO—, —CH₂—CO—CH₂—, —CH₂—CO—NH—, or —NH—CO—CH₂—. In specific embodiments of M32 and M33, L₂ is absent. In specific embodiments of M32, each R_{N} is hydrogen or a methyl group. In specific embodiments of M34, M35 or M36, each R_{C} is hydrogen. In specific embodiments of M1, M4, M6, M10, M14, M15, M16, M17, M20, m21, M23, M30 or M31, R_{C} represents substitution of the indicated ring with a single non-hydrogen substituent or only one indicated R_{C} is a non-hydrogen substituent. In specific embodiments of M1, M4, M6, M10, M14, M15, M16, M17, M20, M21, M23, M30 or M31, R_{C} represents substitution of the indicated ring with a single non-hydrogen substituent or only one indicated R_{C} is a non-hydrogen substituent wherein the substituent is OH, a halogen, a nitro group, or a methyl group.

Scheme 8 illustrates a number of compounds of the invention which exhibit antibacterial activity as described herein. The compounds in Scheme 8 are shown with a positively charged nitrogen and no specific salt is illustrated. These compounds may be in zwitterionic form or may be in the form of a ammonium cation with an appropriate anion, such as a halide (e.g., Cl⁻, Br or I⁻), an organic anion, such as sulfate, bisulfate, acetate or trifluoroacetate, or any pharmaceutically acceptable anion, such as described herein.

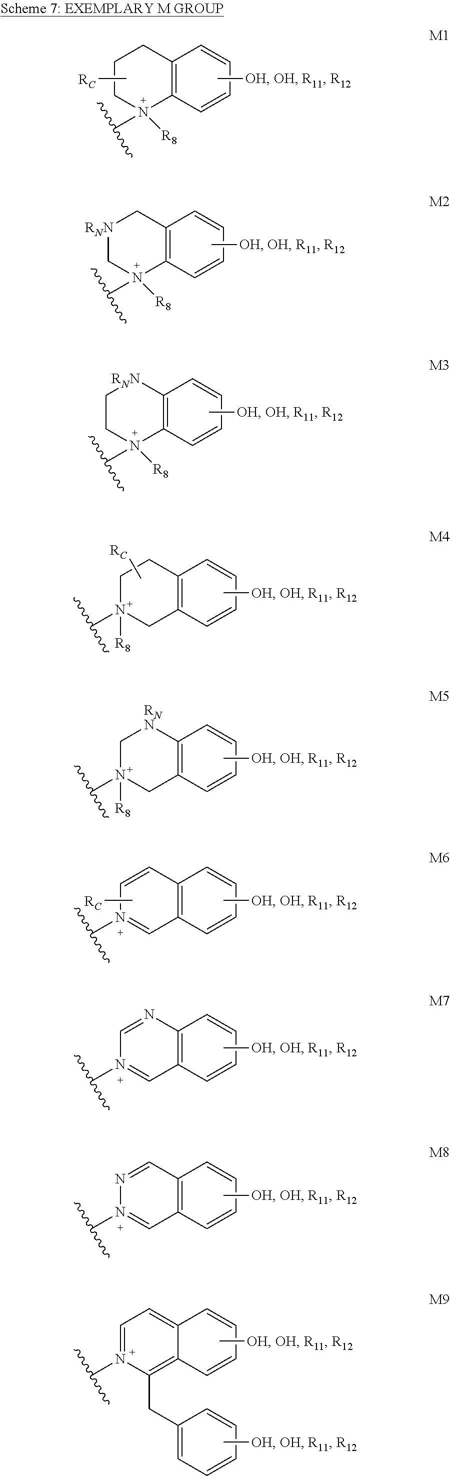

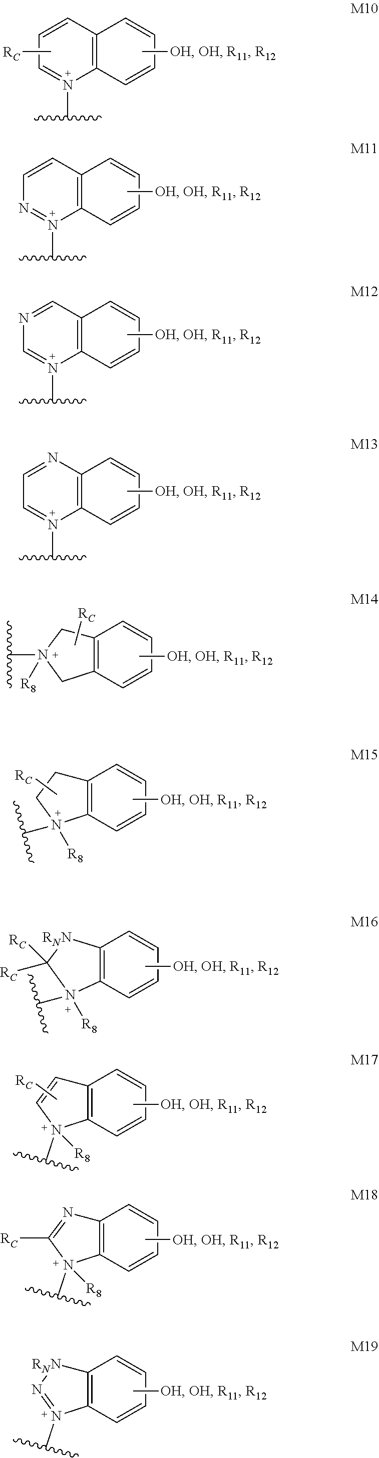

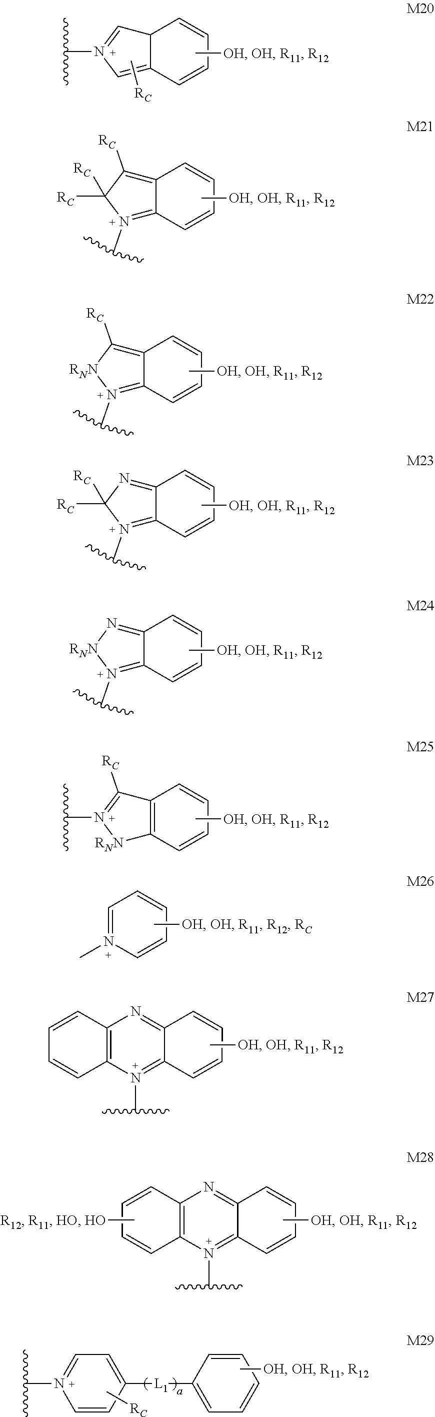

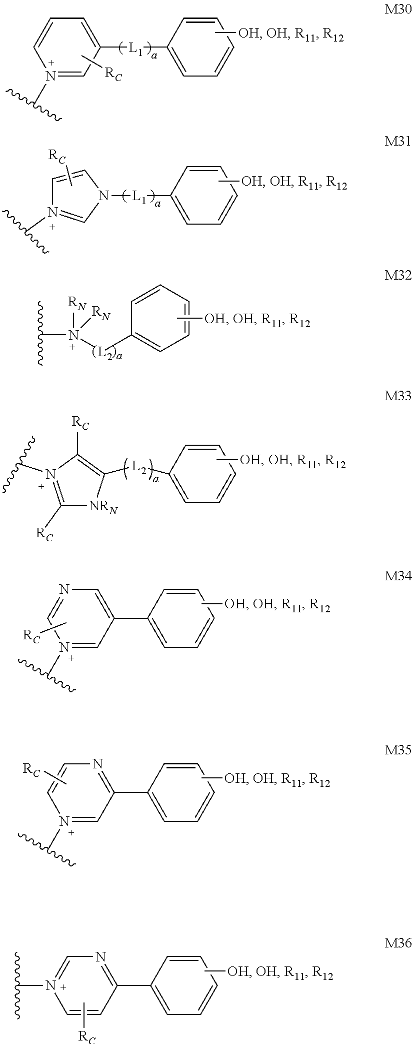

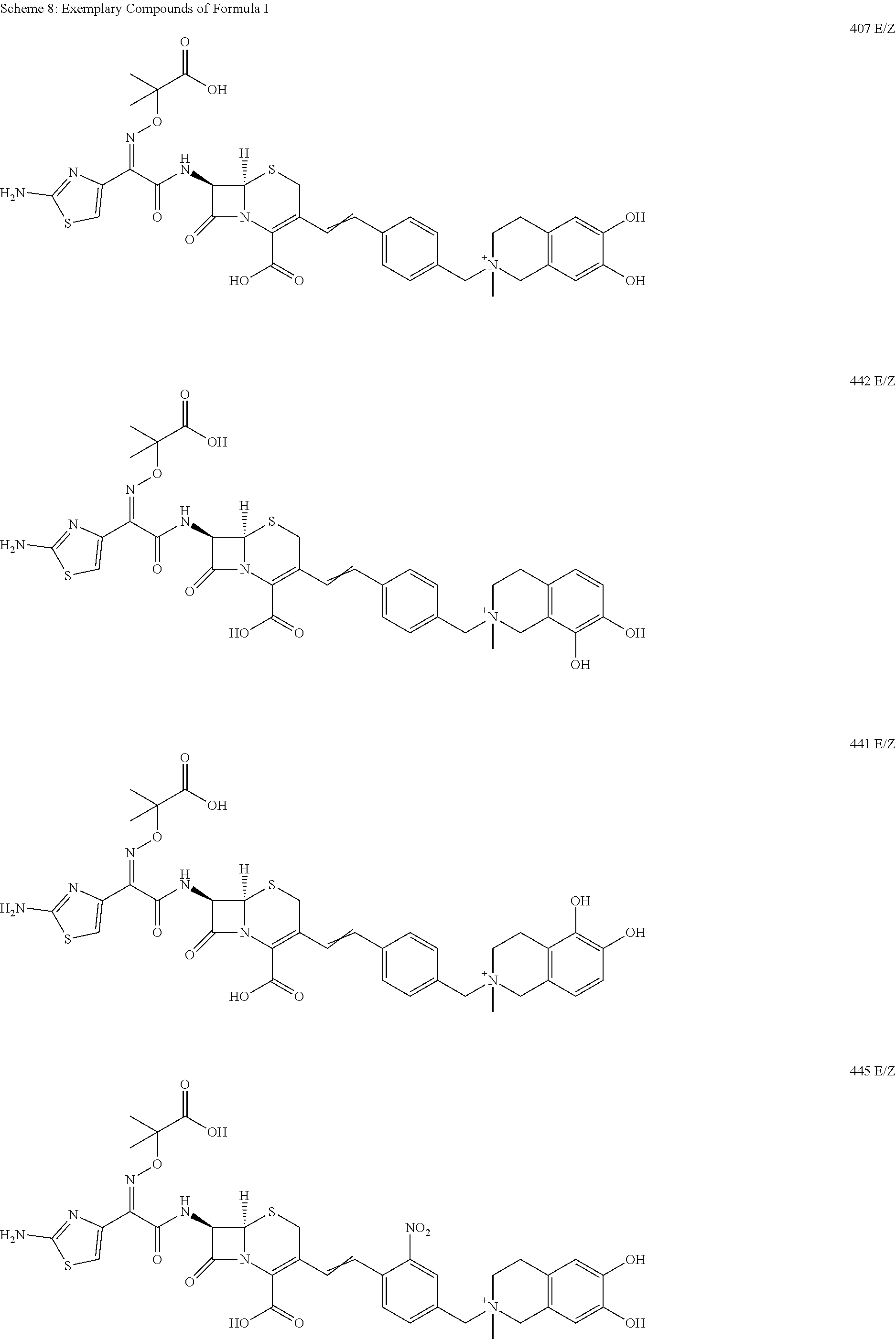

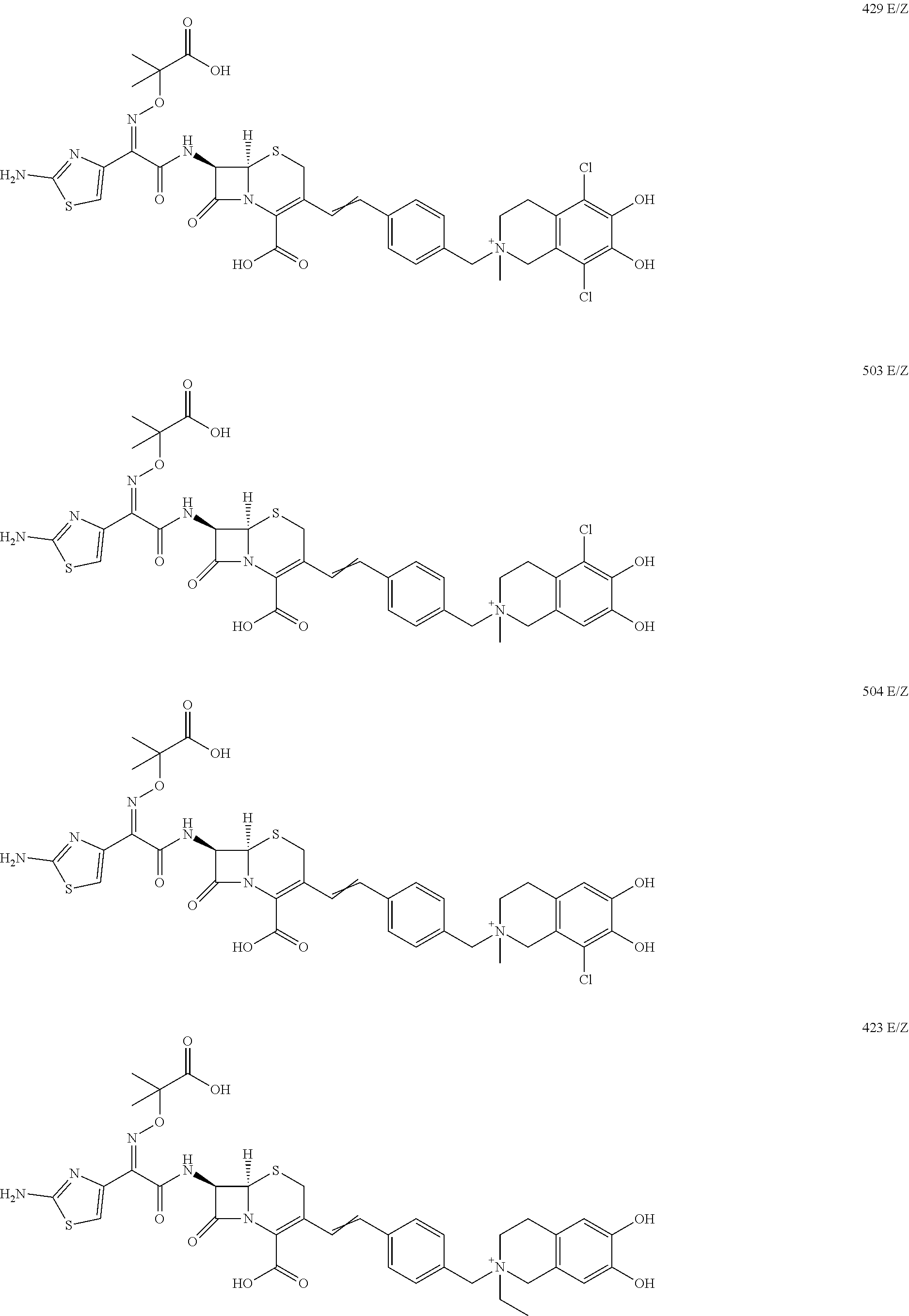

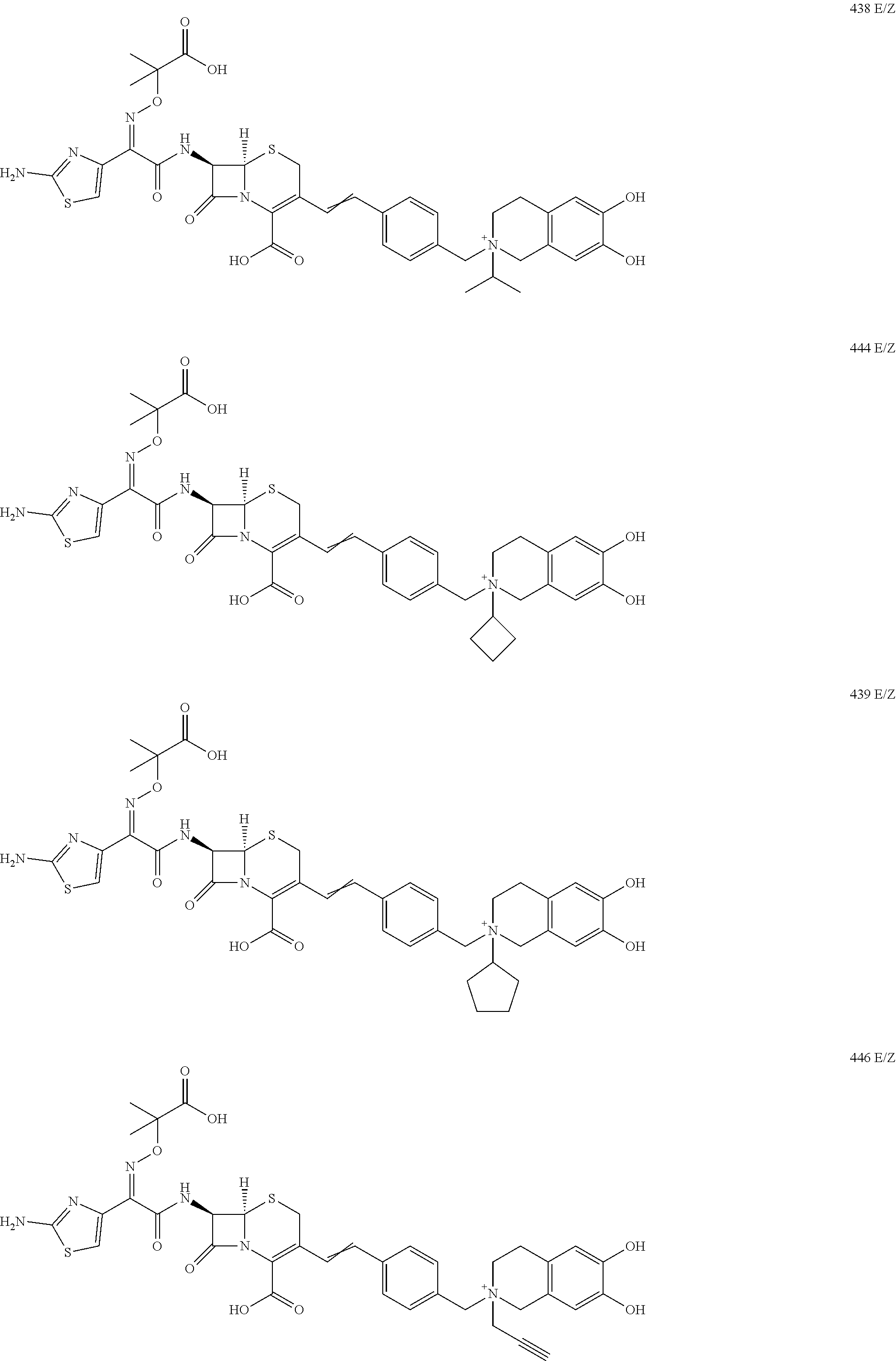

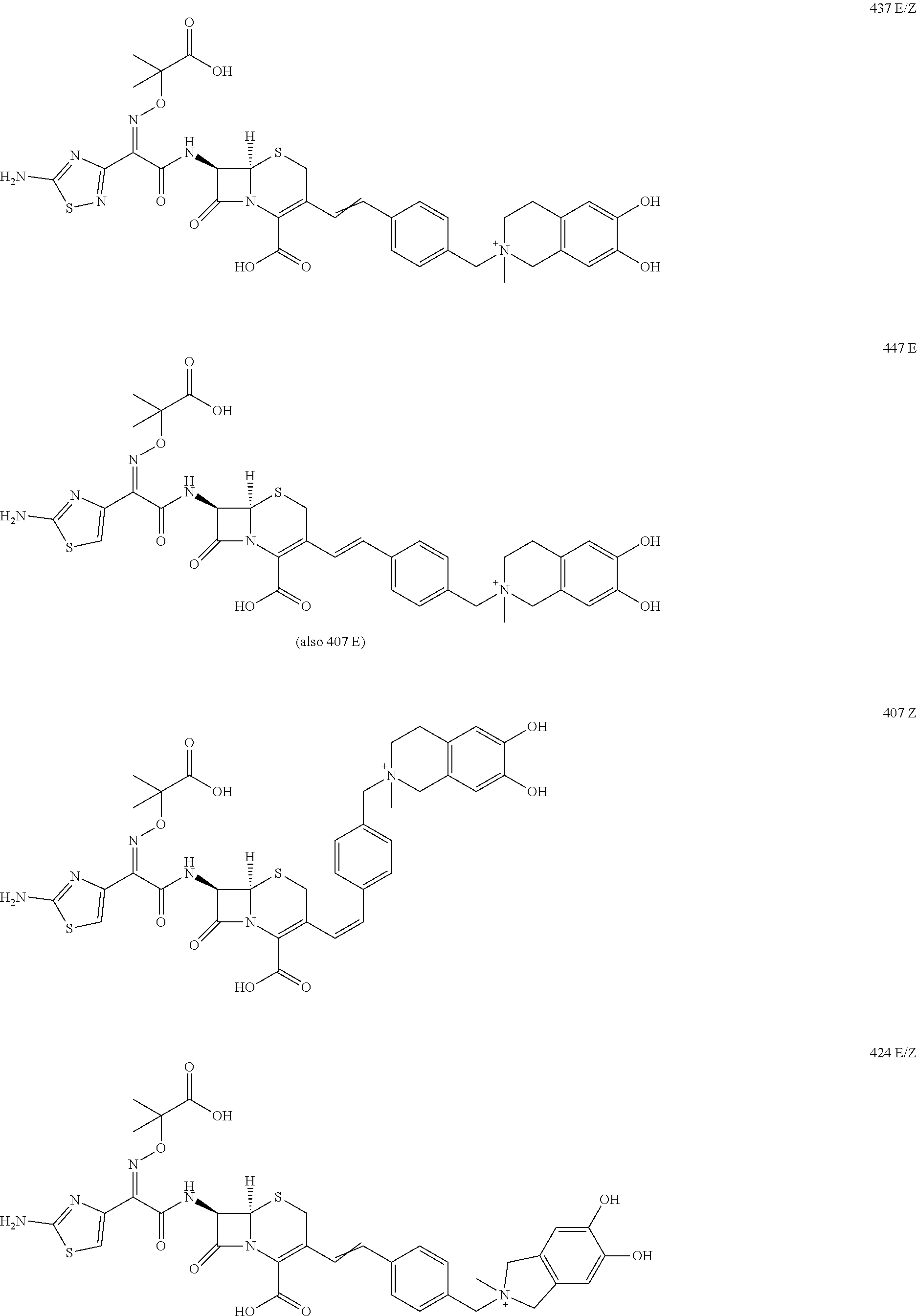

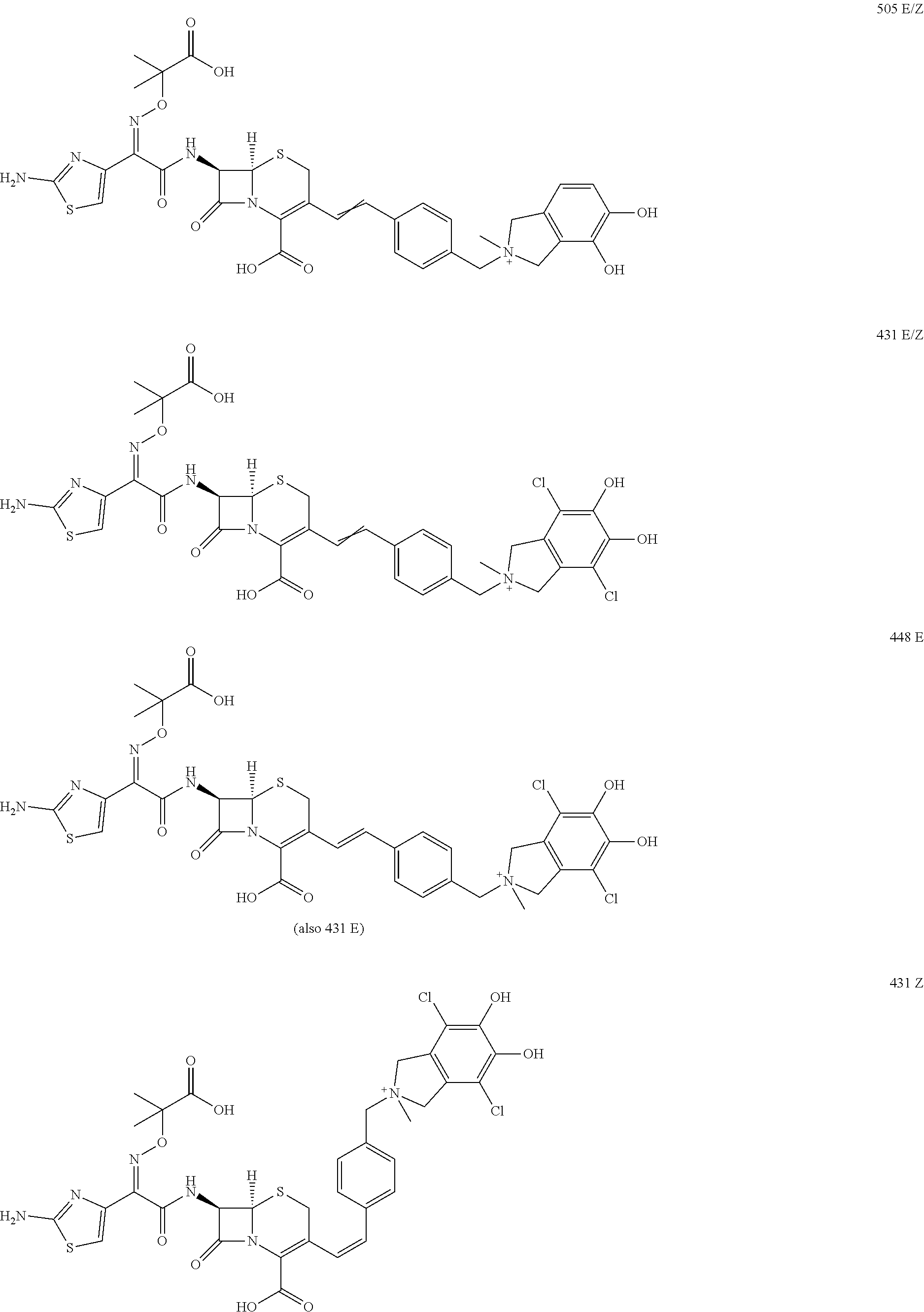

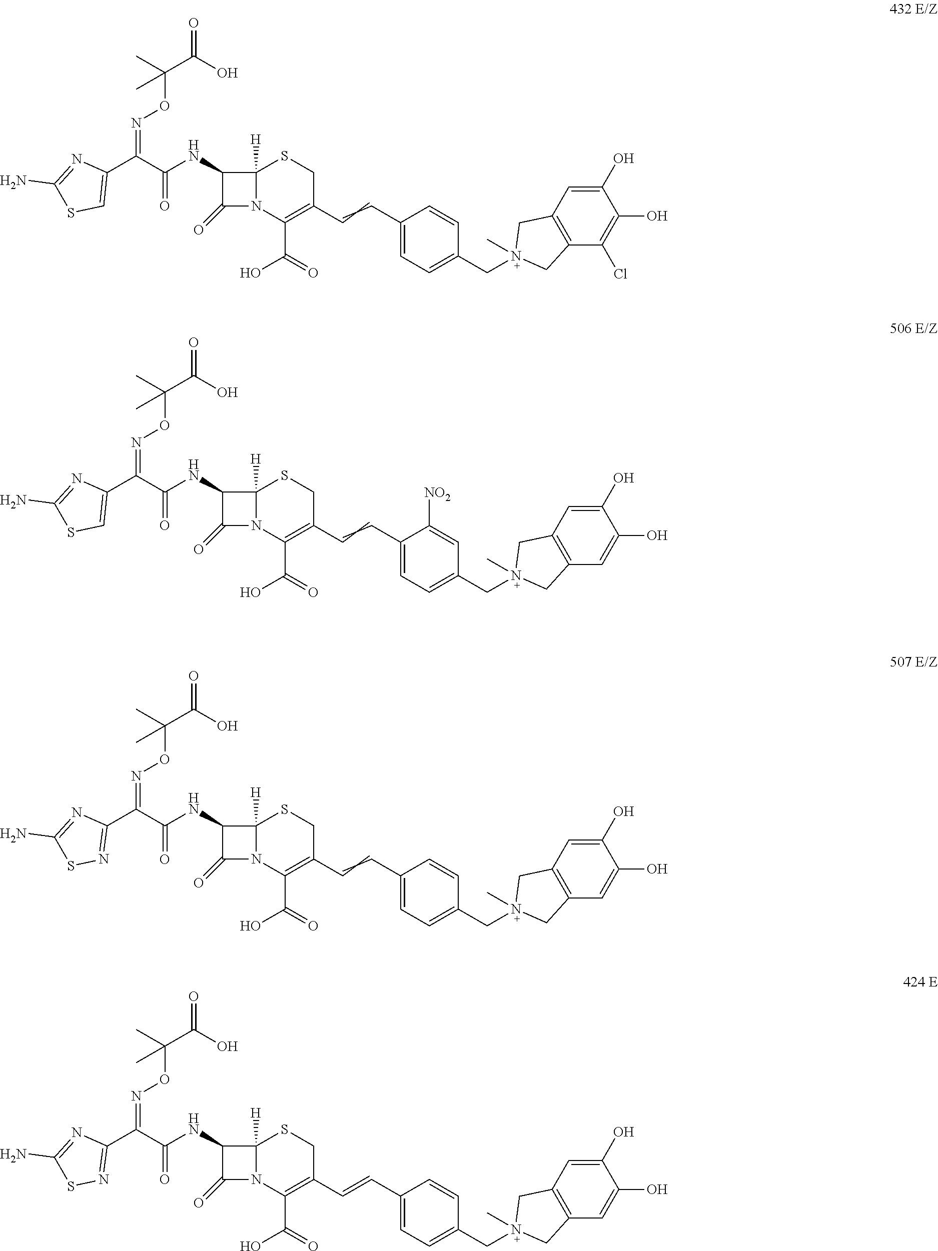

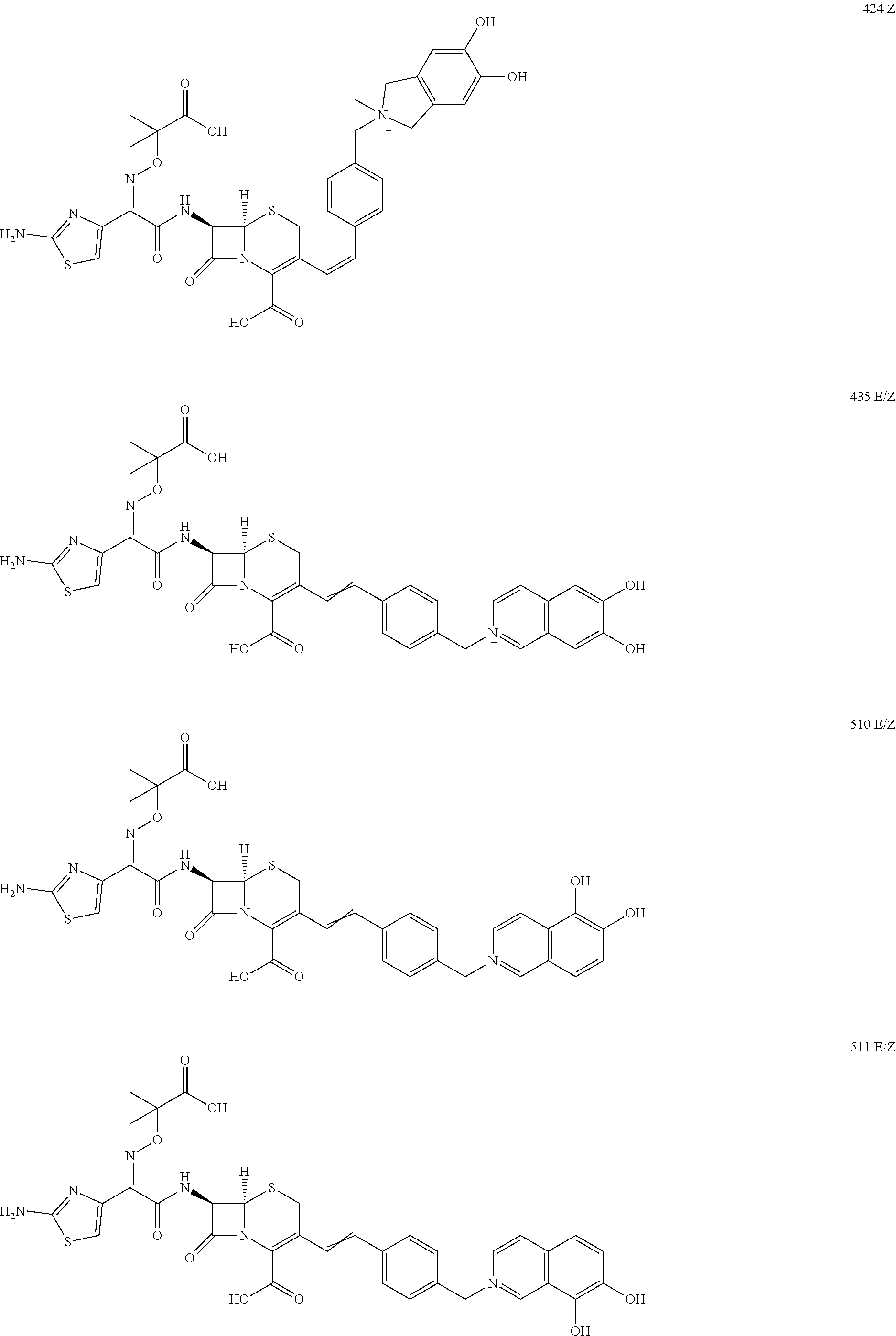

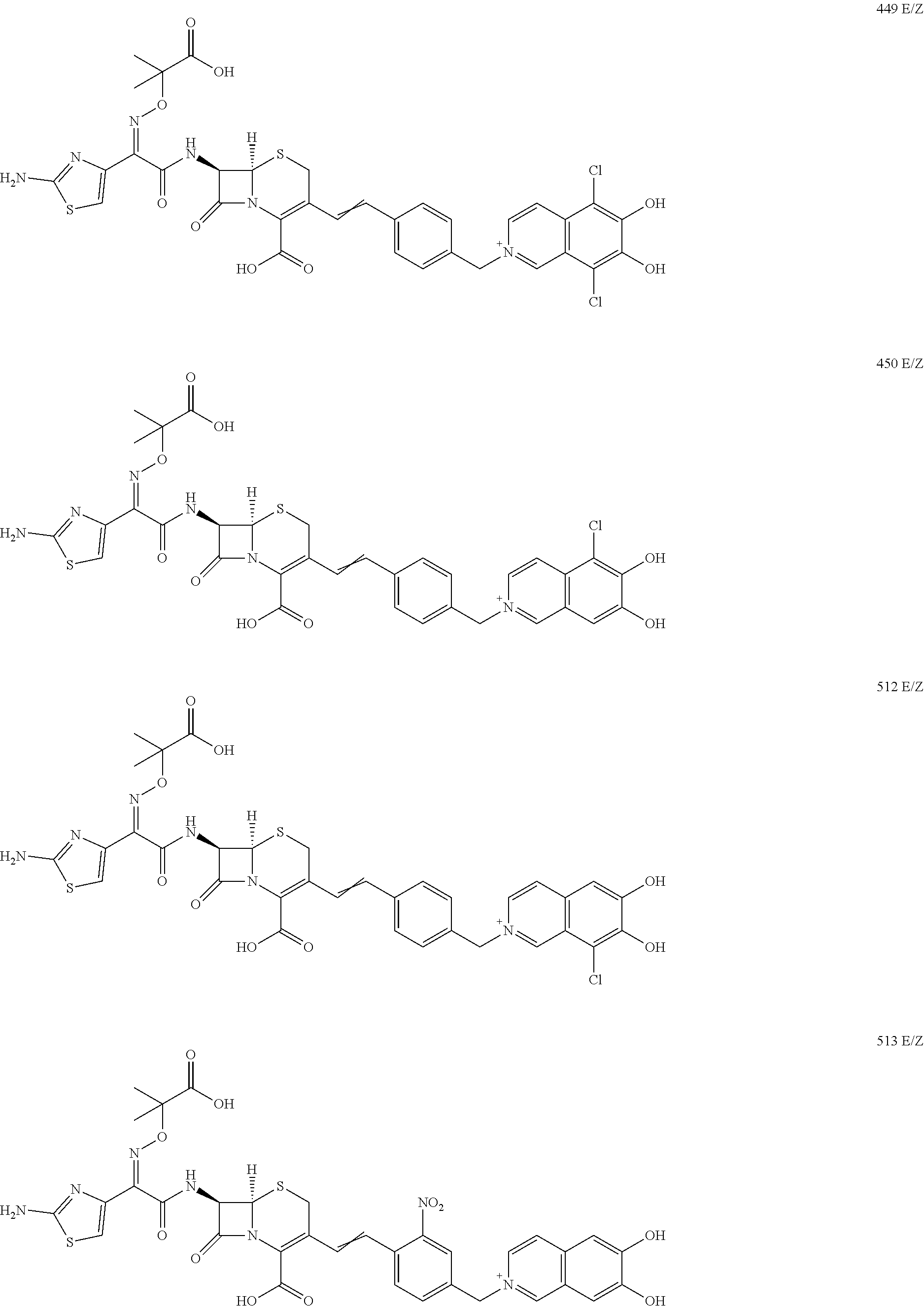

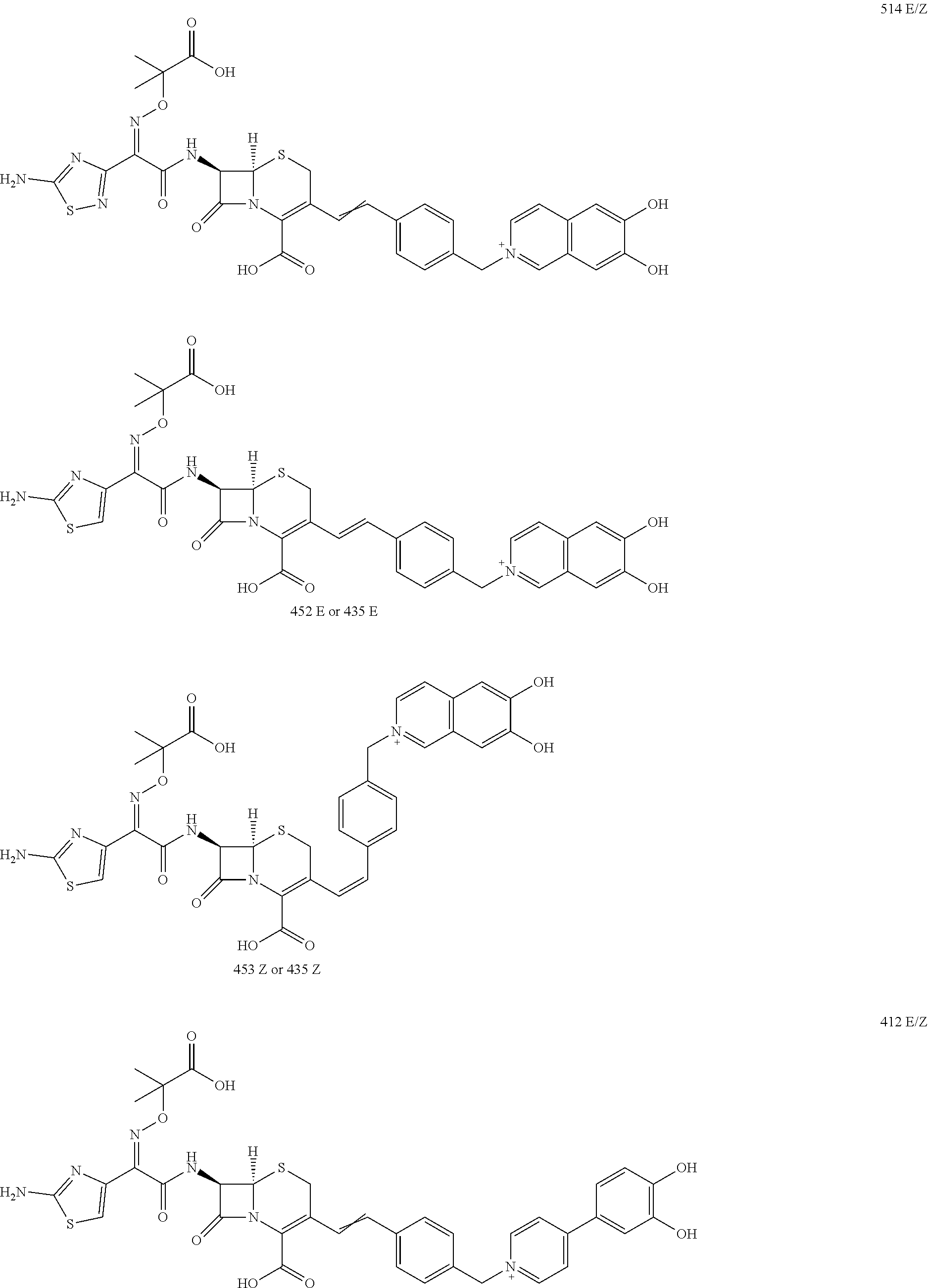

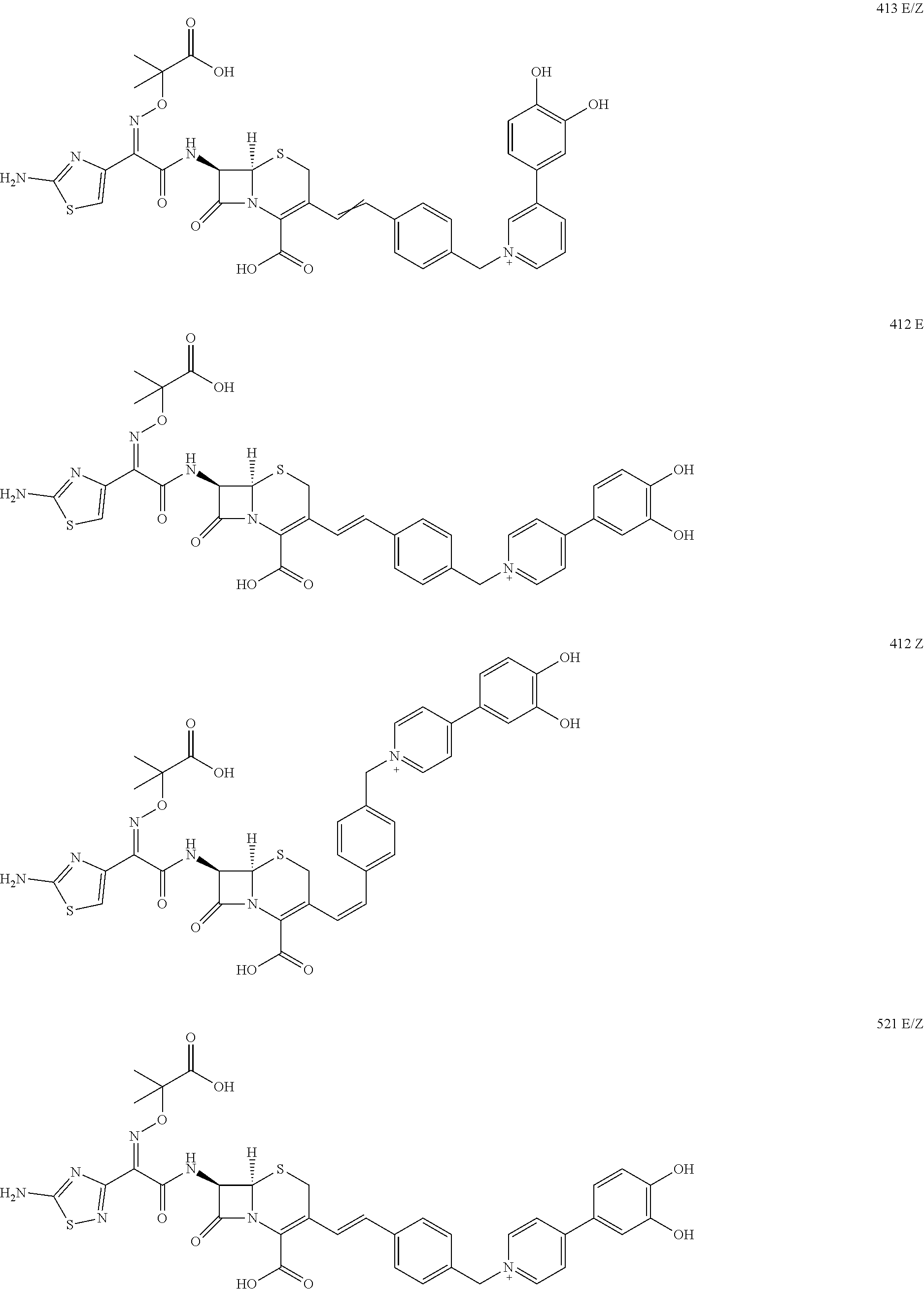

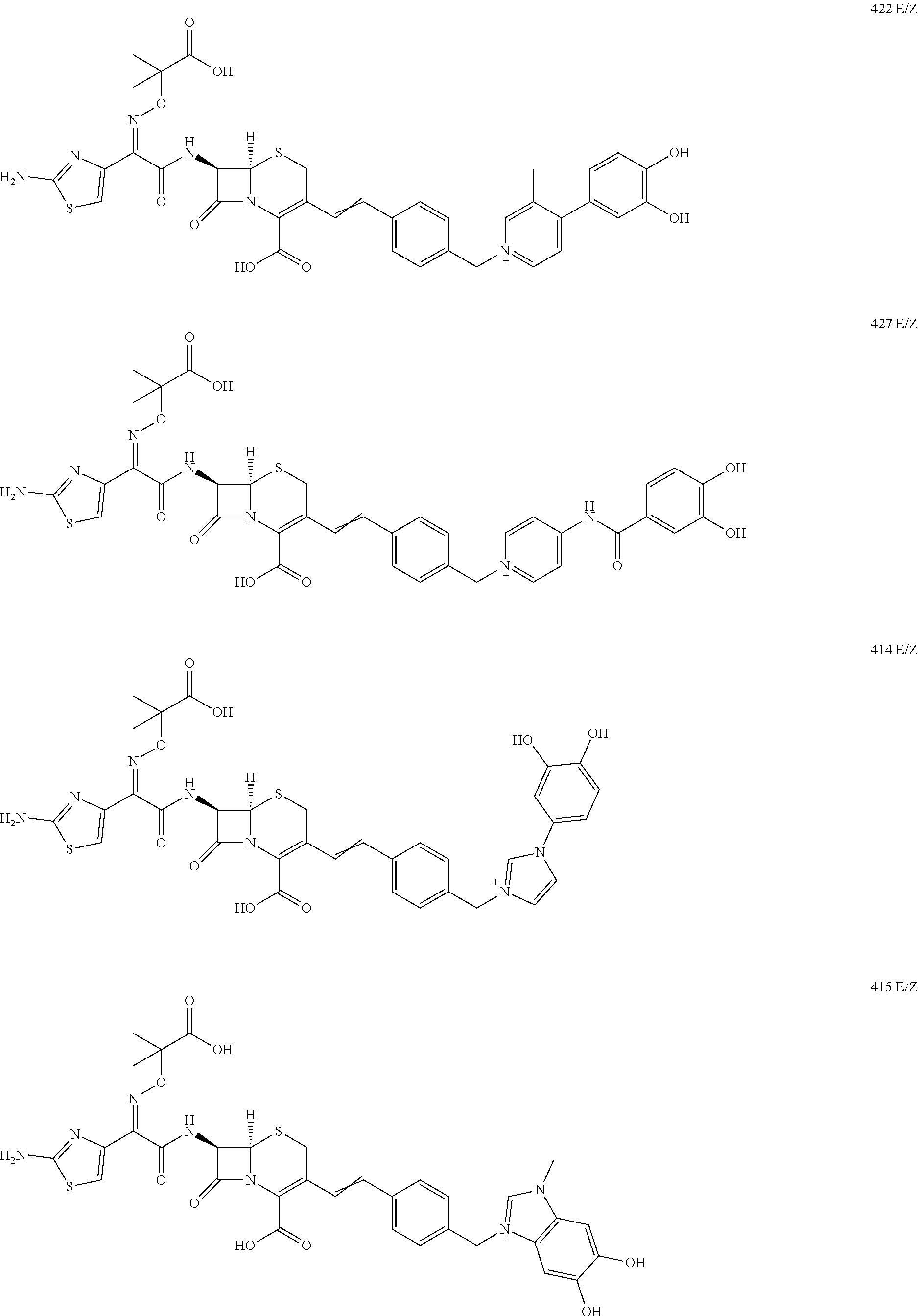

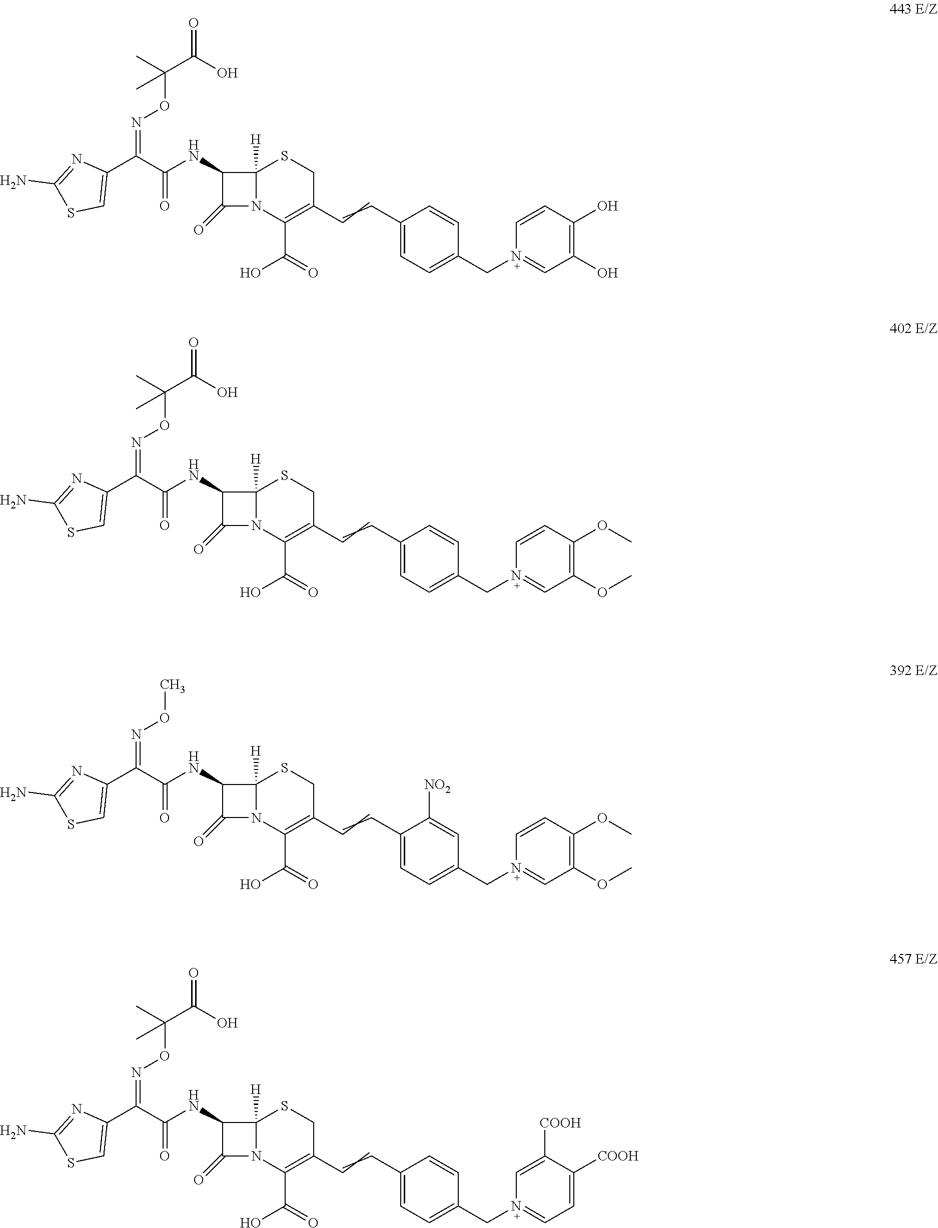

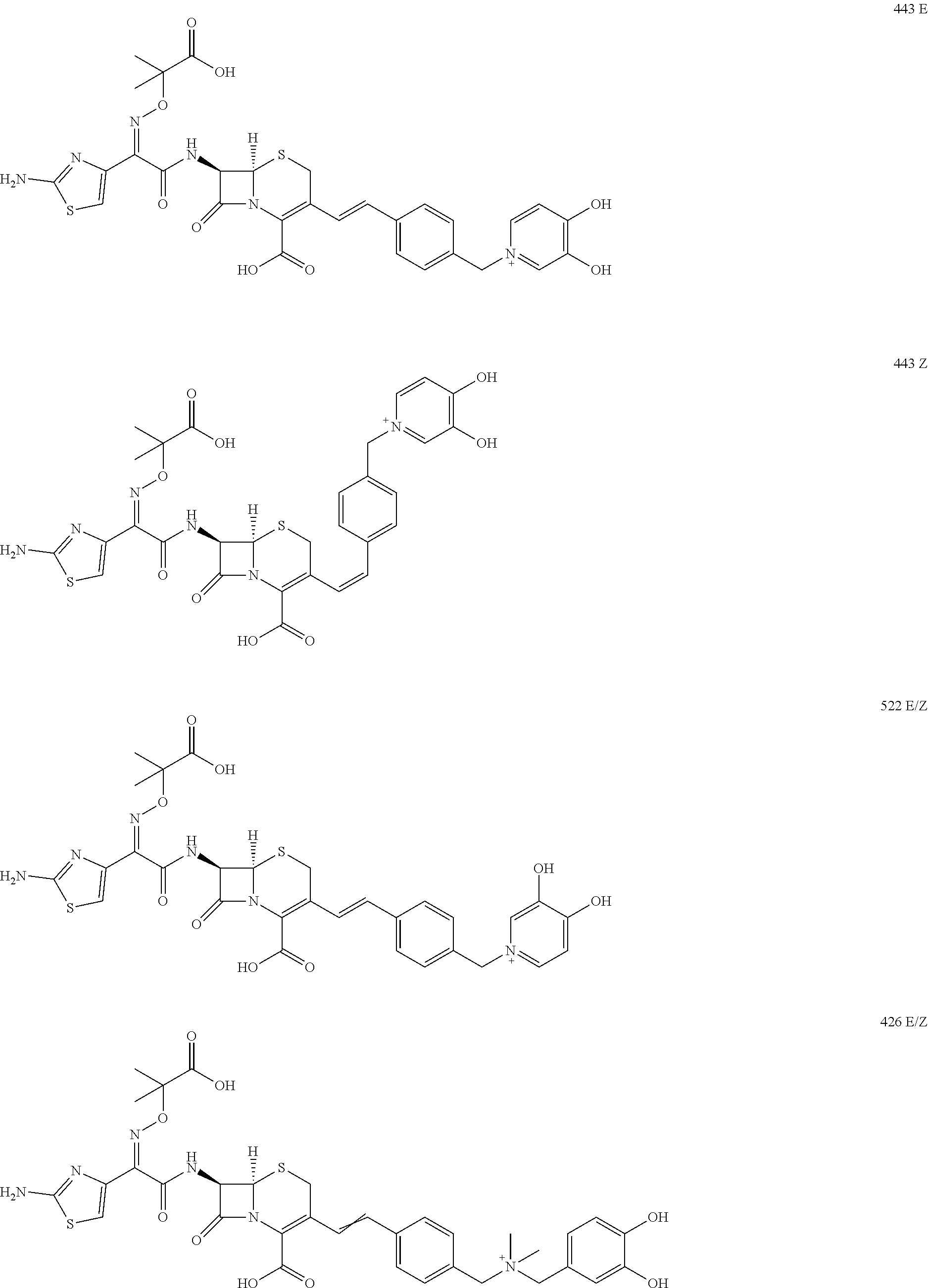

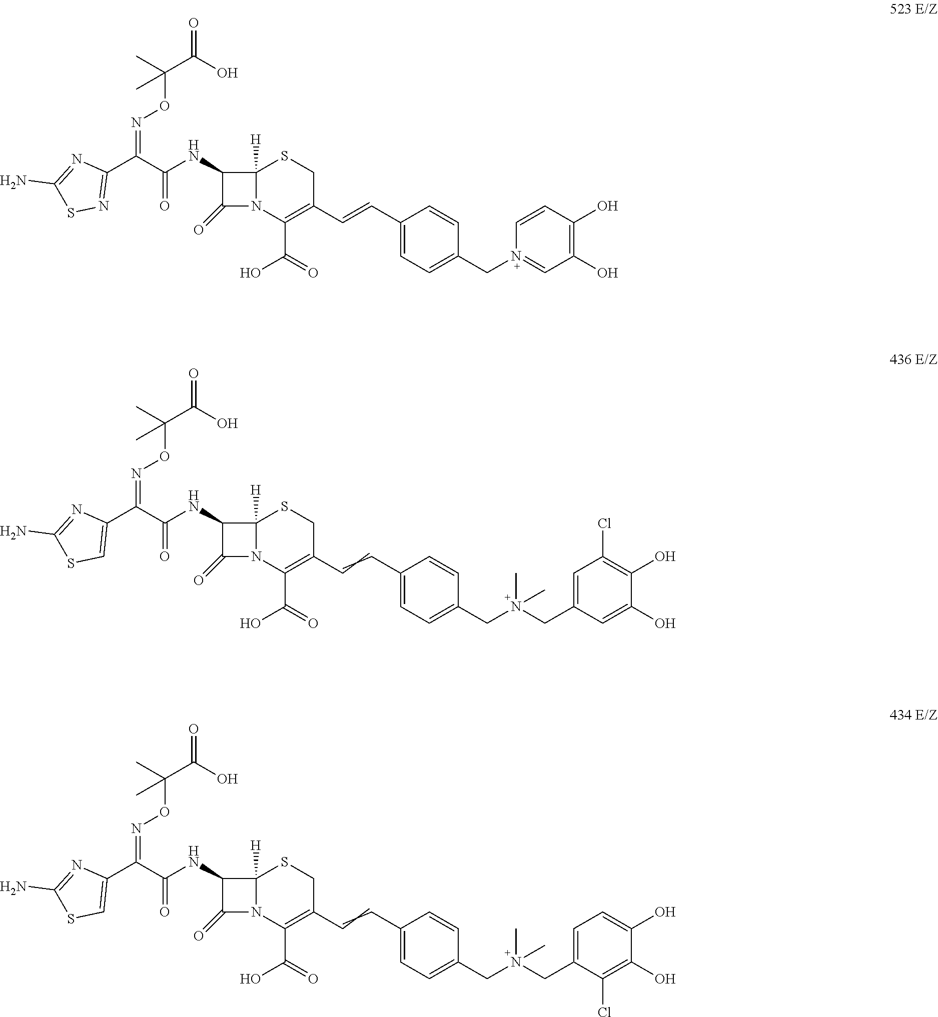

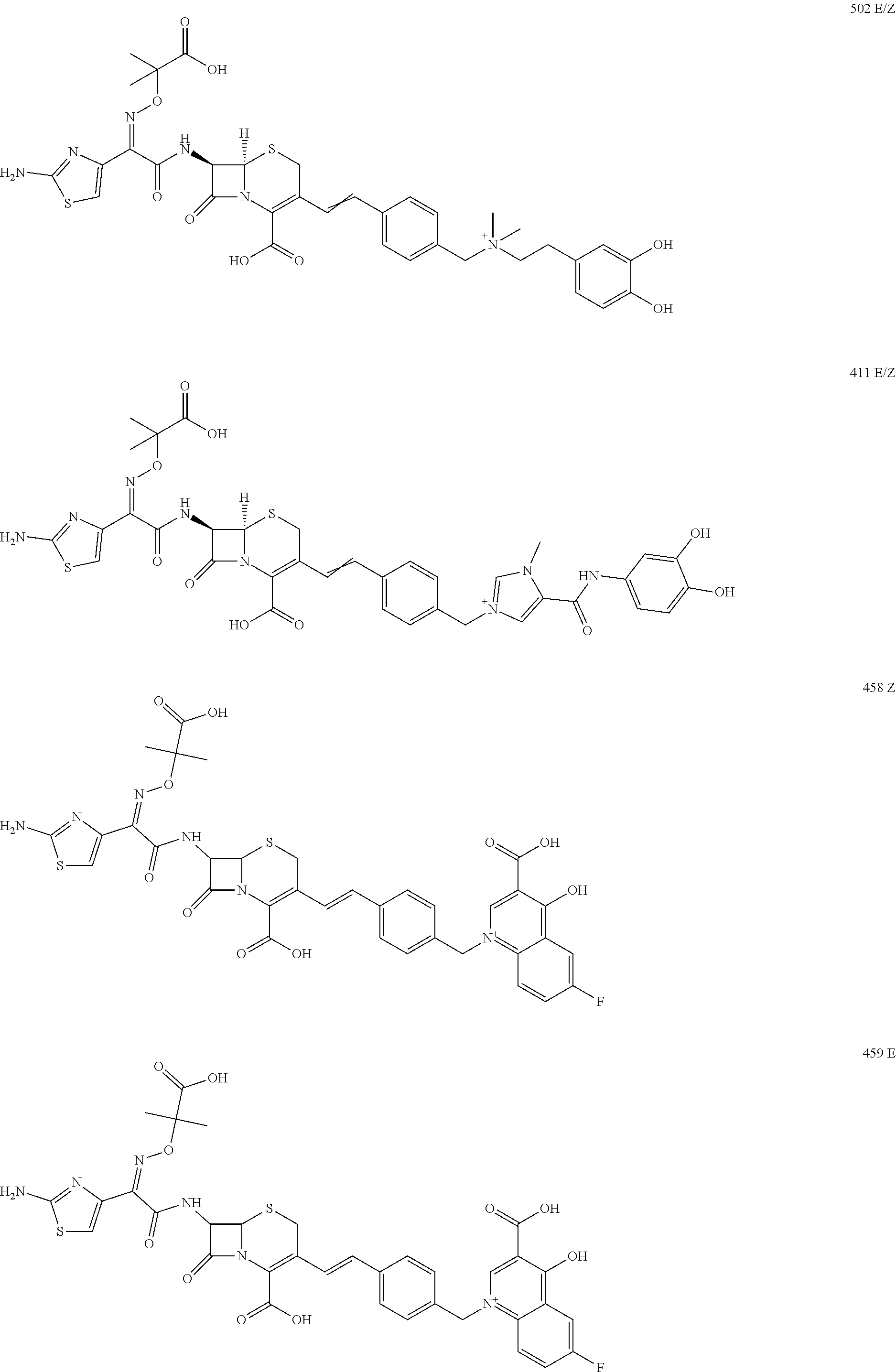

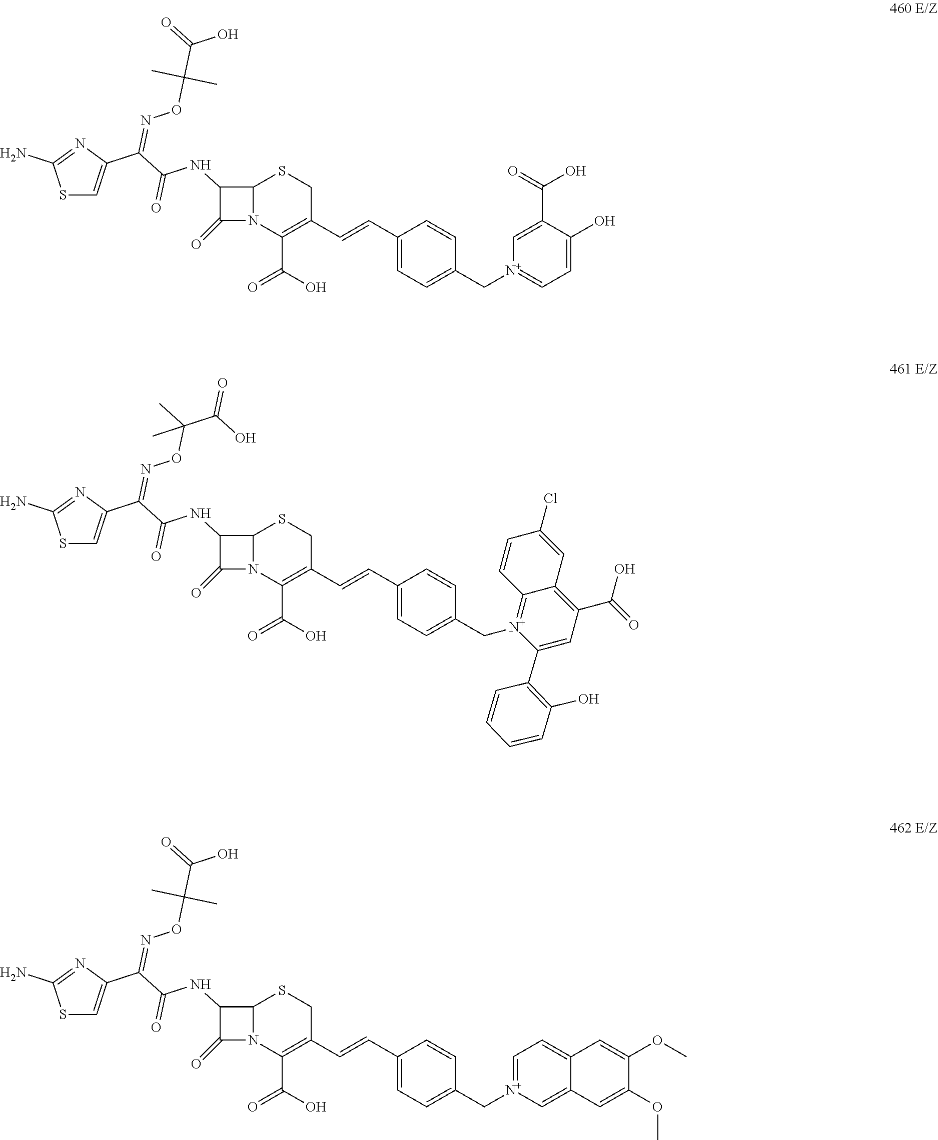 [SMILES: CC(C)(O/N=C(\C(=O)NC1C(=O)N2C(C(=O)O)=C(/C=C/c3ccc(C[n+]4ccc(O)c(C(=O)O)c4)cc3)CSC12)c1csc(N)n1)C(=O)O ; CC(C)(O/N=C(\C(=O)NC1C(=O)N2C(C(=O)O)=C(/C=C/c3ccc(C[n+]4c(-c5ccccc5O)cc(C(=O)O)c5cc(Cl)ccc54)cc3)CSC12)c1csc(N)n1)C(=O)O ; COc1cc2cc[n+](Cc3ccc(/C=C/C4=C(C(=O)O)N5C(=O)C(NC(=O)/C(=N\OC(C)(C)C(=O)O)c6csc(N)n6)C5SC4)cc3)cc2cc1OC]

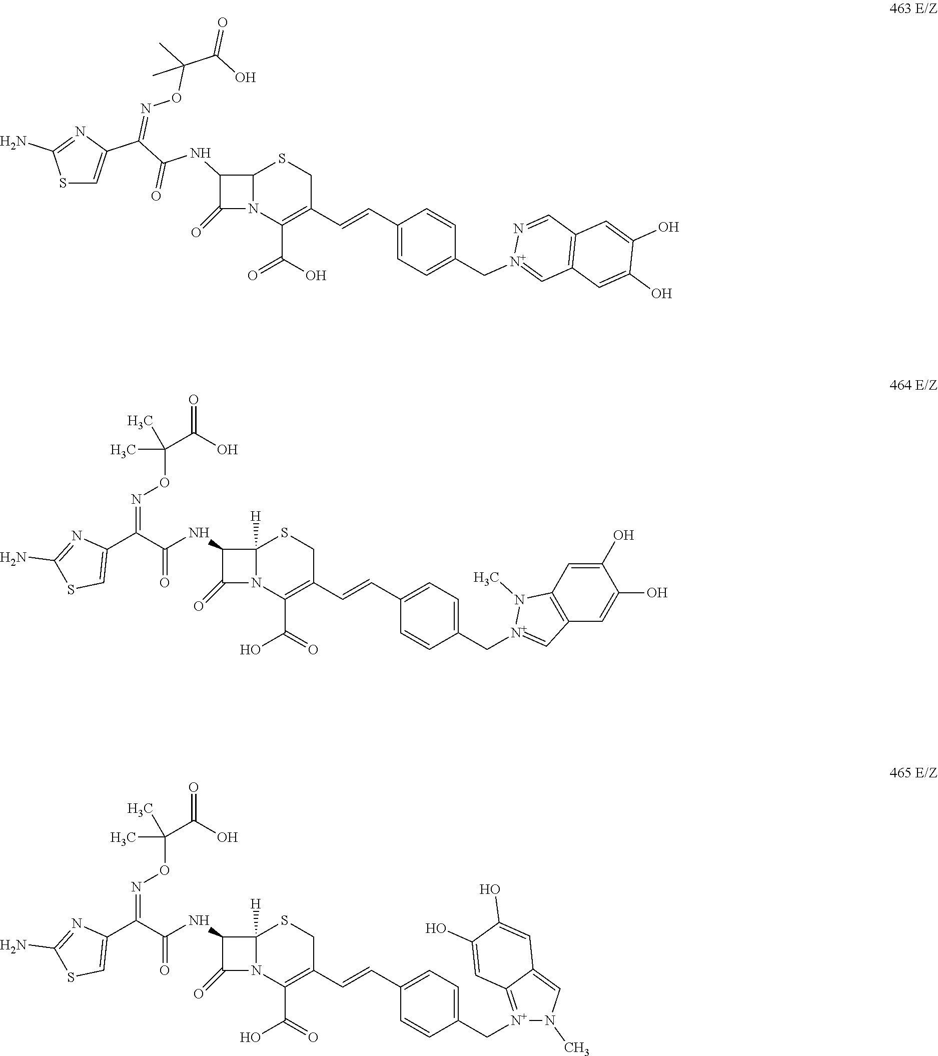 [SMILES: CC(C)(O/N=C(\C(=O)NC1C(=O)N2C(C(=O)O)=C(/C=C/c3ccc(C[n+]4cc5cc(O)c(O)cc5cn4)cc3)CSC12)c1csc(N)n1)C(=O)O ; Cn1c2cc(O)c(O)cc2c[n+]1Cc1ccc(/C=C/C2=C(C(=O)O)N3C(=O)[C@@H](NC(=O)/C(=N\OC(C)(C)C(=O)O)c4csc(N)n4)[C@H]3SC2)cc1 ; Cn1cc2cc(O)c(O)cc2[n+]1Cc1ccc(/C=C/C2=C(C(=O)O)N3C(=O)[C@@H](NC(=O)/C(=N\OC(C)(C)C(=O)O)c4csc(N)n4)[C@H]3SC2)cc1]

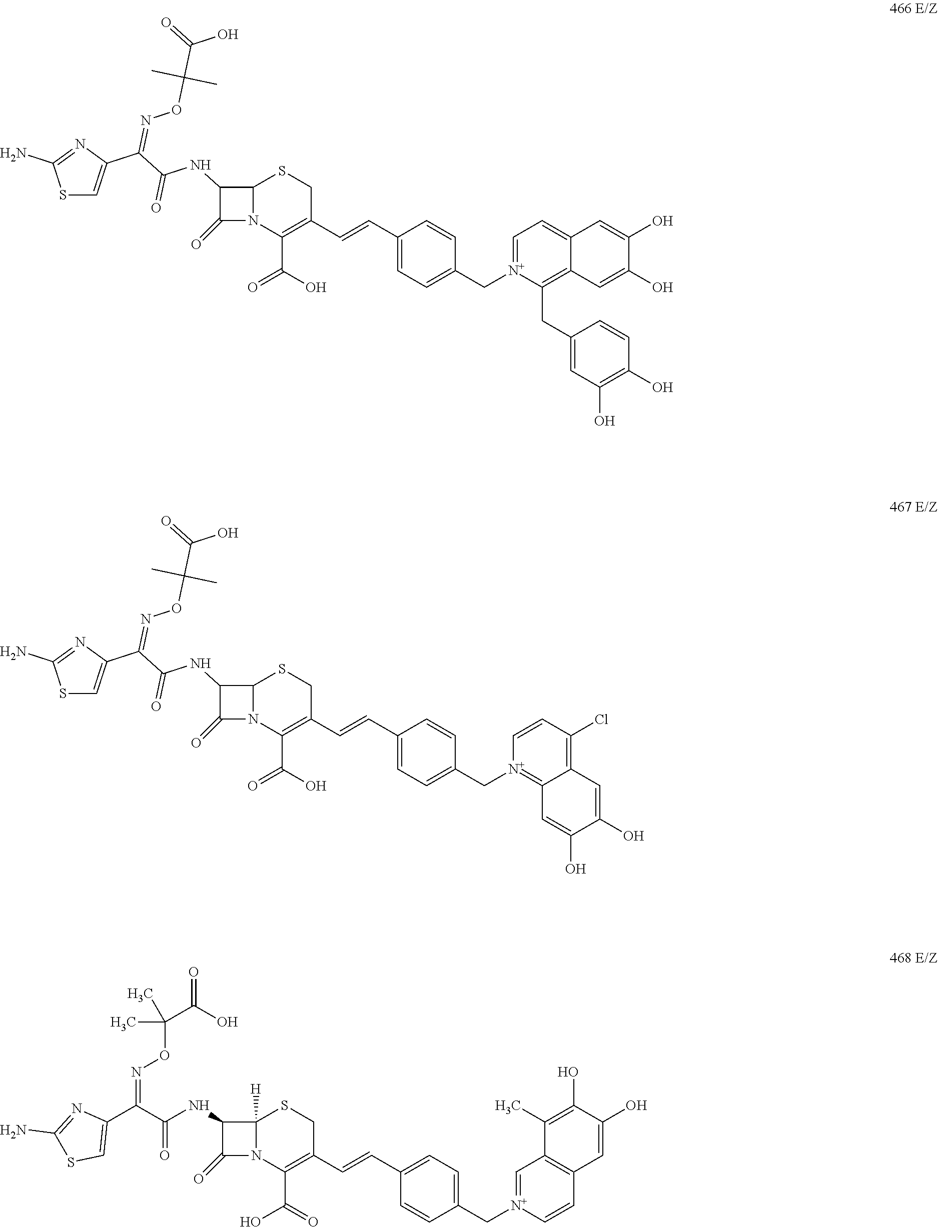 [SMILES: CC(C)(O/N=C(\C(=O)NC1C(=O)N2C(C(=O)O)=C(/C=C/c3ccc(C[n+]4ccc5cc(O)c(O)cc5c4Cc4ccc(O)c(O)c4)cc3)CSC12)c1csc(N)n1)C(=O)O ; CC(C)(O/N=C(\C(=O)NC1C(=O)N2C(C(=O)O)=C(/C=C/c3ccc(C[n+]4ccc(Cl)c5cc(O)c(O)cc54)cc3)CSC12)c1csc(N)n1)C(=O)O ; Cc1c(O)c(O)cc2cc[n+](Cc3ccc(/C=C/C4=C(C(=O)O)N5C(=O)[C@@H](NC(=O)/C(=N\OC(C)(C)C(=O)O)c6csc(N)n6)[C@H]5SC4)cc3)cc12]

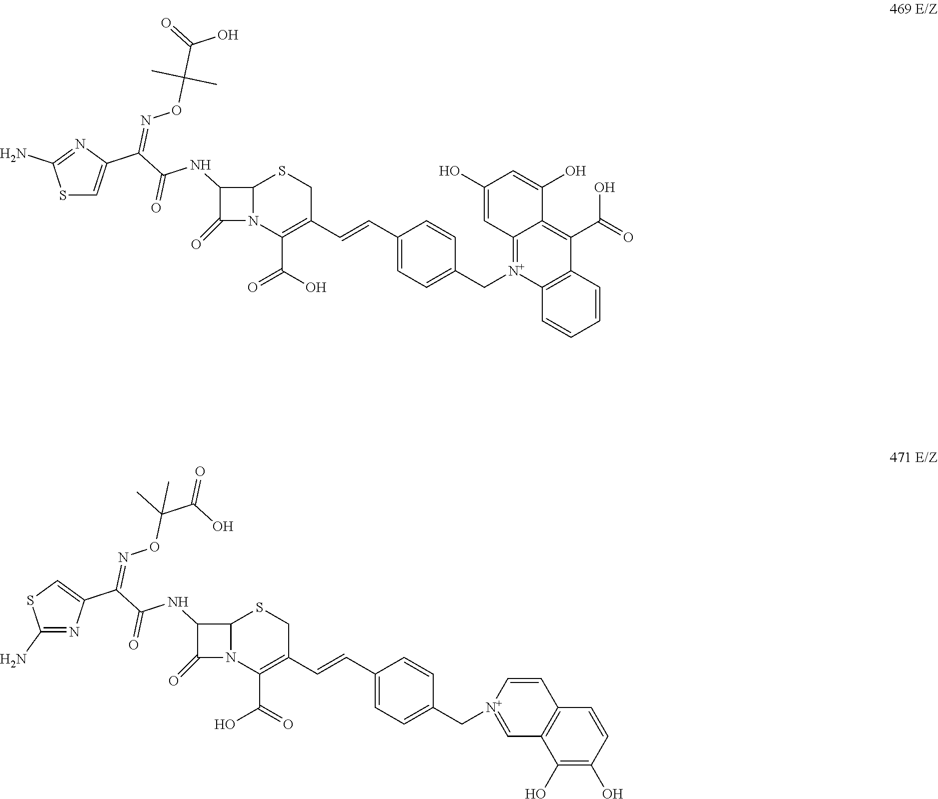 [SMILES: CC(C)(O/N=C(\C(=O)NC1C(=O)N2C(C(=O)O)=C(/C=C/c3ccc(C[n+]4c5ccccc5c(C(=O)O)c5c(O)cc(O)cc54)cc3)CSC12)c1csc(N)n1)C(=O)O ; CC(C)(O/N=C(\C(=O)NC1C(=O)N2C(C(=O)O)=C(/C=C/c3ccc(C[n+]4ccc5ccc(O)c(O)c5c4)cc3)CSC12)c1csc(N)n1)C(=O)O]

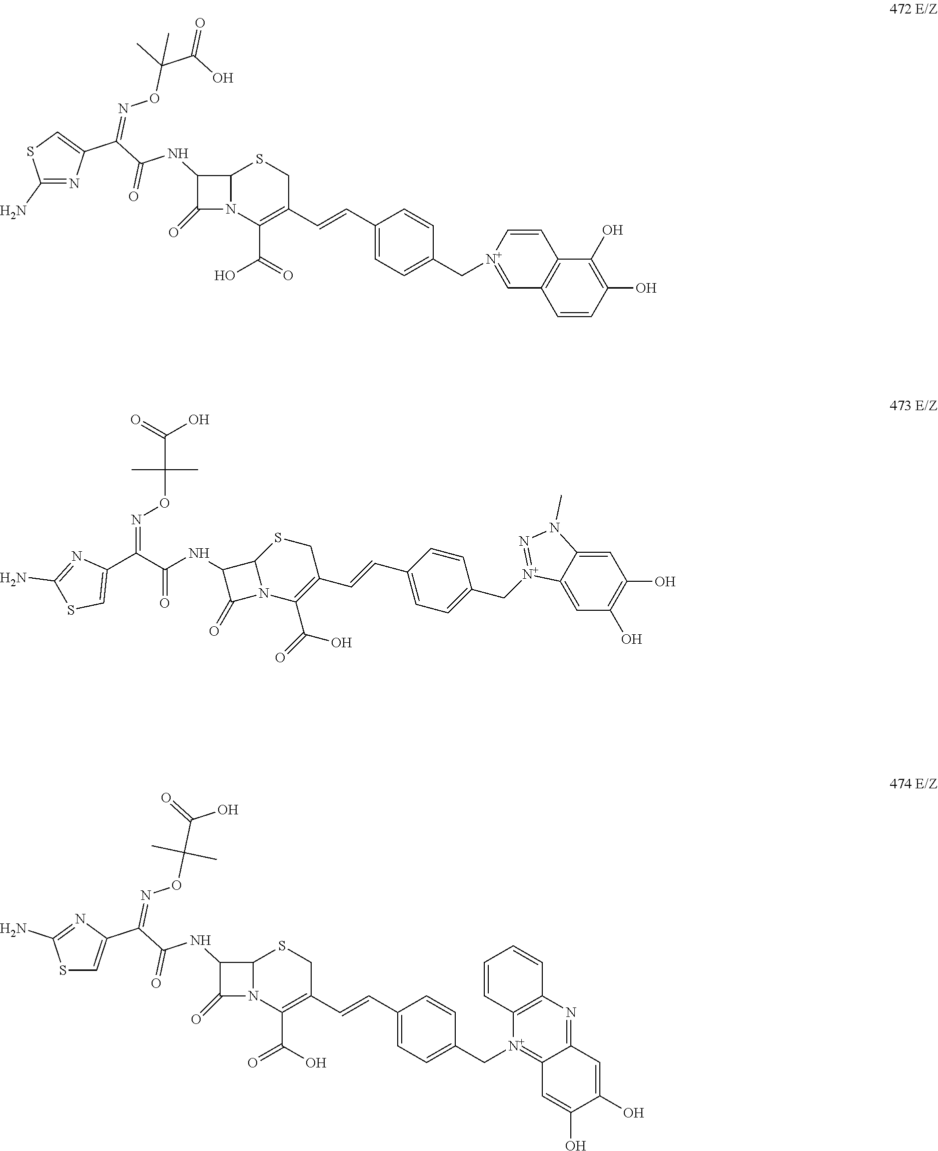 [SMILES: CC(C)(O/N=C(\C(=O)NC1C(=O)N2C(C(=O)O)=C(/C=C/c3ccc(C[n+]4ccc5c(O)c(O)ccc5c4)cc3)CSC12)c1csc(N)n1)C(=O)O ; Cn1n[n+](Cc2ccc(/C=C/C3=C(C(=O)O)N4C(=O)C(NC(=O)/C(=N\OC(C)(C)C(=O)O)c5csc(N)n5)C4SC3)cc2)c2cc(O)c(O)cc21 ; CC(C)(O/N=C(\C(=O)NC1C(=O)N2C(C(=O)O)=C(/C=C/c3ccc(C[n+]4c5ccccc5nc5cc(O)c(O)cc54)cc3)CSC12)c1csc(N)n1)C(=O)O]

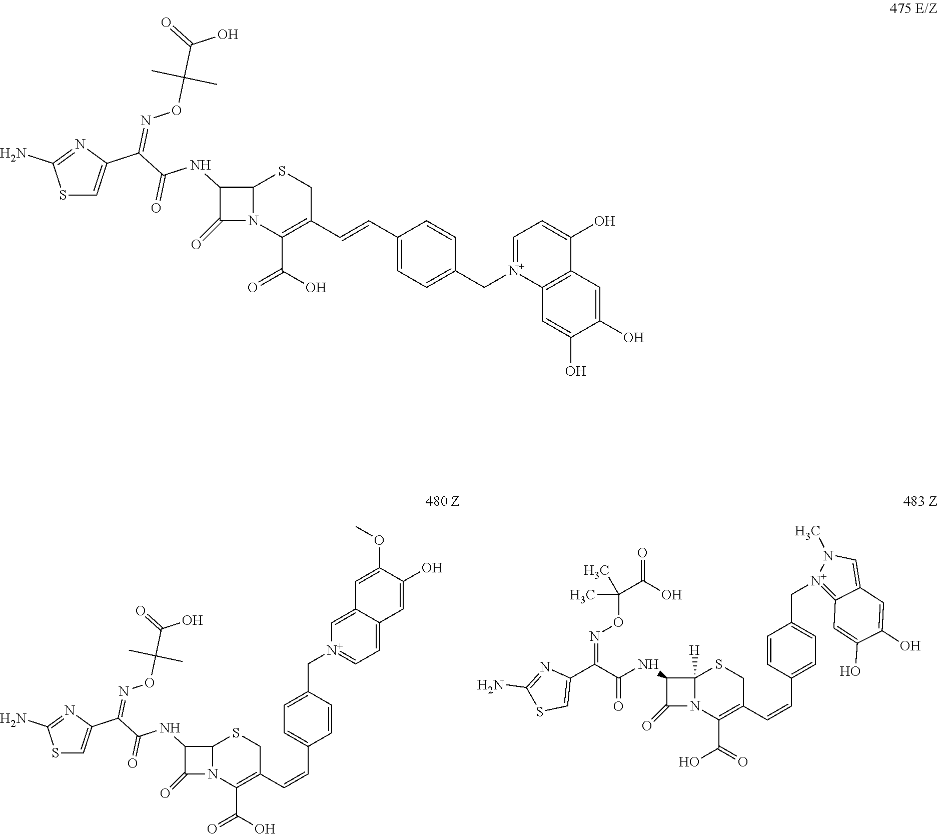 [SMILES: CC(C)(O/N=C(\C(=O)NC1C(=O)N2C(C(=O)O)=C(/C=C/c3ccc(C[n+]4ccc(O)c5cc(O)c(O)cc54)cc3)CSC12)c1csc(N)n1)C(=O)O ; COc1cc2c[n+](Cc3ccc(/C=C\C4=C(C(=O)O)N5C(=O)C(NC(=O)/C(=N\OC(C)(C)C(=O)O)c6csc(N)n6)C5SC4)cc3)ccc2cc1O ; Cn1cc2cc(O)c(O)cc2[n+]1Cc1ccc(/C=C\C2=C(C(=O)O)N3C(=O)[C@@H](NC(=O)/C(=N\OC(C)(C)C(=O)O)c4csc(N)n4)[C@H]3SC2)cc1]

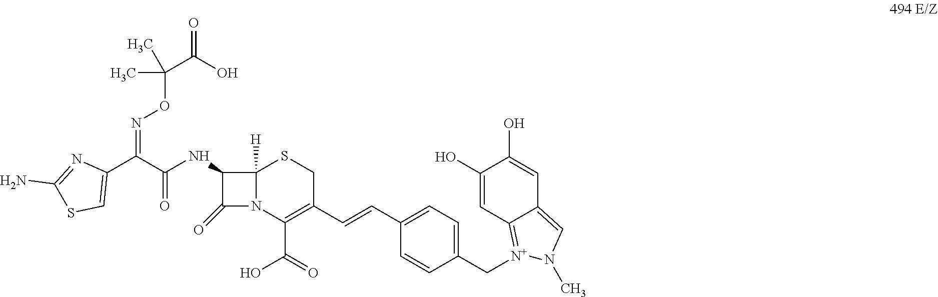 [SMILES: Cn1cc2cc(O)c(O)cc2[n+]1Cc1ccc(/C=C/C2=C(C(=O)O)N3C(=O)[C@@H](NC(=O)/C(=N\OC(C)(C)C(=O)O)c4csc(N)n4)[C@H]3SC2)cc1]

The number of carbons in a given group is designated herein using the terminology CX—CY, where X and Y are integers representing the lowest number and the highest number of carbons in the references group, as in C1-C4 alkyl which refers to an alkyl group having 1-4 carbon atoms. In a number of the formulas of this invention reference is made to the definition of variables in a given patent or patents or published U.S. patent application. In these instances, the variable definition from the patent document listed applies. In other cases, formula variables are specifically defined in the present specification and the definitions of such variables is defined herein or employs the broadest definition in the art of a given chemical moiety or group.

The terms alkyl or alkyl group, alone or in combination, refer to a monoradical of a straight chain or branched saturated hydrocarbon. Alkyl groups include straight-chain and branched alkyl groups. Unless otherwise indicated alkyl groups have 1-12 carbon atoms (C1-C12 alkyl groups) and preferred are those that contain 1-6 carbon atoms (C1-C6 alkyl groups) and more preferred are those that contain 1-4 carbon atoms (C1-C4 alkyl groups) and those that contain 1-3 carbon atoms (C1-C3 alkyl groups). Unless otherwise indicated alkyl groups are optionally substituted with one or more non-hydrogen substituents as described herein. However any alkyl group designated herein can be unsubstituted. The designation of an alkyl group having a range of carbon atoms includes all isomers having that number of carbon atoms. Exemplary alkyl groups include methyl, ethyl, n-propyl, iso-propyl, n-butyl, s-butyl, t-butyl, n-pentyl, branched pentyl, n-hexyl, branched hexyl, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl.

The term alkoxy refers to an —O-alkyl group, where alkyl is as defined above. Alkoxy groups include among others methoxy, ethoxy, n-propoxy, isopropoxy, n-butoxy, isobutoxy, sec-butoxy, and tert butoxy. Alkoxy groups are optionally substituted. The term cycloalkyl, alone or in combination, means an alkyl radical which contains at least one carbon ring. These groups may be monocyclic, bicyclic or tricyclic. Unless otherwise indicated a cycloalkyl contains from 3 to 12 carbons and the carbon ring contains 3-10 carbons and more preferably 3-7 carbon atoms. Examples of such cycloalkyl groups include cyclopropyl, cyclobutyl, cyclopentyl, and cyclohexyl. Cycloalkyl groups are optionally substituted.

The term alkenyl, alone or in combination, refers to a straight-chain or branched-chain mono-, di- or poly-unsaturated aliphatic hydrocarbon radical containing one or more double bonds and the specified number of carbon atoms, or where no number is specified, preferably from 2-10 carbon atoms, more preferably, from 2-6 carbon atoms and also from 2-4 carbon atoms. Unless specifically stated, all isomers of the given number of carbon atoms are included. Examples of alkenyl radicals include, but are not limited to, ethenyl, E- and Z-propenyl, isopropenyl, E- and Z-butenyl, E- and Z-isobutenyl, E- and Z-pentenyl, E- and Z-hexenyl, E,E-, E,Z-, Z,E- and Z,Z hexadienyl. Alkenyl groups are optionally substituted.

The term alkenoxy refers to an —O-alkenyl group, where alkenyl is defined above. Alkenoxy groups are optionally substituted.

The term cycloalkenyl means an alkyl radical which contains at least one carbon ring and at least one double bond. These groups may be monocyclic, bicyclic or tricyclic. Unless otherwise indicated a cycloalkyl contains from 3 to 12 carbons and the carbon ring contains 3-10 carbons and more preferably 3-7 carbon atoms. Examples of such cycloalkenyl groups include cyclopentenyl, cyclohexenyl, cycloheptenyl and cyclooctenyl. Cycloalkenyl groups are optionally substituted.

The term alkynyl, alone or in combination, refers to a straight-chain or branched-chain aliphatic hydrocarbon radical containing one or more triple bonds and the specified number of carbon atoms, or where no number is specified, preferably from 2-10 carbon atoms, more preferably, from 2-6 carbon atoms and also from 2-4 carbon atoms. Specific alkynyl groups contain one triple bond or two triple bonds. Unless specifically stated, all isomers of the given number of carbon atoms are included. Examples of alkynyl radicals include, but are not limited to, ethynyl, propynyl, butynyl, pentynyl, etc. Alkynyl groups are optionally substituted. The term alkynoxy refers to an —O-alkynyl group, where alkynyl is defined above. Alkynoxy groups are optionally substituted.

An acyl group is an R′—CO— group where R′ in general is a hydrogen, an alkyl, alkenyl or alkynyl, aryl, heterocycyl, or heteroaryl group as described herein. In specific embodiments, R′ is a substituted methylene group. In specific embodiments, acyl groups have 1-20, 1-12, or 1-6 carbon atoms and optionally 1-3 heteroatoms, optionally one double bond or one triple bond. In specific embodiments, R is a C1-C6 alkyl, alkenyl group. cyclic configuration or a combination thereof, attached to the parent structure through a carbonyl functionality. Examples include acetyl, benzoyl, propionyl, isobutyryl, or oxalyl. The R′ group of an acyl group is optionally substituted as described herein. When R′ is hydrogen, the group is a formyl group. An acetyl group is a CH₃—CO— group. Another exemplary acyl group is a benzyloxy group.

An acylalkoxy group is an alkoxy group as defined above substituted with an acyl group as defined above, e.g., —OCH₂—COR. An alkyl group substituted with an acylalkoxy group is an acylalkoxyalkyl group.

An acyloxy group is an acyl group as defined above bonded to an oxygen, e.g., —O—COR. An alkyl group substituted with an acyloxy group is an acyloxyalkyl group.

An acylamino group is an R′—CO—NH— group, where R′ is as defined for the acyl group above. A number of acylamino groups are known in the art as suitable for use in cephem and penem antibiotics.

An amino group is —NH₂. An alkylamino group is —NHR′, where R′ is an alkyl group, preferably which is a C1-C4 alkyl or a C1-C3 alkyl. An alkylamino group is —NR₂′, where R′ is an alkyl group, preferably which is a C1-C4 alkyl or a C1-C3 alkyl. Amino, alkylamino and dialkyl amino groups can be protonated or quaternized for example with bonding of another alkyl group resulting in a positively charged amino, alkyl amino, dialkyamino, trialky amino or a quaternary amino group. Certain compounds herein have cyclic amino groups where the nitrogen is in a ring. The ring may otherwise contain only carbon ring atoms or the ring may contain an additional heteroatom, such as O, N or S. The ring may be saturated (containing no double bonds), partially unsaturated or fully unsaturated. The nitrogen in the ring can have the form —NH—, or ═N— or be a ring member of an aromatic ring (e.g., such as a pyridine). As is illustrated in formulas herein, the ring N can be protonated or alkylated (with a C1-C4 alkyl group) to form a positively charged ring amino group.

The term “aryl,” alone or in combination, refers to a carbocyclic aromatic radical (such as phenyl or naphthyl) containing the specified number of carbon atoms. If not specified aryl groups contain from 6-15 carbon atoms, preferably from 6-10 carbon atoms, and particularly contain from 6-10 ring carbons. Aryl groups unless otherwise stated are optionally substituted among others with one or more substituents selected from alkyl, alkoxy, nitro, halogen, (for example chloro), amino, alkyl amino, dialkylamino, carboxylate and hydroxy. In specific embodiments, aryl groups are optionally substituted phenyl groups. Aryl groups may contain two rings that are fused (naphthyl) or two rings which are bonded together by a C—C bond (biphenyl). Examples of aryl groups include, among others, phenyl, p-tolyl, 4-hydroxyphenyl, 1-naphthyl, 2-naphthyl, indenyl, indanyl, azulenyl, fluorenyl, and anthracenyl.

The term arylalky, alone or in combination, refers to an alkyl group as described above which is substituted with an aryl group as defined above. In specific embodiments, the aryl group is an optionally substituted phenyl, naphthyl or biphenyl group. In specific embodiments, the arylalkyl is a phenalkyl group. Specific phenalkyl groups are benzyl and phenethyl groups.

The term heterocyclyl or heterocyclic refers to monoradical having a ring of a specified number of ring atoms, where the ring atoms include one or more heteroatoms (N, O, S) or heteroatom groups (e.g., —NH—, or —N(alkyl)-). More specifically, the term includes groups having a stable 3-7 membered monocyclic heterocyclic ring or a 8-11 membered bicyclic heterocyclic ring. The ring can be saturated, or partially or fully unsaturated, and may be optionally benzofused, if monocyclic and is optionally substituted unless otherwise stated on one or more carbon atoms by halogen, alkyl, alkoxy, oxo (═O) or on a secondary nitrogen atom by alkyl, phenyl or phenylalkyl. A number of heterocyclic groups are exemplified in the specification. Each heterocycle consists of one or more carbon atoms and from one to four heteroatoms selected from the group consisting of nitrogen, oxygen and sulfur and oxidized forms thereof. Heterocycles include 5-7 membered monocyclic heterocycles and 8-10 membered bicyclic heterocycles.

The term heteroaryl refers to a group having at least one aromatic ring wherein the ring contains at least one heteroatom or heteroatom group, as defined above. More specifically, the term refers to stable 5-6 membered monocyclic or 8-11 membered bicyclic aromatic hetero cycles where heterocycles is as defined above. Non-limiting examples of such groups include imidazolyl, quinolyl, isoquinolyl, indolyl, indazolyl, pyridazyl, pyridyl, pyrrolyl, pyrazolyl, pyrazinyl, quinoxolyl, pyranyl, pyrimidinyl, furyl, thienyl, triaZolyl, thiaZolyl, carbolinyl, tetrazolyl, benzofuranyl, thiamorpholinyl sulfone, oxazolyl, benzoxazolyl, benzimidazolyl, benzthiazolyl, oxopiperidinyl, oxoppyrrolidinyl, oxoazepinyl, azepinyl, isoxazolyl, isothiazolyl, furazanyl, thiazolyl, thiadiazolyl, and oxathiolyl.

A carbocyclyl group is a group having one or more saturated or unsaturated carbon rings. Carbocyclyl groups, for example, contain one or two double bonds. One or more carbons in a carbocyclic ring can be —CO— groups. Carbocyclyl groups include those having 3-12 carbon atoms, and optionally replacing 1 or 2 carbon atoms with a —CO— group and optionally having 1, 2 or 3 double bonds. Carbocyclyl groups include those having 5-6 ring carbons. Carbocyclyl groups can contain one or more rings each of which is saturated or unsaturated. Carbocyclyl groups include bicyclic and tricyclic groups. Preferred carbocyclic groups have a single 5- or 6-member ring. Carbocyclyl groups are optionally substituted as described herein. Specifically, carbocyclic groups can be substituted with one or more alkyl groups. Carbocyclyl groups include among others cycloalkyl and cycloalkenyl groups.

In certain compounds of the invention, two variables substituted on adjacent carbons in an alkyl group or on a ring can optionally together with the atoms (typically carbons or notrogens) to which they are bonded form a carbocyclic or heterocyclic ring. In embodiments, the ring can be a 5-member ring or a 6-member ring and may contain 0, 1 2 or 3 heteroatoms. Preferred heteroatoms for such rings are nitrogen, oxygen and sulfur. Such rings are optionally substituted, for example with one or more halogens, hydroxyl, C1-C3 alkyl, or carboxylate or esters thereof, or contain an oxo group (—CO—) in the ring. In an embodiment, such rings are unsubstituted. Such rings may be unsaturated (i.e., contain no double or triple bonds), partially unsaturated, i.e., contain one or two double bonds or may be aromatic (as understood in the art).

A styryl group or moiety is a group or moiety in which a vinyl group is bonded to a phenyl ring. The group or moiety may be substituted with one or more non-hydrogen substituents on the phenyl ring or most generally on the vinyl moiety. In formulas herein the styryl group can be in the E or Z configuration (or a mixture of E/Z configurations) having formulas:

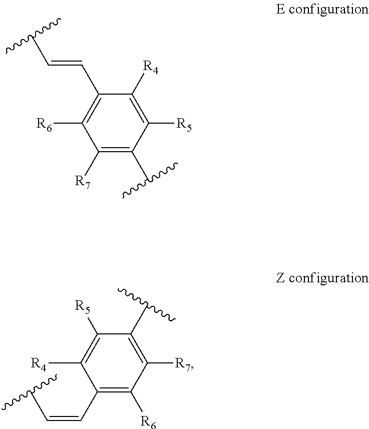

R₄-R₇ are optional substituents as defined herein. The formulas illustrate bonding to other groups at the para-position of the phenyl ring. The vinyl group can be written as a cross double bond to indicate that the double bond is a mixture of the E and Z configurations. Compounds herein can be a mixture of isomers having the E or Z configuration at the styryl group. Mixture of E and Z isomers may contain approximately equal amounts of E and Z isomers or may contain an excess of the E or Z an excess of the Z isomer. The amounts of E and Z isomers in a given mixture can be readily determined by standard analytical techniques, such as NMR (nuclear magnetic resonance) methods. A compound that is given isomer (E or Z) that is free of the other isomer contains less than 2% by weight of the other isomer. A compound that is given isomer (E or Z) that is free of the other isomer preferably contains less than 1% by weight of the other isomer. A compound that is given isomer (E or Z) that is free of the other isomer more preferably contains less than 0.5% by weight of the other isomer. It will be appreciated in the art that when it is desired to employ a compound or intermediate that is a given E or Z isomer that it can be also desirable to minimize the amount of the other isomer present. It will however also be understood that small, albeit detectible levels of the other isomer that may be present may not be detrimental to the utility of the desired isomer.

The term “cephem” refers generally to the ring system:

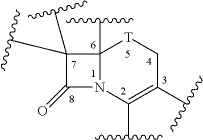

where ring atoms are numbered as indicated and T is —S—, —SO—, —SO₂—, —CHR₁₇—, or —O—. This numbering is used throughout for labeling of ring substituents. It will be appreciated by one of ordinary skill in the art that different ring numbering conventions may be employed in the art.

The term “penem” refers generally to the ring system:

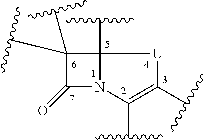

where ring atoms are numbered as indicated and U is —S—, —CHR₁₇— or —O—. This numbering is used throughout for labeling of ring substituents. It will be appreciated by one of ordinary skill in the art that different ring numbering conventions may be employed in the art. It is noted that the term penem is used generically herein. The term penem is also used in the art to refer to compounds of this ring system where U is —S—.

Certain groups in the compounds of the invention are optionally protected, for example, hydroxyl, carboxylate and amine groups. Methods of protection of such groups is known in the art and employed generally during synthesis again as is known in the art. T. W. Green et al. (1999) Protecting Groups in Organic Synthesis (Wiley Interscience) provides an overview of the types of protecting groups and their use. This reference is incorporated by reference herein for descriptions of types of protecting groups and methods for protecting and deprotecting. For example, compounds of the invention may contain a protected hydroxyl group or a protected carboxyl group.

Exemplary hydroxyl protecting groups include alkoxyalkyl groups (e.g., methoxymethyl, forming an ether at the hydroxy), C1-C3 alkyl or C2-C6 alkenyl or arylalkyl or silyl groups (again forming ethers), or —CO-alkyl or —CO-arylalkyl (forming esters. Specific hydroxyl protecting groups include, among others, —COCH₃, —CO-t-butyl, —CO-phenyl (optionally substituted at the phenyl ring), —CO-benzyl (optionally substituted at the phenyl ring), optionally substituted benzyl, allyl, silyl (trialkylsilyl, diarylalkysilyl, aryldialkylsilyl, e.g., trimethylsily, diphenylmethylsilyl, phenyldimethylsilyl, and the like). Hydroxy groups may also be protected by formation of acetals. Carboxylate groups may be protected as esters.

Carboxylate esters (—CO₂-E) include those where E is an optionally substituted C1-C6 or C1-C3 alkyl (optional substation includes among others substitution with one or more halogen), an optionally substituted phenyl or phenylalkyl group (where substitution includes among others ring substitution with one or more halogen or with a nitro group). Carboxylate esters further include those in which E is an alkoxyalkyl group, more specifically where the alkoxy and the alkyl group is C1-C6 or C1-C3 alkoxy and C1-C6 or C1-C3 alkyl, e.g., methyoxymethyl, ethoxyethyl, methoxyethyl and the like). Carboxylate esters (—CO₂-E) include those where E is an acylalkoxyalkyl group, more specifically where the acyl, alkoxy and alkyl groups are C1-C6 or C1-C3 acyl, alkoxy or alkyl groups.

Certain compounds of the invention may contain one or more stereogenic centers resulting in possible stereoisomers. It will be understood that for any such compound of the invention that all individual stereoisomer, any diastereomers and all mixtures thereof are included in the compounds as claimed. For any such compound the compound may be a racemic mixture of stereoisomers or may be a mixture which contains an excess of one stereoisomer and is thus optically active.

The term styrylmethylene refers to the moiety:

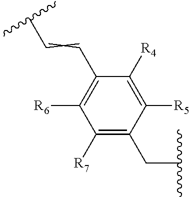

The formula again illustrates bonding to other groups at the para-position of the phenyl ring. The formula indicates the E or Z or mixture of E/Z configuration. In compounds of the invention the styrylmethylene moiety is bonded between the cephem or penem ring and a good leaving group.

R₁—CO—NH— in formulas herein is an acylamino group. In general, R₁—CO—NH— can be any pharmaceutically acceptable A such group that is known in the art to be compatible with a cephem antibiotic. A number of examples of R₁ useful groups are provided in the specification. Additional useful R₁ groups are known in the art. In specific embodiments, useful R₁ groups are substituted methylene groups. In other embodiments, useful R₁ groups are substituted oximes. In other specific embodiments, useful R₁ groups are substituted vinyl ether groups.

Groups herein are optionally substituted. Most generally alky, alkenyl, and aryl, heteroaryl, and heterocyclyl groups are optionally substituted, for example, with one or more oxo group, thioxo group, halogen, nitro, cyano, cyanate, azido, thiocyano, isocyano, isothiocyano, sulfhydryl, hydroxyl, alkyl, alkoxy, alkenyl, alkenyloxy, alkynyl, alkynyloxy, aryl, aryloxy, amino (—NH₂), heteroaryl, heteroaryloxy, carbocyclyl, carbocyclyloxy, heterocyclyl, heterocyclyloxy, alkylthio, alkenylthio, alkynylthio, arylthio, thioheteroaryl, thioheteroaryl, thiocarbocyclyl, thioheterocyclyl, —COR′, —COH, —OCOR′, —OCOH, —CO—OR′, —CO—OH, —CO—O—CO—R′, —CON(R′)₂, —CONHR′, —CONH₂, —NR—COR′, —NHCOR′, —NHR′, —N(R′)₂, —O—SO₂—R′, —SO₂—R′, —SO₂—NHR′, —SO₂—N(R″)₂, —NR—SO₂—R′, —NH—SO₂—R′, —NR′CO—N(R′)₂, —NH—CO—NHR′, —O—PO(OR′)₂, —O—PO(OR′)(N(R′)₂), —O—PO(N(R′)₂)₂, where each R′ independently is an organic group and more specifically is an alkyl, cyclolkyl, alkenyl, cycloalkenyl, aryl, heteroaryl, or heterocyclyl group or two R′ within the same substitutent can together form a carbocyclic (containing only carbon ring members) or heterocyclic ring having 3 to 10 ring atoms. Organic groups of non-hydrogen substituents are in turn optionally substituted with one or more halogens, nitro, cyano, isocyano, isothiocyano, hydroxyl, sulfhydryl, haloalkyl, hydroxyalkyl, amino, alkylamino, dialkylamino, arylalkyl, unsubstituted alkyl, unsubstituted alkenyl, unsubstituted alkynyl alkylalkenyl, alkylalkynyl, haloaryl, hydroxylaryl, alkylaryl, unsubstituted aryl, unsubstituted carbocylic, halo-substituted carbocyclic, hydroxyl-substituted carbocyclic, alkyl-substituted carbocyclic, unsubstituted heterocyclic, unsubstituted heteroaryl, alkyl-substituted heteroaryl, or alkylsubstituted heterocyclic. In specific embodiments, organic groups of non-hydrogen substituents are not further substituted. In specific embodiments, R′ groups of substituents are independently selected from alkyl groups, haloalkyl groups, phenyl groups, benzyl groups and halo-substituted phenyl and benzyl groups. In specific embodiments, non-hydrogen substituents have 1-10 carbon atoms, 1-7 carbon atoms, 1-5 carbon atoms or 1-3 carbon atoms. In specific embodiments, non-hydrogen substituents have 1-10 heteroatoms, 1-6 heteroatoms, 1-4 heteroatoms, or 1, 2, or 3 heteroatoms. Heteroatoms preferably are O, N or S. In specific embodiments, substituent groups include heterocyclic groups having a single 5- or 6-member ring containing 1, 2 or 3 heteroatoms selected from N, O or S and in which one or more ring carbons or nitrogens are optionally substituted with a C1-C3 alkyl group.

In specific embodiments, optional substitution is substitution with 1, 2, 3, 4 or 5 non-hydrogen substituents. In specific embodiments, optional substitution is substitution with 1 or 2 non hydrogen substituents. In specific embodiments, optional substitution is substitution with 1 non hydrogen substituents. In specific embodiments, optional substitution is substitution by one or more halogen, hydroxyl group, cyano group, nitro group, oxo group, thioxo group, unsubstituted C1-C6 alkyl group or unsubstituted aryl group. The term oxo group refers to substitution of a carbon atom with a ═O, for example to form a —CO— (carbonyl)] group.

In specific embodiments, optional substitution of phenyl rings, includes substitution with one or more halogen, one or more hydroxyl, one or more C1-C3 alkyl, one or more C1-C3 alkoxy, one or more nitro, or one or more cyano. In specific embodiments, optional substitution of phenyl rings, includes substitution with one or more halogen, one or more hydroxyl, one or more C1-C3 alkyl, or one or more C1-C3 alkoxy. In specific embodiments, optional substitution of phenyl rings, includes substitution with one or more halogen, one or more hydroxyl, or one or more C1-C3 alkyl. In specific embodiments, optional substitution of phenyl rings, includes substitution with one halogen, one hydroxyl, one C1-C3 alkyl, one C1-C3 alkoxy, one nitro, and/or one cyano.

As to any of the above groups which contain one or more substituents, it is understood, that such groups do not contain any substitution or substitution patterns which are sterically impractical and/or synthetically non-feasible. In addition, the compounds of this invention include all stereochemical isomers arising from the substitution of these compounds.

The invention provides methods for treating or preventing bacterial infection and the symptoms and disorders associated therewith. The invention provides pharmaceutical compositions comprising a pharmaceutically effective amount of one or more compounds and/or salts and/or solvates of the invention and a pharmaceutically acceptable carrier or excipient. The compounds of the invention are of particular use in the treatment of infections of bacteria which exhibit resistance to or more beta-lactam antibiotics, particularly those that exhibit resistance to one or more cephalosporins and particularly those that exhibit resistance to a carbapenem. Compounds of the invention are of particular use in the treatment of infections of bacteria which generate one or more ESBL. Compounds of the invention exhibit resistance to one or more ESBL.

Compounds of the invention are of particular use in the treatment of infections of bacteria which generate one or more ESBL. Compounds of the invention are of particular use in the treatment of infections of Gram-negative bacteria. Compounds of the invention are of particular use in the treatment of infections of Pseudomonas strains and particularly strains of Pseudomonas aeruginosa.

The compounds, salts and solvates thereof of the invention can be used to prepare medicaments for the treatment and prevention of bacterial infection and the symptoms and disorders associated therewith. The compounds, salts and solvates thereof of the invention can be used in particular to prepare medicaments for the treatment and prevention of bacterial infection of bacteria which produce one or more ESBL and the symptoms and disorders associated therewith.

The term “pharmaceutically effective amount” refers to an amount effective in treating a bacterial infection, or a symptom or complication thereof, in a patient (human or other mammal) either by administration of a single compound of Formulas I-III, or a salt or solvate thereof or in combination with other agents. The pharmaceutically effective amount of a given compound when administered as the only active ingredient may differ from its pharmaceutically effective amount when administered with other active ingredients. It will be appreciated that the pharmaceutically effective amount of a compound may differ from that of a salt of the same compound. Treating includes the alleviation of symptoms of a particular disorder in a patient or a measurable improvement of a parameter associated with a particular disorder. Treating includes treatment to prevent a bacterial infection and to delay the progress of an infection. The term “prophylactically effective amount” refers to an amount of a compound or salt of the invention effective in preventing a bacterial infection in a patient. The compounds of the present invention are useful in the treatment of individuals infected by various bacteria (which include infections of combinations of different bacteria) for the prophylaxis of these individuals. It will be appreciated in the art, that individuals at risk for bacterial infection can be treated employing one or more of the compounds or salts of the invention to prevent or delay infection.

As used herein, the term “patient” refers to any animal and more specifically to a mammal, including a human.

Compounds of the invention may contain chemical groups (acidic or basic groups) that can be in the form of salts. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), bisulfate, sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines [formed with N,N-bis(dehydro-abietyl)ethylenediamine], N-methyl-D-glucamines, N-methyl-D-glucamides, t-butyl amines, and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others.

Compounds of the invention may be in the zwitterionic form.

Salts of the invention include “pharmaceutically acceptable salts” which refers to those salts which retain the biological effectiveness and properties of the free bases or free acids, and which are not biologically or otherwise undesirable. Pharmaceutically acceptable salts comprise pharmaceutically-acceptable anions and/or cations.

Compounds of the invention can be administered in the form of pharmaceutically acceptable salts which include the following non-limiting examples: alkali metal salts, such as those of lithium, potassium and sodium; alkali earth metal salts, such as those of barium, calcium and magnesium; transition metal salts, such as those of zinc; and other metal salts, such as those of aluminum, sodium hydrogen phosphate and disodium phosphate; salts of nitrates, borates, methanesulfonates, benzene sulfonates, toluenesulfonates, salts of mineral acids, such as those of hydrochlorides, hydrobromides, hydroiodides and sulfates; salts of organic acids, such as those of acetates, trifuoroacetates, maleates, oxalates, lactates, malates, tartrates, citrates, benzoates, salicylates, ascorbates, succinates, butyrates, valerates and fumarates, amine salts, such as those of N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane.

In specific embodiments, compound of any formula herein are salts wherein the anion of the salt is selected from halide, more specifically chloride or bromide, acetate, tosylate, tartrate, sulfate, bisulfate, succinate, phosphate, oxalate, nitrate, mesylate, maleate, malate, and citrate. In more specific embodiments, compounds of any formula herein are salts of chloride, bromide, sulfate, bisulfate, or acetate.

In specific embodiments, compounds of the invention are hydrates of the compounds herein and more specifically are hydrates of salts of the compounds herein.

Pharmaceutically acceptable salts of the compounds of the invention can be derived from inorganic or organic acids or can be derived from inorganic or organic bases as is known in the art. Basic amino acids useful for salt formation include arginine, lysine and ornithine. Acidic amino acids useful for salt formation include aspartic acid and glutamic acid.

Compound of the invention can be administered in the form of pharmaceutically acceptable esters which include, among others, alkyl, alkenyl, alkynyl, aryl, heteroaryl, cycloalkyl and heterocyclyl esters of acidic groups, including, but not limited to, carboxylic acids, phosphoric acids, phosphinic acids, sulfonic acids, sulfinic acids and boronic acids.

Those of ordinary skill in the art will appreciate that many organic compounds, including salts, can form complexes with solvents in which they are reacted or from which they are precipitated or crystallized. These complexes are known as “solvates”. For example, a complex with water is known as a “hydrate”. Solvates, and more particularly hydrates, of the cephem compounds of the invention are within the scope of the invention. Pharmaceutically acceptable solvates and hydrates are complexes of a compound of the invention with one or more solvent or water molecules, or 1 to about 100, or 1 to about 10, or one to about 2, 3 or 4, solvent or water molecules. Compounds and salts of the invention in the form of pharmaceutical compositions or dosage forms can be administered by any known route that is appropriate for the patient being treated and for the treatment or prophylaxis that is desired. Specifically administration can be orally or non-orally in the form of, for example, granules, powders, tablets, capsules, syrup, suppositories, injections, emulsions, elixir, suspensions or solutions, by mixing these effective components, individually or simultaneously, with pharmaceutically acceptable carriers, excipients, binders, diluents or the like.

A solid formulation for oral administration can comprise one or more of the compounds or salts, or solvates thereof, of the invention alone or in appropriate combination with other active ingredients. Solid formulations can be in the form of powders, granules, tablets, pills and capsules. In these cases, the instant compounds can be mixed with at least one additive, for example, sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. These formulations can contain, as in conventional cases, further additives, for example, an inactive diluent, a lubricant such as magnesium stearate, a preservative such as paraben or sorbic acid, an anti-oxidant such as ascorbic acid, tocopherol or cysteine, a disintegrator, a binder, a thickening agent, a buffer, a sweetener, flavoring agent and/or a perfuming agent. Tablets and pills can also be prepared with enteric coating. Standard methods of formulation can be applied to preparation of formulations of the compounds and salts of the invention.

Non-oral administration includes subcutaneous injection, intravenous injection, intramuscular injections, intraperitoneal injection or instillation. Injectable preparations, for example, sterile injectable aqueous suspensions or oil suspensions can be prepared by known methods.

The instant pharmaceutical compositions may be formulated as known in the art for administration by inhalation, such as in the form of a nasal aerosol or nasal spray, a dry powder spray or as a solution or suspension for inhalation and may be prepared as solutions or suspensions in saline or other suitable carrier, and benzyl alcohol or other suitable preservatives, absorption promoters, fluorocarbons, or solubilizing or dispersing agents.

Rectal suppositories can be prepared by mixing the drug with a suitable vehicle, for example, cocoa butter and polyethylene glycol, which is in the solid state at ordinary temperatures, in the liquid state at temperatures in intestinal tubes and melts to release the drug.

Examples of liquid preparations for oral administration include pharmaceutically acceptable emulsions, syrups, elixirs, suspensions and solutions, which may contain an inactive diluent, for example, pharmaceutically acceptable water.

The pharmaceutical composition herein can be formulated for topical administration with a suitable ointment containing one or more of the compounds or salts of the invention suspended or dissolved in a carrier, which include mineral oil, liquid petroleum, white petroleum, propylene glycol, polyoxyethylene polyoxypropylene compound, emulsifying wax and pharmaceutically acceptable water. In addition, topical formulations can be formulated with a lotion or cream containing the active compound suspended or dissolved in a carrier. Suitable carriers include mineral oil, sorbitan monostearate, polysorbate 60, cetyl esters wax, cetearyl alcohol, 2-octyldodecanol, benzyl alcohol and pharmaceutically acceptable water.

As is understood in the art, dosages of therapeutic compounds are dependent on age, body weight, general health conditions, sex, diet, dose interval, administration routes, excretion rate, combinations of drugs and conditions of the diseases treated. While taking these and other necessary factors into consideration, generally, dosage levels of between about 10 pg per day to about 5000 mg per day, preferably between about 100 mg per day to about 1000 mg per day of the compound are useful in the treatment of bacterial infection. Typically, the pharmaceutical compositions of this invention will be administered from about 1 to about 5 times per day or alternatively, as a continuous infusion. Such administration can be used as a chronic or acute therapy.

The amount of active ingredient that may be combined with the carrier or excipient materials to produce a single dosage form will vary depending upon the patient/individual treated and the particular mode of administration. A typical preparation will contain from about 5% to about 95% active compound (W/W). Preferably, such preparations contain from about 20% to about 80% active compound.

While these dosage ranges can be adjusted by a necessary unit base for dividing a daily dose, as described above, such doses are decided depending on the diseases to be treated, conditions of such diseases, the age, body weight, general health conditions, sex, diet of the patient then treated, dose intervals, administration routes, excretion rate, and combinations of drugs. While taking these and other necessary factors into consideration., for example, a typical preparation will contain from about 0.05% to about 95% active compound (W/W). Preferably, such preparations contain from about 10% to about 80% active compound. The unit dose of the composition of this invention is administered once or multiple times daily.

A desirable dosage form for antibiotic compounds of the invention is an oral dosage form to be administered once a day or twice a day.

Another desirable dosage form for antibiotic compounds of the invention is an parenteral dosage form to be administered once a day or twice a day.

To treat bacterial infections and complications thereof, the antibacterial compounds of this invention may be co-administered in combination with one or more antibacterial compounds, including antibacterial compounds which are cephems or penems other than those of Formulas I-III herein and including antibacterial compounds which are not beta-lactam antibiotics. In addition, the antibacterial compounds of this invention may be co-administered in combination with one or more beta-lactamase inhibitors other than the compounds of Formula I herein. Co-administration includes among others separate administration of active ingredients at about the same time, combined administration in the same formulation, as well as sequential separate administration.

Compounds of the invention may be co-administered with a beta-lactamase inhibitor. In a specific embodiment, compounds of the invention may be co-formulated with a beta-lactamase inhibitor. A number of such inhibitors are known in the art and one of ordinary skill in the art understands how to co-formulate such inhibitors with a given beta-lactam antibiotic. Beta-Lactamase inhibitors include among others avibactam, tazobactam, sulbactam, clavulanic acid, and relebactam.

Compounds of the invention may be co-administered with a monobactam, such as aztreonam. Compounds of the invention may be co-administered with a carbapenem, other than a compound of Formula I, such as meropenem.

Compounds of the invention may be co-administered with a non-beta lactam antibiotic, such as an aminoglycoside antibiotic. Compounds of the invention may be co-administered with an aminoglycoside antibiotic, such as amikacin, gentamicin, kanamycin, neomycin, or tobramycin.

The invention also relates to the use of one or more compound, salt or solvate of Formula I and any sub formula thereof for the treatment of a bacterial infection or a complication of a bacterial infection. The invention further relates to the use of one or more pharmaceutical compositions comprising a compound, salt or solvate of Formula I and any sub formula thereof for the treatment of a bacterial infection or a complication of a bacterial infection.

The invention also relates to the use of one or more compound, salt or solvate of Formula I and any sub formula thereof for the preparation of a medicament for the treatment of a bacterial infection or a complication thereof. The invention further relates to the use of one or more pharmaceutical compositions comprising a compound, salt or solvate of Formula I and any sub formula thereof for the preparation of a medicament for the treatment of a bacterial infection or a complication thereof.

All references throughout this application, for example patent documents including issued or granted patents or equivalents; patent application publications; and non-patent literature documents or other source material; are hereby incorporated by reference herein in their entireties, as though individually incorporated by reference.

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

When a group of substituents is disclosed herein, it is understood that all individual members of that group and all subgroups, including any isomers and enantiomers of the group members, and classes of compounds that can be formed using the substituents described are disclosed separately.

When compounds are claimed using a generic formula, it should be understood that compounds known in the art including the compounds disclosed in the references disclosed herein are not intended to be included. When a Markush group or other grouping is used herein, all individual members of the group and all combinations and subcombinations possible of the group are intended to be individually included in the invention.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomers and enantiomer of the compound described individual or in any combination.

One of ordinary skill in the art will appreciate that methods, device elements, starting materials, and synthetic methods other than those specifically exemplified can be employed in the practice of the invention without resort to undue experimentation. All art-known functional equivalents, of any such methods, device elements, starting materials, and synthetic methods are intended to be included in this invention. Whenever a range is given in the specification, for example, a temperature range, a time range, or a composition range, all intermediate ranges and subranges, as well as all individual values included in the ranges given are intended to be included in the invention.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein. Without wishing to be bound by any particular theory, there can be discussion herein of beliefs or understandings of underlying principles relating to the invention. It is recognized that regardless of the ultimate correctness of any mechanistic explanation or hypothesis, an embodiment of the invention can nonetheless be operative and useful.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention.

### THE EXAMPLES

### Synthetic Examples

### Example 1: Preparation of Exemplary Cephem Intermediates for Synthesis of Compounds of Formula I

### IA. Synthesis of 4-methoxybenzyl (6R,7R)-3-((E/Z)-4-(chloromethyl)styryl)-8-oxo-7-(2-phenylacetamido)-5-thia-1 azabicyclo[4.2.0]oct-2-ene-2-carboxylate (1), a Compound of Formula X

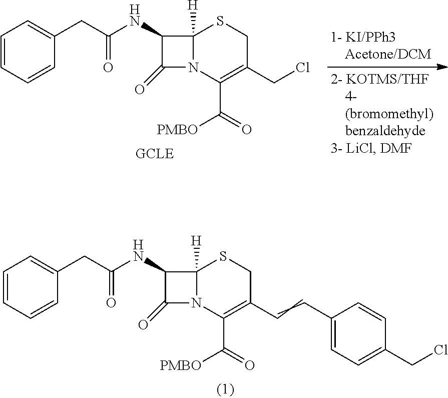

GCLE (8.742 g, 18.0 mmol) is dissolved in acetone (420 mL) and dichloromethane (85 mL) under a nitrogen atmosphere. (PMB is 4-methoxybenzyl). Triphenylphosphine (4.709 g, 18.0 mmol) is added, followed by potassium iodide (3.278 g, 19.8 mmol). The reaction flask is covered with aluminum foil and the mixture is stirred at room temperature overnight. The reaction is filtered through a pad of Celite and the solids are washed twice with a minimum amount of ethyl acetate. The filtrate is concentrated to give a crude mixture as a light-yellow foam, which is used in the next reaction without further purification. This crude mixture (18.0 mmol) is dissolved in THF (230 mL) under a nitrogen atmosphere. The mixture is stirred for 30 minutes, and is then filtered. The filtrate is cooled to 0° C. in an ice bath and potassium trimethylsilanoate (2.533 g, 19.8 mmol) is added. The reaction is stirred at 0° C. for 1 h and is then filtered through a pad of Celite. The solids are washed twice with a minimum amount of THF. A solution of 4-(bromomethyl)benzaldehyde (3.573 g, 18.0 mmol) in dichloromethane (89 mL) is added to the filtrate at room temperature. The reaction is covered with aluminum foil and stirred for 65 h. The reaction is then cooled to 0° C., and ethyl acetate and saturated NaHCO₃ are added. The layers are separated and the aqueous layer is extracted with ethyl acetate (3×). The combined organic phases are washed with brine, dried over Na₂SO₄ and concentrated. The residue is purified by flash column chromatography using a gradient of ethyl acetate/dichloromethane to give a mixture of compound 1 and its -iodo analog (1-Cl/I mixture) as an orange-brown solid (0.74 g, 6.4%).

The solid obtained (0.738 g, 1.25 mmol) from the previous reaction is dissolved in anhydrous DMF (12 mL) under a nitrogen atmosphere. Lithium chloride (0.3187 g, 7.52 mmol) is added and the mixture is stirred at room temperature overnight. The reaction is diluted with water and then extracted with ethyl acetate (3×). The combined organic phases are washed with brine, dried over Na₂SO₄ and concentrated. The residue is purified by flash column chromatography using a gradient of ethyl acetate/dichloromethane to give compound 1 as a golden-yellow solid (0.55 g, 74%), m/z=613, [M+H+Na]⁺.

### IB. Synthesis of 4-methoxybenzyl 96R, 7R)-7-amino-3-((E/Z)-4-(chloromethyl)styryl)-8-oxo-5-thia-1-azabicyclo[4.2.0]oct-2-ene-2-carboxylate, Compound 2, a Compound of Formula X

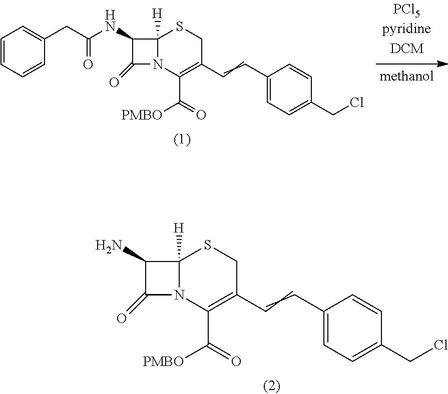

Phosphorus pentachloride (1.25 g, 6.00 mmol) is suspended in anhydrous dichloromethane (26 mL) under a nitrogen atmosphere and the mixture is cooled with an ice bath for 20 minutes. Anhydrous pyridine (0.48 mL, 6.00 mmol) is added and the reaction is stirred an additional 30 minutes at 0° C. A solution of 1 (0.6 g, 1.0 mmol) in dichloromethane (13 mL) is added and the mixture is stirred at 0° C. for 3 h. The reaction is then cooled to −78° C., methanol (4.0 mL) is added, and the reaction is stirred at this temperature for 10 min before being warmed to 0° C. and stirred for an additional 10 min and then concentrated. The residue is taken up into dichloromethane and washed with a saturated solution of NaHCO₃. The layers are separated and the aqueous layer is extracted with dichloromethane (2×). The combined organic phases are washed with brine, dried over Na₂SO₄ and filtered. The filtrate is partially concentrated to provide a solution of 2, which is used in syntheses without further purification.

### IC. Synthesis of tert-butyl (Z)-2-(((1-(2-((tert-butoxycarbonyl)amino)thiazol-4-yl)-2-chloro-2-oxoethylidene)amino)oxy)-2-methylpropanoate, Compound (4), an Acyl Chloride Compound of Formula XIII

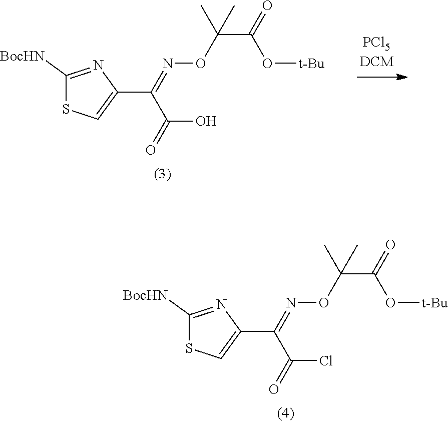 [SMILES: CC(C)(C)OC(=O)Nc1nc(/C(=N/OC(C)(C)C(=O)OC(C)(C)C)C(=O)O)cs1 ; CC(C)(C)OC(=O)Nc1nc(/C(=N/OC(C)(C)C(=O)OC(C)(C)C)C(=O)Cl)cs1]

Phosphorus pentachloride (0.4903 g, 2.35 mmol) is dissolved in anhydrous dichloromethane (11 mL) under a nitrogen atmosphere, and (Z)-2-(((1-(tert-butoxy)-2-methyl-1-oxopropan-2-yl)oxy)imino)-2-(2-((tert-butoxycarbonyl)amino)thiazol-4-yl)acetic acid, compound 3 (0.84 g, 1.96 mmol) is added as a solid. The resulting mixture is stirred at room temperature for 2 h. The reaction mixture is concentrated to dryness to provide compound 4, which is used in syntheses without further purification.

### ID. Synthesis of 4-methoxybenzyl (6R,7R)-7-((Z)-2-(((1-(tert-butoxy)-2-methyl-1-oxopropan-2 yl)oxy)imino)-2-(2-((tert-butoxycarbonyl)amino)thiazol-4-yl)acetamido)-3-((E/Z)-4 (chloromethyl)styryl)-8-oxo-5-thia-1-azabicyclo[4.2.0]oct-2-ene-2-carboxylate, Compound (5), a Compound of Formula X

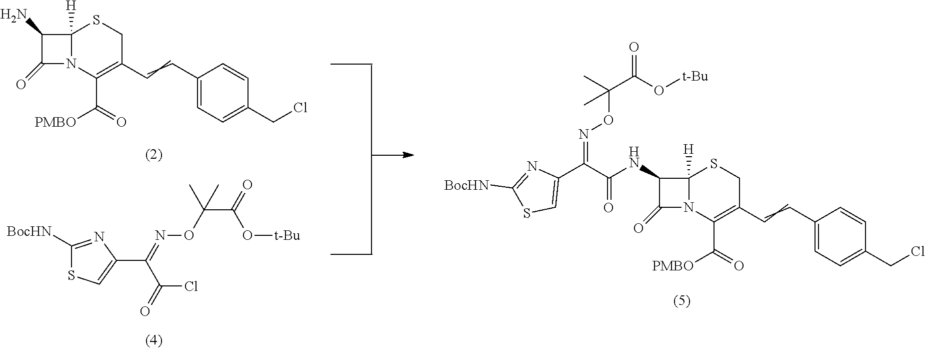

The solution of 2 in dichloromethane (43 mL) is treated with N-(trimethylsilyl)acetamide (2.36 g, 18 mmol) and cooled in an ice bath under a nitrogen atmosphere. The solution of 4 in anhydrous dichloromethane (4 mL) is added. The reaction is stirred for 10 minutes at 0 C, and then warmed to room temperature and stirred for 19 h. The reaction is then cooled back to 0° C. and a saturated solution of NaHCO₃ is added. The layers are separated and the aqueous layer is extracted with dichloromethane (3×). The combined organic layers are dried over Na₂SO₄, filtered through a pad of Celite, and concentrated. The residue is purified by flash column chromatography using a gradient of ethyl acetate/toluene to give compound 5 (0.5 g, 56%) as a yellow film, m/z=726, [M+H-(Boc)-(t-Bu)]⁺.

Intermediates 5-N (E/Z), 5-3 (E/Z) and 5-3N (E/Z), which differ from each other in the R₁ group of Formula I:

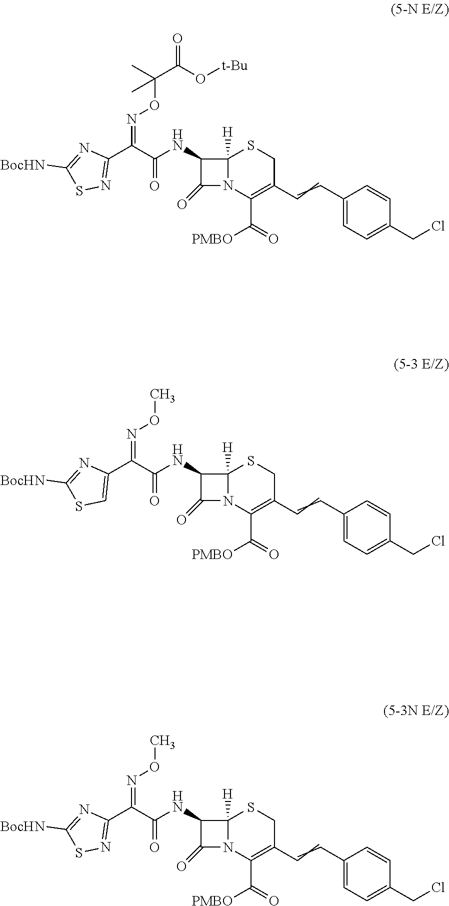

are prepared as described for compound 5 (E/Z) with appropriate choice of starting materials.

Intermediates 5-N E, 5-3 E and 5-3N E (E isomers) free of corresponding Z isomers and 5-N Z, 5-3 Z and 5-3N Z (Z isomers) free of corresponding E isomers are prepared as described herein for preparation of 5-E and 5-Z, again by appropriate choice of starting materials.

### IE. Synthesis of (6R,7R)-7-((Z)-2-(2-aminothiazol-4-yl)-2-(((2-carboxypropan-2-yl)oxy)imino)acetamido)-3-((E/Z)-4-(chloromethyl)styryl)-8-oxo-5-thia-1-azabicyclo[4.2.0]oct-2-ene-2-carboxylic Acid, Compound 6, a Compound of Formula X

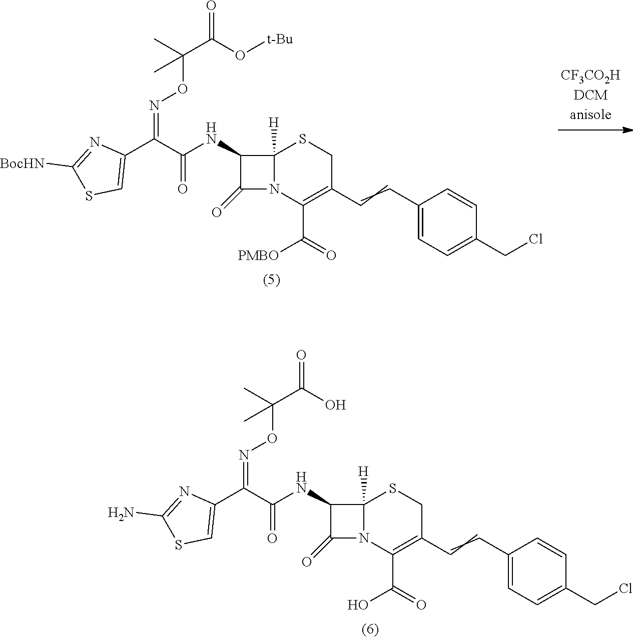

A solution of compound 5 (0.50 g, 0.57 mmol) in dry dichloromethane (5.6 mL) is cooled in an ice bath. Anisole (1.2 mL, 11 mmol) is added, the reaction is stirred for 10 minutes, and then trifluoroacetic acid (5.6 mL, 73 mmol) is added. The reaction is stirred in an ice bath for 40 min. and stored in a refrigerator overnight. The reaction is then concentrated, the residue is triturated with hexanes and the solid is isolated and washed with hexanes (3×) to provide compound 6 (quantitative) as a yellow solid. The solid mixture of E and Z isomers is used in further reactions without further purification, m/z=606, [M+H]⁺.

### Partially Deprotected and Deprotected Intermediates

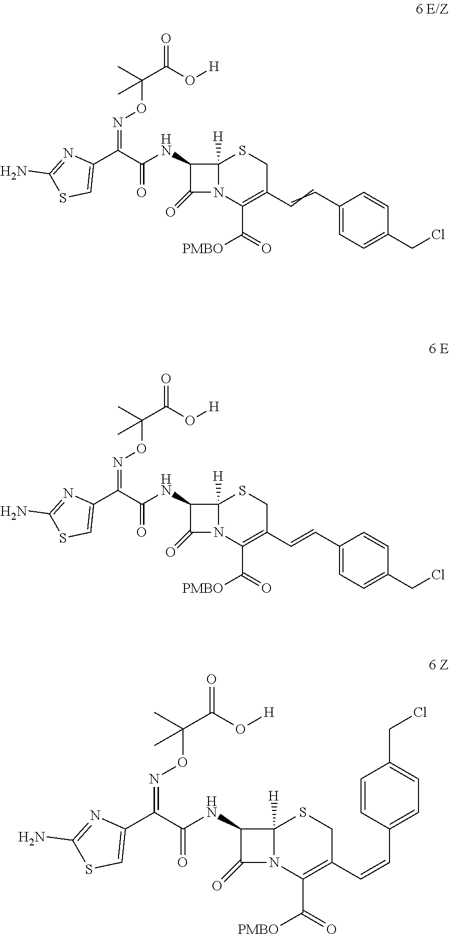

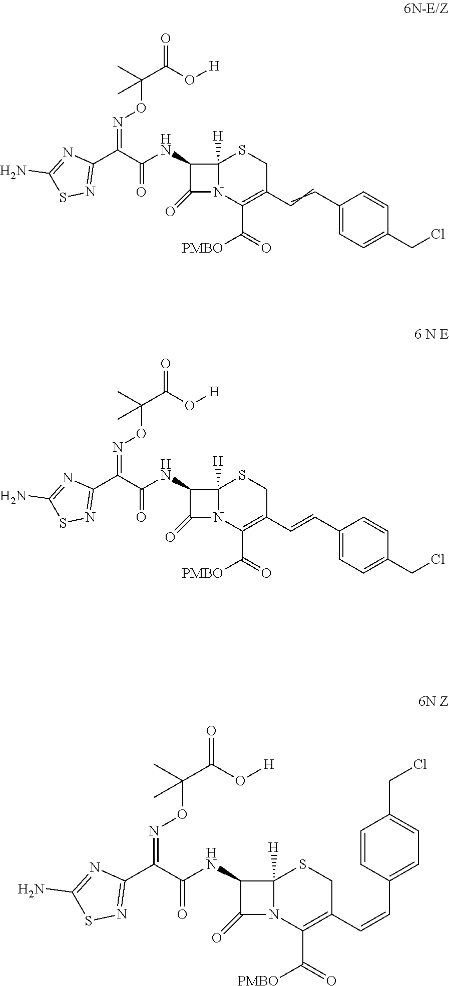

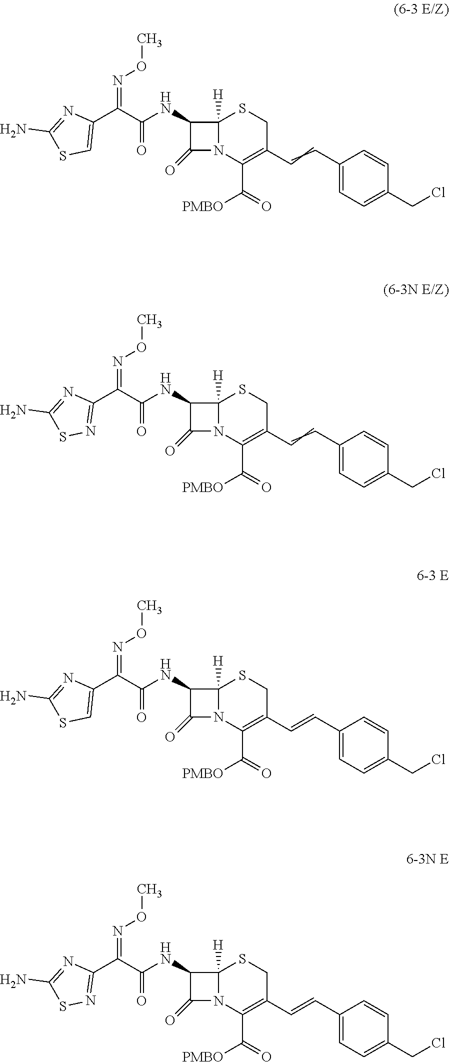

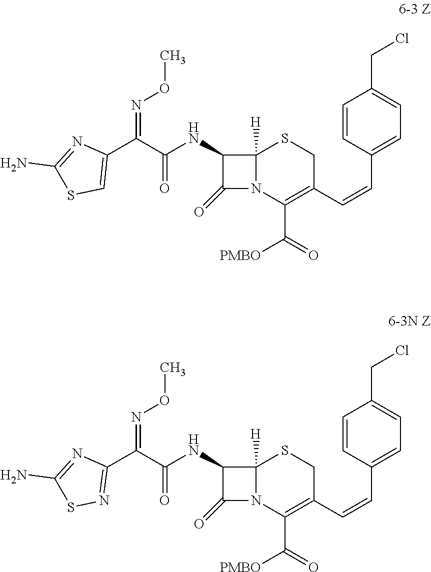

and the fully deprotected analog there of where PMBO is replaced with H) are prepared as described for compound 6 (E/Z) with appropriate choice of starting materials. Intermediates 6-N E, 6-3 E and 6-3N E (E isomers) free of corresponding Z isomers and 6-N Z, 6-3 Z and 6-3N Z (Z isomers) free of corresponding E isomers are prepared as described herein for preparation of 6-E and 6-Z, respectively, again by appropriate choice of starting materials.

### 1F. Synthesis of 4-methoxybenzyl (6R,7R)-3-((E)-4-(chloromethyl)styryl)-8-oxo-7-(2-phenylacetamido)-5-thia-1-azabicyclo[4.2.0]oct-2-ene-2-carboxylate (1-E)

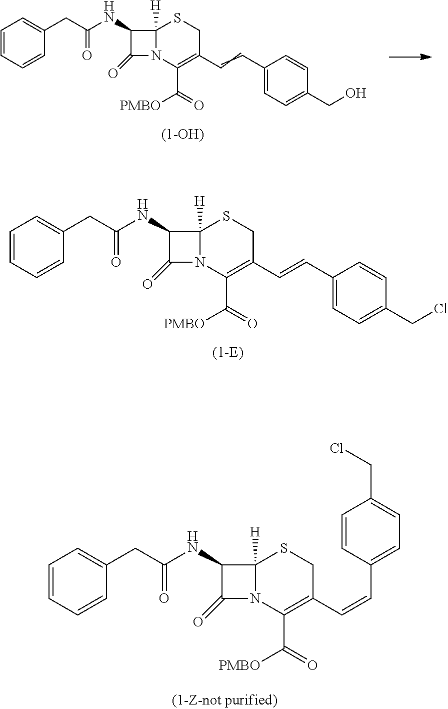

To a stirred suspension of crude E/Z compound 1-OH (19.0 g, 33.3 mmol) in DMA (950.0 mL), PCl₅ (10.4 g, 49.9 mmol) is added at 0° C. The mixture is stirred for 2 h at 0° C. The progress of the reaction is monitored by TLC. The reaction mixture is quenched with water and extracted with EtOAc (2×). The combined organic layers are washed with brine (3×), dried over sodium sulfate and concentrated under reduced pressure to obtain a crude mixture of E and Z-isomers (1-E and 1-Z). The crude mixture is treated with ethyl acetate (60.0 mL), stirred for 30 min at room temperature and then filtered to afford compound 1-E (E isomer, 5.9 g, 30.1%) as a pale yellow solid. The filtrate is concentrated to dryness to afford a dark brown oily mass (9.8 g), which contained mostly a mixture of E and Z isomers. The Z isomer is not isolated from this material as a pure compound.

### 1G. Synthesis of 4-methoxybenzyl (6R,7R)-3-((E/Z)-4-(hydroxymethyl) styryl)-8-oxo-7-(2-phenylacetamido)-5-thia-1-azabicyclo[4.2.0]oct-2-ene-2-carboxylate (1-OH)

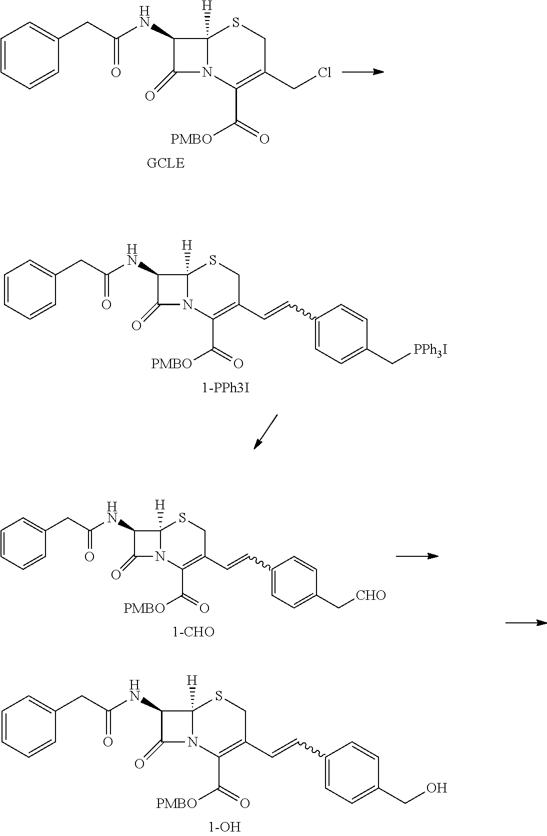

### Synthesis of 4-methoxybenzyl (6R,7R)-3-((iodotriphenylphosphanyl) methyl)-8-oxo-7-(2-phenylacetamido)-5-thia-1-azabicyclo[4.2.0]oct-2-ene-2-carboxylate (1-PPH₃I)

To a stirred suspension of GCLE (50.0 g, 103.0 mmol) in acetone (2.55 L) is added triphenylphosphine (33.7 g, 128.0 mmol) and potassium iodide (25.6 g, 154.0 mmol) at RT. The resulting mixture is stirred at RT for 8 h in the dark. TLC is checked to ensure completion of reaction. The off-white suspended mixture is concentrated under reduced pressure at 65° C. to afford compound 1-PPh3I (103.0 g, 119%) as a creamy foamy solid, m/z=713 [M+H]⁺.

### Synthesis of 4-methoxybenzyl (6R,7R)-3-((E/Z)-4-formylstyryl)-8-oxo-7-(2-phenylacetamido)-5-thia-1-azabicyclo[4.2.0]oct-2-ene-2-carboxylate (1-CHO)

To a stirred suspension of phosphonium iodide salt 1-PPH3I (83%, 102.0 g, 101.0 mmol) in THF (1.063 L) and DCM (561.0 mL) is added potassium trimethylsilanolate (95.0%, 16.3 g, 121.0 mmol) at −10° C. After stirring for 30 min at −10° C., a solution of terephthaldehyde (67.5 g, 503.0 mmol) in DCM (561.0 mL) is added at −10° C. The reaction mixture is allowed to warm to RT and stirred overnight. The progress of reaction is monitored by TLC. The mixture is concentrated to dryness and partitioned between water and EtOAc. The aqueous layer is further extracted with EtOAc. The combined organic layers are washed with brine (1.0 L), dried over sodium sulfate and concentrated to dryness. The crude product is purified through column chromatography eluted with 0-20% EtOAC in DCM to afford compound 1-CHO (29.6 g, 49.9%) as golden yellow solid, m/z=567 [M−H]⁻.

### Synthesis of 4-methoxybenzyl (6R,7R)-3-((E/Z)-4-(hydroxymethyl) styryl)-8-oxo-7-(2-phenylacetamido)-5-thia-1-azabicyclo[4.2.0]oct-2-ene-2-carboxylate (1-OH)

To a stirred solution of sodium borohydride (1.10 g, 29.0 mmol) in methanol (360 mL) and THF (360 mL) is slowly added a solution of compound 1-CHO (30.0 g, 52.8 mmol) in THF (1.20 L) at 0° C. over a period of 45 min. The mixture is stirred for another 30 min at 0° C. The progress of reaction is monitored by TLC to ensure the completion of reaction. The reaction mixture is quenched with 2 N AcOH (22.0 mL) at 0° C. The mixture is concentrated to dryness. The residue is partitioned between EtOAC and water. The aqueous layer is further extracted with EtOAc. The combined organic extracts are washed with brine, dried over sodium sulfate and concentrated to obtain crude compound 1-OH (19.5 g, 64.8%) as a semi solid mass, m/z=569 [M−H]⁻.

### 1H. Synthesis of 4-methoxybenzyl (6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-3-((E)-4-(chloromethyl)styryl)-8-oxo-5-thia-1-azabicyclo[4.2.0]oct-2-ene-2-carboxylate (5-E)

### 1I. Synthesis of (6R,7R)-7-((Z)-2-(2-aminothiazol-4-yl)-2-(((2-carboxypropan-2-yl)oxy)imino)acetamido)-3-((E)-4-(chloromethyl)styryl)-8-oxo-5-thia-1-azabicyclo[4.2.0]oct-2-ene-2-carboxylic acid (6-E)

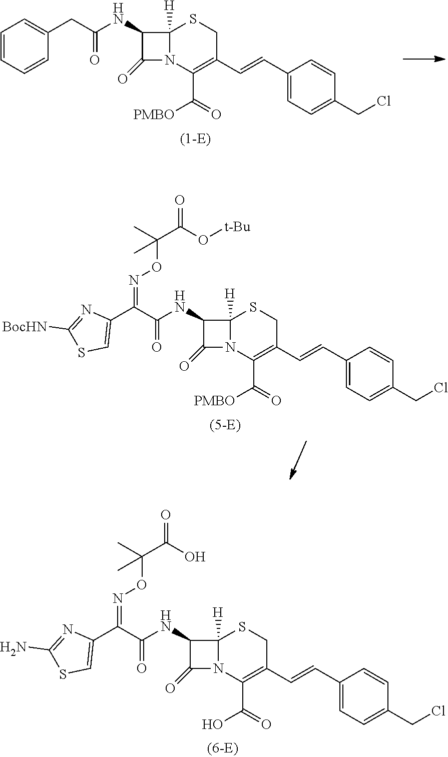

Compound 1-E (Pure E isomer) is used following the same procedure as described for the synthesis of compound 5 to provide compound 5-E as a yellow solid. Intermediate 5-E is used following the same procedure as described for the synthesis of compound 6 to provide compound 6-E as a yellow.

### 1J. Synthesis of 4-methoxybenzyl (6R,7R)-3-((E)-4-(chloromethyl)styryl)-8-oxo-7-(2-phenylacetamido)-5-thia-1-azabicyclo[4.2.0]oct-2-ene-2-carboxylate (1-E)

### 1K. Synthesis of 4-methoxybenzyl (6R,7R)-3-((Z)-4-(chloromethyl)styryl)-8-oxo-7-(2-phenylacetamido)-5-thia-1-azabicyclo[4.2.0]oct-2-ene-2-carboxylate (1-Z)

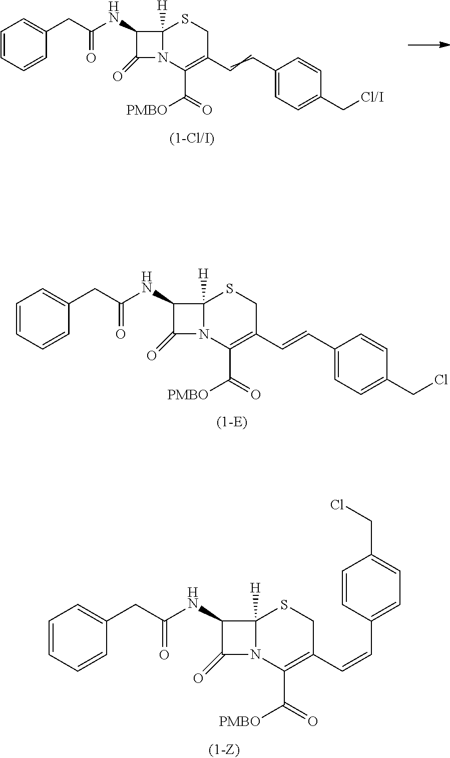

A mixture of E and Z isomers of compound 1-Cl/I (a mixture of Cl and I compounds) (2.8 g, 4.12 mmol) is dissolved in DMF (27 mL) and LiCl (1.31 g, 31.0 mmol) is added. The reaction mixture is stirred at room temperature overnight. The reaction mixture is then added into a mixture of water and ethyl acetate cooled in an ice bath. Two layers form and are separated. The aqueous layer is extracted with ethyl acetate (2×). The combined organic layers are washed with brine, dried over Na₂SO₄ and filtered. The filtrate is concentrated and the residue is purified by flash column chromatography using a gradient of ethyl acetate/toluene. Fractions are concentrated to dryness and the residue is triturated with minimum quantities of ethyl acetate to obtain compound 1-1 (E isomer, 0.5 g, 18%) as a pale-yellow solid. The mother liquid is re-purified by flash column chromatography using a gradient of ethyl acetate/toluene to give compounds 1-Z (1.2 g, 43%).

### 1L. Synthesis of 4-methoxybenzyl (6R,7R)-7-((Z)-2-(((1-(tert-butoxy)-2-methyl-1-oxopropan-2-yl)oxy)imino)-2-(2-((tert-butoxycarbonyl)-amino)thiazol-4-yl)acetamido)-3-((Z)-4-(chloromethyl)styryl)-8-oxo-5-thia-1-azabicyclo[4.2.0]oct-2-ene-2-carboxylate (5-Z)

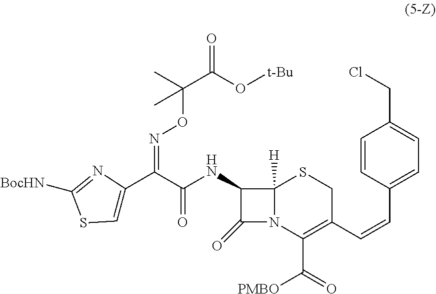

Compound 1-Z (1.0 equiv, 0.85 mmol) is employed to prepare 5-Z following the same procedure as described for the synthesis of compound 5. The crude product is purified by flash column chromatography using a gradient of ethyl acetate/toluene to give compound 5-Z (0.11 g, 18%) as a pale-yellow solid.

### 1M. Synthesis of (6R,7R)-7-((Z)-2-(2-aminothiazol-4-yl)-2-(((2-carboxypropan-2-yl)oxy)imino)acetamido)-3-((Z)-4-(chloromethyl)styryl)-8-oxo-5-thia-1-azabicyclo[4.2.0]oct-2-ene-2-carboxylic Acid (6-Z)

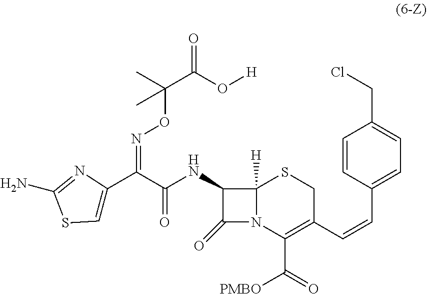

Compound 5-Z is employed to prepare 6-Z following the same procedure as described for the synthesis of compound 6 from compound 5. The crude product is purified by flash column chromatography using a gradient of ethyl acetate/toluene to give compound 6-Z.

It will be appreciated that compounds of Formula:

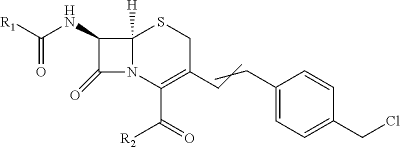

where R₁ and R₂ are as defined in Formulas I, IA and IIA herein and which are a mixture of E and Z isomers and which may be protected or unprotected (on the R₁ or R₂ groups) can be prepared by methods described herein for preparation of compounds 5 E/Z and 6 E/Z with appropriate choice of starting materials. These compounds are at least useful for the preparation of compounds of Formula I, IA, IIA and X herein. Corresponding E isomers of the above formula can be prepared free of the corresponding Z isomer as described herein for preparation of 5-E and 6-E herein. Corresponding E isomers of the above formula can be prepared free of the corresponding Z isomer as described herein for preparation of 5-E and 6-E herein. It will be appreciated in the art that protecting groups other than those specifically described herein can be used to protect various potentially reactive R₁ or R₂ groups.

### Example 2: Synthesis of Amines (Compounds of Formula XI) for Preparation of Compounds of Formulas I and II

### 2A. Synthesis of 2-methylisoindoline-5,6-diol, a Compound of Formula XI

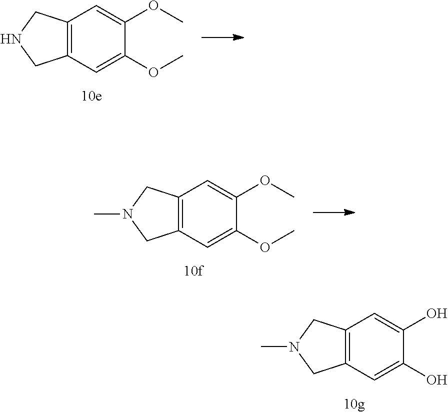

### Synthesis of 5,6-dimethoxy-2-methylisoindoline (10f)

A solution of 5,6-dimethoxyisoindoline 10e (0.20 g, 1.13 mmol) in 1,2-dichloroethane (6 mL) is vigorously stirred with 37% aqueous formaldehyde (1.9 mL, 26 mmol) for 5 min. Sodium triacetoxyborohydride (0.72 g, 3.4 mmol) is added in 6 portions over 5 min. The resulting mixture is stirred at room temperature for 3 h. Saturated sodium bicarbonate and dichloromethane are added to the reaction mixture, and the layers are separated. The aqueous layer is extracted with additional dichloromethane (3×). The combined organic phases are washed with water and then brine, dried over Na₂SO₄ and concentrated under reduced pressure to give crude compound 10f (0.21 g, 100%) as a light-brown solid, m/z=194, [M+H]⁺.

### Synthesis of 2-methylisoindoline-5,6-diol (10g) HBr Salt

A solution of 5,6-dimethoxy-2-methylisoindoline 10f (0.11 g, 0.57 mmol) in 48% hydrobromic acid (1.1 mL) is heated at reflux for 3 h. The reaction is cooled and concentrated to dryness. The residue is triturated with diethyl ether and the solid isolated to give crude compound 1f (0.136 g, 98%) as its HBr salt as a grey-tan solid, m/z=166 [M+H]⁺ and m/z=207 [M+H+ACN]⁺.

### 2B. Synthesis of 4-chloro-2-methylisoindoline-5,6-diol (10j) HBr Salt

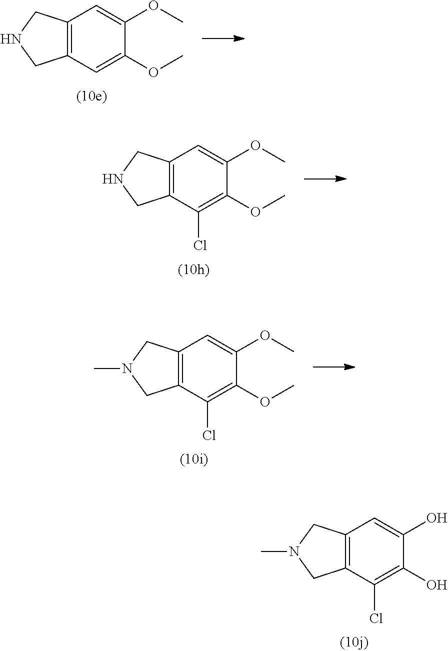

### Synthesis of 4-chloro-5,6-dimethoxyisoindoline (10h) HCl Salt

A solution of 5,6-dimethoxyisoindoline 10e (0.12 g, 0.69 mmol) in acetic acid (1.4 mL) is vigorously stirred while cooling with a cold-water bath. Sulfuryl chloride (0.073 mL, 0.90 mmol) is added drop-wise. The resulting mixture is then warmed to room temperature and stirred for 3 h. The reaction mixture is diluted with diethyl ether (1.4 mL) and stirred for 30 minutes. The mixture is filtered and the solid isolated to give a mixture of the HCl salt of product 10h (0.134 g, 76%) and starting material (3:1 ratio respectively). The mixture was carried forward to the next step without further purification. 10h HCl salt m/z=214/216 [M+H]⁺.

### Synthesis of 4-chloro-5,6-dimethoxy-2-methylisoindoline (10i)

Employing the mixture containing 10h HCl salt, a mixture of 10h HCl salt (0.14 g, 0.56 mmol), sodium hydroxide (0.02 g, 0.56 mmol), formic acid (0.021 mL, 0.56 mmol), and 37% aqueous formaldehyde (0.05 mL, 0.67 mmol) in 50% aqueous formic acid (0.20 mL) is prepared in a round bottomed flask equipped with a condenser. The reaction mixture is heated in at 80° C. for 3 h. The reaction is concentrated under reduced pressure, and water is added to the residue. The mixture is adjusted to pH 12 with 2 N NaOH, and is then extracted with dichloromethane (3×). The combined organic phases are dried over Na₂SO₄ and concentrated under reduced pressure. The crude sample is purified by flash column chromatography using a gradient of methanol/336582: dichloromethane/triethylamine to give compound 10i (0.093 g, 74%) as a light-brown film, m/z=228, [M+H]⁺.

### Synthesis of 4-chloro-2-methylisoindoline-5,6-diol (10j) HBr Salt

4-chloro-5,6-dimethoxy-2-methylisoindoline was used following the same procedure as described for the synthesis of compound 10g. The residue was triturated with diethyl ether and the solid isolated to give crude 10j (0.093 g, 99%) as its HBr salt as a pink-brown solid, m/z=200 [M+H]⁺ and m/z=241 [M+H+ACN]⁺.

### 2C. Synthesis of 4,7-dichloro-2-methylisoindoline-5,6-diol (10m)

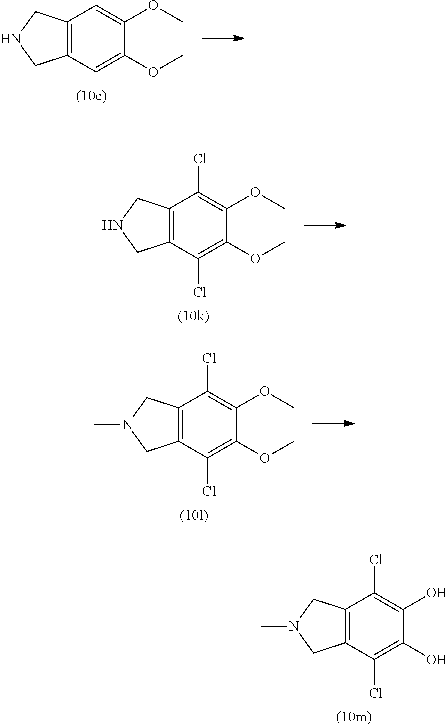

### Synthesis of 4,7-dichloro-5,6-dimethoxyisoindoline (10k) HCl Salt

A solution of 5,6-dimethoxyisoindoline 10e (0.08 g, 0.46 mmol) in acetic acid (0.92 mL) is stirred at room temperature. Sulfuryl chloride (0.11 mL, 1.4 mmol) is added drop-wise, and the resulting mixture is stirred at room temperature for 4 h. The reaction is concentrated under reduced pressure to give compound 10k as its HCl salt (0.130 g, quantitative yield) as a light-brown foam, m/z=248/250/252 [M+H]⁺.

### Synthesis of 4,7-dichloro-5,6-dimethoxy-2-methylisoindoline (101)

4,7-dichloro-5,6-dimethoxyisoindoline hydrochloride salt 10k is used following the same procedure as described for the synthesis of compound 10i. After flash column chromatography using a gradient of methanol/dichloromethane/triethylamine, compound 101 (0.072 g, 60%) is obtained as a light-brown crystalline solid, m/z=262/264/266 [M+H]⁺.

### Synthesis of 4,7-dichloro-2-methylisoindoline-5,6-diol (10m)

4,7-dichloro-5,6-dimethoxy-2-methylisoindoline 101 (0.0723 g, 0.28 mmol) is used following the same procedure as described for the synthesis of compound 10g. The residue was triturated with diethyl ether and the solid isolated to give crude 10m (0.085 g, 98%) as its HBr salt as a light-beige solid, m/z=234/236/238 [M+H]⁺.

### 2D. Synthesis of 2-methyl-1,2,3,4-tetrahydroisoquinoline-6,7-diol (10p)

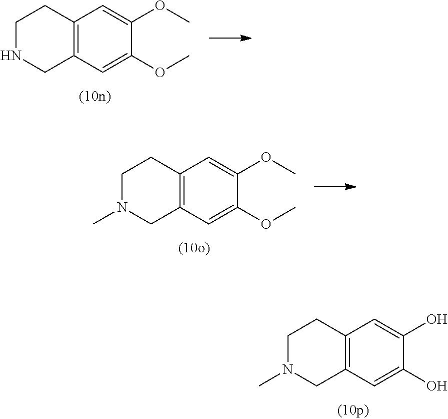

### Synthesis of 6,7-dimethoxy-2-methyl-1,2,3,4-tetrahydroisoquinoline (10o)

A mixture of 6,7-dimethoxy-1,2,3,4-tetrahydroisoquinoline hydrochloride salt 10n (2.0 g, 8.7 mmol) and sodium hydroxide (0.35 g, 8.7 mmol) is stirred in methanol (21 ml) for 20 minutes. Aqueous formaldehyde (37%) (0. 68 mL, 8.7 mmol) is added drop-wise over 5 minutes. The reaction is stirred for 30 min before sodium borohydride (0.33 g, 8.7 mmol) is added portion-wise over 5 min. The reaction is stirred overnight. Reaction is reduced to dryness and ethyl acetate and water are added. Organic layers are separated and the aqueous layer is extracted with ethyl acetate (2×). The combined organic layers are washed with brine and dried over Na₂SO₄. The solution is reduced to dryness to give compound 10 (quantitative yield) as a white solid, m/z=208 [M+H]⁺.

### Synthesis of 2-methyl-1,2,3,4-tetrahydroisoquinoline-6,7-diol (10p) HBr salt

Dimethoxy-2-methyl-1,2,3,4-tetrahydroisoquinoline 10o (0.8 g, 3.8 mmol) is used following the same procedure as described for the synthesis of compound 10g. The residue was triturated with ethanol and the solid isolated to give a crude sample of 10p (0.76 g, 76%) as its HBr salt as a white solid, m/z=180 [M+H]⁺.

### 2E. Synthesis of 2-ethyl-1,2,3,4-tetrahydroisoquinoline-6,7-diol (10r)

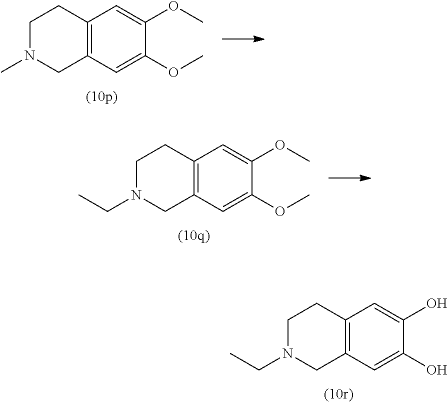

### Synthesis of 2-ethyl-6,7-dimethoxy-1,2,3,4-tetrahydroisoquinoline (10q)

6,7-Dimethoxy-2-methyl-1,2,3,4-tetrahydro-isoquinoline hydrochloride salt 10p (2.0 g, 8.7 mmol) is dissolved in 1,2-dimethoxyethane (43.5 mL) under nitrogen at 0° C. Triethylamine (12.0 mL, 87.1 mmol) and NaH (0.9 g, 21.8 mmol) are added to the solution. The mixture is stirred for 30 minutes at 0° C. Then iodoethane (1.05 mL, 13.06 mmol) is added to the mixture and reaction is warmed to room temperature and stirred overnight. A saturated aqueous solution of NH₄C1 is added and then reaction is made basic by addition of 5% aqueous NaOH and extracted with dichloromethane. The organic layers are washed with brine and dried over Na₂SO₄. After flash column chromatography using a gradient of methanol/dichloromethane/triethylamine, compound 10q (1.5 g, 81%) was obtained as a yellow oil, m/z=221 [M+H]⁺.

### Synthesis of 2-ethyl-1,2,3,4-tetrahydroisoquinoline-6,7-diol (10r)

2-Ethyl-6,7-dimethoxy-1,2,3,4-tetrahydroisoquinoline 10q (0.4 g, 3.8 mmol) is used following the same procedure as described for the synthesis of compound 10g. The residue was triturated with diethyl ether to give crude 10r (0.4 g, 3.8 mmol) as its HBr salt as a beige powder, m/z=194 [M+H]⁺.

### 2F. Synthesis of 5,8-dichloro-2-methyl-1,2,3,4-tetrahydroisoquinoline-6,7-diol (10t)

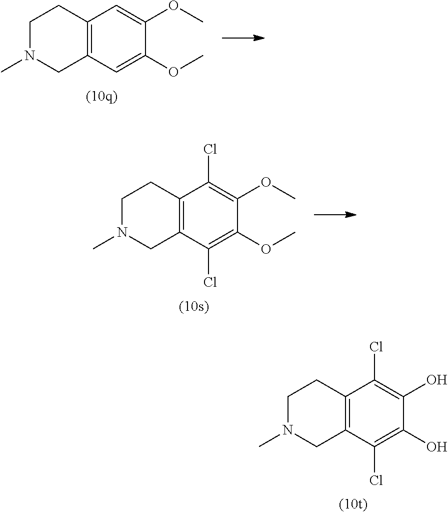

### Synthesis of 5,8-dichloro-6,7-dimethoxy-2-methyl-1,2,3,4-tetrahydro-isoquinoline (10s) HCl Salt

6,7-dimethoxy-2-methyl-1,2,3,4-tetrahydroisoquinoline 10q (0.2 g, 0.96 mmol) is used following the same procedure as described for the synthesis of compound 10k. The reaction mixture is stirred overnight and then is concentrated under reduced pressure. The residue is re-crystallized with ethanol and ether to give crude sample 10s (0.18 g, 62%) as its HCl salt as a light-grey solid, m/z=276/278 [M+H]⁺.

### Synthesis of 5,8-dichloro-2-methyl-1,2,3,4-tetrahydroisoquinoline-6,7-diol (10t) HBr Salt

5,8-dichloro-6,7-dimethoxy-2-methyl-1,2,3,4-tetrahydroisoquinoline hydrochloride salt 10s (0.16 g, 0.58 mmol) is used following the same procedure as described for the synthesis of compound 10g. The residue was triturated with diethyl ether to give crude 10t (0.18 g, 94%) as its HBr salt as a light-grey solid, m/z=248/250 [M+H]⁺.

### 2G. Synthesis of 2-(prop-2-yn-1-yl)-1,2,3,4-tetrahydroisoquinoline-6,7-diol

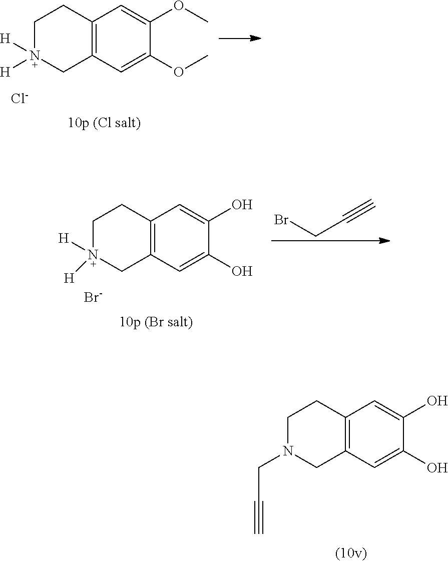

### Synthesis of 1,2,3,4-tetrahydroisoquinoline-6,7-diol hydrobromide Salt (10p Br Salt)

6,7-Dimethoxy-1,2,3,4-tetrahydroisoquinoline hydrochloride salt (10p (CI salt) (0.2 g, 0.87 mmol) is used following the same procedure as described for the synthesis of compound 10g (see 2A) and the reaction mixture is heated for 24 h. Solvent is concentrated under reduced pressure to obtain a crude sample of 10p (Br salt) (0.022 g, quantitative yield) as its HBr salt, m/z=166 [M+H]⁺.

Compound 10p (Br salt) (0.12 g, 0.48 mmol), propargyl bromide (0.043 mL, 0.48 mmol), potassium iodide (0.08 g, 0.48 mmol), and potassium carbonate (0.033 g, 0.24 mmol) are dissolved in N,N-dimethylformamide (4.8 mL). The reaction is stirred at room temperature for 5 h and then concentrated under reduced pressure. Water and ethyl acetate are added to the reaction mixture. The two phases are separated and the aqueous phase is extracted with ethyl acetate (2×). The combined organic layers are dried over MgSO₄, filtered, and concentrated under reduced pressure. The residue is purified by flash column chromatography using a gradient of methanol/dichloromethane to elute compound 10v (0.037 g, 37%), m/z=204 [M+H]⁺.

Amines and salts thereof of formulas:

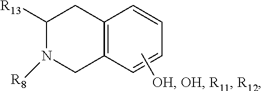

and more specifically

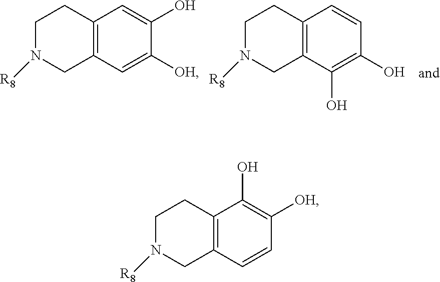

where variables are as defined in Formula III can be prepared as described for compounds 10r or 10v with appropriate choice of alkyl halide, cycloalkyl halide or other halide with appropriate choice of starting amine.

### 2H. Synthesis of 3,4-bis((4-methoxybenzyl)oxy)-N-(pyridin-4-yl)benzamide (10aa)

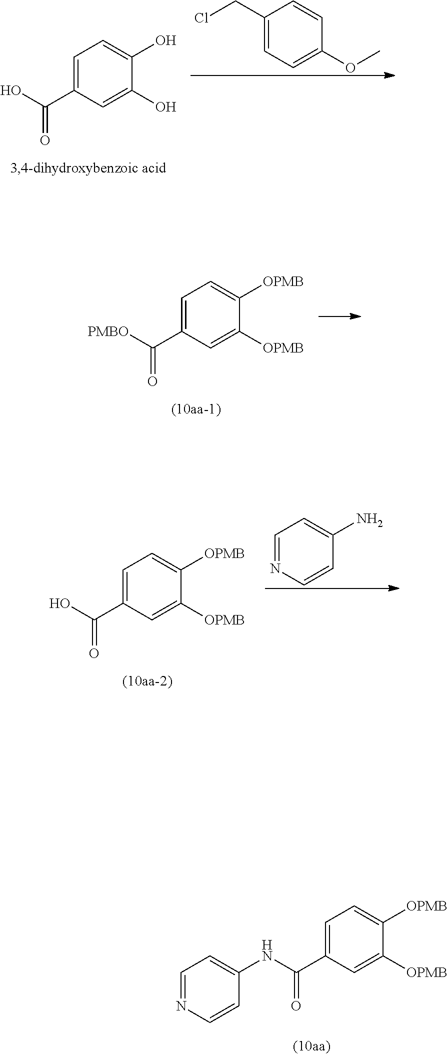

### Synthesis of 4-methoxybenzyl 3,4-bis((4-methoxybenzyl)oxy)benzoate (10aa-1)

3,4-Dihydroxybenzoic acid (3.0 g, 19.46 mmol) is dissolved in N,N-dimethylformamide (39 mL) and potassium carbonate (9.4 g, 68.13 mmol), 4-methoxybenzyl chloride (10.56 mL, 77.86 mmol) and sodium iodide (2.9 g, 19.46 mmol) were added to the solution. The solution is heated to 70° C. for 3 h. Another portion of 4-methoxybenzyl chloride (2.6 mL, 19.46 mmol) and potassium carbonate (2.3 g, 17.0 mmol) is then added and heating is continued overnight. The reaction was cooled down to room temperature and water and ethyl acetate were added. The organic phase was washed with brine (2×), dried over Na₂SO₄ and solvent is evaporated. The residue was purified by flash column chromatography using gradients of ethyl acetate/toluene to elute compound 10aa-1 (7.1 g, 71%) as a white solid.

### Synthesis of 3,4-bis((4-methoxybenzyl)oxy)benzoic Acid (10aa-2)

Compound 10aa-1 is dissolved in MeOH (31 mL) and THF (31 mL) and NaOH (aq. 8 M, 2.0 mL) are added. The mixture is heated to 70° C. for 2h. Reaction is cooled to room temperature and solvent is evaporated. DCM, HCl (2M) and water were added. Aqueous phase is extracted in DCM (2×), dried over Na₂SO₄ and solvent is evaporated to obtain compound 10aa-2 (1.99 g, 65%) as a white solid, m/z=393 [M−H]⁻. Compound 10aa-2 (0.03 g, 0.076 mmol) is dissolved in DCM (0.4 mL) and oxalyl chloride (0.01 mL, 0.076 mmol) is added drop-wise at room temperature. Then N,N-dimethylformamide (2 drops) is added. Reaction is stirred at room temperature for 2 h. Solvent is removed under reduced pressure and the residue is re-dissolved in DCM (0.4 mL) and cooled in the ice bath. 4-aminopyridine (0.007 g, 0.076 mmol) and DMAP (0.009 g, 0.076 mmol) are added. Reaction is stirred at room temperature overnight. The reaction mixture is diluted in dichloromethane, washed with saturated sodium bicarbonate solution and then with brine and dried over Na₂SO₄. Crude product is purified by column chromatography using gradients of methanol/DCM (dichloromethane) to elute compound 10aa (0.02 g, 55%) as a white solid, m/z=471 [M+H]⁺.

### 21. Synthesis of N-(3,4-dihydroxyphenyl)-1-methyl-1H-imidazole-5-carboxamide (10bb)

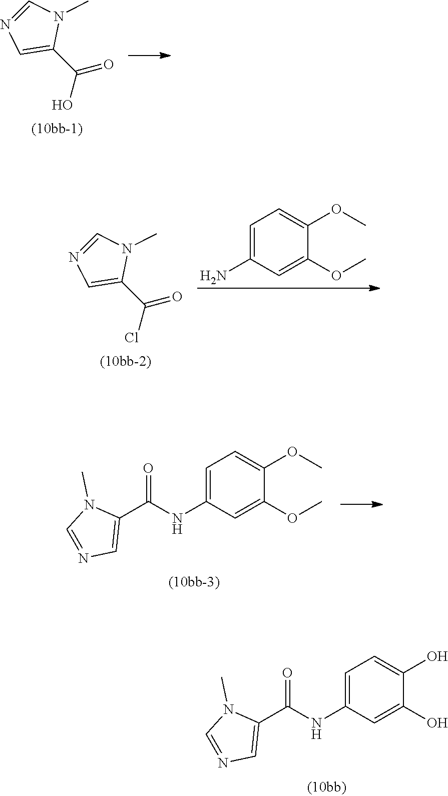

### Synthesis of 1-methyl-1H-imidazole-5-carbonyl chloride (10bb-2)

Oxalyl chloride (1.1 equiv, 1.1 mL) and 1-methyl-1H-imidazole-5-carboxylic acid 10bb-1 (0.2 g, 1.6 mmol) are added to dry acetonitrile (2.6 mL) in a flame-dried flask under nitrogen atmosphere. N,N-dimethylformamide (2 drops) is added to the mixture. The reaction stirred at room temperature for 4 h. A large volume of diethyl ether is added to precipitate the product and the precipitated product is collected. The solid collected (10bb-2) is used in the next step without further purification.

### Synthesis of N-(3,4-dimethoxyphenyl)-1-methyl-1H-imidazole-5-carboxamide (10bb-3)

Intermediate 10bb-2 (0.23 g, 1.6 mmol) and 3,4-dimethoxyaniline (0.48 g, 3.2 mmol) are mixed in dry acetonitrile (5.3 mL) and pyridine is added (0.38 mL, 4.7 mmol). The reaction is stirred at room temperature for 2 days. The reaction is diluted in DCM and washed with saturated sodium bicarbonate solution (2×). The aqueous phase is extracted with DCM (2×). Combined organic phases are dried over MgSO₄, and solvent is removed to give crude product. Crude product is purified by column chromatography using gradients of methanol/dichloromethane to elute compound 10bb-3 (0.19 g, 46%).

### Synthesis of N-(3,4-dihydroxyphenyl)-1-methyl-1H-imidazole-5-carboxamide (10bb)

Compound 10bb-3 (0.19 g, 0.72 mmol) is dissolved in DCM (4.0 mL) and the reaction is cooled to 0° C. BBr₃ (1 M solution in DCM, 0.8 mL, 0.8 mmol) is added drop-wise and reaction is stirred at room temperature for 4 h. Water is added to the reaction mixture. The resultant two phases are separated and the organic phase is washed again with water. The combined aqueous phases are washed with DCM (3×). The crude product obtained was purified by reverse-phase chromatography using acetonitrile/water gradients to obtain compound 10bb (48.6, 29%), m/z=234 [M+H]⁺.

### 2J. Synthesis of 4-(1H-imidazol-1-yl)-1,2-phenylene di-tert-butyl bis(carbonate)

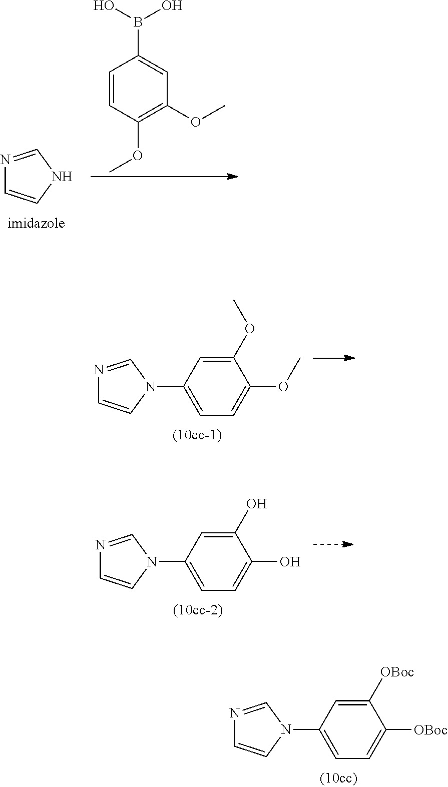

### Synthesis of 1-(3,4-dimethoxyphenyl)-1H-imidazole (10cc-1)

Imidazole (0.55 g, 8.1 mmol), (3,4-dimethoxyphenyl)boronic acid (2.9 g, 16.2 mmol), Copper(II) acetate (Cu(OAc)₂, 2.1 g, 12.1 mmol) and triethylamine (5.6 mL, 40.4 mmol) are mixed in DCM (40.5 mL) under nitrogen atmosphere and molecular sieves (1.0 g) are added. The reaction is stirred at room temperature for 2 days. The reaction mixture is filtered through a pad of Celite® and washed with DCM. Combined organic phases are washed with saturated sodium bicarbonate solution (2×) and dried over MgSO₄. Solvent was removed to obtain crude product. Crude product was purified by column chromatography using gradients of methanol/dichloromethane to elute compound 10cc-1 (0.7 g, 42%).

### Synthesis of 4-(1H-imidazol-1-yl)benzene-1,2-diol (10cc-2)

Compound 10cc-1 (0.7 g, 3.4 mmol) was used following the same procedure as in the synthesis of compound 10bb (21, above). The crude product was purified by reverse-phase chromatography using acetonitrile/water gradients to obtain compound 10cc-2 (0.3, 50%), m/z=177 [M+H]⁺.

### Synthesis of 4-(1H-imidazol-1-yl)-1,2-phenylene di-tert-butyl bis(carbonate)

Compound 10cc-2 (0.13 g, 0.76 mmol) is dissolved in DCM (4.0 mL) and the mixture is cooled at 0° C. Then triethylamine (0.4 mL, 0.3 mmol) and DMAP (0.009 g, 0.08 mmol) are added. After 15 minutes di-tert-butyl dicarbonate (0.66 g, 0.3 mmol) is added and reaction mixture is warmed to room temperature and stirred for 5h. Water and DCM were added to the reaction mixture. Two phases were separated and the organic phase was washed with water and then brine. Solvent was removed to give crude product. Crude product was purified by column chromatography twice using gradients of methanol/DCM to elute compound 10cc (0.095 g, 33%), m/z=377 [M+H]⁺.

### 2 K. Synthesis of 4-(3-methylpyridin-4-yl)benzene-1,2-diol hydrobromide Salt 10dd (Br Salt)

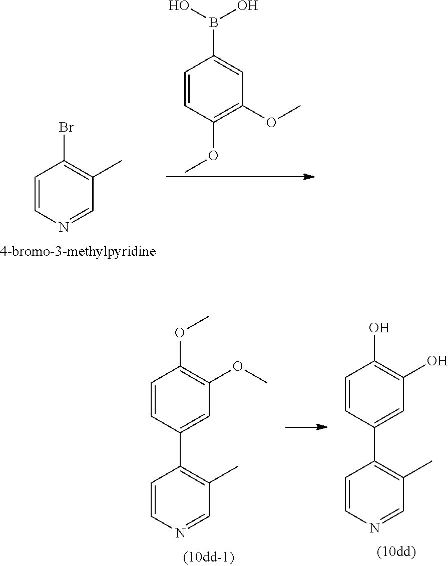

### Synthesis of 4-(3,4-dimethoxyphenyl)-3-methylpyridine (10dd-1)

4-Bromo-3-methylpyridine (0.083 g, 0.48 mmol) is dissolved in toluene (5.5 mL) and ethanol (0.27 mL) in a microwave vial under a nitrogen atmosphere. 3,4-Dimethoxyphenylboronic acid (0.087 g, 0.48 mmol) is added, followed by Pd(dppf)Cl₂ dichloromethane complex (0.0197 g, 0.024 mmol) and sodium carbonate (2 M in water, 0.48 mL, 0.96 mmol). The vial is capped and the mixture is heated with in a 110° C. oil bath for 19 h. The reaction is cooled to room temperature, diluted with water, and the layers are separated. The aqueous layer is extracted with ethyl acetate (3×). The combined organic layers are dried over Na₂SO₄, filtered, and concentrated under reduced pressure. The residue is purified by flash column chromatography using a gradient of ethyl acetate/dichloromethane as the eluent to give compound 10dd-1 (0.062 g, 56%) as colorless solid, m/z=230 [M+H]⁺.

### Synthesis of 4-(3-methylpyridin-4-yl)benzene-1,2-diol hydrobromide salt (10dd HBr)

Compound 10dd-1 (0.06 g, 0.27 mmol) is used following the same procedure as synthesis of compound 10bb, except that reaction mixture was first cooled down to −78° C. and then warmed to room temperature. The reaction mixture is cooled to 0° C. and quenched with methanol (2 mL), and is then concentrated under reduced pressure. The residue is triturated with diethyl ether and the solid is isolated to give a crude sample of 10dd (0.072 g, 95%) as its HBr salt as a light brown solid, m/z=202 [M+H]⁺.

### Example 3: Preparation of Compounds of Formulas I-Ill

### 3A. Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-6,7-dihydroxyisoquinolin-2-ium, Compound 435 E/Z, a Compound of Formula II

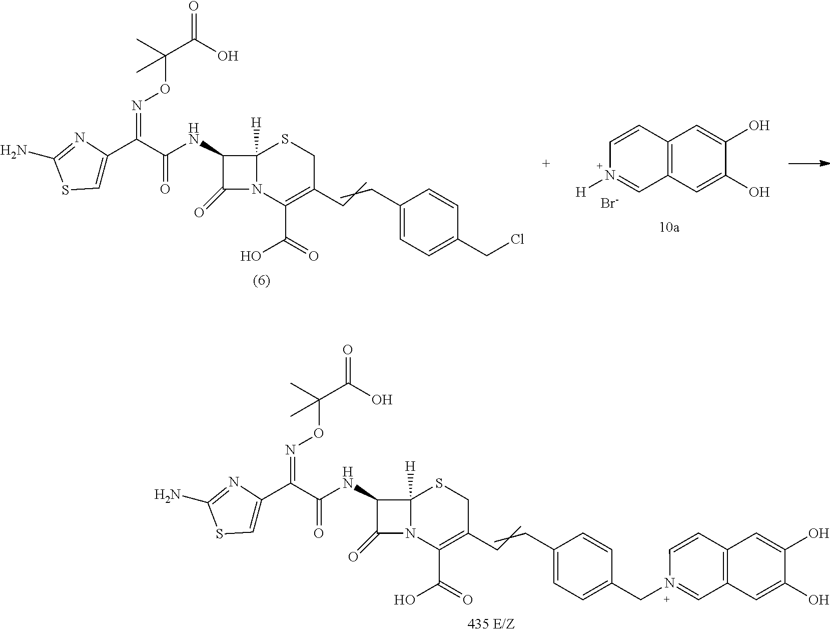

Intermediate 6 (mixture of E/Z isomers) (0.19 g, 0.32 mmol) is combined with the HBr salt of isoquinoline-6,7-diol 10a (0.085 g, 0.35 mmol), potassium iodide (0.05 g, 0.32 mmol) and anhydrous N,N-dimethylformamide (3.3 mL) under a nitrogen atmosphere. Potassium carbonate (0.066 g, 0.48 mmol) is added, the reaction is protected from light with aluminum foil and is stirred at room temperature for 22 h. Formic acid (0.65 mL) is added, and the reaction is concentrated under reduced pressure. Diethyl ether is added to the residue, and the mixture is triturated, sonicated, and stirred until a solid formed. The supernatant is removed, and the solid is washed with diethyl ether (3×) and then dried under reduced pressure. The solid is then triturated with water (3×), and the solid is isolated after centrifugation. The solid is suspended in water, and the resulting mixture is lyophilized. A portion of the lyophilized compound (0.093 g out of 0.1497 g crude) is purified by preparative scale reverse phase HPLC, eluting with mixtures of water (containing 0.1% TFA) and acetonitrile to give compound 435 E/Z (a mixture of E and Z isomers) (0.03 g, 22%) as a light-tan solid, m/z=731 [M]⁺.

### 3B. Synthesis of 2-(4-((Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-6,7-dihydroxyisoquinolin-2-ium, Compound 435-Z, a Compound of Formula III

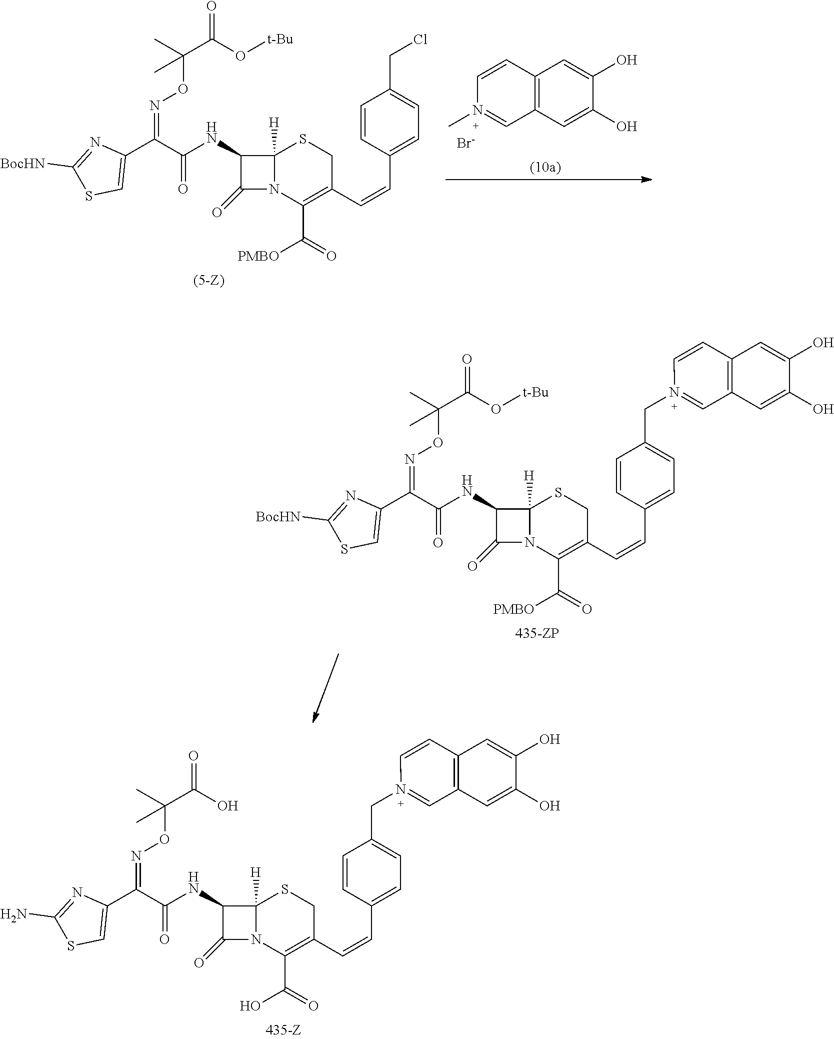

Intermediate 5-Z and isoquinoline-6,7-diol hydrobromide salt 10a (1.6 equiv, 0.09 mmol) are used following the same procedure as described for the synthesis of compound 435 E/Z (See 3A). Ethyl acetate and aqueous 1 N HCl are added to the cooled reaction mixture (0° C.), the layers are separated and the organic layer is washed two more times with 1 N HCl, and then with brine. The washed organic layer is dried over sodium sulfate and volatiles are removed under reduced pressure to obtain the Z isomer of compound 435-ZP (protected) as an off-white solid. This solid was used in the next step without further purification.

### 3C. Synthesis of 2-(4-((Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-6,7-dihydroxyisoquinolin-2-ium (435-Z) a Compound of Formula III

Compound 435-ZP (1.0 equiv, 0.06 mmol) is dissolved in dichloromethane (1.1 mL) under a nitrogen atmosphere. The reaction is cooled over an ice bath for 10 minutes. Anisole (0.25 mL) is added drop-wise and the reaction is stirred over an ice bath for 10 minutes. TFA (1.12 ml) is added drop-wise and the reaction is stirred over the ice bath for 2 h. The progress of reaction was followed by LC-MS. Then the reaction is placed in the fridge overnight. The reaction mixture is concentrated under reduced pressure. Acetonitrile is then added to dissolve the residue and the mixture is concentrated. The residue is triturated with diethyl ether for 30 minutes. The resulting suspension is filtered and the solid is washed with diethyl ether to obtain crude compound 435-Z. The crude compound is purified by preparative HPLC, eluting with mixtures of water and acetonitrile, to obtain the Z isomer compound 435-Z (0.013 g, 36%, over two steps) as a white solid, m/z=731 [M]⁺.

### 3D. Synthesis of 2-(4-((E)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-6,7-dihydroxyisoquinolin-2-ium (435-E) a Compound of Formula III

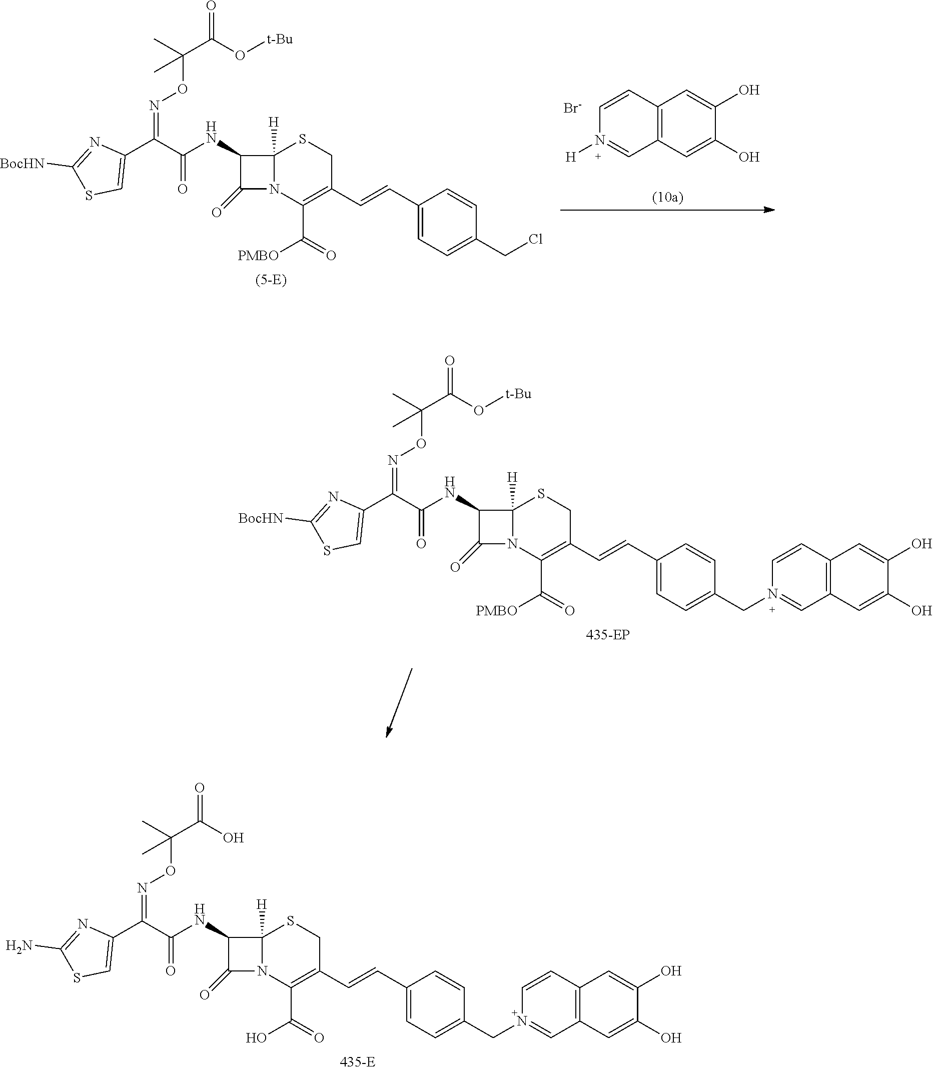

### Synthesis of 2-(4-((E)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4 methoxybenzyl)oxy)carbonyl)-8-oxo-5-thia-1-azabicyclo[4.2.0]oct-2-en-3-yl)vinyl)benzyl) 6,7-dihydroxyisoquinolin-2-ium (435-EP, Protected)

Isoquinoline-6,7-diol hydrobromide salt 10a (1.3 equiv, 0.29 mmol) and E isomer 5-E (1.0 equiv, 0.23 mmol) are employed following the same procedure as described for the synthesis of compound 435 E/Z. To the residue a few drops of TFA is added and DMF is then evaporated under reduced pressure. Ethyl acetate and aqueous 1 N HCl solution are added, the layers are separated and the organic layer is washed two more times with 1 N HCl solution, then with brine. The organic layer is dried over magnesium sulfate and volatiles are removed under reduced pressure. The residue is triturated in diethyl ether to obtain the protected E isomer 435-EP (0.148 g, 65%) as an orange solid. This solid was submitted to the next step without further purification.

### Synthesis of 2-(4-((E)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-6,7-dihydroxyisoquinolin-2-ium (435-E)

Compound 435-EP (1.0 equiv, 0.15 mmol) is dissolved in dichloromethane (1.1 mL) under a nitrogen atmosphere. The reaction is cooled over an ice bath for 10 minutes. Anisole (0.25 mL) is added drop-wise and the reaction is stirred over an ice bath for 10 minutes. TFA (1.12 ml) is added drop-wise and the reaction is stirred over the ice bath for 2 h. The progress of reaction is followed by LC-MS. Then the reaction is refrigerated overnight. The reaction mixture is concentrated under reduced pressure. Acetonitrile is then added to dissolve the residue and the mixture is concentrated. The residue is triturated with diethyl ether for 30 minutes. The resulting suspension is filtered and the solid is washed with diethyl ether to obtain crude compound 435-E. The crude product is obtained as a yellow solid (0.118 g). A portion of the crude compound (0.050 g) is purified by a preparative HPLC method, eluting with mixtures of water and acetonitrile, to obtain E isomer 435-E (0.032 g, 64%) as off-white solid, m/z=731 [M]⁺.

### 3E. Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-5,8-dichloro-6,7-dihydroxyisoquinolin-2-ium (449 E/Z), a Compound of Formula III

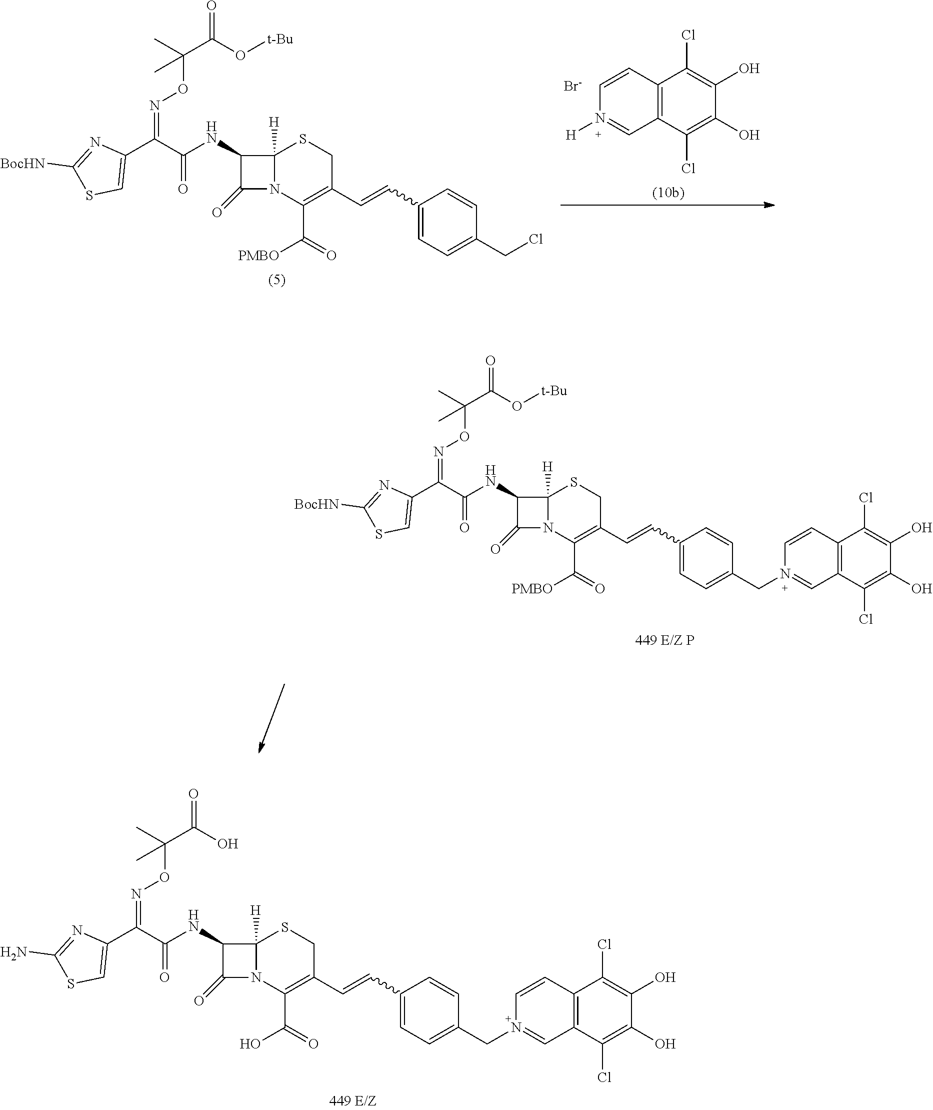

Synthesis of 2-(4-((E/Z)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4-methoxybenzyl)oxy)carbonyl)-8-oxo-5-thia-1-azabicyclo[4.2.0]oct-2-en-3-yl)vinyl)benzyl)-5,8-dichloro-6,7-dihydroxyisoquinolin-2-ium (449 E/Z P) 5,8-Dichloroisoquinoline-6,7-diol hydrobromide salt (10b)(1.3 equiv, 0.09 mmol) and intermediate 5 are used following the same procedure as described for the synthesis of compound 435-EP. The residue was also treated the same as compound 435-EP to give compound 449 E/ZP (0.046 g, 62%) as an orange solid which was used in next step without further purification, m/z=1075 [M]⁺.

### Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-5,8-dichloro-6,7-dihydroxyisoquinolin-2-ium (449 E/Z)

Compound 449-E/Z P (1.0 equiv, 0.02 mmol) is reacted following the same procedure as described for the synthesis of compound 442 E/Z. The reaction is cooled over an ice bath for 10 minutes. Anisole (0.25 mL) is added drop-wise and the reaction is stirred over an ice bath for 10 minutes. TFA (1.12 ml) is added drop-wise and the reaction is stirred over the ice bath for 2 h. The progress of reaction is followed by LC-MS. Then the reaction mixture is refrigerated overnight. The reaction mixture is concentrated under reduced pressure. Acetonitrile is then added to dissolve the residue and the mixture is concentrated. The residue was dissolved in the minimum amount of acetonitrile and water (1:1 mixture) and the resulting solution was lyophilized. After lyophilization of the filtrate, product 449 E/Z (0.005 g, 31%) was obtained as a beige solid.

### 3F. Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-5,8-dichloro-6,7-dihydroxyisoquinolin-2-ium (450 E/Z)

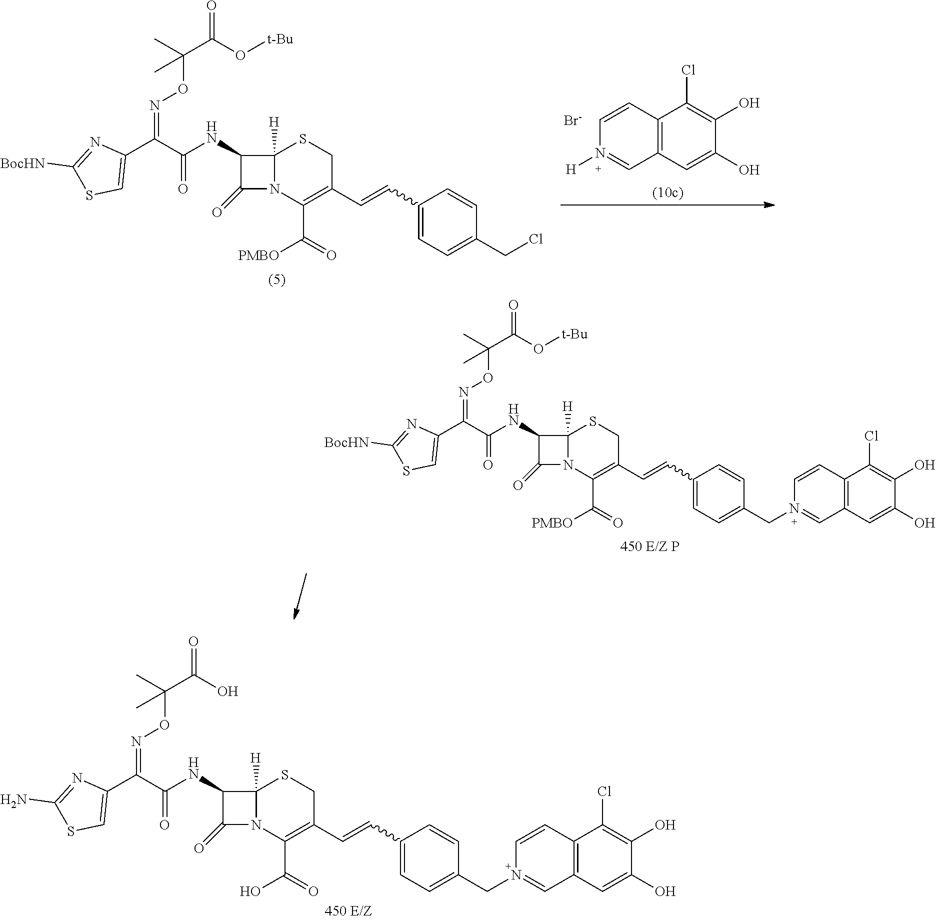

### Synthesis of 2-(4-((E/Z)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4 methoxybenzyl)oxy)carbonyl)-8-oxo-5-thia-1-azabicyclo[4.2.0]oct-2-en-3-yl)vinyl)benzyl) 5,8-dichloro-6,7-dihydroxyisoquinolin-2-ium (450 E/Z P)

5-Chloroisoquinoline-6,7-diol hydrobromide Salt 10c (1.3 equiv, 0.09 mmol) is used following the same procedure as described for the synthesis of compound 435-EP. The residue is also treated the same as compound 435-EP to give 450 E/Z P (0.042 g, 58%) as a yellow solid which is used in next step without further purification, m/z=1041 [M]⁺.

Compound 450 E/ZP (1.0 equiv, 0.04 mmol) is dissolved in dichloromethane (1.1 mL) under a nitrogen atmosphere. The reaction is cooled over an ice bath for 10 minutes. Anisole (0.25 mL) is added drop-wise and the reaction is stirred over an ice bath for 10 minutes. TFA (1.12 ml) is added drop-wise and the reaction is stirred over the ice bath for 2 h. The progress of reaction is followed by LC-MS. Then the reaction is then placed in the fridge overnight. The reaction mixture is concentrated under reduced pressure. Acetonitrile is then added to dissolve the residue and the mixture is concentrated. The residue is dissolved in the minimum amount of acetonitrile and water (1:1 mixture) and the resulting solution is lyophilized, yielding the desired compound 102 E/Z (0.031 g, quantitative yield) as pale yellow solid, m/z=765 [M]⁺.

### 3G: Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-5,6-dihydroxy-2-methylisoindolin-2-ium (424 E/Z)

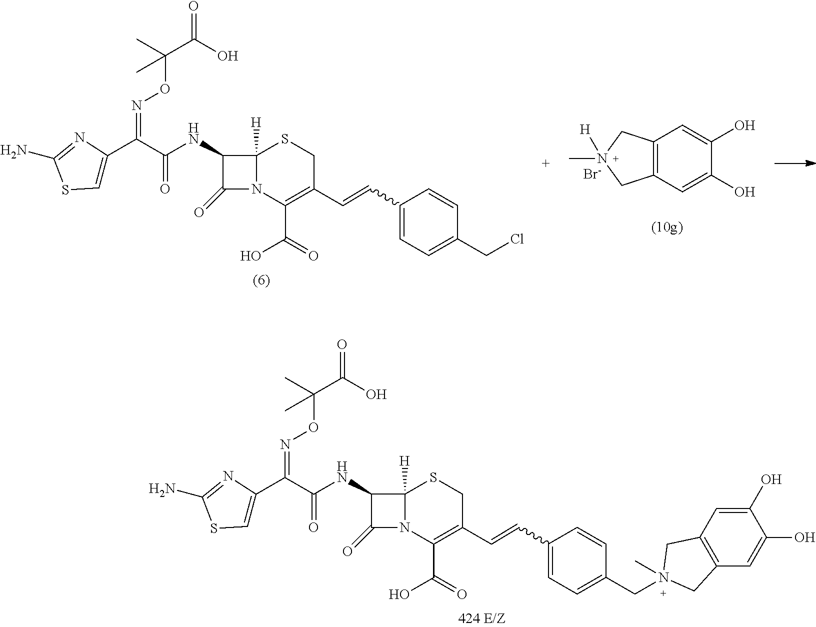

Intermediate 6 and the HBr salt of 2-methylisoindoline-5,6-diol (10g) are reacted following the same procedure as described for the synthesis of compound 435 E/Z. (3A). Formic acid (0.05 mL) is added to the reaction mixture, and the reaction mixture is concentrated under reduced pressure. Diethyl ether is added to the residue, and the mixture is triturated, sonicated, and stirred until a solid formed. The supernatant is removed, and the solid is washed with diethyl ether (3×) and then dried under reduced pressure. The solid is then triturated with water (3×), and the solid is isolated after centrifugation. The solid is suspended in water, and the resulting mixture is lyophilized to give compound 424 E/Z (0.0107 g, 60%) as a pinkish-beige solid, m/z=735, [M]⁺.

### 3H: Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-4-chloro-5,6-dihydroxy-2-methylisoindolin-2-ium (432 E/Z)

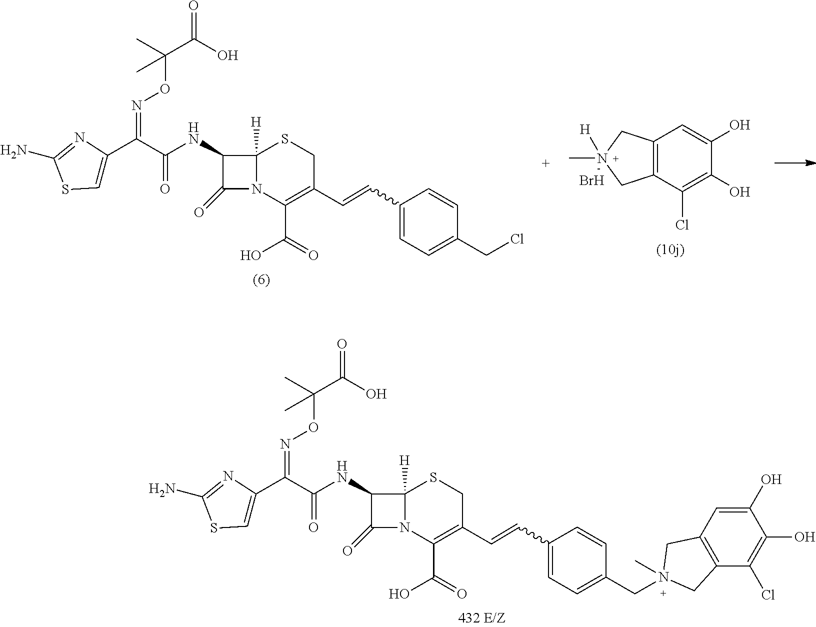

Intermediate 6 and HBr salt of 4-chloro-2-methylisoindoline-5,6-diol (10k) are reacted following the same procedure as described for the synthesis of compound 424 E/Z (3A). Crude product is also triturated the same method as compound 424 E/Z to obtain compound 432 E/Z (0.021 g, 67%) as a light-brown solid, m/z=769, [M]⁺.

### 3I: Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-4,7-dichloro-5,6-dihydroxy-2-methylisoindolin-2-ium (431 E/Z)

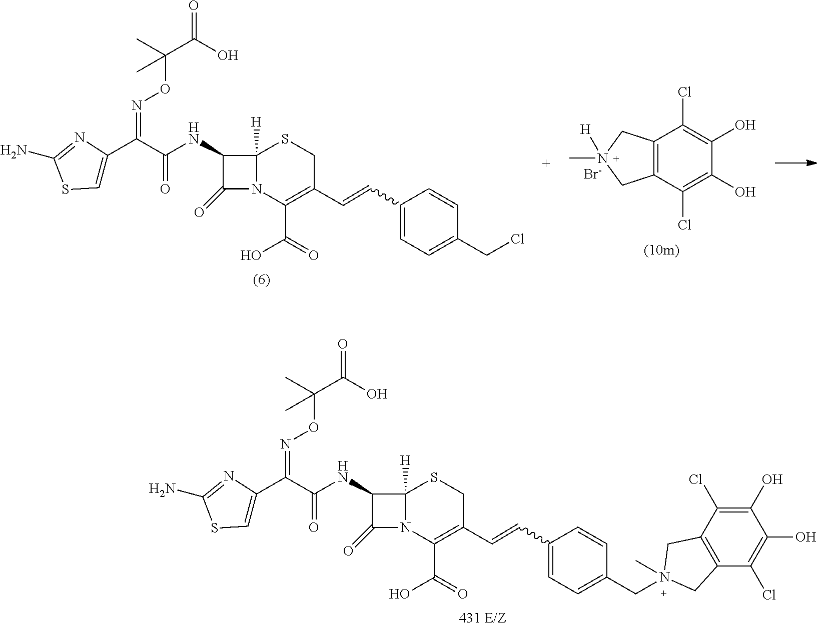

Intermediate 6 and HBr salt of 4,7-dichloro-2-methylisoindoline-5,6-diol (10m) are reacted following the same procedure as described for the synthesis of compound 424 E/Z. Crude product is also triturated the same method as compound 424 E/Z to obtain compound 431 E/Z (0.021 g, 69%) as a beige solid, m/z=803/805 [M]⁺.

### 31A: Synthesis of 2-(4-((E)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-4,7-dichloro-5,6-dihydroxy-2-methylisoindolin-2-ium (431 E)

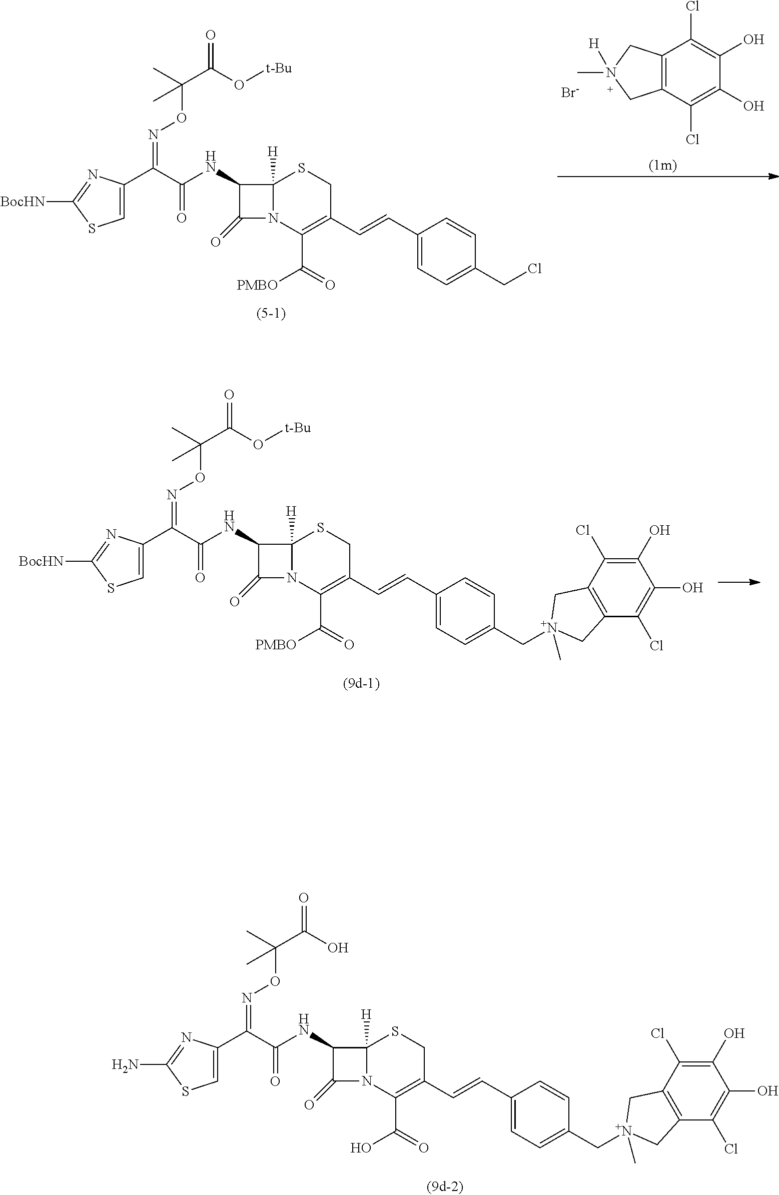

### Synthesis of 2-(4-((E)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4-methoxybenzyl)oxy)carbonyl)-8-oxo-5-thia-1-azabicyclo[4.2.0]oct-2-en-3-yl)vinyl)benzyl)-4,7-dichloro-5,6-dihydroxy-2-methylisoindolin-2-ium

4,7-Dichloro-2-methylisoindoline-5,6-diol hydrobromide salt 10m (1.6 equiv, 0.36 mmol) is used following the same procedure as described for the synthesis of compound 412 E/Z P. Ethyl acetate and aqueous 1 N HCl solution were added and the layers are separated. The organic layer is washed two more times with 1 N HCl solution, then with brine. The organic layer is dried over sodium sulfate and volatiles are removed under reduced pressure to obtain E isomer of compound 431 E P as a white solid. This solid is used in the next step without further purification.

Deprotection of 431 E P

Compound 431 E P (1.0 equiv, 0.23 mmol) is used following the same procedure as described for the synthesis of compound 414 E/Z and the progress of reaction is followed by LC-MS. The residue is triturated with diethyl ether and then is purified by preparative scale reverse phase HPLC, eluting with mixtures of water and acetonitrile to give pure E isomer of compound 431 E (0.047 g, 26%, over two steps) as a white solid, m/z=802 [M]⁺.

### 3J: Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3 yl)vinyl)benzyl)-6,7-dihydroxy-2-methyl-1,2,3,4-tetrahydroisoquinolin-2-ium (407 E/Z)

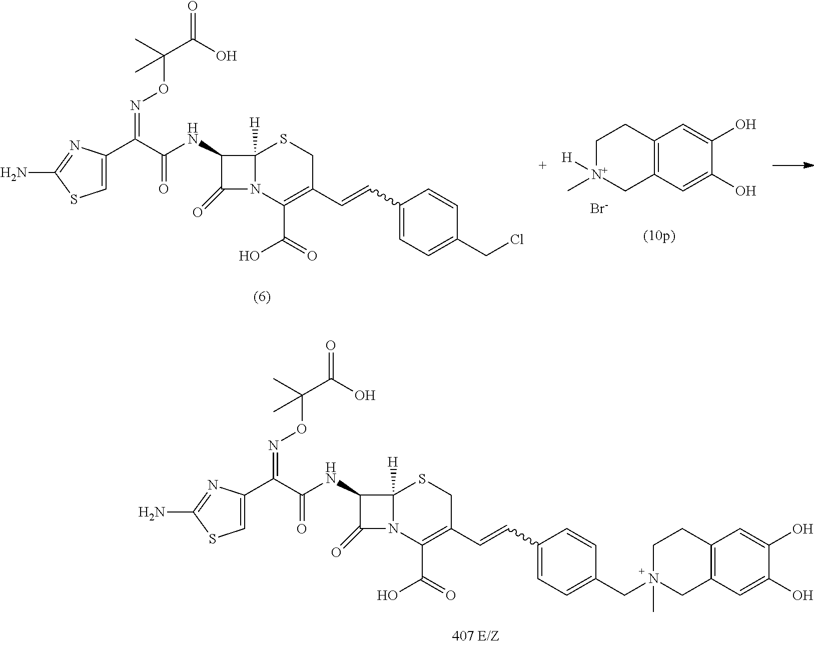

Intermediate 6 and HBr salt of 2-methyl-1,2,3,4-tetrahydroisoquinoline-6,7-diol (10p) are reacted following the same procedure as described for the synthesis of compound 424 E/Z. Crude product is purified through reverse-phase column chromatography using gradients of acetonitrile/water to elute 407 E/Z (0.004 g, 4.4%) as a white solid, m/z=749 [M]⁺.

### 3JA: Synthesis of 2-(4-(€-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3 yl)vinyl)benzyl)-6,7-dihydroxy-2-methyl-1,2,3,4-tetrahydroisoquinolin-2-ium (447 E also designated 407 E)

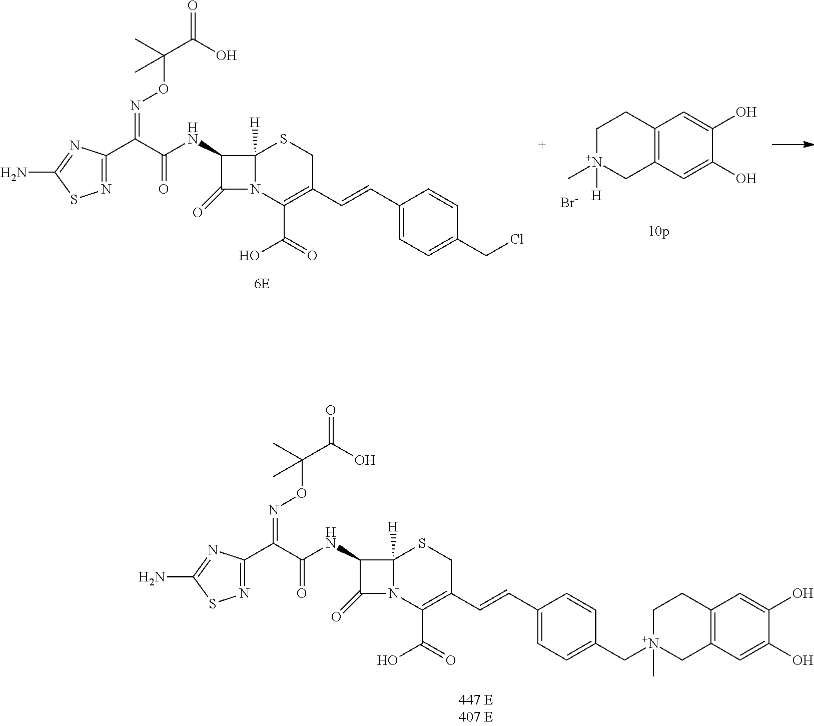 [SMILES: CC(C)(O/N=C(\C(=O)N[C@@H]1C(=O)N2C(C(=O)O)=C(/C=C/c3ccc(CCl)cc3)CS[C@H]12)c1nsc(N)n1)C(=O)O ; [Br-] ; C[NH+]1CCc2cc(O)c(O)cc2C1 ; CC(C)(O/N=C(\C(=O)N[C@@H]1C(=O)N2C(C(=O)O)=C(/C=C/c3ccc(C[N+]4(C)CCc5cc(O)c(O)cc5C4)cc3)CS[C@H]12)c1nsc(N)n1)C(=O)O]

Intermediate 6E and 2-methyl-1,2,3,4-tetrahydroisoquinoline-6, 7-diol hydrobromide salt (1.2 equiv., 0.1 mmol) are reacted following the procedure as described for 424 E/Z. To the reaction is added acetonitrile, TFA (1 mL) and then water to provide a yellow solution. Crude product is purified directly through preparative reverse-phase column chromatography using gradients of acetonitrile/water (containing 0.2% TFA) to elute compound 407 E (also called 447 E) (0.048 g, 21%) as a white solid, m/z=749 [M]⁺.

### 3K: Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-2-ethyl-6,7-dihydroxy-1,2,3,4-tetrahydroisoquinolin-2-ium (423 E/Z)

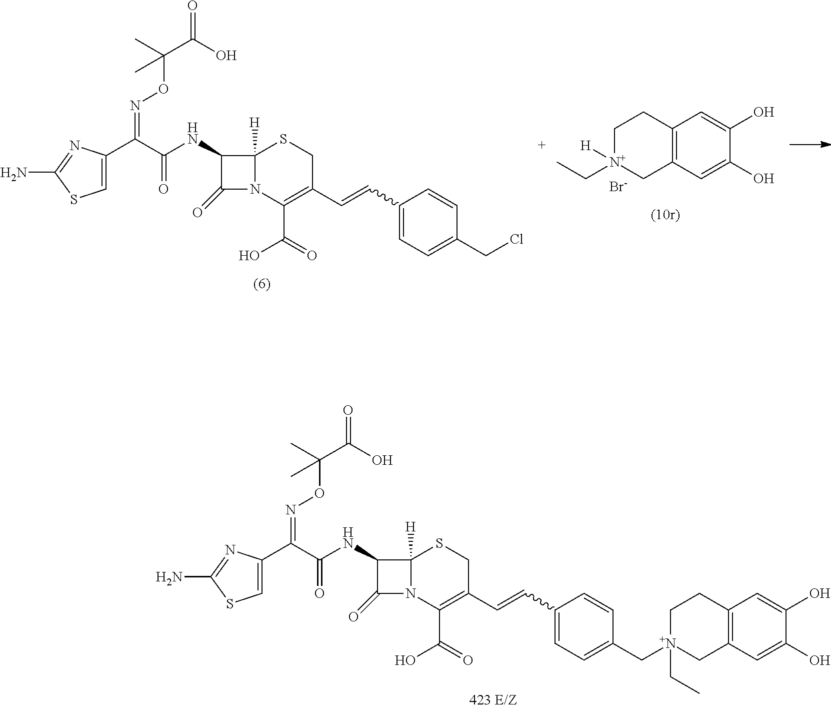 [SMILES: CC(C)(O/N=C(\C(=O)N[C@@H]1C(=O)N2C(C(=O)O)=C(C=Cc3ccc(CCl)cc3)CS[C@H]12)c1csc(N)n1)C(=O)O ; CC[NH+]1CCc2cc(O)c(O)cc2C1 ; [Br-] ; CC[N+]1(Cc2ccc(C=CC3=C(C(=O)O)N4C(=O)[C@@H](NC(=O)/C(=N\OC(C)(C)C(=O)O)c5csc(N)n5)[C@H]4SC3)cc2)CCc2cc(O)c(O)cc2C1]

Intermediate 6 (0.045 g, 0.07 mmol) is dissolved in N,N-dimethylformamide (0.8 mL) under a nitrogen atmosphere. The HBr salt of 2-ethyl-1,2,3,4-tetrahydroisoquinoline-6,7-diol (10r) (0.02 g, 0.07 mmol) is added, followed by addition of potassium iodide (0.012 g, 0.07 mmol) and potassium carbonate (0.015 g, 0.11 mmol). The reaction mixture is stirred at room temperature for 17 hour and then concentrated under reduced pressure. Crude product is purified by reverse-phase column chromatography using gradients of acetonitrile/water to elute 423 E/Z (0.015 g, 25%) as a pale-yellow solid, m/z=763 [M]⁺.

### 3L: Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-5,8 dichloro-6,7-dihydroxy-2-methyl-1,2,3,4-tetrahydroisoquinolin-2-ium

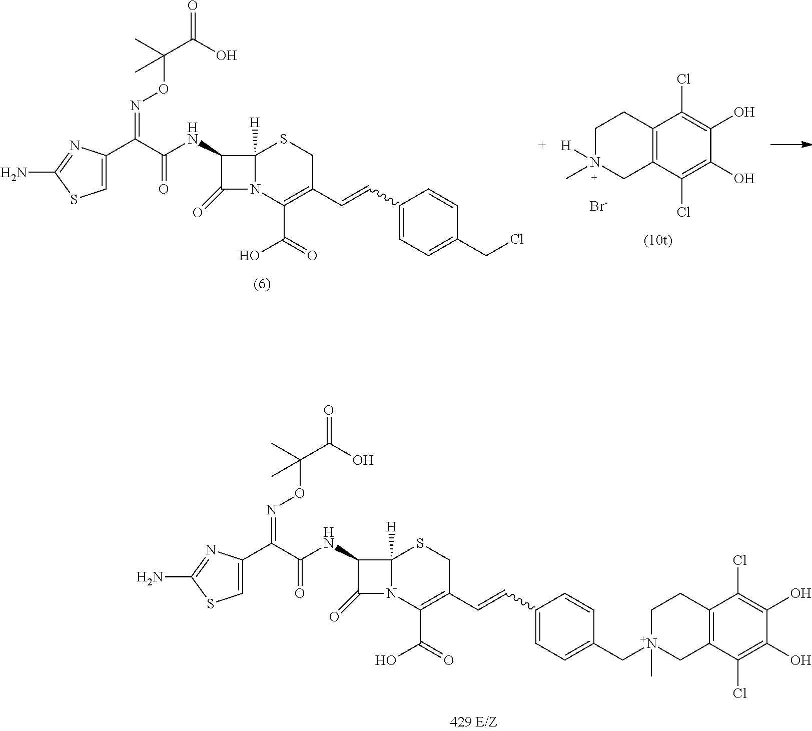

5,8-dichloro-2-methyl-1,2,3,4-tetrahydroisoquinoline-6,7-diol hydrobromide salt (10t) (0.054 g, 0.16 mmol) is used following the same procedure as described for the synthesis of compound 424 E/Z. Crude product is purified through reverse-phase column chromatography using gradients of acetonitrile/water to elute compound 429 E/Z (0.026 g, 22%) as a yellow solid, m/z=817/819 [M]⁺.

### 3M: Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-5,6-dihydroxy-2-methyl-1,2,3,4-tetrahydroisoquinolin-2-ium (441 E/Z)

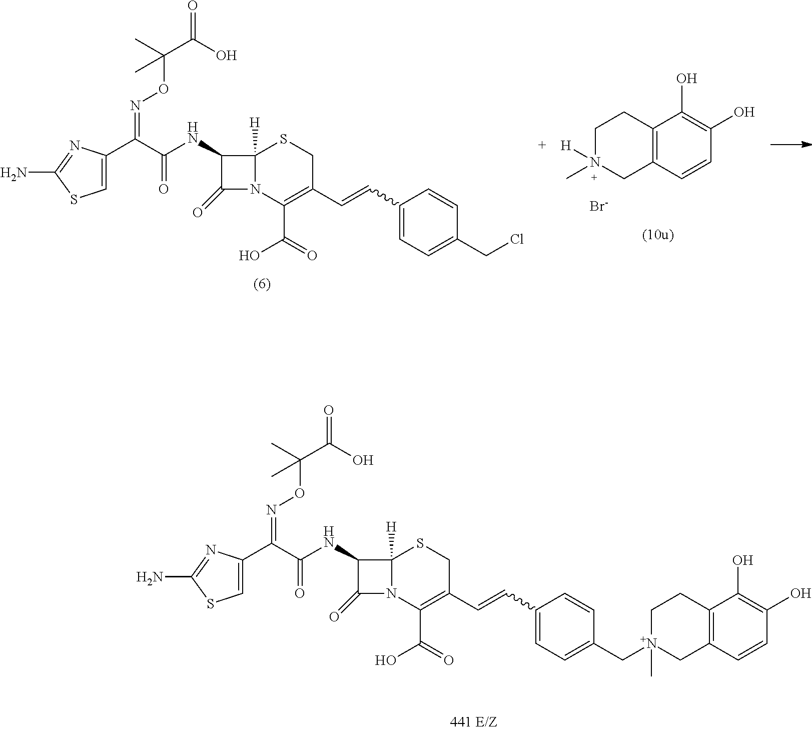

2-Methyl-1,2,3,4-tetrahydroisoquinoline-5,6-diol hydrobromide salt (10u) (0.017 g, 0.07 mmol) is reacted following the same procedure as described for the synthesis of compound 424 E/Z. Crude product is purified through reverse-phase column chromatography using gradients of acetonitrile/water to elute compound 9h (0.0186 g, 41%) as a light-yellow solid, m/z=749 [M]⁺.

### 3N: Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-6,7-dihydroxy-2-(prop-2-yn-1-yl)-1,2,3,4-tetrahydroisoquinolin-2-ium (446 E/Z)

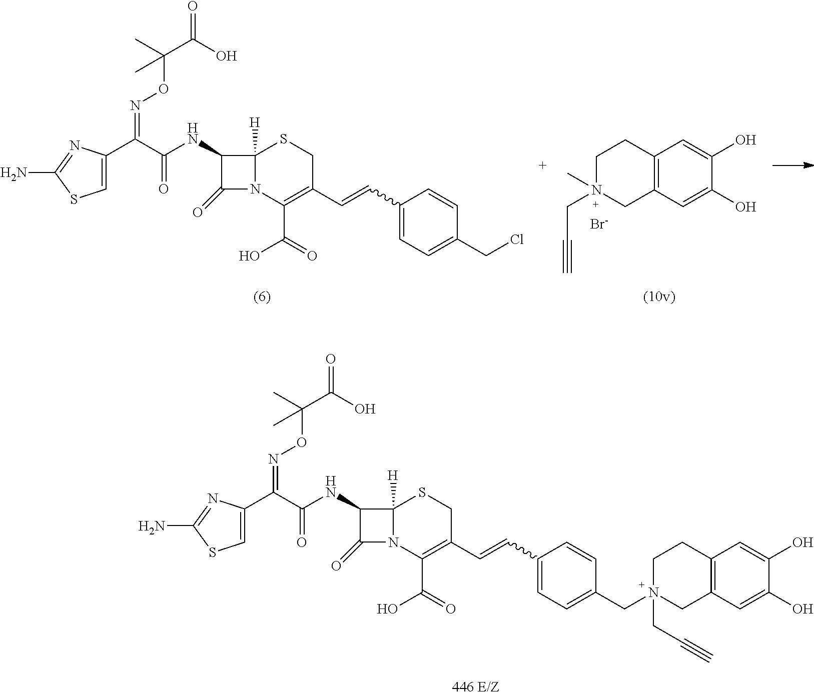

2-(Prop-2-yn-1-yl)-1,2,3,4-tetrahydroisoquinoline-6,7-diol HBr salt (0.011 g, 0.05 mmol) (10v) is reacted following the same procedure as described for the synthesis of compound 424 E/Z, except that no potassium carbonate base is used. Formic acid (0.05 mL) is added, and the reaction is concentrated under reduced pressure. Diethyl ether is added to the residue, and the mixture is triturated, sonicated, and stirred until a solid formed. The supernatant is removed, and the solid is washed with diethyl ether (3×) and then dried under reduced pressure. The solid is then triturated with water (3×), and the solid is isolated after centrifugation. The solid is suspended in water, and the resulting mixture is lyophilized to give compound 446 E/Z. Crude product is purified through reverse-phase column chromatography using gradients of acetonitrile/water to elute compound 9i (0.0063 g, 14%) as a light-yellow solid, m/z=773 [M]⁺ and 387 [M+H]²⁺.

### 3O: Synthesis of 2-(4-((E/Z)-2-((6R,7R)-7-((Z)-2-(5-amino-1,2,4-thiadiazol-3-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-6,7-dihydroxy-2-methyl-1,2,3,4-tetrahydroisoquinolin-2-ium (437 E/Z)

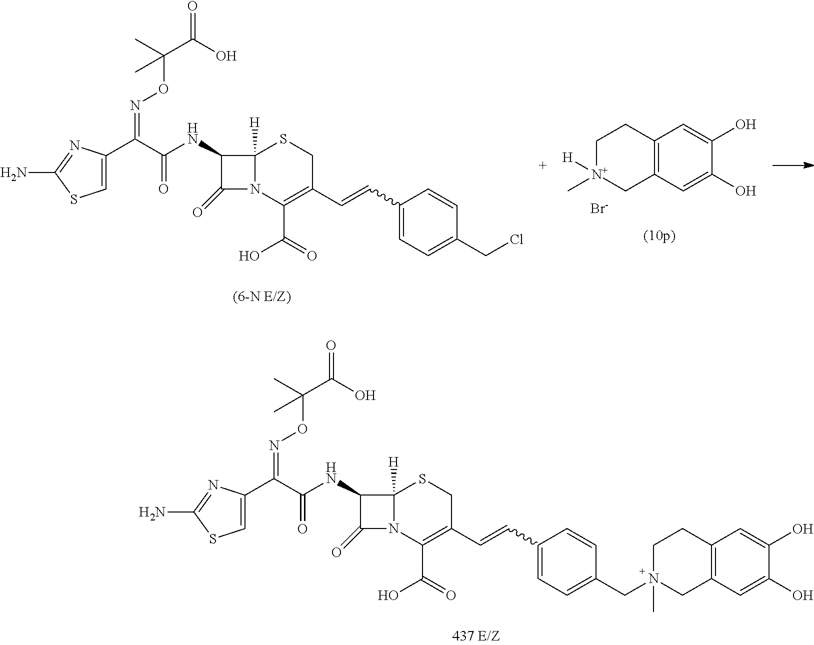

Intermediate 6-N and 2-methyl-1,2,3,4-tetrahydroisoquinoline-6,7-diol hydrobromide salt (10p) (1.1 g, 0.1 mmol) are reacted following the same procedure as described for the synthesis of compound 424 E/Z. Crude product is purified through reverse-phase column chromatography using gradients of acetonitrile/water to elute compound 437 E/Z (0.035 g, 52%) as a pale-yellow solid, m/z=750 [M]⁺.

3P: Synthesis of 1-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-3,4-dihydroxypyridin-1-ium (443 E/Z)

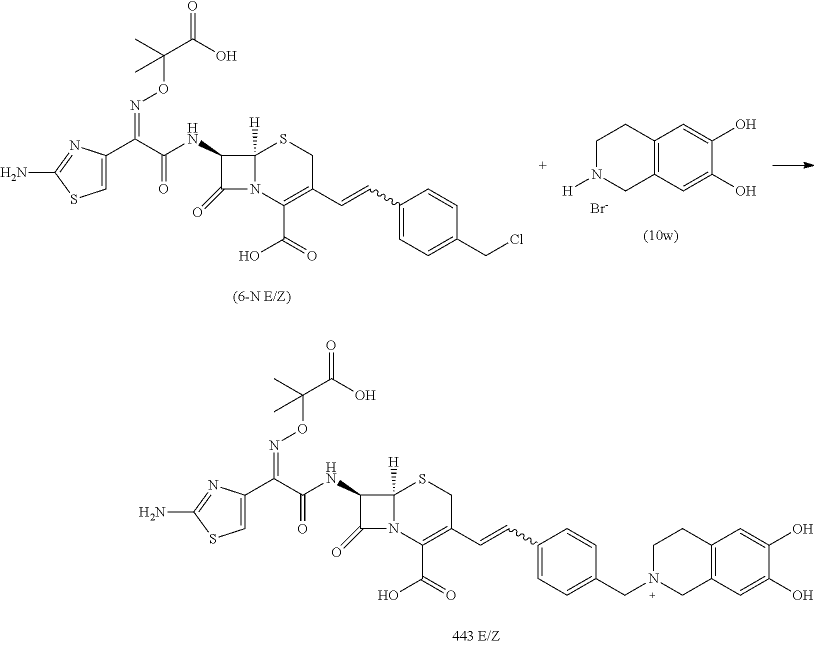 [SMILES: CC(C)(O/N=C(\C(=O)N[C@@H]1C(=O)N2C(C(=O)O)=C(C=Cc3ccc(CCl)cc3)CS[C@H]12)c1csc(N)n1)C(=O)O ; Oc1cc2c(cc1O)CNCC2 ; [Br-] ; CC(C)(O/N=C(\C(=O)N[C@@H]1C(=O)N2C(C(=O)O)=C(C=Cc3ccc(CN4CCc5cc(O)c(O)cc5C4)cc3)CS[C@H]12)c1csc(N)n1)C(=O)O]

Intermediate 6 and pyridine-3,4-diol hydrobromide salt (1.1 g, 0.13 mmol) were used following the same procedure as described for the synthesis of compound 424 E/Z. Crude was purified through reverse-phase column chromatography using gradients of acetonitrile/water to elute compound 443 E/Z (0.011 g, 13%) as a yellow solid, m/z=681 [M]⁺.

### 3Q: Synthesis of 1-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-3,4-dimethoxypyridin-1-ium

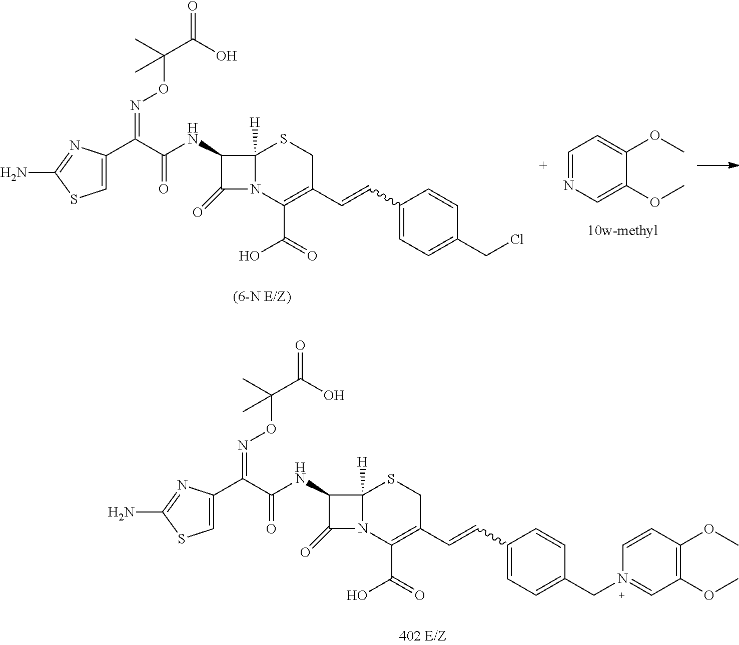

Intermediate 6 (0.07g, 0.12 mmol) is dissolved in acetonitrile (1.1 mL) and water (1.1 mL) under a nitrogen atmosphere. 3,4-dimethoxypyridine (10w-methyl) (0.064 mL, 0.46 mmol) is added, followed by potassium iodide (0.019 g, 0.12 mmol). The reaction is heated to 55° C. with an oil bath for 6 h, allowed to stand overnight at room temperature, and then concentrated under reduced pressure. Crude is purified through reverse-phase column chromatography using gradients of acetonitrile/water to elute compound 402 E/Z (0.016 g, 20%) as a light-yellow solid, m/z=709 [M]⁺.

### 3R: Synthesis of 1-(4-((E/Z)-2-((6R,7R)-7-((Z)-2-(2-aminothiazol-4-yl)-2-(methoxyimino)acetamido)-2-carboxy-8-oxo-5-thia-1-azabicyclo[4.2.0]oct-2-en-3-yl)vinyl)-3-nitrobenzyl)-3,4-dimethoxypyridin-1-ium (392 E/Z)

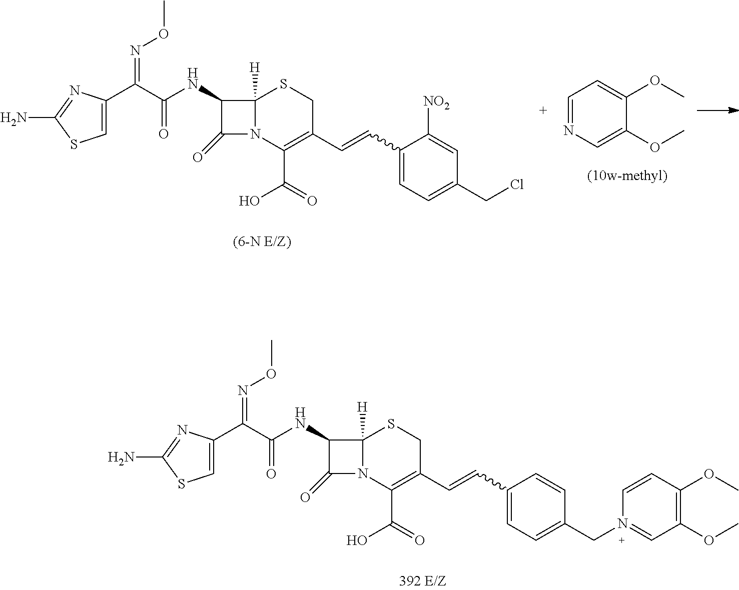

Intermediate 6-3 and 3,4-dimethoxypyridine (4.0 equiv, 0.35 mmol) (10w-methyl) are reacted following the same procedure as described for the synthesis of compound 402 E/Z. Crude product is purified through reverse-phase column chromatography using gradients of acetonitrile/water to elute compound 392 E/Z (0.025 g, 42%) as a dark-yellow solid, m/z=682 [M]⁺.

### 3S: Synthesis of 1-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-4-(3,4-dihydroxyphenyl)pyridin-1-ium (412 E/Z)

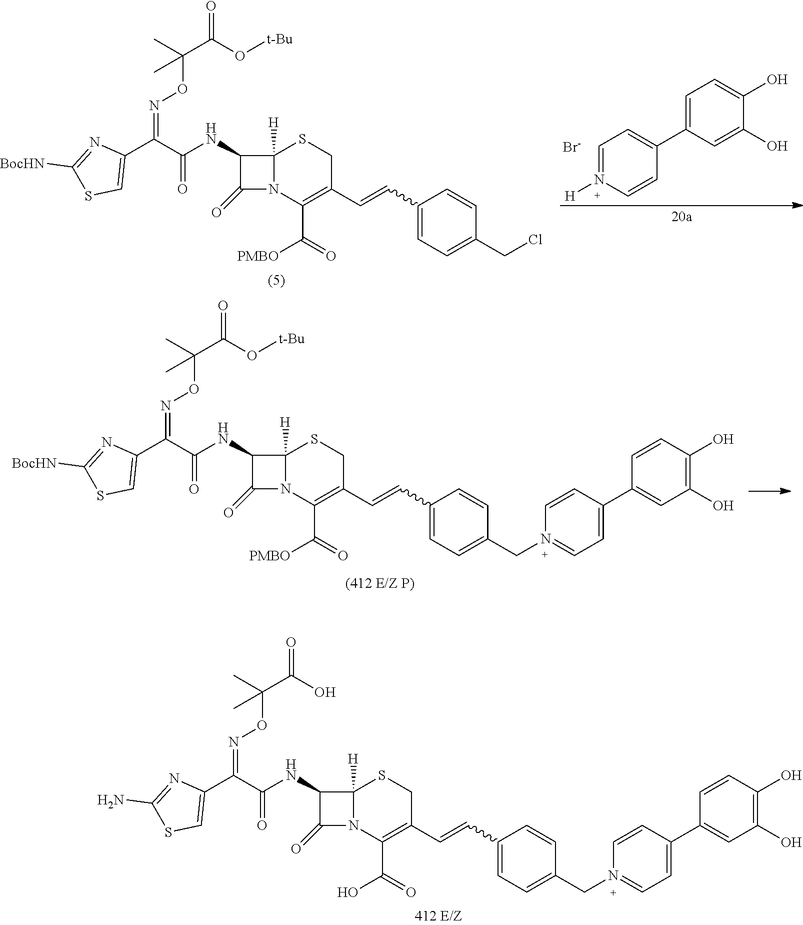

### Synthesis of 1-(4-((E/Z)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4 methoxybenzyl)oxy)carbonyl)-8-oxo-5-thia-1-azabicyclo[4.2.0]oct-2-en-3-yl)vinyl)benzyl)-4-(3,4-dihydroxyphenyl)pyridin-1-ium (412 E/Z P)

4-(Pyridin-4-yl)benzene-1,2-diol hydrobromide salt (20a) (3.0 equiv, 0.25 mmol) is used following the same procedure as described for the synthesis of compound 424 E/Z. The reaction is heated at 55° C. and the progress of reaction is followed by LC-MS. Crude product is purified through reverse-phase column chromatography using gradients of acetonitrile/water to elute compound protected compound 412 E/Z P (0.058 g, 66%) as a bright-orange powder, m/z=1033 [M]⁺.

### Deprotection of Compound 412 E/Z P

Compound 412 E/Z P (1.0 equiv, 0.035 mmol) is dissolved in dichloromethane (1.1 mL) under a nitrogen atmosphere. Reaction solution is cooled over an ice bath for 10 minutes. Anisole (0.25 mL) is added drop-wise and stirred over ice bath for 10 minutes. TFA (1.12 ml) is added drop-wise and stirred over ice bath for 2 h. Then reaction is placed in the fridge and the progress of reaction is followed by LC-MS. The reaction mixture is concentrated with under reduced pressure. Acetonitrile is added to dissolve the residue and concentrated. The residue is triturated with diethyl ether to give a sample of 412 E/Z (0.024 g, 92%) as a pale-yellow solid, m/z=757 [M]⁺.

### 3T: Synthesis of 1-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3 yl)vinyl)benzyl)-3-(3,4-dihydroxyphenyl)pyridin-1-ium 413 E/Z)

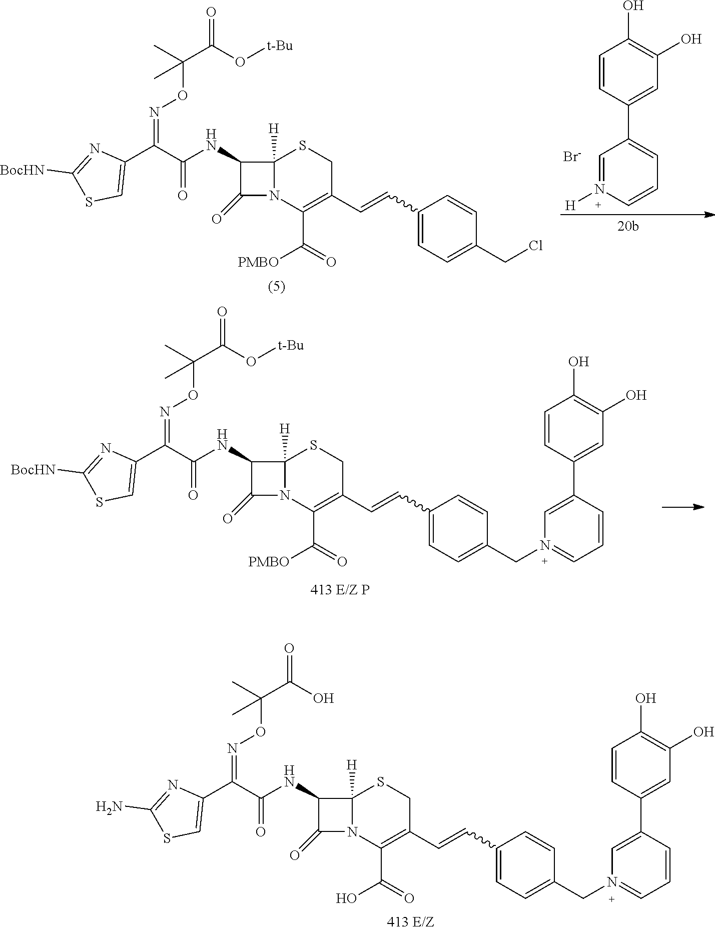

4-(Pyridin-3-yl)benzene-1,2-diol hydrobromide salt (10bb)(3.0 equiv, 0.059 mmol) is reacted following the same procedure as described for the synthesis of compound 412 E/Z P. The residue is triturated with diethyl ether to give 413 E/Z P (quantitative yield) as a light-orange solid which is used in next step without further purification, m/z=1033 [M]⁺.

### Deprotection of Compound 413 E/Z P

Compound 413 E/Z P (1.0 equiv, 0.02 mmol) is reacted as described for compound 412 E/Z P and the progress of reaction is followed by LC-MS. The residue is triturated with diethyl ether, acetonitrile and water to give 413 E/Z (0.0094 g, 63%, over two steps) as a yellow-brown solid, m/z=757 [M]⁺.

### 3U: Synthesis of 3-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-5,6-dihydroxy-1-methyl-1H-benzo[d]imidazol-3-ium (415 E/Z)

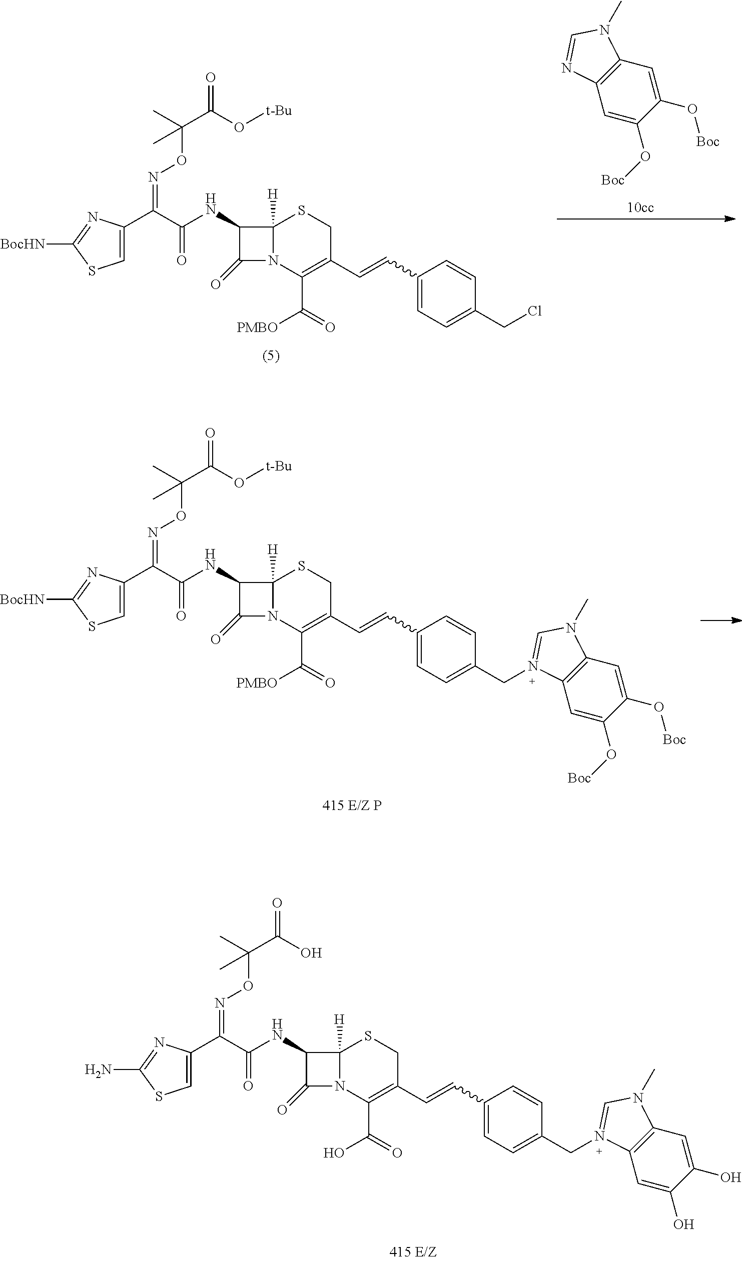

Di-tert-butyl (1-methyl-1H-benzo[d]imidazole-5,6-diyl) bis(carbonate) (3.0 equiv, 0.059 mmol) is used following the same procedure as described for the synthesis of compound 412 E/Z P. The residue is triturated with water to give a sample of 415 E/Z (quantitative yield) as a pale-brown solid, which is used in next step without further purification.

### Deprotection of Compound 415E/Z P

Compound 415 E/Z P (1.0 equiv, 0.06 mmol) is reacted as described for compound 412 E/Z P and the progress of reaction is followed by LC-MS. The residue is triturated with diethyl ether, acetonitrile and water to give a sample of 415 E/Z (0.015 g, 35%, over two steps) as a beige solid, m/z=734 [M]⁺.

### 3V: Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3 yl)vinyl)benzyl)-7,8-dihydroxy-2-methyl-1,2,3,4-tetrahydroisoquinolin-2-ium (442 E/Z)

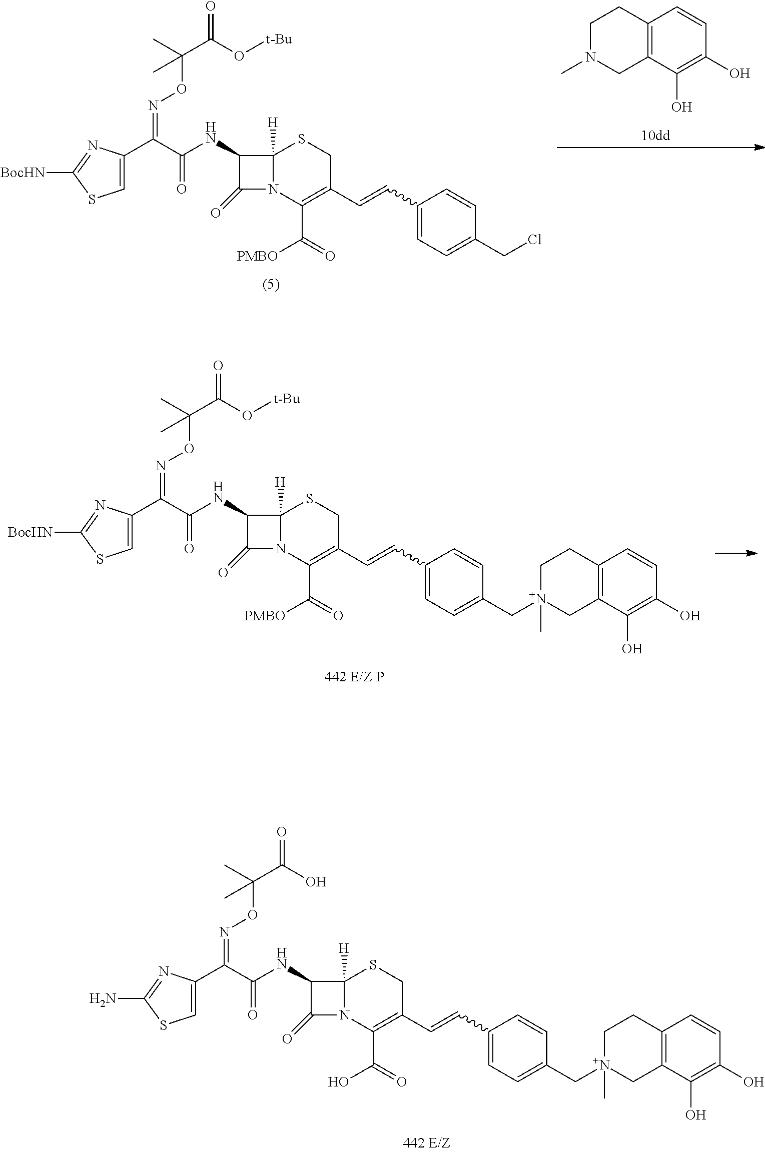

### Synthesis of 2-(4-((E/Z)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4-methoxybenzyl)oxy)carbonyl)-8-oxo-5-thia-1-azabicyclo[4.2.0]oct-2-en-3-yl)vinyl)benzyl)-7,8-dihydroxy-2-methyl-1,2,3,4-tetrahydroisoquinolin-2-ium (442 E/Z P)

2-Methyl-1,2,3,4-tetrahydroisoquinoline-6,7-diol (10dd) (1.3 equiv, 0.088 mmol) is used following the same procedure as described for the synthesis of compound 424 E/Z except that no potassium carbonate base is used. To the residue is added 1N HCl solution to give a sample of 442 E/Z P (quantitative yield) as a yellow solid, which is used in next step without further purification.

### Deprotection of 442 E/Z P

Intermediate 442 E/Z P (0.07 g, 0.068 mmol) is dissolved in anhydrous dichloromethane (0.7 mL). At −40° C., anisole (0.074 mL, 0.68 mmol) is added. To the resulting orange solution is added a 2 M aluminum chloride solution in nitromethane (0.34 mL), dropwise at −40° C. The mixture solidified and is stirred at −40° C. for 5 minutes, then at 0° C. for 1 hour. The mixture is allowed to reach room temperature and aqueous HCl solution (1 N, 20 mL) is added as well as acetonitrile (10 mL). This mixture is transferred into a separation funnel and diethyl ether (25 mL) is added. The layers are separated and to the aqueous layer is added 5.1 g of Diaion HP20 resin. Acetonitrile is evaporated under reduced pressure and resin is filtered on sintered glass. It is washed with water. Product is then desorbed using acetonitrile/water 1:1 (2×25 mL). A portion of crude product (0.023 g out of 0.039 g crude) is purified by reverse phase chromatography, eluting with mixtures of acetonitrile/water to give compound 442 E/Z (0.002 g, 8.7%, over two steps) as a light-yellow solid, m/z=749, [M]⁺.

### 3W: Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-2-cyclobutyl-7,8-dihydroxy-1,2,3,4-tetrahydroisoquinolin-2-ium (444 E/Z)

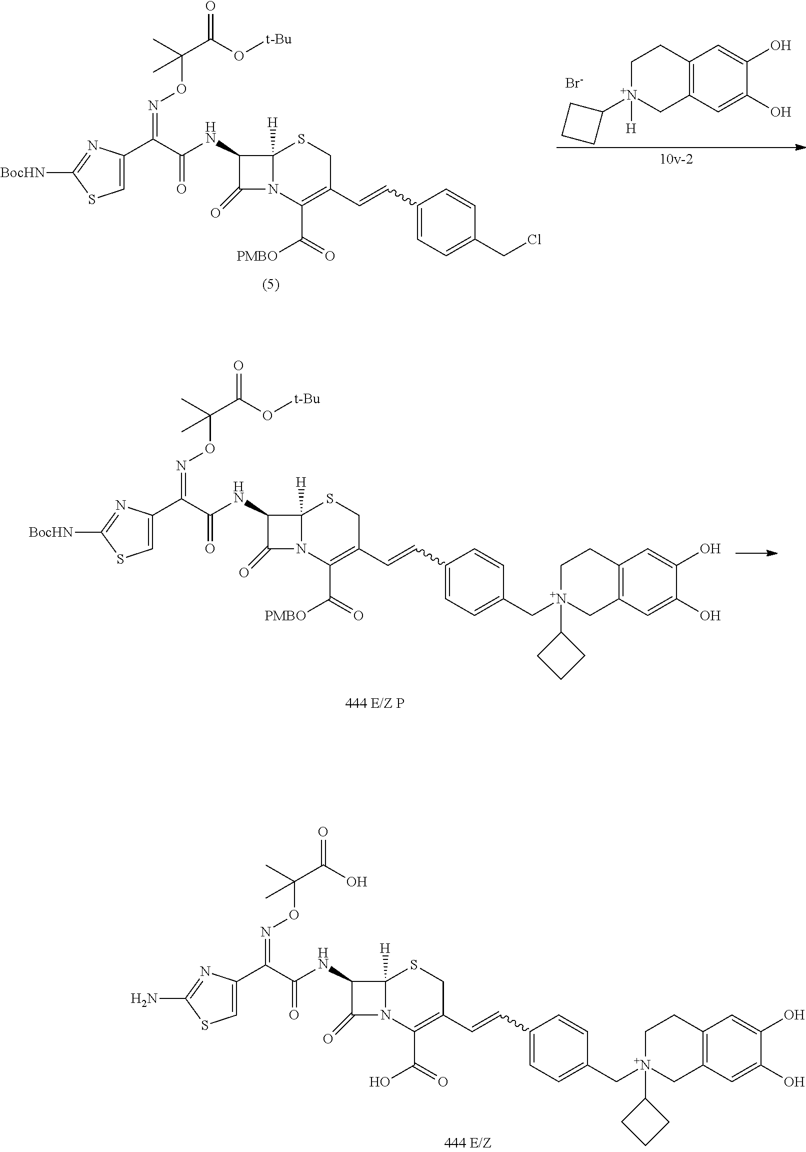

### Synthesis of 2-(4-((E/Z)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4-methoxybenzyl)oxy)carbonyl)-8-oxo-5-thia-1-azabicyclo[4.2.0]oct-2-en-3-yl)vinyl)benzyl)-2-cyclobutyl-7,8-dihydroxy-1,2,3,4-tetrahydroisoquinolin-2-ium (444 E/Z P)

2-Cyclobutyl-1,2,3,4-tetrahydroisoquinoline-6,7-diol hydrobromide salt (10v-2)(1.1 equiv, 0.19 mmol) is used following the same procedure as described for the synthesis of compound 424 E/Z. To the residue is added 1N HCl solution to obtain a solid. The residue is co-evaporated with toluene give a 444 E/Z P (quantitative yield) as a yellow solid which is used in next step without further purification, m/z=1066 [M]⁺.

### Deprotection of 444 E/Z P

Compound 444 E/Z P (1.0 equiv, 0.16 mmol) is reacted following the same procedure as described for the synthesis of compound 442 E/Z P. A portion of the crude product (0.025 g) is purified by reverse phase chromatography, eluting with mixtures of water (containing 0.1% TFA) and acetonitrile to give compound 444 E/Z (0.0086 g, 34%, over two steps) as a pale-yellow solid, m/z=789, [M]⁺.

### 3X: Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)-3-nitrobenzyl)-6,7-dihydroxy-2-methyl-1,2,3,4-tetrahydroisoquinolin-2-ium (445 E/Z)

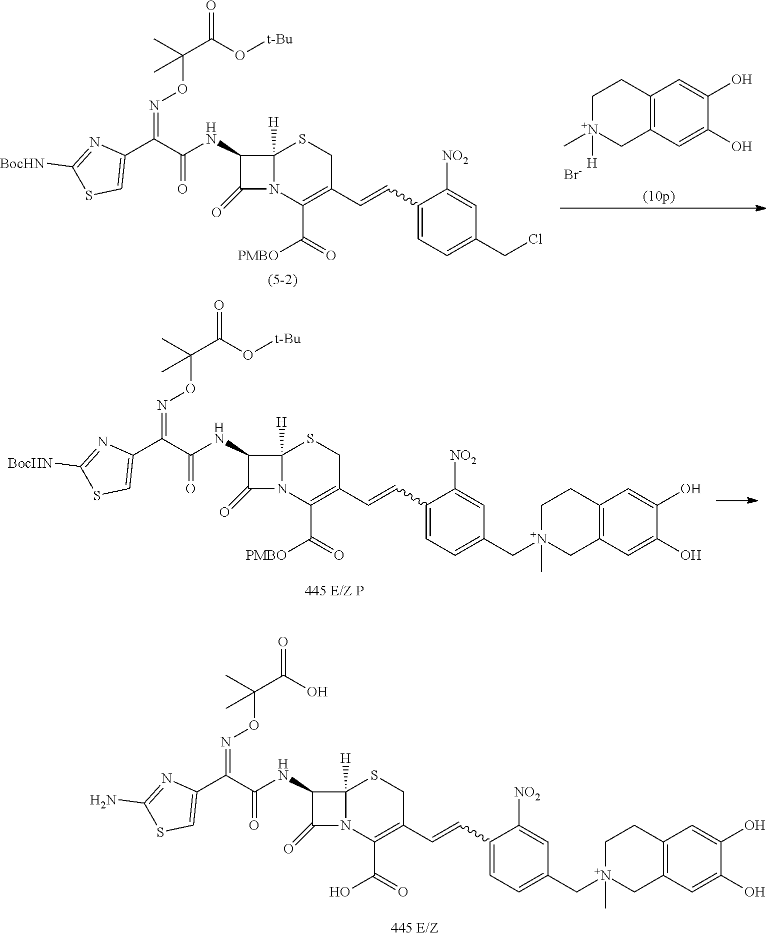

### Synthesis of 2-(4-((E/Z)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4-methoxybenzyl)oxy)carbonyl)-8-oxo-5-thia-1-azabicyclo[4.2.0]oct-2-en-3-yl)vinyl)-3-nitrobenzyl)-6,7-dihydroxy-2-methyl-1,2,3,4-tetrahydroisoquinolin-2-ium (445 E/Z P)

2-Methyl-1,2,3,4-tetrahydroisoquinoline-6,7-diolhydrobromide salt (10p)(1.1 equiv, 0.12 mmol) is reacted following the same procedure as described for the synthesis of compound 424 E/Z. To the residue is added 1N HCl solution to obtain a solid. The residue is co-evaporated with toluene give 445 E/Z P (quantitative yield) as a dark-orange solid, which is used, in next step without further purification.

### Deprotection of 445 E/Z P

Compound 445 E/Z P (1.0 equiv, 0.11 mmol) is reacted following the same procedure as described compound 442 E/Z P. The crude compound is purified by reverse phase chromatography, eluting with mixtures of water (containing 0.1% TFA) and acetonitrile to give compound 445 E/Z (0.010 g, 11%, over two steps) as a dark-yellow solid, m/z=794, [M]⁺.

### 3Y: Synthesis of 2-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-5,8-dichloro-6,7-dihydroxyisoquinolin-2-ium

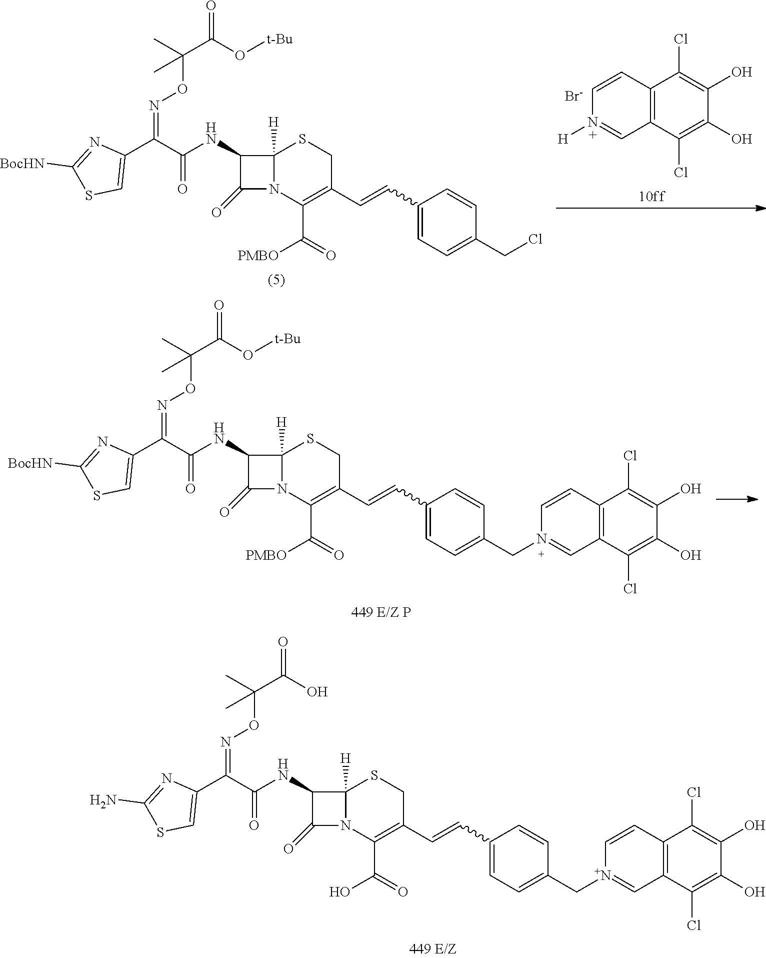

5,8-Dichloroisoquinoline-6,7-diol hydrobromide salt (10ff) 1.3 equiv, 0.09 mmol) is reacted following the same procedure as described for the synthesis of compound 424 E/Z P. To the residue is added few drops of TFA and DMF is evaporated under reduced pressure. Ethyl acetate and aqueous 1 N HCl solution are added and the layers are separated and the organic layer is washed two more times with 1 N HCl solution, then with brine. The organic layer is dried over magnesium sulfate and volatiles are removed under reduced pressure. The residue is triturated in diethyl ether to obtain 449 E/Z P (0.046 g, 62%) as an orange solid which is used in next step without further purification, m/z=1075 [M]⁺.

### Deprotection of 449 E/Z P

Compound 449 E/Z P (1.0 equiv, 0.02 mmol) is reacted following the same procedure as described for the synthesis of compound 442 E/Z. After lyophilization of the filtrate, product 10h (0.005 g, 31%) is obtained as a beige solid.

### 3Z: Synthesis of N-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-1-(3,4-dihydroxyphenyl)-N,N-dimethylmethanaminium (426 E/Z)

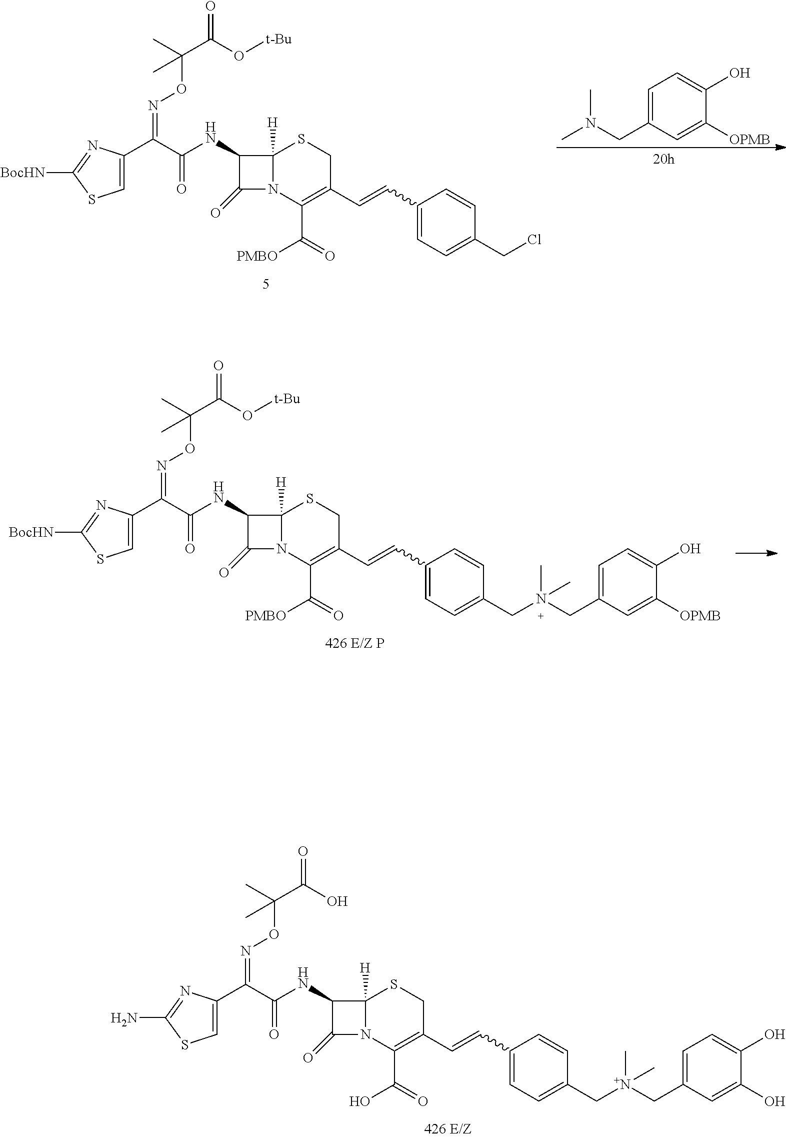

### Synthesis of N-(4-((E/Z)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4-methoxybenzyl)oxy)carbonyl)-8-oxo-5-thia-1-azabicyclo[4.2.0]oct-2-en-3-yl)vinyl)benzyl)-1-(4-hydroxy-3-((4-methoxybenzyl)oxy)phenyl)-N,N-dimethylmethanaminium

4-((Dimethylamino)methyl)-2-((4-methoxybenzyl)oxy)phenol 20h (2.5 equiv, 0.18 mmol) is used following the same procedure as described for the synthesis of compound 424 E/Z. No potassium carbonate base is used. To the residue is triturated in water and 1.2 N HCl solution and finally diethyl ether. Obtained a sample of compound 426 E/Z P (0.098 g, quantitative yield) as a pale-yellow powder, m/z=1133 [M]⁺.

### Deprotection of 426 E/Z P

Compound 426 E/Z P (1.0 equiv, 0.04 mmol) is used following the same procedure as described for the synthesis of compound 442 E/Z. The residue is lyophilized in a mixture of acetonitrile and water to obtain compound 426 E/Z (0.013 g, 45%) as an off-white solid, m/z=737 [M]⁺.

### 3AA: Synthesis of N-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-2-(3,4-dihydroxyphenyl)-N,N-dimethylethan-1-aminium (440 E/Z)

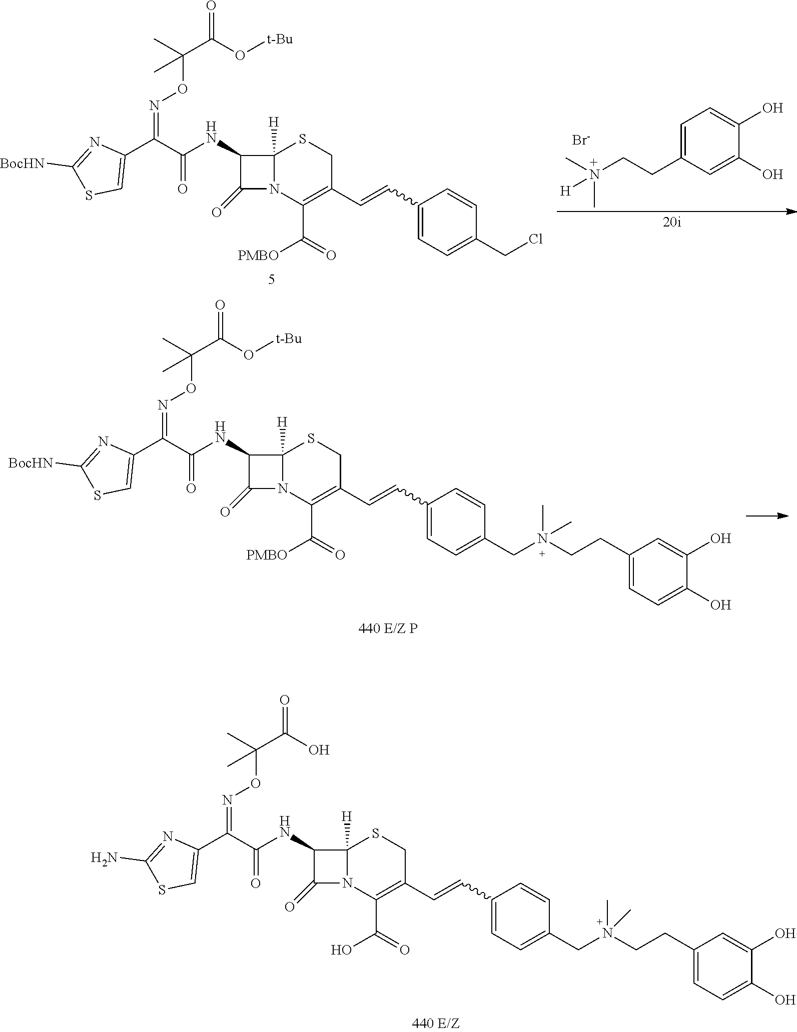

### Synthesis of N-(4-((E/Z)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4-methoxybenzyl)oxy)carbonyl)-8-oxo-5-thia-1-azabicyclo[4.2.0]oct-2-en-3-yl)vinyl)benzyl)-2-(3,4-dihydroxyphenyl)-N,N-dimethylethan-1-aminium

4-(2-(dimethylamino)ethyl)benzene-1,2-diol hydrobromide salt 20i (1.3 equiv, 0.088 mmol) is reacted following the same procedure as described for the synthesis of compound 412 E/Z P. To the residue is added 1 N HCl solution to give a sample of 440 E/Z P (quantitative yield) as a yellow solid, which is used in next step without further purification.

### Deprotection of 440 E/Z P

Compound 440 E/Z P (1.0 equiv, 0.068 mmol) is used following the same procedure as described for the synthesis of compound 412 E/Z. The residue is purified by reverse phase chromatography, eluting with mixtures of water and acetonitrile to give compound 440 E/Z (0.012 g, 23%, over two steps) as a yellow solid, m/z=751 [M]⁺.

### 3BB: Synthesis of N-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-1-(2-chloro-3,4-dihydroxyphenyl)-N,N-dimethylmethanaminium (434 E/Z)

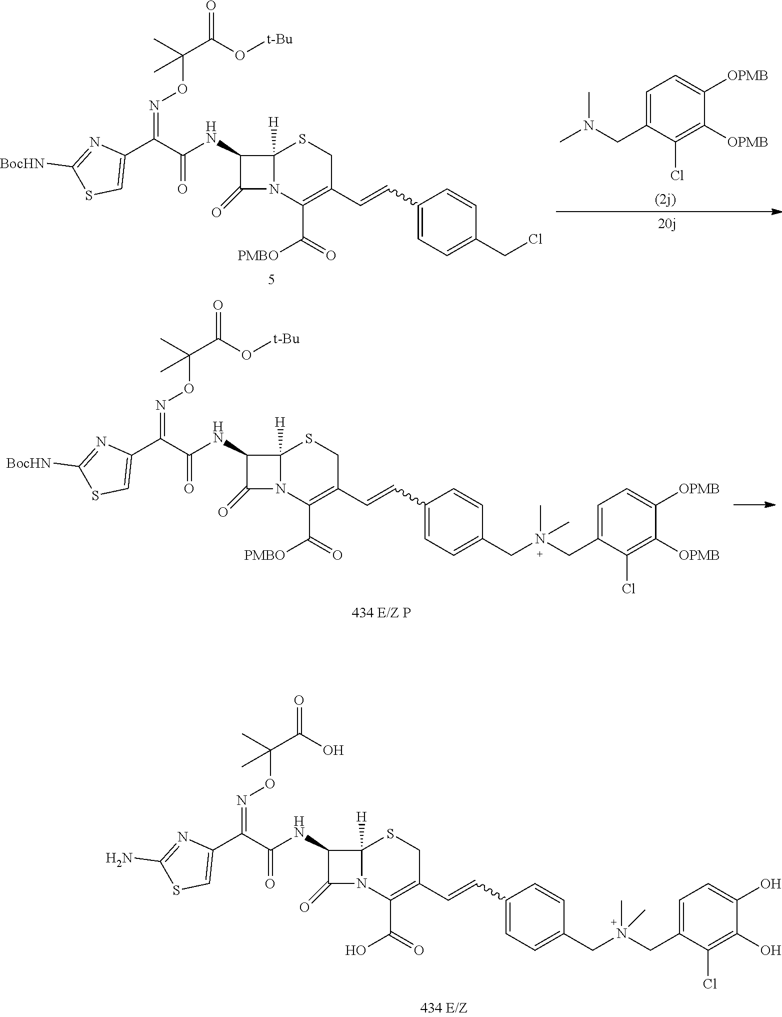

### Synthesis of N-(4-((E/Z)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4-methoxybenzyl)oxy)carbonyl)-8-oxo-5-thia-1-azabicyclo[4.2.0]oct-2-en-3-yl)vinyl)benzyl)-1-(2-chloro-3,4-bis((4-methoxybenzyl)oxy)phenyl)-N,N-dimethylmethanaminium

1-(2-Chloro-3,4-bis((4-methoxybenzyl)oxy)phenyl)-N,N-dimethylmethanamine 20j (2.0 equiv, 0.1 mmol) is used following the same procedure as described for the synthesis of compound 424 E/Z. No potassium carbonate base is used. The residue is triturated in 0.5 N HCl solution to obtain compound 434 E/Z P (quantitative yield) as an orange powder, which is used in next step without further purification.

### DeProtection of 434 E/Z P

Compound 434 E/Z P (1.0 equiv, 0.051 mmol) is reacted following the same procedure as described for the synthesis of compound 442 E/Z. The residue is lyophilized in a mixture of acetonitrile and water to obtain compound 434 E/Z (0.028 g, 72%, over two steps) as an off-white solid, m/z=771 [M]⁺.

### 3CC: Synthesis of N-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-1-(3-chloro-4,5-dihydroxyphenyl)-N,N-dimethylmethanaminium (436 E/Z)

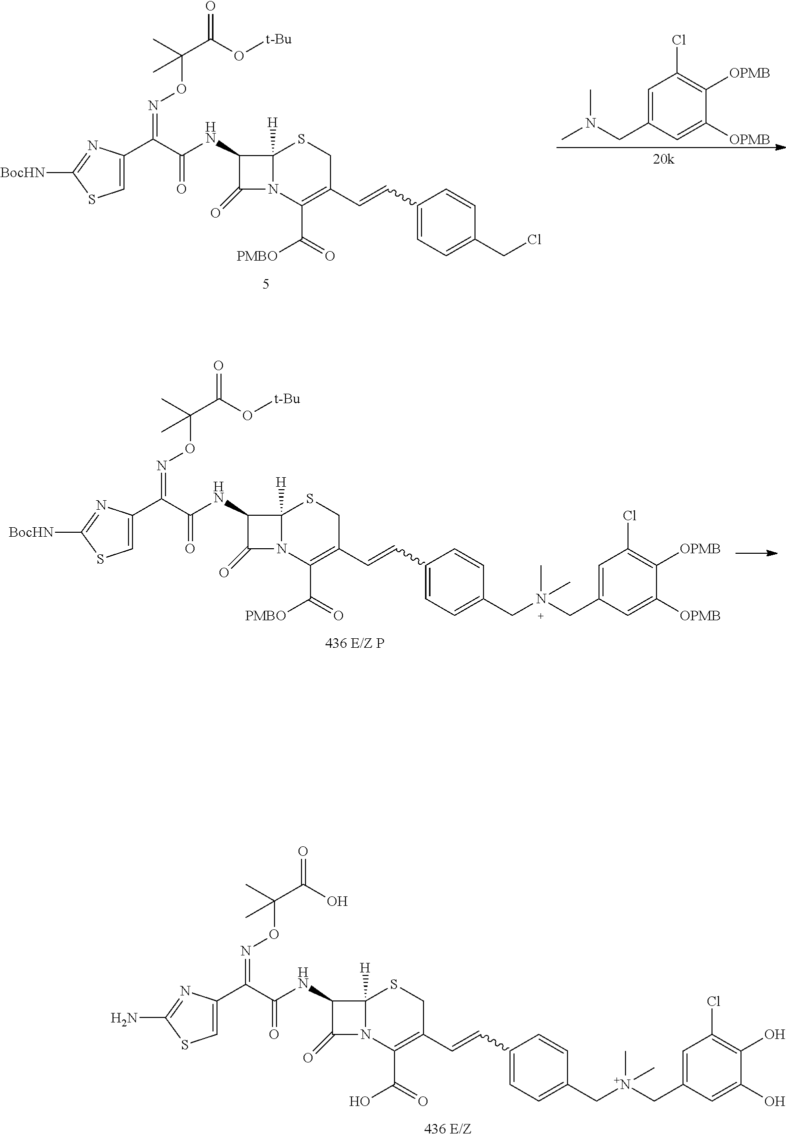

### Synthesis of N-(4-((E/Z)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4 methoxybenzyl)oxy)carbonyl)-8-oxo-5-thia-1-azabicyclo[4.2.0]oct-2-en-3-yl)vinyl)benzyl)-1-(3-chloro-4,5-bis((4-methoxybenzyl)oxy)phenyl)-N,N-dimethylmethanaminium

1-(3-Chloro-4,5-bis((4-methoxybenzyl)oxy)phenyl)-N,N-dimethylmethanamine 20k (1.3 equiv, 0.14 mmol) is reacted following the same procedure as described for the synthesis of compound 424 E/Z. No potassium carbonate base is used. The residue is triturated in 0.5 N HCl solution to obtain a sample of compound 436 E/Z P (quantitative yield) as an orange solid, which was used in next step without further purification.

### Deprotection of 436 E/Z P

Compound 436 E/Z P (1.0 equiv, 0.10 mmol) is used following the same procedure as described for the synthesis of compound 442 E/Z. The residue is lyophilized in a mixture of acetonitrile and water to obtain compound 436 E/Z (0.067 g, 82%, over two steps) as a beige solid, m/z=771 [M]⁺.

### 3DD: Synthesis of 3-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-5-((3,4-dihydroxyphenyl)carbamoyl)-1-methyl-1H-imidazol-3-ium (411 E/Z)

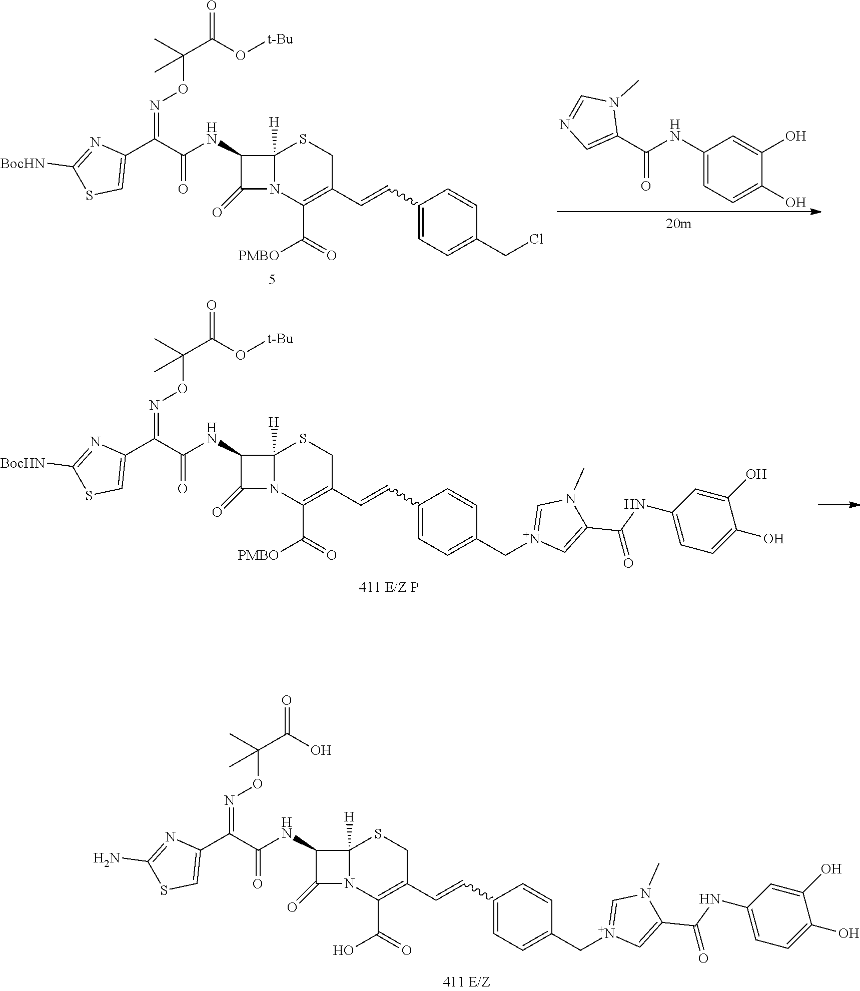

### Synthesis of 3-(4-((E/Z)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4 methoxybenzyl)oxy)carbonyl)-8-oxo-5-thia-1-azabicyclo[4.2.0]oct-2-en-3-yl)vinyl)benzyl)-5-((3,4-dihydroxyphenyl)carbamoyl)-1-methyl-1H-imidazol-3-ium

N-(3,4-Dihydroxyphenyl)-1-methyl-1H-imidazole-5-carboxamide 20m (4.0 equiv, 0.2 mmol) is used following the same procedure as described for the synthesis of compound 412 E/Z P No potassium carbonate base was used. The residue is triturated in water to obtain compound 411 E/Z P (quantitative yield), which was used in next step without further purification.

### Deprotection of 411 E/Z P

Compound 411 E/Z P (1.0 equiv, 0.05 mmol) is dissolved in dichloromethane (1.1 mL) under a nitrogen atmosphere. Reaction solution is cooled over an ice bath for 10 minutes. Anisole (0.25 mL) is added drop-wise and stirred over ice bath for 10 minutes. TFA (1.12 ml) is added drop-wise and stirred over ice bath for 2 h. Then reaction is placed in the fridge and the progress of reaction is followed by LC-MS. The reaction mixture is concentrated with under reduced pressure. Acetonitrile is added to dissolve the residue and concentrated. The crude compound is purified by reverse phase chromatography, eluting with mixtures of water and acetonitrile to give compound 411 E/Z (0.016 g, 36%, over two steps) as a beige solid, m/z=803 [M]⁺.

### 3EE: Synthesis of 3-(4-((E)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-1-(3,4-dihydroxyphenyl)-1H-imidazol-3-ium (414 E/Z)

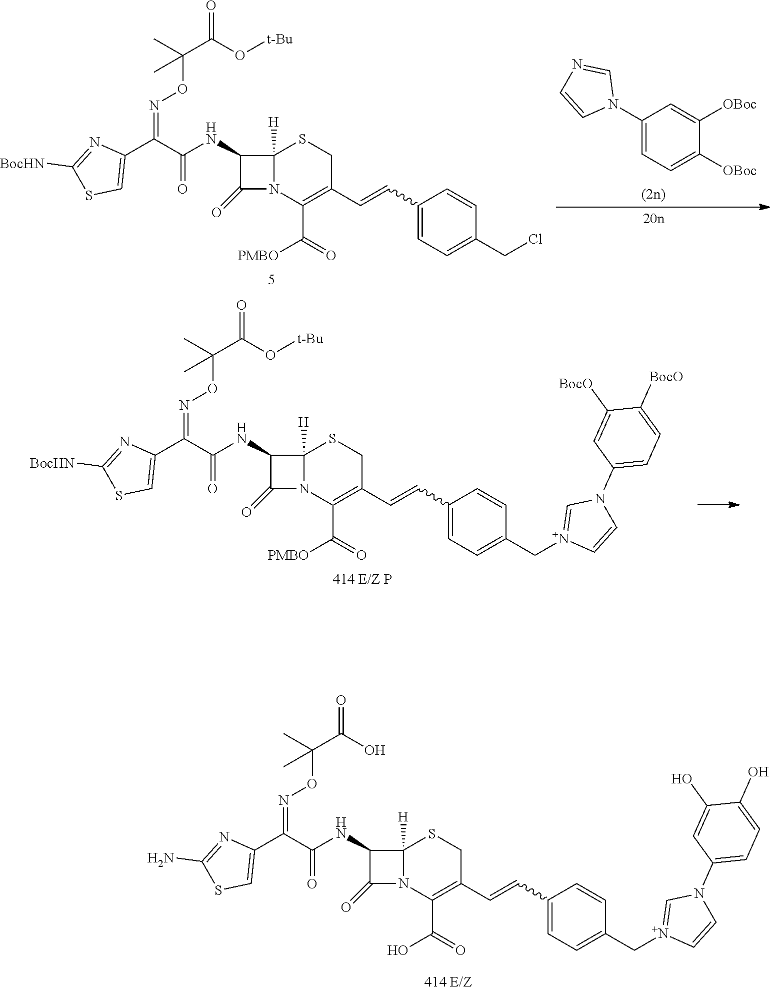

### Synthesis of 1-(3,4-bis((tert-butoxycarbonyl)oxy)phenyl)-3-(4-((E/Z)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4-methoxybenzyl)oxy)carbonyl)-8-oxo-5-thia-1-azabicyclo[4.2.0]oct-2-en-3-yl)vinyl)benzyl)-1H-imidazol-3-ium

4-(1H-imidazol-1-yl)-1,2-phenylene di-tert-butyl bis(carbonate) (4.0 equiv, 0.25 mmol) is used following the same procedure as described for the synthesis of compound 412 E/Z P. No potassium carbonate base is used. To the residue is triturated in water to obtain compound 414 E/Z P (0.76 g, quantitative yield), which was used in next step without further purification.

### Deprotection of Compound 414 E/Z P

Compound 414 E/Z P (1.0 equiv, 0.06 mmol) is used following the same procedure as described for the synthesis of compound 411 E/Z and the progress of reaction is followed by LC-MS. The residue is triturated with diethyl ether and water to give 414 E/Z (0.045 g, 97%) as a beige solid, m/z=746 [M]⁺ and 373 [M+H]²⁺.

### 3FF: Synthesis of 2-(4-((E)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-6-carboxy-5-hydroxyisoquinolin-2-ium

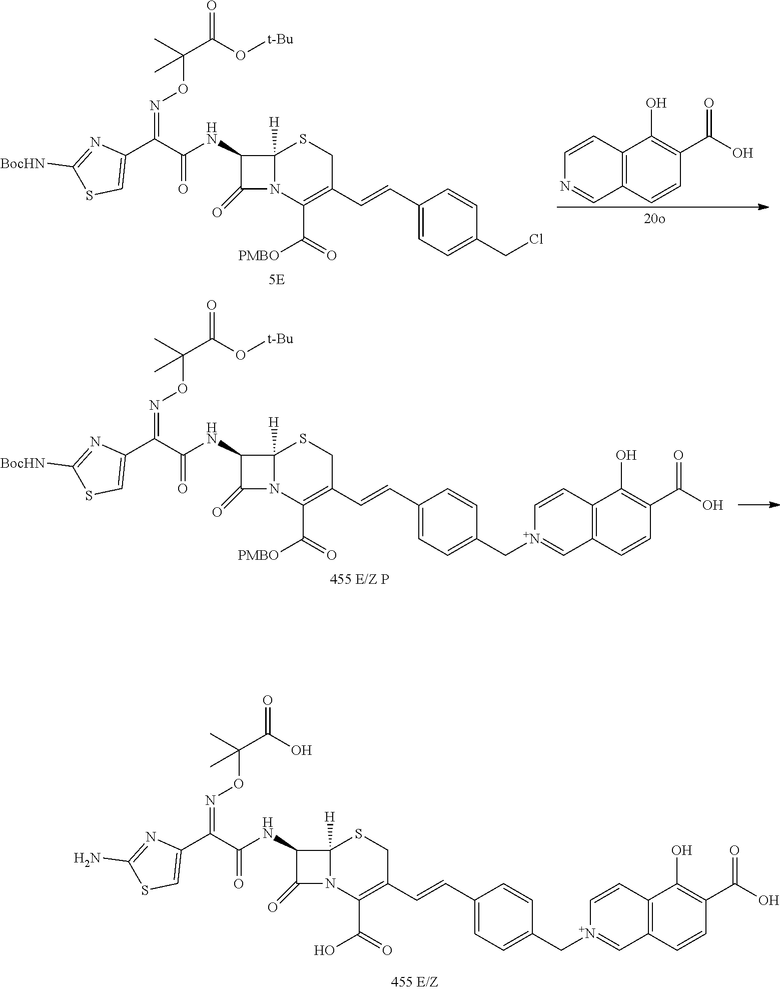

### Synthesis of 2-(4-((E)-2-((6R,7R)-7-((Z)-2-(((1-(tert-butoxy)-2-methyl-1-oxopropan-2 yl)oxy)imino)-2-(2-((tert-butoxycarbonyl)amino)thiazol-4-yl)acetamido)-2-(((4-methoxybenzyl)oxy)carbonyl)-8-oxo-5-thia-1-azabicyclo[4.2.0]oct-2-en-3-yl)vinyl)benzyl)-6-carboxy-5-hydroxyisoquinolin-2-ium

5-hydroxyisoquinoline-6-carboxylic acid (1.3 equiv, 0.147 mmol) is used following the same procedure as described for the synthesis of compound 412 E/Z P. Ethyl acetate and aqueous 1 N HCl solution are added and layers are separated. The organic layer is washed two more times with a saturated solution of NH₄Cl and 1 N HCl solution, then with brine and dried over MgSO₄. The crude product is purified by reverse phase chromatography, eluting with mixtures of water and acetonitrile to give compound 455 E P (E isomer, 0.045 g, 38%) as a yellow solid, m/z=1036 [M]⁺.

### Deprotection of Compound 455 E P

To pure E isomer of compound 455 E P (E isomer, 1.0 equiv, 0.046 mmol) are added TFA (1.0 mL), water (0.025 mL) and triisopropylsilane (0.025 mL). The reaction mixture is stirred at room temperature for one hour. Solvent is removed and the residue is triturated with diethyl ether to give 455 E (E isomer, 0.024, 68%) as a yellow solid, m/z=759 [M]⁺.

### 3GG: Synthesis of 1-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-6,7-dihydroxyquinolin-1-ium (456 E/Z)

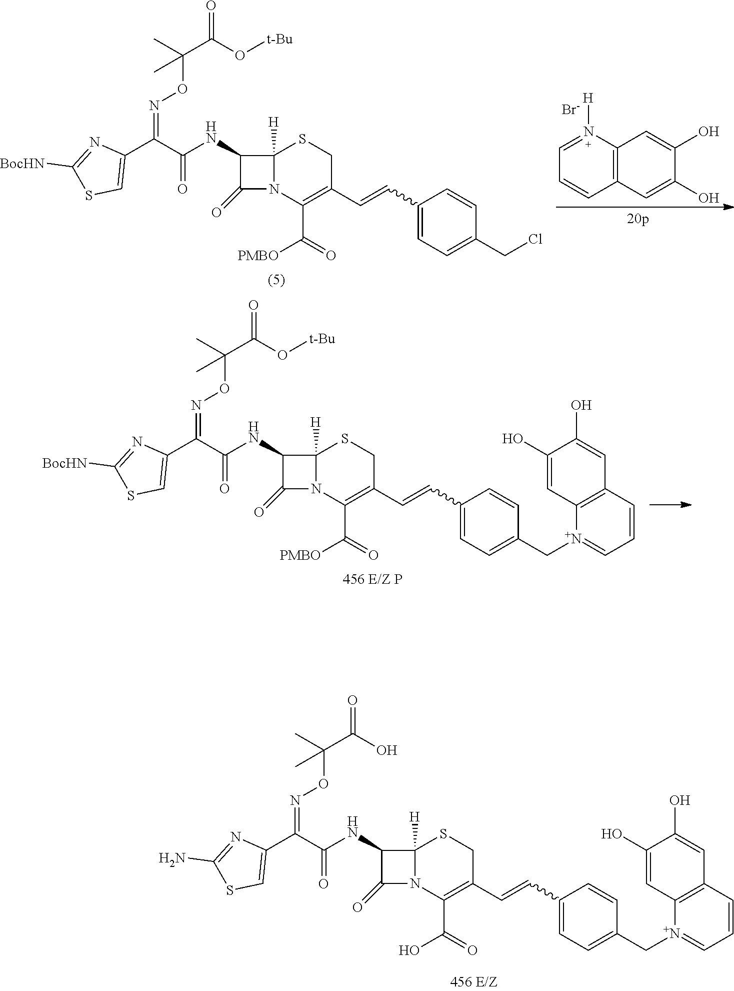

### Synthesis of 1-(4-((E/Z)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4-methoxybenzyl)oxy)carbonyl)-8-oxo-5-thia-1-azabicyclo[4.2.0]oct-2-en-3-yl)vinyl)benzyl)-6,7-dihydroxyquinolin-1-ium

Quinoline-6,7-diol hydrobromide salt 20p (1.3 equiv, 0.147 mmol) is used following the same procedure as described for the synthesis of compound 412 E/Z P. The residue is purified directly by reverse phase chromatography, eluting with mixtures of water and acetonitrile to give compound 456 E/Z P (0.084 g, 74%) as a yellow solid, m/z=1007 [M]⁺.

### Deprotection of Compound 456 E/Z P

Compound 456 E/Z P (1.0 equiv, 0.082 mmol) is used following the same procedure as described for the synthesis of compound 455 E P. The residue is triturated with diethyl ether to give 456 E/Z (0.057 g, 95%) as a yellow solid, m/z=731 [M]⁺, 366 [M+H]²⁺.

### 3HH: Synthesis of 1-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-4-(3,4-dihydroxyphenyl)-3-methylpyridin-1-ium (422 E/Z)

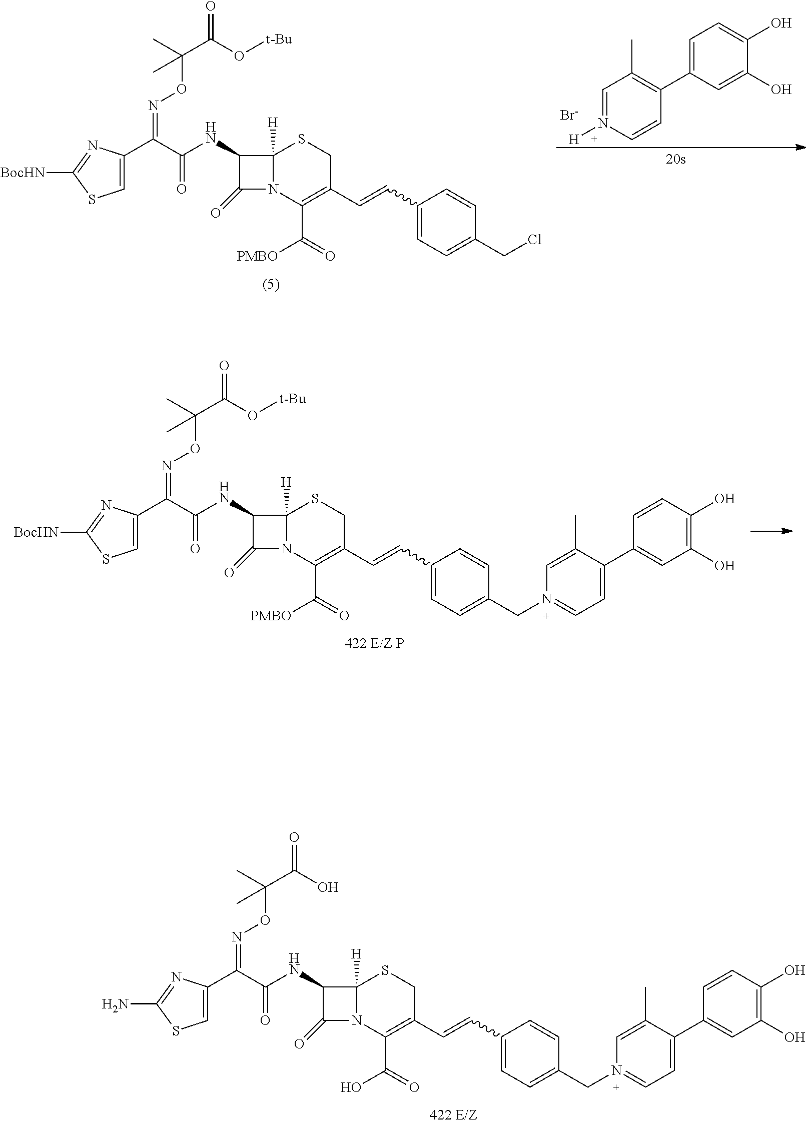

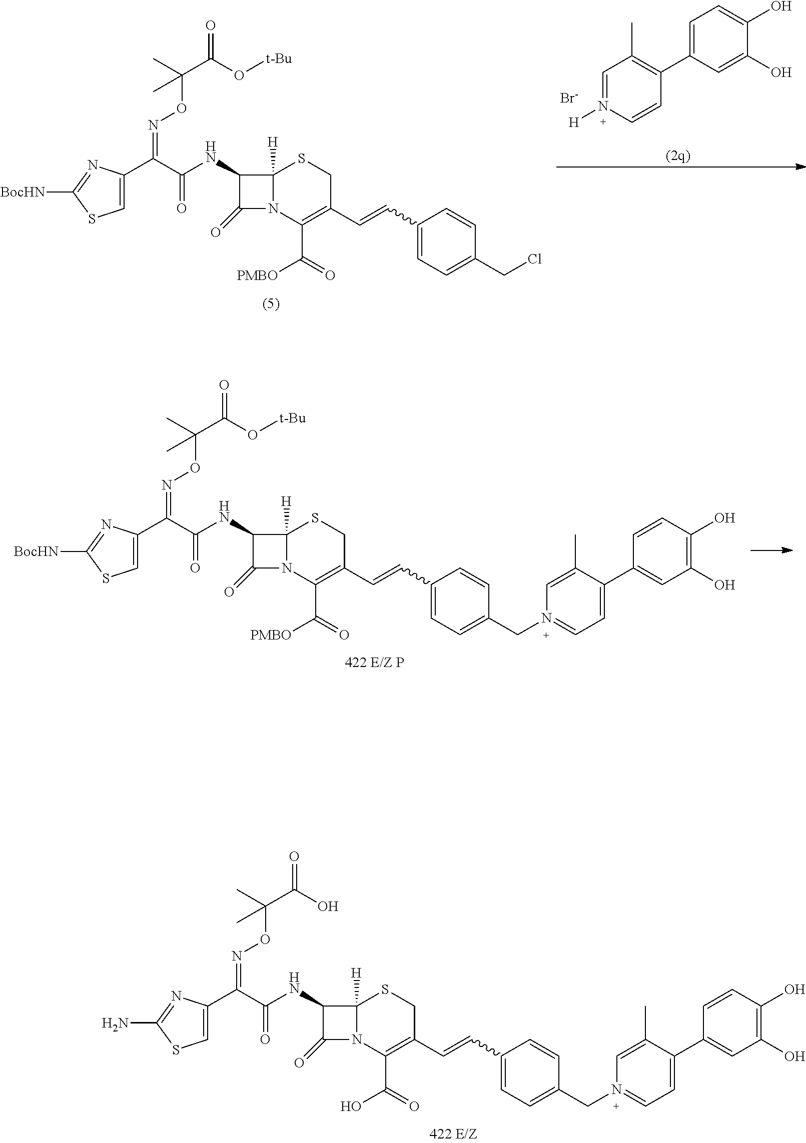

### Synthesis of 1-(4-((E/Z)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4 methoxybenzyl)oxy)carbonyl)-8-oxo-5-thia-1-azabicyclo[4.2.0]oct-2-en-3-yl)vinyl)benzyl)-4-(3,4-dihydroxyphenyl)-3-methylpyridin-1-ium

4-(3-Methylpyridin-4-yl)benzene-1,2-diol hydrobromide salt 20s (3.0 equiv, 0.137 mmol) is used following the same procedure as described for the synthesis of compound 412 E/Z P. The residue is triturated with diethyl ether to give 422 E/Z P (quantitative yield) as an orange solid which is used in next step without further purification, m/z=1047 [M]⁺.

### Deprotection of 422 E/Z P

Compound 422 E/Z P (1.0 equiv, 0.047 mmol) is used following the same procedure as described for the synthesis of compound 414 E/Z and the progress of reaction is followed by LC-MS. The residue is triturated with diethyl ether and water to give a sample of 422 E/Z (0.019 g, 52%, over two steps) as a brown solid, m/z=771 [M]⁺.

### 3II: Synthesis of 1-(4-((E/Z)-2-((6R,7R)-7-((Z)-2-(2-aminothiazol-4-yl)-2-(((2-carboxypropan-2-yl)oxy)imino)acetamido)-2-carboxy-8-oxo-5-thia-1-azabicyclo[4.2.0]oct-2-en-3-yl)vinyl)benzyl)-4-(3,4-dihydroxybenzamido)pyridin-1-ium (427 E/Z)

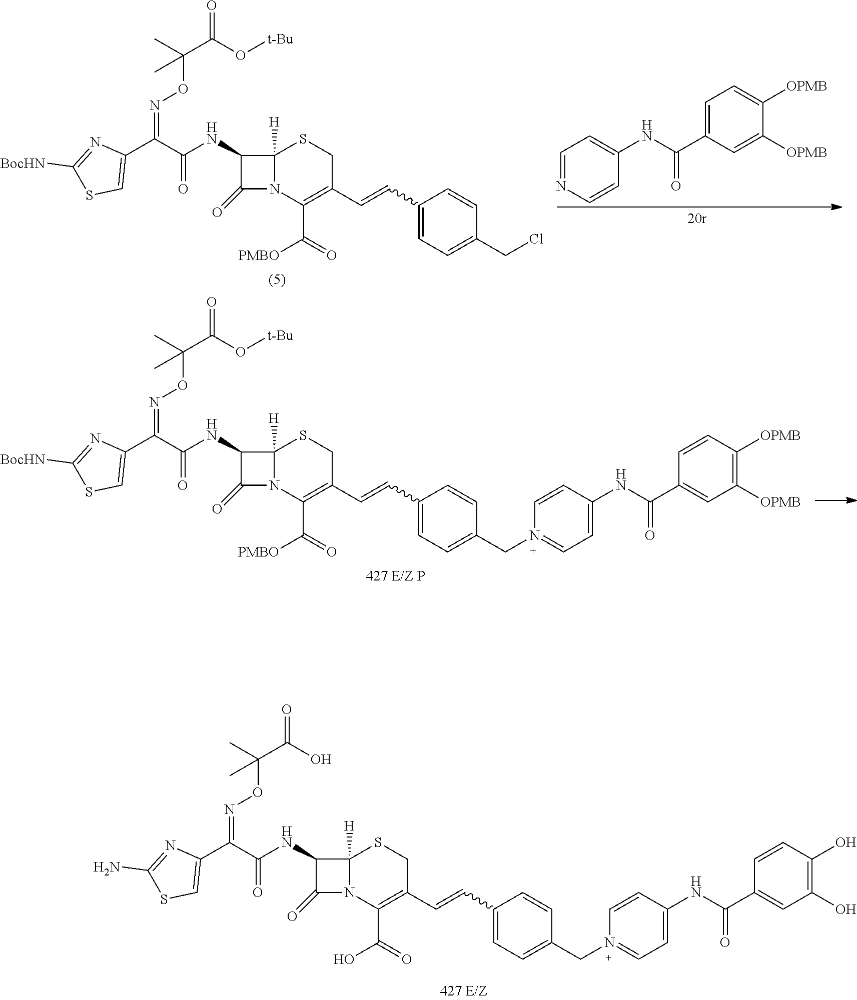

### Synthesis of 4-(3,4-bis((4-methoxybenzyl)oxy)benzamido)-1-(4-((E/Z)-2-((6R,7R)-7-((Z)-2-(((1-(tert-butoxy)-2-methyl-1-oxopropan-2-yl)oxy)imino)-2-(2-((tert-butoxycarbonyl)amino)thiazol-4-yl)acetamido)-2-(((4-methoxybenzyl)oxy)carbonyl)-8-oxo-5-thia-1-azabicyclo[4.2.0]oct-2-en-3-yl)vinyl)benzyl)pyridin-1-ium

3,4-bis((4-methoxybenzyl)oxy)-N-(pyridin-4-yl)benzamide 20r (1.1 equiv, 0.042 mmol) is used following the same procedure as described for the synthesis of compound 424 E/Z. The compound 427 E/Z P (quantitative yield) is obtained as a dark-brown solid and is used in next step without any purification.

### Deprotection of 427 E/Z P

Compound 427 E/Z P (1.0 equiv, 0.04 mmol) is used following the same procedure as described for the synthesis of compound 414 E/Z and the progress of reaction is followed by LC-MS. The residue is triturated with diethyl ether to give a sample of 427 E/Z (0.013 g, 43%, over two steps) as a yellow powder, m/z=800 [M]⁺.

### Example 4: Antibiotic Activity

### Measurement of Minimum Inhibitory Concentrations (MIC)

### Inoculant

Sterile technique was used throughout these experiments. Suspensions of organisms were removed from storage (−80° C.) and partially thawed. Partially thawed organisms (1 μL) were inoculated into 3 mL sterile LB Broth in a sterile culture tube and capped, then cultured at 37° C. with shaking for 4-5 hours until the optical density at 600 nm (OD₆₀₀) was greater than 0.1 on a single beam spectrophotometer.

The concentration of organisms was adjusted to 0.08-0.1 OD₆₀₀ (0.5 McFarland units) by dilution with sterile cation-adjusted Meuller-Hinton (CAMH) broth. The suspensions were used within 30 minutes of preparation.

### Test Compound Stock Solution

A stock solution was made of each test compound by measuring 6.4 mg of the compound on an analytical balance and dissolving it in DMSO (1 mL) to get a transparent solution. The solutions were stored at −20° C. and thawed prior to use.

### MIC Determination

Sterile CAMH broth (200 μL) containing 20 μM Apo-Transferrin was pipetted into the first row of 8 columns of a sterile, round-bottomed, transparent, 96-well microplate using an 8-channel pipette equipped with sterile tips. One hundred μL was pipetted into the wells of the remaining 11 rows.

To each well of the first row was pipetted 2.0 μL of the 6.4 mg/mL stock test compound stock solution to give 1:100 dilutions and a final concentration of 64 μg/mL and mixed.

With an 8-channel pipette equipped with sterile tips, 100 μL of the first row solution was transferred to the second row and mixed. The process was repeated for the second to third rows and continued through row 11. One hundred μL from row 11 was removed and discarded so that all 12 rows contained 100 μL. Row 12 contained no compound and rows 1-11 contained 64, 32, 16, 8, 4, 2, 1, 0.5, 0.25, 0.125, 0.6 μg/mL of a given compound.

Inoculant suspensions (0.5 μL) were added to the 12 wells of each column. The plates were then covered and incubated at 37° C. overnight for 18 hours.

For each organism the MIC was determined by identifying the transparent well with the lowest test compound concentration.

MIC (microg/mL) results are provided for exemplary compounds of Formulas I and II in Tables 1-1, 1-2 (various Pseudomonas aeruginosa strains as indicated); Table 2 for two representative Acinetobacter baumannii strains, one producing 0)(A-58 (a class D carbapenemase); and Tables 3-1 to Table 3-6 (various Enteric bacterium as indicated).

Organisms for which MICs were measured are known in the art, can be prepared by well-known methods from available starting materials are available from commercial sources or are clinical isolates.

Additional MIC results are provided in Tables 4-1 and 4-2 for E. coli J58 isogenic strains producing ESBL (Extended Spectrum beta-Lactamases) and other beta-lactamases as indicated below [23, 24].

With respect to Tables 4-1 and 4-2 the following beta-lactamases were employed:

TEM-26 (Group 2be beta-lactamase, hydrolyzes extended spectrum cephalosporins and monobactams), [25];

SHV-1 (Class A, Group 2b beta-lactamase, hydrolyzes penicillins and early cephalosporins) [26];

DHA-1 (beta-lactamase, intermediate or resistant to all penicillins, alone or in combination with beta-lactamase inhibitors, and to cephalothin, cefuroxime, cefoxitin, ceftazidime, aztreonam, and cefotaxime) [27, 28];

CTX-M-14 and CTX-M-15 (Class A, Group 2be beta-lactamases, hydrolyzes extended spectrum cephalosporins and monobactams) [29];

KPC-2 and KPC-3 (Class A, Group 2f, carbapenemases) [30, 31];

NDM-1 (Group 3a carbapenemase, broad spectrum divalent Zn⁺⁺ metallo-Beta-Lactamase) [32, 33];

VIM-2 (Group 3a carbapenemase, metallo-beta-lactamase) [34]; and

IMP-1 (Group 3a carbapenemase, metallo-beta-Lactamase).

The gene sequence for each beta-lactamase enzyme was synthesized and inserted into the pBC-SK(+) plasmid by GenScript, a commercial service. E. coli J53 was transformed with each plasmid via electroporation and resistant transformants were selected on agar plates containing 50 micrograms/mL ampicillin. The selected transformants were stored at −80 degrees C. in 20% glycerol.

The references cited above with respect to beta-lactamases are incorporated by reference herein for descriptions of various beta-lactamases and provide beta-lactamases in addition to those described herein which can be employed by one of ordinary skill in the art to further characterize the activity of compounds of Formulas I and II against bacteria producing such beta-lactamases.

It is noted on review of Tables 1-1 and 1-2 that a number of the compounds of the invention exhibit low microgram/mL MICs for various Pseudomonas strains. Of particular note are compounds 407 (E/Z), 431 (E/Z) and 435 (E/Z).

It is noted on review of Table 2 that several compounds of the invention, including, for example, compounds 431 (E/Z), 432 (E/Z), 435 (E/Z) and 442 (E/Z) exhibit low microgram/mL MICs for an Acinetobacter baumannii strain and that compound 407(E/Z) exhibits lower microgram/mL MICs for both Acinetobacter baumannii strains tested.

It is noted on review of Tables 3-1 to 3-6 that a number of the compounds of the invention exhibit low microgram/mL MICs for various enteric bacteria. Of particular note are compounds 431 (E/Z), 432 (E/Z) and 435 (E/Z).

It is noted on review of Tales 4-1 and 4-2 that compounds of the invention exhibit microgram/mL MICs against E. coli strains carrying genes for certain ESBL and other beta-lactamases. Of particular note, compounds exhibit microgram/mL MICs for certain metallo beta-lactamases. Of additional note, compounds 435 (E/Z), and 444 E/Z) exhibit lower MICS compared to other compounds tested for an E. coli strain carrying the gene for NDM-1. Of additional note, compounds 429 (E/Z), 431 (E/Z), 432 (E/Z) and 435 (E/Z) exhibit low microgram/mL MICs for an E. coli strain carrying the gene for carbapenemase VIM-1. Of additional note, compounds 432 (E/Z), 435 (E/Z), 437 (E/Z), 439 (E/Z), 440 (E/Z) and 442 (E/Z) exhibit low microgram/mL MICs for an E. coli strain carrying the gene for carbapenemase IMP-1.

TABLE 1-1
Minimum Inhibitory Concentrations (microgram/mL) of Exemplary Compounds of Formulas
I and II for Representative Pseudomonas aeruginosa (PA)strains.

| | Bacterium | | | | | |
|---|---|---|---|---|---|---|
| Compound | | PA | PA | PA | PA | PA-RR- |
| No. | PA | (URMC-2) | (URMC-3) | (RR-1) | (RR-2) | (G7304100) |
| 352 | 64 | 32 | >64 | 16 | 32 | ND |
| 353 | 32 | 16 | 64 | 4 | 8 | ND |
| 373 | 4 | 2 | 32 | 1 | 2 | ND |
| 388 | 32 | 16 | >64 | 16 | 1 | ND |
| 389 | 2 | 1 | 16 | 1 | 2 | ND |
| 394 | 8 | 2 | 16 | 2 | 2 | ND |
| 407 | 2 | 4 | 0.25 | 4 | 2 | 0.03 |
| 411 | 8 | 4 | 0.5 | 4 | 4 | 1 |
| 412 | 2 | 2 | ≤0.06 | 4 | 2 | ≤0.06 |
| 413 | 4 | 4 | 0.13 | 4 | 2 | ≤0.06 |
| 422 | 8 | 4 | ≤0.06 | 4 | 2 | ≤0.06 |
| 423 | 2 | 2 | ≤0.06 | 2 | 2 | ≤0.06 |
| 424 | 2 | 4 | 0.13 | 4 | 4 | ≤0.06 |
| 426 | 4 | 4 | 64 | 4 | 2 | 0.25 |
| 427 | 0.06 | 0.06 | 0.25 | 4 | ≤0.06 | ≤0.06 |
| 429 | 0.25 | 0.5 | 0.5 | 0.25 | ≤0.06 | ≤0.06 |
| 431 | 0.25 | ≤0.06 | 1 | 0.13 | ≤0.06 | ≤0.06 |
| 432 | 0.25 | 0.5 | 1 | 1 | 0.13 | ≤0.06 |
| 434 | 4 | 8 | 16 | 1 | 2 | 2 |
| 435 | ≤0.06 | 0.25 | ≤0.06 | 0.25 | ≤0.06 | ≤0.06 |
| 436 | 8 | 8 | 64 | 2 | 4 | 4 |
| 442 | 0.13 | 0.25 | 0.5 | 0.5 | ≤0.06 | ≤0.06 |

TABLE 1-2
Minimum Inhibitory Concentrations (microgram/mL) of Exemplary Compounds
of Formulas I and II for Representative Pseudomonas aeruginosa (PA)strains.

| | Bacterium | | | | | |
|---|---|---|---|---|---|---|
| | | PA-RR- | | | | |
| Compound | PA-RR- | (purple | PA-RR- | PA-RR- | PA-RR- | PA-RR- |
| No. | (G7305586) | Fre D) | (980151052) | (98023895) | (G8062859) | (G7294437) |
| 352 | ND | ND | ND | ND | ND | ND |
| 353 | ND | ND | ND | ND | ND | ND |
| 373 | ND | ND | ND | ND | ND | ND |
| 388 | ND | ND | ND | ND | ND | ND |
| 389 | ND | ND | ND | ND | ND | ND |
| 394 | ND | ND | ND | ND | ND | ND |
| 407 | 0.03 | 0.06 | 0.06 | 0.13 | 0.06 | 0.03 |
| 411 | 16 | 2 | 8 | 8 | 2 | 1 |
| 412 | 16 | 2 | 4 | 4 | 1 | 1 |
| 413 | 8 | 2 | 4 | 4 | 2 | 1 |
| 422 | 16 | 1 | 8 | 8 | 2 | 2 |
| 423 | 8 | 1 | 2 | 8 | 2 | 0.5 |
| 424 | 8 | 2 | 4 | 8 | 2 | 0.25 |
| 426 | 4 | 1 | 4 | 8 | 2 | 0.5 |
| 427 | 8 | 0.25 | 0.5 | 4 | 0.25 | ≤0.06 |
| 429 | 1 | 2 | 0.5 | 4 | 0.5 | ≤0.06 |
| 431 | 0.5 | 0.5 | 0.25 | 4 | 0.5 | ≤0.06 |
| 432 | 1 | 1 | 0.25 | 4 | 0.5 | ≤0.06 |
| 434 | 8 | 2 | 4 | 8 | 2 | 0.5 |
| 435 | 0.25 | 0.5 | 0.13 | 1 | 0.5 | ≤0.06 |
| 436 | 8 | 2 | 4 | 4 | 5 | 0.5 |
| 442 | 2 | 1 | 2 | 8 | 0.25 | ≤0.06 |

TABLE 2
Minimum Inhibitory Concentrations (microgram/mL)
of Exemplary Compounds of Formulas I and II for Representative
Acinetobacter baumannii strains.

| Compound | | A. baumannii |
|---|---|---|
| No. | A. baumannii | (JMI OXA-58) |
| 352 | 32 | 32 |
| 353 | 32 | >64 |
| 373 | 16 | >64 |
| 388 | >64 | >64 |
| 389 | 32 | >64 |
| 394 | 16 | 64 |
| 407 | 2 | 16 |
| 412 | 8 | >64 |
| 428 | 4 | >64 |
| 429 | 2 | >64 |
| 431 | 1 | >64 |
| 432 | 1 | >64 |
| 435 | 1 | >64 |
| 442 | 1 | >64 |
| 446 | 2 | >64 |

TABLE 3-1
Minimum Inhibitory Concentrations (microgram/mL) of Exemplary
Compounds of Formulas I and II for Representative Enteric Bacteria¹

| | Bacterium | | | | | | |
|---|---|---|---|---|---|---|---|
| Compound No. | EC | KP | EC | KP | KOXY | PM | CF |
| 252 | 0.5 | 0.25 | 0.5 | 0.25 | 32 | ≤0.06 | 0.5 |
| 353 | 0.5 | 0.25 | 0.5 | 0.25 | 32 | ≤0.06 | 0.5 |
| 373 | 0.13 | 0.13 | 0.13 | 0.13 | 0.5 | ≤0.06 | 0.13 |
| 388 | 2 | 4 | 2 | 4 | 8 | ≤0.06 | 1 |
| 389 | 0.13 | 0.25 | 0.13 | 0.25 | 1 | ≤0.06 | ≤0.06 |
| 394 | 0.25 | 0.25 | 0.25 | 0.25 | 2 | ≤0.06 | 0.25 |
| 395 | 0.5 | 0.5 | 0.5 | 0.5 | 1 | ≤0.06 | 0.5 |
| 407 | 0.25 | 0.25 | 0.25 | 0.25 | 1 | ≤0.06 | ≤0.06 |
| 412 | ND | 2 | ND | 2 | 0.5 | ≤0.06 | ≤0.06 |
| 429 | ND | 1 | ND | 1 | 0.5 | ND | ND |
| 431 | 0.5 | 0.13 | 0.5 | 0.13 | 0.13 | ND | ND |
| 432 | 0.25 | 1 | 0.25 | 1 | 0.5 | ND | ND |
| 435 | ≤0.06 | ≤0.06 | ≤0.06 | ≤0.06 | ≤0.06 | ≤0.06 | ≤0.06 |
| 442 | 0.25 | ND | 0.25 | ND | ND | ND | ND |
| 446 | 0.5 | ND | 0.5 | ND | ND | ND | ND |

¹EC = Escherichia coli; KP = Klebsiella pneumoniae; KOXY = Klebsiella oxytoca; PM = Proteus mirabilis; CF = Citrobacter freundii; SM = Serratia marcescens; ENA = Enterobacter aerogenes; ENC = Enterobacter cloacae.

TABLE 3-2
Minimum Inhibitory Concentrations (microgram/mL) of Exemplary
Compounds of Formulas I and II for Representative Enteric Bacteria¹

| | Bacterium | | | | | | |
|---|---|---|---|---|---|---|---|
| | | | | KP(GJ | KP (GJ | KP | KP(ATCC |
| Compound | | EC(JMI | KP(JMI | 5934 | 5659 | (ATCC | TEM- |
| No. | SM | OXA-48) | KPC-2) | TEM-26) | TEM-26) | KPC-3) | 10/TEM-12) |
| 252 | 2 | >64 | >64 | 4 | 16 | 64 | 32 |
| 353 | 1 | >64 | >64 | 0.5 | 0.5 | >64 | 2 |
| 373 | 0.5 | 16 | >64 | 0.25 | 2 | >64 | 64 |
| 388 | 8 | 64 | >64 | 32 | 8 | >64 | >64 |
| 389 | 0.5 | >64 | >64 | 8 | 0.25 | >64 | 64 |
| 394 | 1 | 64 | >64 | 1 | 32 | >64 | >64 |
| 395 | 1 | 64 | >64 | 16 | >64 | >64 | 64 |
| 407 | 0.25 | 16 | 16 | 2 | 2 | 64 | 0.25 |
| 412 | 0.5 | 2 | 64 | 0.5 | 2 | >64 | 32 |
| 429 | ND | 2 | 16 | 0.5 | 0.5 | >64 | 4 |
| 431 | ND | 0.5 | 8 | 0.5 | 0.5 | >64 | 16 |
| 432 | ND | 0.5 | 64 | 0.13 | 1 | >64 | 8 |
| 435 | 0.25 | 0.5 | 4 | <0.06 | 0.13 | >64 | 2 |
| 442 | ND | 4 | 64 | 0.5 | 0.5 | >64 | 4 |
| 446 | ND | 16 | 32 | 0.5 | 2 | 64 | 16 |

¹EC = Escherichia coli; KP = Klebsiella pneumoniae; KOXY = Klebsiella oxytoca; PM = Proteus mirabilis; CF = Citrobacter freundii; SM = Serratia marcescens; ENA = Enterobacter aerogenes; ENC = Enterobacter cloacae.

TABLE 3-3
Minimum Inhibitory Concentrations (microgram/mL) of Exemplary Compounds
of Formulas I and II for Representative Enteric Bacteria¹

| | Bacterium | | | | | | |
|---|---|---|---|---|---|---|---|
| Compound | KP(ATCC | EC(ATCC | EC(KB1 | EC(KB2 | EC(KB1 | EC(KB2 | EC |
| No. | SHV-18) | NDM-1) | CTX-M-14) | CTX-M-14) | CTX-M-15) | CTX-M-15) | (URMC-1) |
| 252 | >64 | >64 | >64 | >64 | >64 | >64 | 0.5 |
| 353 | 4 | >64 | >64 | >64 | >64 | >64 | 0.25 |
| 373 | 1 | >64 | 4 | 4 | 32 | 64 | ≤0.06 |
| 388 | 16 | >64 | 32 | 32 | 64 | 64 | 1 |
| 389 | 4 | >64 | >64 | >64 | >64 | >64 | 0.25 |
| 394 | 4 | >64 | 32 | 32 | >64 | >64 | 0.25 |
| 395 | 2 | >64 | 16 | 16 | 64 | 64 | 0.25 |
| 407 | 2 | >64 | 2 | 64 | >64 | >64 | 0.25 |
| 412 | 1 | >64 | 0.5 | 1 | 16 | 32 | 0.25 |
| 429 | ND | >64 | 0.5 | 1 | 8 | 8 | 0.5 |
| 431 | ND | >64 | 0.25 | 0.5 | 2 | 2 | 0.25 |
| 432 | ND | >64 | 0.5 | 0.5 | 1 | 1 | 0.5 |
| 435 | 0.25 | >64 | ≤0.06 | ≤0.06 | 0.13 | 0.5 | ≤0.06 |
| 442 | 0.5 | >64 | 1 | 4 | 32 | 32 | 0.25 |
| 446 | 1 | >64 | 2 | 8 | 64 | >64 | ND |

¹EC = Escherichia coli; KP = Klebsiella pneumoniae; KOXY = Klebsiella oxytoca; PM = Proteus mirabilis; CF = Citrobacter freundii; SM = Serratia marcescens; ENA = Enterobacter aerogenes; ENC = Enterobacter cloacae.

TABLE 3-4
Minimum Inhibitory Concentrations (microgram/mL) of Exemplary
Compounds of Formulas I and II for Representative Enteric Bacteria¹

| | Bacterium | | | | | | |
|---|---|---|---|---|---|---|---|
| Compound | EC | EC | KP | KP | KP | ENA | ENA |
| No. | (URMC-2) | (URMC-3) | (URMC-1) | (URMC-2) | (URMC-3) | (URMC-1) | (URMC-2) |
| 252 | 0.25 | 0.13 | 0.5 | 0.5 | 0.5 | 1 | 0.5 |
| 353 | 0.13 | ≤0.06 | 0.5 | 0.25 | 0.25 | 0.25 | 0.25 |
| 373 | 0.13 | ≤0.06 | 0.13 | 0.13 | 0.13 | 0.25 | 0.25 |
| 388 | 1 | 0.5 | 1 | 2 | 2 | 2 | 2 |
| 389 | 0.25 | 0.13 | 0.25 | 0.13 | 0.13 | 0.13 | 0.13 |
| 394 | 0.25 | 0.25 | 0.25 | 0.25 | 0.13 | 0.25 | 0.25 |
| 395 | 0.25 | 0.25 | 0.5 | 1 | 1 | 2 | 0.5 |
| 407 | 0.25 | ≤0.06 | 0.13 | 0.25 | 0.13 | 0.5 | 0.5 |
| 412 | 4 | ≤0.06 | ≤0.06 | ≤0.06 | 0.25 | 0.25 | 0.13 |
| 429 | 0.13 | 0.5 | ≤0.06 | ≤0.06 | ≤0.06 | 2 | 1 |
| 431 | ≤0.06 | 0.25 | 0.25 | 0.13 | ≤0.06 | 0.5 | 0.5 |
| 432 | ≤0.06 | 0.13 | 0.13 | ≤0.06 | ≤0.06 | 1 | 0.25 |
| 435 | ≤0.06 | 0.5 | ≤0.06 | ≤0.06 | ≤0.06 | 0.13 | 0.13 |
| 442 | 0.13 | 0.13 | 0.13 | 0.25 | ≤0.06 | 0.5 | 1 |
| 446 | ND | ND | ND | ND | ND | ND | ND |

¹EC = Escherichia coli; KP = Klebsiella pneumoniae; KOXY = Klebsiella oxytoca; PM = Proteus mirabilis; CF = Citrobacter freundii; SM = Serratia marcescens; ENA = Enterobacter aerogenes; ENC = Enterobacter cloacae.

TABLE 3-5
Mimimum Inhibitory Concentrations (microgram/mL)
of Exemplary Compounds of Formulas I and II for
Representative Enteric Bacteria¹

Bacterium

| Compound | EA | EC | EC | PM |
|---|---|---|---|---|
| No. | (URMC -3) | (RR -1) | (RR -2) | (RR -1) |
| 252 | 16 | 0.5 | 0.25 | 0.5 |
| 353 | 4 | 0.25 | 0.25 | 0.25 |
| 373 | 2 | 0.13 | ≤0.06 | ≤0.06 |
| 388 | 8 | 1 | 1 | 0.25 |
| 389 | 0.5 | 0.13 | 0.13 | ≤0.06 |
| 394 | 2 | 0.13 | 0.25 | 0.25 |
| 395 | 4 | 0.5 | 0.5 | 0.25 |
| 407 | 2 | 0.13 | 0.13 | ≤0.06 |
| 412 | ≤0.06 | 0.25 | ≤0.06 | ≤0.06 |
| 429 | 2 | ≤0.06 | ≤0.06 | ≤0.06 |
| 431 | 1 | ≤0.06 | ≤0.06 | ≤0.06 |
| 432 | 0.5 | ≤0.06 | ≤0.06 | ≤0.06 |
| 435 | 0.5 | ≤0.06 | ≤0.06 | ≤0.06 |
| 442 | 1 | 0.25 | 0.13 | ≤0.06 |
| 446 | ND | ND | ND | ND |

¹EC = Escherichia coli; KP = Klebsiella pneumoniae; KOXY = Klebsiella oxytoca; PM = Proteus mirabilis; CF = Citrobacter freundii; SM = Serratia marcescens; ENA = Enterobacter aerogenes; ENC = Enterobacter cloacae.

TABLE 3-6
Mimimum Inhibitory Concentrations (microgram/mL)
of Exemplary Compounds of Formulas I and II for
Representative Enteric Bacteria¹

Bacterium

| Compound | CF | KP | | ENC |
|---|---|---|---|---|
| No. | (RR -1) | (RR -1) | ENC | (ATCC NDM-1) |
| 252 | 0.5 | 0.5 | 8 | >64 |
| 353 | 0.5 | 0.13 | 2 | >64 |
| 373 | 0.25 | ≤0.06 | 4 | >64 |
| 388 | 4 | 1 | 16 | >64 |
| 389 | 0.13 | 0.13 | 1 | >64 |
| 394 | ND | 0.25 | 2 | >64 |
| 395 | 0.5 | 0.25 | >64 | >64 |
| 407 | 0.13 | 0.5 | 4 | >64 |
| 412 | ≤0.06 | 0.5 | 4 | >64 |
| 429 | ≤0.06 | 0.25 | ND | >64 |
| 431 | 1 | 0.13 | ND | >64 |
| 432 | 0.5 | 0.25 | ND | >64 |
| 435 | ≤0.06 | ≤0.06 | 16 | >64 |
| 442 | 0.25 | 0.25 | 8 | >64 |
| 446 | ND | ND | 4 | >64 |

¹EC = Escherichia coli; KP = Klebsiella pneumoniae; KOXY = Klebsiella oxytoca; PM = Proteus mirabilis; CF = Citrobacter freundii; SM = Serratia marcescens; ENA = Enterobacter aerogenes; ENC = Enterobacter cloacae.

TABLE 4-1
Mimimum Inhibitory Concentrations (microgram/mL) of Exemplary Compounds
of Formulas I and II for Isogenic E. coli J58 strains carrying Extended Spectrum beta-
Lactamases, carbapenemases, or other beta-lactamases.

| Comp. | | | | | | |
|---|---|---|---|---|---|---|
| No | J53 Alone | TEM-26 | SHV-1 | DHA-1 | CTX-M-14 | CTX-M-15 |
| 352 | 0.5 | 4 | 8 | 2 | 16 | >64 |
| 353 | 0.25 | 0.5 | 4 | 2 | >64 | >64 |
| 373 | | | | | | |
| T | 0.13 | 4 | 1 | 1 | 1 | 32 |
| 392 | 64 | 32 | >64 | >64 | >64 | >64 |
| 394 | 0.25 | 16 | 8 | 4 | 4 | >64 |
| 402 | 0.5 | 4 | 2 | 1 | 4 | >64 |
| 407 | 0.13 | 1 | 0.5 | 0.25 | 1 | 64 |
| 411 | 0.5 | 8 | 4 | 1 | 4 | 0.5 |
| 412 | ≤0.06 | 2 | 2 | 0.25 | 0.5 | 0.13* |
| 413 | 0.25 | 1 | 2 | 0.13 | 2 | 2* |
| 414 | 0.25 | 1 | 1 | 0.5 | 2 | 1* |
| 415 | 0.5 | 1 | 1 | 0.5 | 2 | 1* |
| 421 | 0.5 | 2 | 4 | 1 | 16 | 16 |
| 422 | 0.25 | 4 | 2 | 0.25 | 4 | 4 |
| 423 | 0.25 | 2 | 2 | 1 | 2 | 2 |
| 424 | 0.25 | 4 | 2 | 0.5 | 1 | 2 |
| 426 | 1 | 8 | 0.5 | 0.5 | 0.5 | 0.5 |
| 427 | ≤0.06 | 2 | 1 | 1 | 1 | 1 |
| 429 | ≤0.06 | 0.5 | 0.25 | 0.25 | 0.5 | 0.13 |
| 431 | ≤0.06 | 0.25 | 0.13 | 1 | 0.13 | 0.13 |
| 432 | ≤0.06 | 0.25 | 0.25 | ≤0.06 | ≤0.06 | ≤0.06 |
| 434 | 0.25 | 2 | 1 | 1 | 2 | 0.5 |
| 435 | ≤0.06 | ≤0.06 | 0.13 | ≤0.06 | ≤0.06 | ≤0.06 |
| 436 | 0.13 | 2 | 1 | 0.25 | 1 | 0.5 |
| 437 | 0.25 | 1 | 2 | 0.5 | 4 | 0.5 |
| 438 | 1 | 2 | 2 | 4 | 2 | 1 |
| 439 | 0.25 | 1 | 0.5 | 1 | 0.5 | 0.5 |
| 440 | 0.25 | 1 | 1 | 0.25 | 2 | 0.25 |
| 441 | 0.25 | 1 | 1 | 0.5 | 1 | 0.5 |
| 442 | 0.25 | 0.5 | 0.5 | 0.25 | 0.25 | 0.25 |
| 443 | 0.13 | 2 | 1 | 1 | 0.25 | 1 |
| 444 | 0.5 | 0.25 | 1 | 1 | 0.25 | 1 |
| 445 | 1 | 1 | 1 | 1 | 4 | 4 |
| 446 | 0.13 | 0.25 | 0.5 | 0.25 | 0.25 | 0.25 |

TABLE 4-2
Mimimum Inhibitory Concentrations (microgram/mL) of Exemplary
Compounds of Formulas I and II for Isogenic E. coli J58 strains carrying
Extended Spectrum beta-Lactamases

| Comp. | ESBL | | | | | |
|---|---|---|---|---|---|---|
| No | J53 Alone | KPC-2 | KPC-3 | NDM-1 | VIM-2 | IMP-1 |
| 352 | 0.5 | 64 | >64 | >64 | 2 | 32 |
| 353 | 0.25 | >64 | 64 | >64 | 4 | 8 |
| 373 | 0.13 | 0.25 | 8 | >64 | 1 | >64 |
| 392 | 64 | >64 | >64 | >64 | >64 | >64 |
| 394 | 0.25 | 64 | 32 | >64 | 32 | >64 |
| 402 | 0.5 | 64 | 16 | >64 | 4 | >64 |
| 407 | 0.13 | 8 | 4 | >64 | 0.5 | 32 |
| 411 | 0.5 | 32 | 8 | >64 | 2 | 64 |
| 412 | ≤0.06 | 4 | 4 | >64 | 0.5 | 16 |
| 413 | 0.25 | 8 | 4 | >64 | 0.5 | 64 |
| 414 | 0.25 | 8 | 4 | >64 | 1 | 8 |
| 415 | 0.5 | 16 | 8 | >64 | 1 | 16 |
| 421 | 0.5 | >64 | 32 | >64 | 1 | >64 |
| 422 | 0.25 | 16 | 8 | >64 | 1 | >64 |
| 423 | 0.25 | 8 | 4 | >64 | 0.5 | 32 |
| 424 | 0.25 | 32 | 8 | >64 | 2 | >64 |
| 426 | 1 | 64 | 8 | >64 | 8 | 32 |
| 427 | ≤0.06 | 16 | 4 | >64 | 1 | 16 |
| 429 | ≤0.06 | 4 | 1 | >64 | ≤0.06 | ≤0.06 |
| 431 | ≤0.06 | 0.5 | 0.25 | >64 | ≤0.06 | 0.25* |
| 432 | ≤0.06 | 1 | 0.25 | >64 | ≤0.06 | 0.13* |
| 434 | 0.25 | 16 | 64 | >64 | 2 | 0.25 |
| 435 | ≤0.06 | 0.25 | 0.13 | 2 | ≤0.06 | 0.13 |
| 436 | 0.13 | 64 | 8 | >64 | 1 | 8 |
| 437 | 0.25 | 16 | 2 | >64 | 1 | 0.13 |
| 438 | 1 | 32 | 4 | >64 | 1 | 1 |
| 439 | 0.25 | 1 | 0.25 | >64 | 0.13 | 0.13 |
| 440 | 0.25 | 16 | 4 | >64 | 0.25 | 0.13 |
| 441 | 0.25 | 4 | 1 | >64 | 0.25 | 1 |
| 442 | 0.25 | 2 | 1 | >64 | 0.13 | 0.13 |
| 443 | 0.13 | 8 | 4 | >64 | 0.25 | 4 |
| 444 | 0.5 | 8 | 2 | 32 | 0.5 | 0.5 |
| 445 | 1 | 64 | 16 | >64 | 0.25 | 1 |
| 446 | 0.13 | 2 | 1 | >64 | 0.25 | 1 |

### REFERENCES

(1) ANTIBIOTIC RESISTANCE THREATS in the United States, 2013; CDC available at the web site www.cdc.gov/drugresistance
(2) Spellberg, B.; Guidos, R.; Gilbert, D.; Bradley, J.; Boucher, H. W.; Scheld, W. M.; Bartlett, J. G.; Edwards, J. The Epidemic of Antibiotic-Resistant Infections: A Call to Action for the Medical Community from the Infectious Diseases Society of America. Clin. Infect. Dis. 2008, 46 (2), 155-164.
(3) Livermore, D. M. Has the Era of Untreatable Infections Arrived? J. Antimicrob. Chemother. 2009, 64 Suppl 1, i29-36.
(4) Neu, H. C. The Crisis in Antibiotic Resistance. Science 1992, 257 (5073), 1064-1073.
(5) Projan, S. J. Why Is Big Pharma Getting out of Antibacterial Drug Discovery? Curr. Opin. Microbiol. 2003, 6 (5), 427-430.
(6) Payne, D. J.; Gwynn, M. N.; Holmes, D. J.; Pompliano, D. L. Drugs for Bad Bugs: Confronting the Challenges of Antibacterial Discovery. Nat. Rev. Drug Discov. 2007, 6 (1), 29-40.
(7) Magiorakos, a; Srinivasan, A.; Carey, R. B.; Carmeli, Y.; Falagas, M. E.; Giske, C. G.; Harbarth, S.; Hindler, J. F. Bacteria: An International Expert Proposal for Interim Standard Definitions for Acquired Resistance. Microbiology 2011, 18 (3), 268-281.
(8) Craig, W. A. State-of-the-Art Clinical Article: Pharmacokinetic/Pharmacodynamic Parameters: Rationale for Antibacterial Dosing of Mice and Men. Clin. Infect. Dis. 1998, 26 (1), 1-10.
(9) Montgomery, a B. The Antibiotic Pipeline. N. Engl. J. Med. 2004, 351 (19), 2019-2020.
(10) Bakker-Woudenberg, I. a J. M.; ten Kate, M. T.; Goessens, W. H. F.; Mouton, J. W. Effect of Treatment Duration on Pharmacokinetic/pharmacodynamic Indices Correlating with Therapeutic Efficacy of Ceftazidime in Experimental Klebsiella Pneumoniae Lung Infection. Antimicrob. Agents Chemother. 2006, 50 (9), 2919-2925.
(11) Boucher, H. W.; Talbot, G. H.; Bradley, J. S.; Edwards, J. E.; Gilbert, D.; Rice, L. B.; Scheld, M.; Spellberg, B.; Bartlett, J. Bad Bugs, No Drugs: No ESKAPE! An Update from the Infectious Diseases Society of America. Clin. Infect. Dis. 2009, 48 (1), 1-12.
(12) World Health Organization. Antimicrobial Resistance Global Report on Surveillance; Geneva, 2014.
(13) Liu, Y. Y.; Wang, Y.; Walsh, T. R.; Yi, L. X.; Zhang, R.; Spencer, J.; Doi, Y.; Tian, G.; Dong, B.; Huang, X.; et al. Emergence of Plasmid-Mediated Colistin Resistance Mechanism MCR-1 in Animals and Human Beings in China: A Microbiological and Molecular Biological Study. Lancet Infect. Dis. 2015, 16 (2), 161-168.
(14) Johnson, J. W.; Gretes, M.; Goodfellow, V. J.; Marrone, L.; Heynen, M. L.; Strynadka, N. C. J.; Dmitrienko, G. I. Cyclobutanone Analogues of Beta-Lactams Revisited: Insights into Conformational Requirements for Inhibition of Serine- and Metallo-Beta-Lactamases. J. Am. Chem. Soc. 2010, 132 (8), 2558-2560.
(15) Tioni, M. F.; Llarrull, L. I.; Poeylaut-Palena, A. a; Marti, M. a; Saggu, M.; Periyannan, G. R.; Mata, E. G.; Bennett, B.; Murgida, D. H.; Vila, A. J. Trapping and Characterization of a Reaction Intermediate in Carbapenem Hydrolysis by B. Cereus Metallo-Beta-Lactamase. J. Am. Chem. Soc. 2008, 130 (47), 15852-15863.
(16) Yuan, Q.; He, L.; Ke, H. A Potential Substrate Binding Conformation of beta-Lactams and Insight into the Broad Spectrum of NDM-1 Activity. Antimicrob. Agents Chemother. 2012, 56 (10), 5157-5163.
(17) E. P. Abraham, “A Glimpse of the Early History of the Cephalosporins,” Reviews of Infectious Diseases, Vol. 1, No. 1. (January-February, 1979), pp. 99-105.
(18) E. P. Abraham, “Cephalosporins 1945-1986,” Drugs 34 (Suppl. 2): 1-14 (1987).
(19) Long, T. E.; Williams, J. T. Cephalosporins Currently in Early Clinical Trials for the Treatment of Bacterial Infections. Expert Opin. Investig. Drugs 2014, 23 (10), 1375-1387.
(20) Zaffiri, L.; Gardner, J.; Toledo-Pereyra, L. H. History of Antibiotics. From Salvarsan to Cephalosporins. J. Investig. Surg. 2012, 25, 67-77.
(21) (a) Peterson, L. R. Bad Bugs, No Drugs: No ESCAPE Revisited. Clin. Infect. Dis. 2009, 49, 992-993; (b) IDSA. The 10 X '20 Initiative: Pursuing a Global Commitment to Develop 10 New Antibacterial Drugs by 2020. Clin. Infect. Dis. 2010, 50 (8), 1081-1083; (c) Boucher, H. W.; Talbot, G. H.; Benjamin, D. K.; Bradley, J.; Guidos, R. J.; Jones, R. N.; Murray, B. E.; Bonomo, R. a; Gilbert, D. 10 X '20 Progress—Development of New Drugs Active Against Gram-Negative Bacilli: An Update From the Infectious Diseases Society of America. Clin. Infect. Dis. 2013, 1-10; (d) Pendleton, J. N.; Gorman, S. P.; Gilmore, B. F. Clinical Relevance of the ESKAPE Pathogens. Expert Rev. Anti. Infect. Ther. 2013, 11 (3), 297-308; (e) WHO|WHO Publishes List of Bacteria for Which New Antibiotics Are Urgently Needed. WHO 2017; (f) Biggest Threats|Antibiotic/Antimicrobial Resistance|CDC see website www.cdc.gov/drugresistance/biggest_threats.html (accessed Jul. 23, 2017); (g) NIAID Emerging Infectious Diseases/Pathogens|NIH: National Institute of Allergy and Infectious Diseases see website www.niaid.nih.gov/research/emerging-infectious-diseases-pathogens.
(22) G. A. Jacoby (2009) AmpC Beta-Lacatamses Clin., Microbiol Rev. 22(1) 161-182.
(23) Bush K and Jacoby G A (2010) Antimicro Agents Chemother, Vol. 54: p. 969-976.
(24) Shaikh S. et al. (2015) Saudi Journal Biol, Sciences 22:90-101.
(25) Urban C. et al. (1994) Antimicrob. Agent. Chemother. 38:392-395.
(26) Chaves J. et al. (2001) Antimicrob. Agent. Chemother. 45:2856-2861.
(27) Hennequin C. et al. (October 2011) Antimicrob. Agent. Chemother. 56:288-294.
(28) Aghamiri S. et al. (2014) ISRN Microbiology Article IS 941507 6 pages (available from web site: dx.doi.org/10.1155/2014/941507).
(29) Canton R. et al. (2012) Frontiers Microbiology, Vol. 3, article 110: 1-1.
(30) Papp-Wallace K. et al (2015) Antimicrob. Agent. Chemother. 59:3710-3717.
(31) Alba, J. et al. (2005) Antimicrob. Agent. Chemother. 49:4760-4762.
(32) Raghunath d. (November 2010) IJMR (Indian J. Med. Res.) 132(5):478-481.
(33) Saini A and Barsal, R. (2012) Advances Biol Chem 2:323-334.
(34) Poirel L. et al. (2000) Antimicrob. Agent. Chemother. April:891-897.

1. A compound of formula: 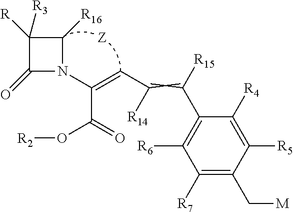
or salts, or solvates thereof,
where M is: 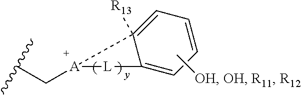
R is an acyl amino group (R₁CO—NH—) or an alkyl group optionally substituted with a group selected from a halogen, a hydroxy group or a protected hydroxyl group;
R₂ is hydrogen, unsubstituted or substituted alkyl, unsubstituted or substituted aryl, a pharmaceutically acceptable cation, when the CO₂ group to which R₂ is attached is negatively charged, or a carboxylate protecting group;
R₃ is hydrogen, a C1-C3-alkoxy group;
—Z— is a linker between the two indicated atoms, which forms a 5- or 6-member carbocyclic or heterocyclic ring with the atoms to which it is linked;
R₁₆ is hydrogen, a C1-C3 alkyl group or a C1-C3 alkoxy group;
R₄, R₅, R₆, and R₇ are independently selected from hydrogen, halogen, cyano, nitro, C1-C3 alkyl, C1-C3 haloalkyl, amino, C1-C3 alkylamino, and C1-C3 dialkylamino;
R₁₁ and R₁₂ are independently selected from hydrogen, hydroxyl, halogen, C1-C3 alkyl, cyano, and nitro groups,
R₁₃, if present, is selected from hydrogen, hydroxyl, halogen, C1-C3 alkyl, cyano, and nitro groups;
A⁺ is a leaving group containing a positively charged nitrogen;
L is an optional divalent linker moiety containing 1-6 carbon atoms and optionally one, two or three heteroatoms (N, S or O), where y is 1 or 0 to indicate presence or absence of the linker;
wherein the phenyl ring of the styryl group is cis or trans or a mixture of is or trans with respect to the cepham ring;
wherein the A⁺ group is bonded to the indicated phenyl ring of X through linker L or is a partially or fully unsaturated heterocyclic ring fused to the indicated phenyl ring, and
wherein the indicated phenyl ring in X is a catechol having two hydroxyl group substituted on adjacent ring carbons.

2. The compound, salt or solvate of claim 1, wherein —Z— is —S—CH₂—, —SO—CH₂—, or —SO₂—CH₂—.

3. The compound, salt or solvate of claim 1, wherein —Z— is —CH₂—CH₂— or —O—CH₂—.

4. The compound, salt or solvate of claim 1, wherein R is an acylamino group.

5. The compound, salt or solvate of claim 1, wherein —Z— is —S—, —CR₁₇— or —O—, where R₁₇ is hydrogen or C1-C3 alkyl.

6. The compound, salt or solvate of claim 5, where R is a hydroxy-substituted alkyl group.

7. The compound, salt or solvate of claim 1 of formula: 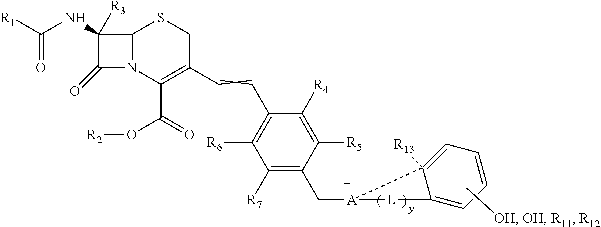
or a salt, or solvate thereof,
wherein:
R₁ is:
(1) methylene, substituted with two groups selected from hydrogen, halogen, cyano, amino, alkyl amino, dialkylamino, unsubstituted or substituted aryl group, unsubstituted or substituted heterocyclic group, unsubstituted or substituted thioalkyl group, unsubstituted or substituted thioaryl group, or unsubstituted or substituted thioheterocyclic group, wherein at least one of the groups on the methylene is a group other than hydrogen;
(2) —C(R₂₀)═C—(O)_{z}—R₂₁, where z is 1 or 0 to indicate the presence or absence of the oxygen; or
(3) —C(R₂₀)=N˜O—R₂₂, wherein the N˜O bond is in the syn or anti conformation;
where:
R₂₀ is an unsubstituted or substituted heterocyclic group;
R₂₁ is hydrogen, unsubstituted or substituted C1-C4 straight chain or branched alkyl, unsubstituted or substituted C3-C6 cycloalkyl, unsubstituted or substituted aryl, unsubstituted or substituted heterocycyl, unsubstituted or substituted aryl-substituted C1-C4 alkyl or unsubstituted or substituted heterocycyl-substituted C1-C4 alkyl;
R₂₂ is hydrogen, hydroxyl, unsubstituted or substituted C1-C4 straight chain or branched alkyl, unsubstituted or substituted C3-C6 cycloalkyl, unsubstituted or substituted C1-C4 straight chain or branched alkoxy, unsubstituted or substituted aryl, unsubstituted or substituted heterocycyl, unsubstituted or substituted aryl-substituted C1-C4 alkyl, unsubstituted or substituted heterocycyl-substituted C1-C4 alky,
or R₂₂ is:
a Z-substituted C1-C6 straight-chain or branched alkyl or C(Ra)(Rb)Z, where Z is COOH, or a salt thereof, or CH₂—N(OH)COR_{C}, where:
Ra, Rb and Rc are independently hydrogen, C1-C6 alkyl, C2-C7 alkenyl, C2-C7 alkynyl, C3-C6 cycloalkyl, unsubstituted or substituted phenyl, unsubstituted or substituted benzyl, or
Ra and Rb together form a 3-6 member carbocyclic or heterocyclic ring, wherein the heterocyclic can contain one or two heteroatoms, N, NR_{N}, O, or S. and
Rc can further be OH, amino, C-C6 alkylamino, or C1-C6 alkoxy;
R₂ is hydrogen, unsubstituted or substituted alkyl, unsubstituted or substituted aryl, a pharmaceutically acceptable cation, when the CO₂ group to which R₂ is attached is negatively charged, or an OH protecting group;
R₃ is hydrogen or a C1-C3-alkoxy group;
R₄, R₅, R₆, and R₇ are independently selected from hydrogen, halogen, cyano, nitro, C1-C3 alkyl, C1-C3 haloalkyl, amino, C1-C3 alkylamino, and C1-C3 dialkylamino;
R₁₁ and R₁₂ are independently selected from hydrogen, hydroxyl, halogen, C1-C3 alkyl, cyano, and nitro groups,
R₁₃ is selected from hydrogen, hydroxyl, halogen, C1-C3 alkyl, cyano, and nitro;
A⁺ is a leaving group containing a positively charged nitrogen;
L is an optional divalent linker moiety containing 1-6 carbon atoms and optionally one, two or three heteroatoms (N, S or O), where y is 1 or 0 to indicate the presence or absence of the linker;
wherein the A⁺ group is bonded to the indicated phenyl ring through linker L or is a partially or fully unsaturated heterocyclic ring fused to the indicated phenyl ring. and wherein the double bond of the styryl group is the configuration E or the Z configuration or is a mixture of E/Z configurations, wherein the crossed double bond in the formula indicates the E or Z configuration; and
wherein the indicated phenyl ring is a catechol having two hydroxyl group substituted on adjacent ring carbons.

8. The compound, salt or solvate of claim 7, wherein:
R₃ is hydrogen;
R₃ is methoxy;
R₁ is —C(R₂₀)═C—(O)_{z}—R₂₁, and z is 1;
R₁ is —C(R₂₀)=N˜O—R₂₂,
R₄, R₅, R₆ and R₇ are all hydrogens;
R₅, R₆ and R₇ are all hydrogen; or
R₄ is a nitro group or a cyano group.

9. The compound, salt or solvate of claim 1, wherein:
R₁ is of formula: 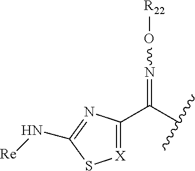
where:
X is N or CR_{X};
R_{X} is hydrogen, C1-C3 alkyl or halogen, and
Re is hydrogen or (Rp)₂PO—, and
each Rp is independently hydrogen, or C1-C3 alkyl; or
R₁ is of formula: 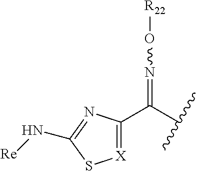
where X is CH and Re is hydrogen; or
R₁ is of formula: 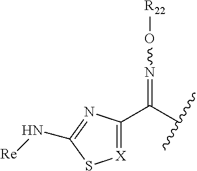
where X is N and Re is hydrogen.

10. The compound of claim 1, wherein M is M_{X} M is M_{X}: 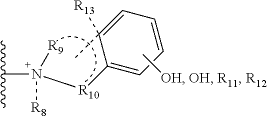
where:
R₈ is hydrogen or unsubstituted or substituted C1-C6 alkyl;
R₉ is selected from hydrogen, unsubstituted or substituted C1-C6 alkyl;
R₁₀ is a divalent —(CH₂)ₙ— moiety, where n is 1-6 wherein one or two CH₂ groups are replaced with —O—, —S—, —CO—, —N(R_{N})CO—, or —CON(R_{N})—, where R_{N} is hydrogen or a C1-C3 alkyl;
or R₉ and R₁₀ together with the nitrogen to which they are attached form a 5- or 6-member heterocyclic ring as shown by the dotted line, which ring is partially or fully unsaturated, unsubstituted or substituted, and optionally contains one or two additional heteroatoms: O, S, N, NR_{N} or a combination thereof in the ring, where R_{N} is hydrogen or a C1-C3 alkyl;
where R₁₁ and R₁₂ are independently selected from hydrogen, hydroxyl, halogen, carboxyl or ester thereof, acyl, optionally substituted aryl, optionally substituted aryl alkyl, C1-C3 alkyl, C1-C3 alkoxy, cyano, and nitro groups or, if R₁₁ and R₁₂ are substituted on adjacent ring carbons, R₁₁ and R₁₂ together with the carbons to which they are attached form an optionally substituted 5- or 6-member carbocyclic, or heterocyclic ring, which may be saturated, partially unsaturated or aromatic; and
R₁₃, if present, is selected from hydrogen, hydroxyl, halogen, C1-C3 alkyl, cyano, and nitro groups, or, if R₁₁ or R₁₂ is substituted on a ring carbon adjacent to R₁₃, R₁₃ and R₁₁ or R₁₂ together with the carbons to which they are attached form an optionally substituted 5- or 6-member carbocyclic, or heterocyclic ring which may be saturated, partially unsaturated or aromatic;
where dotted lines indicate optional bonds; and
where the phenyl ring at the right of the formula is substituted with at least two hydroxyl groups on adjacent ring carbons and is bonded to the positively charged N through the R₁₀ moiety or is fused to the 5- or 6-member ring formed by R₉ and R₁₀.

11. The compound of claim 1 of formula: 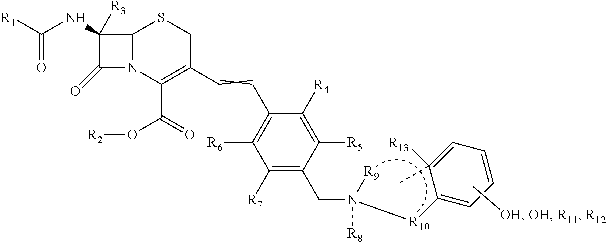
or a salt or solvate thereof
where:
R₈, if present, is hydrogen or unsubstituted or substituted C1-C6 alkyl;
R₉ is selected from hydrogen, unsubstituted or substituted C1-C6 alkyl;
R₁₀ is a divalent —(CH₂)ₙ— moiety, where n is 1-6, wherein one or two CH₂ groups are optionally replaced with —O—, —S—, —CO—, —N(R_{N})CO—, or —CON(R_{N})—, and where R_{N} is hydrogen or a C1-C3 alkyl;
or R₉ and R₁₀ together with the nitrogen to which they are attached form a 5- or 6-member heterocyclic ring as shown by the dotted line, which ring is partially or fully unsaturated, unsubstituted or substituted, and optionally contains one or two additional heteroatoms: O, S, N, NR_{N} or a combination thereof in the ring, where R_{N} is hydrogen or a C1-C3 alkyl;
where dotted lines indicate optional bonds;
where the phenyl ring at the right of the formula is substituted with two hydroxyl groups on adjacent ring carbons and is bonded to the positively charged N through the R₁₀ moiety or is fused to the 5- or 6-member ring formed by R₉ and R₁₀.

12. The compound, salt or solvate of claim 11, wherein R₉ and R₁₀ together with the nitrogen to which they are attached form a 5- or 6-member heterocyclic ring as shown by the dotted line, which ring is partially or fully unsaturated, unsubstituted or substituted, and optionally contains one or two additional heteroatoms: O, S, N, NR_{N} or a combination thereof in the ring, where R_{N} is hydrogen or a C1-C3 alkyl.

13. The compound of claim 1 of formula:

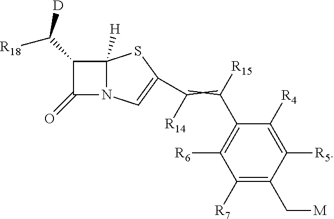

14. The compound, salt or solvate of claim 13, wherein D is hydroxyl and R₁₈ is C1-C2 alkyl.

15. The compound, salt or solvate of claim 1, which is zwitterionic.

16. The compound, salt or solvate of claim 1, which is a salt of an anion selected from a halide, sulfate, bisulfate, acetate or trifluoroacetate.

17. The compound, salt or solvate of claim 1, wherein the double bond of the styryl group is in the E configuration.

18. The compound, salt or solvate of claim 1, wherein the double bond of the styryl group is in the Z configuration.

19. A pharmaceutical composition thereof comprising one or more compounds, salts or solvates thereof of claim 1 with a pharmaceutically acceptable carrier.

20. A method for treating or preventing an infection of a bacterium which comprises administering to a subject in need of such treatment or prophylaxis a therapeutically effective amount of one or more compounds, salts or solvates thereof, of claim 1.
